SEQUENCE LISTING (75 sequences)

SEQ ID NO: 1 - protein, 1048 aa, Bacillus megaterium
  cytochrome P450NADPH-P-450 reductase precursor, cytochrome P450 (BM3), CYP102A1
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 2 - protein, 1049 aa, Bacillus megaterium
  NADPH-cytochrome P450 reductase 102A1V9, CYP102A1
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPREGAVLIVTASYNGHPPDNAKEFVDWLDQASADEVKGVRYSVFGCGDKNWATTYQKVPAFIDETLAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNQPKTYVQHVMEQDGKKLIELLDQGAHFYICGDGSQMAPDVEATLMKSYAEVHQVSEADARLWLQQLEEKGRYAKDVWAG

SEQ ID NO: 3 - protein, 1049 aa, Bacillus megaterium
  NADPH-cytochrome P450 reductase 102A1V10, CYP102A1
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPREGAVLIVTASYNGHPPDNAKEFVDWLDQASADEVKGVRYSVFGCGDKNWATTYQKVPAFIDETFAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNQPKTYVQHVMEQDGKKLIELLDQGAHFYICGDGSQMAPDVEATLMKSYAEVHQVSEADARLWLQQLEEKGRYAKDVWAG

SEQ ID NO: 4 - protein, 1049 aa, Bacillus megaterium
  NADPH-cytochrome P450 reductase 102A1V4, CYP102A1
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEATRVLVDPVPSYKQVKQLKYVGMVLNEALRLWPTAPAFSLYAKEDTVLGGEYPLEKGDELMVLIPQLHRDKTIWGEDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKVENAHNTPLLVLYGSNMGTAEGTARDLADIAMSKGFAPQVATLDSHAGNLPREGAVLIVTASYNGHPPDNAKQFVDWLDQASADDVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSANVVASKELQQPGSERSTRHLEIALPKEASYQEGDHLGVIPRNYEGIVNRVTARFGLDASQQIRLEAEEEKLAHLPLGKTVSVEELLQYVELQDPVTRTQLRAMAAKTVCPPHKVELEALLEKQAYKEQVLAKRLTMLELLEKYPACEMEFSEFIALLPSIRPRYYSISSSPRVDEKQASITVSVVSGEAWSGYGEYKGIASNYLANLQEGDTITCFVSTPQSGFTLPKDSETPLIMVGPGTGVAPFRSFVQARKQLKEQGQSLGEAHLYFGCRSPHEDYLYQEELENAQNEGIITLHTAFSRVPNQPKTYVQHVMEQDGKKLIELLDQGAHFYICGDGSQMAPDVEATLMKSYADVYEVSEADARLWLQQLEEKGRYAKDVWAG

SEQ ID NO: 5 - protein, 1049 aa, Bacillus megaterium
  NADPH-cytochrome P450 reductase 102A1V8, CYP102A1
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPRVATLDSHAGNLPREGAVLIVTASYNGHPPDNAKEFVDWLDQASADEVKGVRYSVFGCGDKNWATTYQKVPAFIDETLAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNQPKTYVQHVMEQDGKKLIELLDQGAHFYICGDGSQMAPDVEATLMKSYAEVHQVSEADARLWLQQLEEKGRYAKDVWAG

SEQ ID NO: 6 - protein, 1049 aa, Bacillus megaterium
  NADPH-cytochrome P450 reductase 102A1V3, CYP102A1
MTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKVENAHNTPLLVLYGSNMGTAEGTARDLADIAMSKGFAPQVATLDSHAGNLPREGAVLIVTASYNGHPPDNAKQFVDWLDQASADDVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSANVVASKELQQLGSERSTRHLEIALPKEASYQEGDHLGVIPRNYEGIVNRVTARFGLDASQQIRLEAEEEKLAHLPLGKTVSVEELLQYVELQDPVTRTQLRAMAAKTVCPPHKVELEALLEKQAYKEQVLAKRLTMLELLEKYPACEMEFSEFIALLPSISPRYYSISSSPHVDEKQASITVSVVSGEAWSGYGEYKGIASNYLANLQEGDTITCFVSTPQSGFTLPKDSETPLIMVGPGTGVAPFRGFVQARKQLKEQGQSLGEAHLYFGCRSPHEDYLYQEELENAQNEGIITLHTAFSRVPNQPKTYVQHVMERDGKKLIELLDQGAHFYICGDGSQMAPDVEATLMKSYADVYEVSEADARLWLQQLEEKGRYAKDVWAG

SEQ ID NO: 7 - protein, 1049 aa, Bacillus megaterium
  NADPH-cytochrome P450 reductase 102A1V7, CYP102A1
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPPEGAVLIVTASYNGHPPDNAKEFVDWLDQASADEVKGVRYSVFGCGDKNWATTYQKVPAFIDETLAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNEPKTYVQHVMEQDGKKLIELLDQGAHFYICGDGSQMAPDVEATLMKSYAEVHQVSEADARLWLQQLEEKGRYAKDVWAG

SEQ ID NO: 8 - protein, 1049 aa, Bacillus megaterium
  NADPH-cytochrome P450 reductase 102A1V2, CYP102A1
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEATRVLVDPVPSYKQVKQLKYVGMVLNEALRLWPTAPAFSLYAKEDTVLGGEYPLEKGDELMVLIPQLHRDKTIWGEDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKVENAHNTPLLVLYGSNMGTAEGTARDLADIAMSKGFAPQVATLDSHAGNLPREGAVLIVTASYNGHPPDNAKQFVDWLDQASADDVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSANVVASKELQQLGSERSTRHLEIALPKEASYQEGDHLGVIPRNYEGIVNRVTARFGLDASQQIRLEAEEEKLAHLPLGKTVSVEELLQYVELQDPVTRTQLRAMAAKTVCPPHKVELEALLEKQAYKEQVLAKRLTMLELLEKYPACEMEFSEFIALLPSISPRYYSISSSPHVDEKQASITVSVVSGEAWSGYGEYKGIASNYLANLQEGDTITCFVSTPQSGFTLPKDSETPLIMVGPGTGVAPFRGFVQARKQLKEQGQSLGEAHLYFGCRSPHEDYLYQEELENAQNEGIITLHTAFSRVPNQPKTYVQHVMERDGKKLIELLDQGAHFYICGDGSQMAPDVEATLMKSYADVYEVSEADARLWLQQLEEKGRYAKDVWAG

SEQ ID NO: 9 - protein, 1049 aa, Bacillus megaterium; identical to SEQ ID NO: 2
  cytochrome P450NADPH P450 reductase, CYP102A1

SEQ ID NO: 10 - protein, 1049 aa, Bacillus megaterium
  NADPH-cytochrome P450 reductase 102A1V6, CYP102A1
MTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNADEHIEVPEDMTRLTLDTIGLCGFNYRFNSFYRDQPHPFITSMVRALDEAMNKLQRANPDDPAYDENKRQFQDDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLSAKGAENIAERGEADASDDFEGTYEEWREHMWSDLAAYFNLNIENSEDNASTLSLQFVDSAADMPLAKMHGAFSANVVASKELQQPGSARSTRHLEIELPKEASYQEGDHLGVIPRNYEGIVNRVTTRFGLDASQQIRLEAEEEKLAHLPLGKTVSVEELLQYVELQDPVTRTQLRAMAAKTVCPPHKVELEALLEKQAYKEQVLTKRLTMLELLEKYPACEMEFSEFIALLPSMRPRYYSISSSPRVDEKQASITVSVVSGEAWSGYGEYKGIASNYLAELQEGDTITCFVSTPQSGFTLPKDPETPLIMVGPGTGVAPFRGFVQARKQLKEQGQSLGEAHLYFGCRSPHEDYLYQEELENAQNEGIITLHTAFSRVPNQPKTYVQHVVEQDGKKLIELLDQGAHFYICGDGSQMAPDVEATLMKSYAEVHKVSEADARLWLQQLEEKSRYAKDVWAG

SEQ ID NO: 11 - protein, 1049 aa, Bacillus megaterium; identical to SEQ ID NO: 10
  NADPH-cytochrome P450 reductase 102A1V5, CYP102A1

SEQ ID NO: 12 - protein, 420 aa, Mycobacterium sp. HXN-1500
  cytochrome P450 alkane hydroxylase, CYP153A6
MTEMTVAASDATNAAYGMALEDIDVSNPVLFRDNTWHPYFKRLREEDPVHYCKSSMFGPYWSVTKYRDIMAVETNPKVFSSEAKSGGITIMDDNAAASLPMFIAMDPPKHDVQRKTVSPIVAPENLATMESVIRQRTADLLDGLPINEEFDWVHRVSIELTTKMLATLFDFPWDDRAKLTRWSDVTTALPGGGIIDSEEQRMAELMECATYFTELWNQRVNAEPKNDLISMMAHSESTRHMAPEEYLGNIVLLIVGGNDTTRNSMTGGVLALNEFPDEYRKLSANPALISSMVSEIIRWQTPLSHMRRTALEDIEFGGKHIRQGDKVVMWYVSGNRDPEAIDNPDTFIIDRAKPRQHLSFGFGIHRCVGNRLAELQLNILWEEILKRWPDPLQIQVLQEPTRVLSPFVKGYESLPVRINA

SEQ ID NO: 13 - protein, 496 aa, Tetrahymena thermophile
  cytochrome P450 monooxygenase CYP5013C2
MIFELILIAVALFAYFKIAKPYFSYLKYRKYGKGFYYPILGEMIEQEQDLKQHADADYSVHHALDKDPDQKLFVTNLGTKVKLRLIEPEIIKDFFSKSQYYQKDQTFIQNITRFLKNGIVFSEGNTWKESRKLFSPAFHYEYIQKLTPLINDITDTIFNLAVKNQELKNFDPIAQIQEITGRVIIASFFGEVIEGEKFQGLTIIQCLSHIINTLGNQTYSIMYFLFGSKYFELGVTEEHRKFNKFIAEFNKYLLQKIDQQIEIMSNELQTKGYIQNPCILAQLISTHKIDEITRNQLFQDFKTFYIAGMDTTGHLLGMTIYYVSQNKDIYTKLQSEIDSNTDQSAHGLIKNLPYLNAVIKETLRYYGPGNILFDRIAIKDHELAGIPIKKGTIVTPYAMSMQRNSKYYQDPHKYNPSRWLEKQSSDLHPDANIPFSAGQRKCIGEQLALLEARIILNKFIKMFDFTCPQDYKLMMNYKFLSEPVNPLPLQLTLRKQ

SEQ ID NO: 14 - protein, 394 aa, Nonomuraea dietziae
  cytochrome P450 hydroxylase sb8
VNIDLVDQDHYATFGPPHEQMRWLREHAPVYWHEGEPGFWAVTRHEDVVHVSRHSDLFSSARRLALFNEMPEEQRELQRMMMLNQDPPEHTRRRSLVNRGFTPRTIRALEQHIRDICDDLLDQCSGEGDFVTDLAAPLPLYVICELLGAPVADRDKIFAWSNRMIGAQDPDYAASPEEGGAAAMEVYAYASELAAQRRAAPRDDIVTKLLQSDENGESLTENEFELFVLLLVVAGNETTRNAASGGMLTLFEHPDQWDRLVADPSLAATAADEIVRWVSPVNLFRRTATADLTLGGQQVKADDKVVVFYSSANRDASVFSDPEVFDIGRSPNPHIGFGGGGAHFCLGNHLAKLELRVLFEQLARRFPRMRQTGEARRLRSNFINGIKTLPVTLG

SEQ ID NO: 15 - protein, 501 aa, Homo sapiens
  human vitamin D 25-hydroxylase, CYP2R1
MWKLWRAEEGAAALGGALFLLLFALGVRQLLKQRRPMGFPPGPPGLPFIGNIYSLAASSELPHVYMRKQSQVYGEIFSLDLGGISTVVLNGYDVVKECLVHQSEIFADRPCLPLFMKMTKMGGLLNSRYGRGWVDHRRLAVNSFRYFGYGQKSFESKILEETKFFNDAIETYKGRPFDFKQLITNAVSNITNLIIFGERFTYEDTDFQHMIELFSENVELAASASVFLYNAFPWIGILPFGKHQQLFRNAAVVYDFLSRLIEKASVNRKPQLPQHFVDAYLDEMDQGKNDPSSTFSKENLIFSVGELIIAGTETTTNVLRWAILFMALYPNIQGQVQKEIDLIMGPNGKPSWDDKCKMPYTEAVLHEVLRFCNIVPLGIFHATSEDAVVRGYSIPKGTTVITNLYSVHFDEKYWRDPEVFHPERFLDSSGYFAKKEALVPFSLGRRHCLGEHLARMEMFLFFTALLQRFHLHFPHELVPDLKPRLGMTLQPQPYLICAERR

SEQ ID NO: 16 - protein, 501 aa, Macaca mulatta
  Rhesus monkey vitamin D 25-hydroxylase, CYP2R1
MWKLWGGEEGAAALGGALFLLLFALGVRQLLKLRRPMGFPPGPPGLPFIGNIYSLAASAELPHVYMRKQSQVYGEIFSLDLGGISTVVLNGYDVVKECLVHQSGIFADRPCLPLFMKMTKMGGLLNSRYGQGWVEHRRLAVNSFRYFGYGQKSFESKILEETKFFTDAIETYKGRPFDFKQLITSAVSNITNLIIFGERFTYEDTDFQHMIELFSENVELAASASVFLYNAFPWIGILPFGKHQQLFRNASVVYDFLSRLIEKASVNRKPQLPQHFVDAYFDEMDQGKNDPSSTFSKENLIFSVGELIIAGTETTTNVLRWAILFMALYPNIQGQVQKEIDLIMGPNGKPSWDDKFKMPYTEAVLHEVLRFCNIVPLGIFHATSEDAVVRGYSIPKGTTVITNLYSVHFDEKYWRDPEVFHPERFLDSSGYFAKKEALVPFSLGRRHCLGEQLARMEMFLFFTALLQRFHLHFPHELVPDLKPRLGMTLQPQPYLICAERR

SEQ ID NO: 17 - protein, 501 aa, Canis familiaris
  dog vitamin D 25-hydroxylase, CYP2R1
MRGPPGAEACAAGLGAALLLLLFVLGVRQLLKQRRPAGFPPGPSGLPFIGNIYSLAASGELAHVYMRKQSRVYGEIFSLDLGGISAVVLNGYDVVKECLVHQSEIFADRPCLPLFMKMTKMGGLLNSRYGRGWVDHRKLAVNSFRCFGYGQKSFESKILEETNFFIDAIETYKGRPFDLKQLITNAVSNITNLIIFGERFTYEDTDFQHMIELFSENVELAASASVFLYNAFPWIGIIPFGKHQQLFRNAAVVYDFLSRLIEKASINRKPQSPQHFVDAYLNEMDQGKNDPSCTFSKENLIFSVGELIIAGTETTTNVLRWAILFMALYPNIQGQVQKEIDLIMGPTGKPSWDDKCKMPYTEAVLHEVLRFCNIVPLGIFHATSEDAVVRGYSIPKGTTVITNLYSVHFDEKYWRNPEIFYPERFLDSSGYFAKKEALVPFSLGKRHCLGEQLARMEMFLFFTALLQRFHLHFPHGLVPDLKPRLGMTLQPQPYLICAERR

SEQ ID NO: 18 - protein, 222 aa, Mus musculus
  mouse Cyp2r1 protein, CYP2R1
MGDEMDQGQNDPLSTFSKENLIFSVGELIIAGTETTTNVLRWAILFMALYPNIQGQVHKEIDLIVGHNRRPSWEYKCKMPYTEAVLHEVLRFCNIVPLGIFHATSEDAVVRGYSIPKGTTVITNLYSVHFDEKYWKDPDMFYPERFLDSNGYFTKKEALIPFSLGRRHCLGEQLARMEMFLFFTSLLQQFHLHFPHELVPNLKPRLGMTLQPQPYLICAERR

SEQ ID NO: 19 - protein, 422 aa, Bacillus halodurans
  Bacillus halodurans strain C-125 fatty acid alpha hydroxylase, CYP152A6
MKSNDPIPKDSPLDHTMNLMREGYEFLSHRMERFQTDLFETRVMGQKVLCIRGAEAVKLFYDPERFKRHRATPKRIQKSLFGENAIQTMDDKAHLHRKQLFLSMMKPEDEQELARLTHETWRRVAEGWKKSRPIVLFDEAKRVLCQVACEWAEVPLKSTEIDRRAEDFHAMVDAFGAVGPRHWRGRKGRRRTERWIQSIIHQVRTGSLQAREGSPLYKVSYHRELNGKLLDERMAAIELINVLRPIVAIATFISFAAIALQEHPEWQERLKNGSNEEFHMFVQEVRRYYPFAPLIGAKVRKSFTWKGVRFKKGRLVFLDMYGTNHDPKLWDEPDAFRPERFQERKDSLYDFIPQGGGDPTKGHRCPGEGITVEVMKTTMDFLVNDIDYDVPDQDISYSLSRMPTRPESGYIMANIERKYEHA

SEQ ID NO: 20 - protein, 389 aa, Streptomyces parvus
  cytochrome P450, aryC
MYLGGRRGTEAVGESREPGVWEVFRYDEAVQVLGDHRTFSSDMNHFIPEEQRQLARAARGNFVGIDPPDHTQLRGLVSQAFSPRVTAALEPRIGRLAEQLLDDIVAERGDKASCDLVGEFAGPLSAIVIAELFGIPESDHTMIAEWAKALLGSRPAGELSIADEAAMQNTADLVRRAGEYLVHHITERRARPQDDLTSRLATTEVDGKRLDDEEIVGVIGMFLIAGYLPASVLTANTVMALDEHPAALAEVRSDPALLPGAIEEVLRWRPPLVRDQRLTTRDADLGGRTVPAGSMVCVWLASAHRDPFRFENPDLFDIHRNAGRHLAFGKGIHYCLGAPLARLEARIAVETLLRRFERIEIPRDESVEFHESIGVLGPVRLPTTLFARR

SEQ ID NO: 21 - protein, 414 aa, Pseudomonas putida
  camphor 5-monooxygenase, camC, cyp101, locus CPXA_PSEPU, CYP101A1
TTETIQSNANLAPLPPHVPEHLVFDFDMYNPSNLSAGVQEAWAVLQESNVPDLVWTRCNGGHWIATRGQLIREAYEDYRHFSSECPFIPREAGEAYDFIPTSMDPPEQRQFRALANQVVGMPVVDKLENRIQELACSLIESLRPQGQCNFTEDYAEPFPIRIFMLLAGLPEEDIPHLKYLTDQMTRPDGSMTFAEAKEALYDYLIPIIEQRRQKPGTDAISIVANGQVNGRPITSDEAKRMCGLLLVGGLDTVVNFLSFSMEFLAKSPEHRQELIERPERIPAACEELLRRFSLVADGRILTSDYEFHGVQLKKGDQILLPQMLSGLDERENACPMHVDFSRQKVSHTTFGHGSHLCLGQHLARREIIVTLKEWLTRIPDFSIAPGAQIQHKSGIVSGVQALPLVWDPATTKAV

SEQ ID NO: 22 - protein, 515 aa, Homo sapiens
  human cytochrome P450, CYP2D7
GLEALVPLAMIVAIFLLLVDLMHRHQRWAARYPPGPLPLPGLGNLLHVDFQNTPYCFDQLRRRFGDVFNLQLAWTPVVVLNGLAAVREAMVTRGEDTADRPPAPIYQVLGFGPRSQGVILSRYGPAWREQRRFSVSTLRNLGLGKKSLEQWVTEEAACLCAAFADQAGRPFRPNGLLDKAVSNVIASLTCGRRFEYDDPRFLRLLDLAQEGLKEESGFLREVLNAVPVLPHIPALAGKVLRFQKAFLTQLDELLTEHRMTWDPAQPPRDLTEAFLAKKEKAKGSPESSFNDENLRIVVGNLFLAGMVTTLTTLAWGLLLMILHLDVQRGRRVSPGCSPIVGTHVCPVRVQQEIDDVIGQVRRPEMGDQVHMPYTTAVIHEVQRFGDIVPLGVTHMTSRDIEVQGFRIPKGTTLITNLSSVLKDEAVWEKPFRFHPEHFLDAQGHFVKPEAFLPFSAGRRACLGEPLARMELFLFFTSLLQHFSFSVAAGQPRPSHSRVVSFLVTPSPYELCAVPR

SEQ ID NO: 23 - protein, 532 aa, Rattus norvegicus
  rat cytochrome P450, CYPC27
AVLSRMRLRWALLDTRVMGHGLCPQGARAKAAIPAALRDHESTEGPGTGQDRPRLRSLAELPGPGTLRFLFQLFLRGYVLHLHELQALNKAKYGPMWTTTFGTRTNVNLASAPLLEQVMRQEGKYPIRDSMEQWKEHRDHKGLSYGIFITQGQQWYHLRHSLNQRMLKPAEAALYTDALNEVISDFIARLDQVRTESASGDQVPDVAHLLYHLALEAICYILFEKRVGCLEPSIPEDTATFIRSVGLMFKNSVYVTFLPKWSRPLLPFWKRYMNNWDNIFSFGEKMIHQKVQEIEAQLQAAGPDGVQVSGYLHFLLTKELLSPQETVGTFPELILAGVDTTSNTLTWALYHLSKNPEIQEALHKEVTGVVPFGKVPQNKDFAHMPLLKAVIKETLRLYPVVPTNSRIITEKETEINGFLFPKNTQFVLCTYVVSRDPSVFPEPESFQPHRWLRKREDDNSGIQHPFGSVPFGYGVRSCLGRRIAELEMQLLLSRLIQKYEVVLSPGMGEVKSVSRIVLVPSKKVSLRFLQRQ

SEQ ID NO: 24 - protein, 491 aa, Oryctolagus cuniculus
  rabbit cytochrome P450, CYP2B4
MEFSLLLLLAFLAGLLLLLFRGHPKAHGRLPPGPSPLPVLGNLLQMDRKGLLRSFLRLREKYGDVFTVYLGSRPVVVLCGTDAIREALVDQAEAFSGRGKIAVVDPIFQGYGVIFANGERWRALRRFSLATMRDFGMGKRSVEERIQEEARCLVEELRKSKGALLDNTLLFHSITSNIICSIVFGKRFDYKDPVFLRLLDLFFQSFSLISSFSSQVFELFPGFLKHFPGTHRQIYRNLQEINTFIGQSVEKHRATLDPSNPRDFIDVYLLRMEKDKSDPSSEFHHQNLILTVLSLFFAGTETTSTTLRYGFLLMLKYPHVTERVQKEIEQVIGSHRPPALDDRAKMPYTDAVIHEIQRLGDLIPFGVPHTVTKDTQFRGYVIPKNTEVFPVLSSALHDPRYFETPNTFNPGHFLDANGALKRNEGFMPFSLGKRICLGEGIARTELFLFFTTILQNFSIASPVPPEDIDLTPRESGVGNVPPSYQIRFLAR

SEQ ID NO: 25 - protein, 1061 aa, Bacillus subtilis
  Bacillus subtilis strain 168 probable bifunctional P-450/NADPH-P450 reductase 1, cypD, locus CYPD_BACSU, CYP102A2
MKETSPIPQPKTFGPLGNLPLIDKDKPTLSLIKLAEEQGPIFQIHTPAGTTIVVSGHELVKEVCDEERFDKSIEGALEKVRAFSGDGLFTSWTHEPNWRKAHNILMPTFSQRAMKDYHEKMVDIAVQLIQKWARLNPNEAVDVPGDMTRLTLDTIGLCGFNYRFNSYYRETPHPFINSMVRALDEAMHQMQRLDVQDKLMVRTKRQFRHDIQTMFSLVDSIIAERRANGDQDEKDLLARMLNVEDPETGEKLDDENIRFQIITFLIAGHETTSGLLSFATYFLLKHPDKLKKAYEEVDRVLTDAAPTYKQVLELTYIRMILNESLRLWPTAPAFSLYPKEDTVIGGKFPITTNDRISVLIPQLHRDRDAWGKDAEEFRPERFEHQDQVPHHAYKPFGNGQRACIGMQFALHEATLVLGMILKYFTLIDHENYELDIKQTLTLKPGDFHIRVQSRNQDAIHADVQAVEKAASDEQKEKTEAKGTSVIGLNNRPLLVLYGSDTGTAEGVARELADTASLHGVRTETAPLNDRIGKLPKEGAVVIVTSSYNGKPPSNAGQFVQWLQEIKPGELEGVHYAVFGCGDHNWASTYQYVPRFIDEQLAEKGATRFSARGEGDVSGDFEGQLDEWKKSMWADAIKAFGLELNENADKERSTLSLQFVRGLGESPLARSYEASHASIAENRELQSADSDRSTRHIEIALPPDVEYQEGDHLGVLPKNSQTNVSRILHRFGLKGTDQVTLSASGRSAGHLPLGRPVSLHDLLSYSVEVQEAATRAQIRELAAFTVCPPHRRELEELSAEGVYQEQILKKRISMLDLLEKYEACDMPFERFLELLRPLKPRYYSISSSPRVNPRQASITVGVVRGPAWSGRGEYRGVASNDLAERQAGDDVVMFIRTPESRFQLPKDPETPIIMVGPGTGVAPFRGFLQARDVLKREGKTLGEAHLYFGCRNDRDFIYRDELERFEKDGIVTVHTAFSRKEGMPKTYVQHLMADQADTLISILDRGGRLYVCGDGSKMAPDVEAALQKAYQAVHGTGEQEAQNWLRHLQDTGMYAKDVWAGI

SEQ ID NO: 26 - protein, 1054 aa, Bacillus subtilis
  Bacillus subtilis strain 168 probable bifunctional P-450/NADPH-P450 reductase 2, cypE, CYP102A3
MKQASAIPQPKTYGPLKNLPHLEKEQLSQSLWRIADELGPIFRFDFPGVSSVFVSGHNLVAEVCDESRFDKNLGKGLQKVREFGGDGLFTSWTHEPNWQKAHRILLPSFSQKAMKGYHSMMLDIATQLIQKWSRLNPNEEIDVADDMTRLTLDTIGLCGFNYRFNSFYRDSQHPFITSMLRALKEAMNQSKRLGLQDKMMVKTKLQFQKDIEVMNSLVDRMIAERKANPDDNIKDLLSLMLYAKDPVTGETLDDENIRYQIITFLIAGHETTSGLLSFAIYCLLTHPEKLKKAQEEADRVLTDDTPEYKQIQQLKYTRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQADIKAETKPKETKPKHGTPLLVLYGSNLGTAEGIAGELAAQGRQMGFTAETAPLDDYIGKLPEEGAVVIVTASYNGSPPDNAAGFVEWLKELEEGQLKGVSYAVFGCGNRSWASTYQRIPRLIDDMMKAKGASRLTEIGEGDAADDFESHRESWENRFWKETMDAFDINEIAQKEDRPSLSIAFLSEATETPVAKAYGAFEGVVLENRELQTADSTRSTRHIELEIPAGKTYKEGDHIGIMPKNSRELVQRVLSRFGLQSNHVIKVSGSAHMSHLPMDRPIKVADLLSSYVELQEPASRLQLRELASYTVCPPHQKELEQLVLDDGIYKEQVLAKRLTMLDFLEDYPACEMPFERFLALLPSLKPRYYSISSSPKVHANIVSMTVGVVKASAWSGRGEYRGVASNYLAELNTGDAAACFIRTPQSGFQMPDEPETPMIMVGPGTGIAPFRGFIQARSVLKKEGSTLGEALLYFGCRRPDHDDLYREELDQAEQEGLVTIRRCYSRVENESKGYVQHLLKQDSQKLMTLIEKGAHIYVCGDGSQMAPDVEKTLRWAYETEKGASQEESADWLQKLQDQKRYIKDVWTGN

SEQ ID NO: 27 - protein, 1049 aa, Bacillus megaterium [sequence omitted]

SEQ ID NO: 28 - protein, 1065 aa, Bacillus cereus
  Bacillus cereus strain ATCC 14579 NADPH-cytochrome P450 reductase, CYP102A5
MEKKVSAIPQPKTYGPLGNLPLIDKDKPTLSFIKIAEEYGPIFQIQTLSDTIIVVSGHELVAEVCDETRFDKSIEGALAKVRAFAGDGLFTSETHEPNWKKAHNILMPTFSQRAMKDYHAMMVDIAVQLVQKWARLNPNENVDVPEDMTRLTLDTIGLCGFNYRFNSFYRETPHPFITSMTRALDEAMHQLQRLDIEDKLMWRTKRQFQHDIQSMFSLVDNIIAERKSSGDQEENDLLSRMLNVPDPETGEKLDDENIRFQIITFLIAGHETTSGLLSFAIYFLLKNPDKLKKAYEEVDRVLTDPTPTYQQVMKLKYMRMILNESLRLWPTAPAFSLYAKEDTVIGGKYPIKKGEDRISVLIPQLHRDKDAWGDNVEEFQPERFEELDKVPHHAYKPFGNGQRACIGMQFALHEATLVMGMLLQHFELIDYQNYQLDVKQTLTLKPGDFKIRILPRKQTISHPTVLAPTEDKLKNDEIKQHVQKTPSIIGADNLSLLVLYGSDTGVAEGIARELADTASLEGVQTEVVALNDRIGSLPKEGAVLIVTSSYNGKPPSNAGQFVQWLEELKPDELKGVQYAVFGCGDHNWASTYQRIPRYIDEQMAQKGATRFSKRGEADASGDFEEQLEQWKQNMWSDAMKAFGLELNKNMEKERSTLSLQFVSRLGGSPLARTYEAVYASILENRELQSSSSDRSTRHIEVSLPEGATYKEGDHLGVLPVNSEKNINRILKRFGLNGKDQVILSASGRSINHIPLDSPVSLLALLSYSVEVQEAATRAQIREMVTFTACPPHKKELEALLEEGVYHEQILKKRISMLDLLEKYEACEIRFERFLELLPALKPRYYSISSSPLVAHNRLSITVGVVNAPAWSGEGTYEGVASNYLAQRHNKDEIICFIRTPQSNFELPKDPETPIIMVGPGTGIAPFRGFLQARRVQKQKGMNLGQAHLYFGCRHPEKDYLYRTELENDERDGLISLHTAFSRLEGHPKTYVQHLIKQDRINLISLLDNGAHLYICGDGSKMAPDVEDTLCQAYQEIHEVSEQEARNWLDRVQDEGRYGKDVWAGI

SEQ ID NO: 29 - protein, 1074 aa, Bacillus licheniformis
  Bacillus licheniformis strain DSM 13 = ATCC 14580 cytochrome P450/NADPH-ferrihemoproteinreductase, CYP102A7
MNKLDGIPIPKTYGPLGNLPLLDKNRVSQSLWKIADEMGPIFQFKFADAIGVFVSSHELVKEVSEESRFDKNMGKGLLKVREFSGDGLFTSWTEEPNWRKAHNILLPSFSQKAMKGYHPMMQDIAVQLIQKWSRLNQDESIDVPDDMTRLTLDTIGLCGFNYRFNSFYREGQHPFIESMVRGLSEAMRQTKRFPLQDKLMIQTKRRFNSDVESMFSLVDRIIADRKQAESESGNDLLSLMLHAKDPETGEKLDDENIRYQIITFLIAGHETTSGLLSFAIYLLLKHPDKLKKAYEEADRVLTDPVPSYKQVQQLKYIRMILNESIRLWPTAPAFSLYAKEETVIGGKYLIPKGQSVTVLIPKLHRDQSVWGEDAEAFRPERFEQMDSIPAHAYKPFGNGQRACIGMQFALHEATLVLGMILQYFDLEDHANYQLKIKESLTLKPDGFTIRVRPRKKEAMTAMPGAQPEENGRQEERPSAPAAENTHGTPLLVLYGSNLGTAEEIAKELAEEAREQGFHSRTAELDQYAGAIPAEGAVIIVTASYNGNPPDCAKEFVNWLEHDQTDDLRGVKYAVFGCGNRSWASTYQRIPRLIDSVLEKKGAQRLHKLGEGDAGDDFEGQFESWKYDLWPLLRTEFSLAEPEPNQTETDRQALSVEFVNAPAASPLAKAYQVFTAKISANRELQCEKSGRSTRHIEISLPEGAAYQEGDHLGVLPQNSEVLIGRVFQRFGLNGNEQILISGRNQASHLPLERPVHVKDLFQHCVELQEPATRAQIRELAAHTVCPPHQRELEDLLKDDVYKDQVLNKRLTMLDLLEQYPACELPFARFLALLPPLKPRYYSISSSPQLNPRQTSITVSVVSGPALSGRGHYKGVASNYLAGLEPGDAISCFIREPQSGFRLPEDPETPVIMVGPGTGIAPYRGFLQARRIQRDAGVKLGEAHLYFGCRRPNEDFLYRDELEQAEKDGIVHLHTAFSRLEGRPKTYVQDLLREDAALLIHLLNEGGRLYVCGDGSRMAPAVEQALCEAYRIVQGASREESQSWLSALLEEGRYAKDVWDGGVSQHNVKADCIART

SEQ ID NO: 30 - protein, 1065 aa, Bacillus thuringiensis
  Bacillus thuringiensis serovar konkukian strain 97-27 NADPH-cytochrome P450 reductase, CYPX
MDKKVSAIPQPKTYGPLGNLPLIDKDKPTLSFIKLAEEYGPIFQIQTLSDTIIVVSGHELVAEVCDETRFDKSIEGALAKVRAFAGDGLFTSETDEPNWKKAHNILMPTFSQRAMKDYHAMMVDIAVQLVQKWARLNPNENVDVPEDMTRLTLDTIGLCGFNYRFNSFYRETPHPFITSMTRALDEAMHQLQRLDIEDKLMWRTKRQFQHDIQSMFSLVDNIIAERKSSENQEENDLLSRMLNVQDPETGEKLDDENIRFQIITFLIAGHETTSGLLSFAIYFLLKNPDKLKKAYEEVDRVLTDSTPTYQQVMKLKYIRMILNESLRLWPTAPAFSLYAKEDTVIGGKYPIKKGEDRISVLIPQLHRDKDAWGDDVEEFQPERFEELDKVPHHAYKPFGNGQRACIGMQFALHEATLVMGMLLQHFEFIDYEDYQLDVKQTLTLKPGDFKIRIVPRNQTISHTTVLAPTEEKLKKHEIKKQVQKTPSIIGADNLSLLVLYGSDTGVAEGIARELADTASLEGVQTEVVALNDRIGSLPKEGAVLIVTSSYNGKPPSNAGQFVQWLEELKPDELKGVQYAVFGCGDHNWASTYQRIPRYIDEQMAQKGATRFSTRGEADASGDFEEQLEQWKQSMWSDAMKAFGLELNKNMEKERSTLSLQFVSRLGGSPLARTYEAVYASILENRELQSSSSERSTRHIEISLPEGATYKEGDHLGVLPINNEKNVNRILKRFGLNGKDQVILSASGRSVNHIPLDSPVRLYDLLSYSVEVQEAATRAQIREMVTFTACPPHKKELESLLEDGVYQEQILKKRISMLDLLEKYEACEIRFERFLELLPALKPRYYSISSSPLVAQDRLSITVGVVNAPAWSGEGTYEGVASNYLAQRHNKDEIICFIRTPQSNFQLPENPETPIIMVGPGTGIAPFRGFLQARRVQKQKGMKVGEAHLYFGCRHPEKDYLYRTELENDERDGLISLHTAFSRLEGHPKTYVQHVIKEDRIHLISLLDNGAHLYICGDGSKMAPDVEDTLCQAYQEIHEVSEQEARNWLDRLQEEGRYGKDVWAGI

SEQ ID NO: 31 - protein, 1064 aa, Cupriavidus metallidurans
  Cupriavidus metallidurans (R. metallidurans) strain CH34putative bifunctional P-450NADPH-450 reductase 2, CYP102E1
MSTATPAAALEPIPRDPGWPIFGNLFQITPGEVGQHLLARSRHHDGIFELDFAGKRVPFVSSVALASELCDATRFRKIIGPPLSYLRDMAGDGLFTAHSDEPNWGCAHRILMPAFSQRAMKAYFDVMLRVANRLVDKWDRQGPDADIAVADDMTRLTLDTIALAGFGYDFASFASDELDPFVMAMVGALGEAMQKLTRLPIQDRFMGRAHRQAAEDIAYMRNLVDDVIRQRRVSPTSGMDLLNLMLEARDPETDRRLDDANIRNQVITFLIAGHETTSGLLTFALYELLRNPGVLAQAYAEVDTVLPGDALPVYADLARMPVLDRVLKETLRLWPTAPAFAVAPFDDVVLGGRYRLRKDRRISVVLTALHRDPKVWANPERFDIDRFLPENEAKLPAHAYMPFGQGERACIGRQFALTEAKLALALMLRNFAFQDPHDYQFRLKETLTIKPDQFVLRVRRRRPHERFVTRQASQAVADAAQTDVRGHGQAMTVLCASSLGTARELAEQIHAGAIAAGFDAKLADLDDAVGVLPTSGLVVVVAATYNGRAPDSARKFEAMLDADDASGYRANGMRLALLGCGNSQWATYQAFPRRVFDFFITAGAVPLLPRGEADGNGDFDQAAERWLAQLWQALQADGAGTGGLGVDVQVRSMAAIRAETLPAGTQAFTVLSNDELVGDPSGLWDFSIEAPRTSTRDIRLQLPPGITYRTGDHIAVWPQNDAQLVSELCERLDLDPDAQATISAPHGMGRGLPIDQALPVRQLLTHFIELQDVVSRQTLRALAQATRCPFTKQSIEQLASDDAEHGYATKVVARRLGILDVLVEHPAIALTLQELLACTVPMRPRLYSIASSPLVSPDVATLLVGTVCAPALSGRGQFRGVASTWLQHLPPGARVSASIRTPNPPFAPDPDPAAPMLLIGPGTGIAPFRGFLEERALRKMAGNAVTPAQLYFGCRHPQHDWLYREDIERWAGQGVVEVHPAYSVVPDAPRYVQDLLWQRREQVWAQVRDGATIYVCGDGRRMAPAVRQTLIEIGMAQGGMTDKAASDWFGGLVAQGRYRQDVFN

SEQ ID NO: 32 - protein, 1120 aa, Aspergillus fumigatus
  Aspergillus fumigatus strain Af293 putative P450 family fatty acid hydroxylase, CYP505X
MSESKTVPIPGPRGVPLLGNIYDIEQEVPLRSINLMADQYGPIYRLTTFGWSRVFVSTHELVDEVCDEERFTKVVTAGLNQIRNGVHDGLFTANFPGEENWAIAHRVLVPAFGPLSIRGMFDEMYDIATQLVMKWARHGPTVPIMVTDDFTRLTLDTIALCAMGTRFNSFYHEEMHPFVEAMVGLLQGSGDRARRPALLNNLPTSENSKYWDDIAFLRNLAQELVEARRKNPEDKKDLLNALILGRDPKTGKGLTDESIIDNMITFLIAGHETTSGLLSFLFYYLLKTPNAYKKAQEEVDSVVGRRKITVEDMSRLPYLNAVMRETLRLRSTAPLIAVHAHPEKNKEDPVTLGGGKYVLNKDEPIVIILDKLHRDPQVYGPDAEEFKPERMLDENFEKLPKNAWKPFGNGMRACIGRPFAWQEALLVVAILLQNFNFQMDDPSYNLHIKQTLTIKPKDFHMRATLRHGLDATKLGIALSGSADRAPPESSGAASRVRKQATPPAGQLKPMHIFFGSNTGTCETFARRLADDAVGYGFAADVQSLDSAMQNVPKDEPVVFITASYEGQPPDNAAHFFEWLSALKENELEGVNYAVFGCGHHDWQATFHRIPKAVNQLVAEHGGNRLCDLGLADAANSDMFTDFDSWGESTFWPAITSKFGGGKSDEPKPSSSLQVEVSTGMRASTLGLQLQEGLVIDNQLLSAPDVPAKRMIRFKLPSDMSYRCGDYLAVLPVNPTSVVRRAIRRFDLPWDAMLTIRKPSQAPKGSTSIPLDTPISAFELLSTYVELSQPASKRDLTALADAAITDADAQAELRYLASSPTRFTEEIVKKRMSPLDLLIRYPSIKLPVGDFLAMLPPMRVRQYSISSSPLADPSECSITFSVLNAPALAAASLPPAERAEAEQYMGVASTYLSELKPGERAHIAVRPSHSGFKPPMDLKAPMIMACAGSGLAPFRGFIMDRAEKIRGRRSSVGADGQLPEVEQPAKAILYVGCRTKGKDDIHATELAEWAQLGAVDVRWAYSRPEDGSKGRHVQDLMLEDREELVSLFDQGARIYVCGSTGVGNGVRQACKDIYLERRRQLRQAARERGEEVPAEEDEDAAAEQFLDNLRTKERYATDVFT

SEQ ID NO: 33 - protein, 1083 aa, Aspergillus nidulans
  Aspergillus nidulans strain FGSC A4 hypothetical protein AN6835.2, CYP505A8
MAEIPEPKGLPLIGNIGTIDQEFPLGSMVALAEEHGEIYRLRFPGRTVVVVSTHALVNETCDEKRFRKSVNSALAHVREGVHDGLFTAKMGEVNWEIAHRVLMPAFGPLSIRGMFDEMHDIASQLALKWARYGPDCPIMVTDDFTRLTLDTLALCSMGYRFNSYYSPVLHPFIEAMGDFLTEAGEKPRRPPLPAVFFRNRDQKFQDDIAVLRDTAQGVLQARKEGKSDRNDLLSAMLRGVDSQTGQKMTDESIMDNLITFLIAGHETTSGLLSFVFYQLLKHPETYRTAQQEVDNVVGQGVIEVSHLSKLPYINSVLRETLRLNATIPLFTVEAFEDTLLAGKYPVKAGETIVNLLAKSHLDPEVYGEDALEFKPERMSDELFNARLKQFPSAWKPFGNGMRACIGRPFAWQEALLVMAMLLQNFDFSLADPNYDLKFKQTLTIKPKDMFMKARLRHGLTPTTLERRLAGLAVESATQDKIVTNPADNSVTGTRLTILYGSNSGTCETLARRIAADAPSKGFHVMRFDGLDSGRSALPTDHPVVIVTSSYEGQPPENAKQFVSWLEELEQQNESLQLKGVDFAVFGCFKEWAQTFHRIPKLVDSLLEKLGGSRLTDLGLADVSTDELFSTFETWADDVLWPRLVAQYGADGKTQAHGSSAGHEAASNAAVEVTVSNSRTQALRQDVGQAMVVETRLLTAESEKERRKKHLEIRLPDGVSYTAGDYLAVLPINPPETVRRAMRQFKLSWDAQITIAPSGPTTALPTDGPIAANDIFSTYVELSQPATRKDLRIMADATTDPDVQKILRTYANETYTAEILTKSISVLDILEQHPAIDLPLGTFLLMLPSMRMRQYSISSSPLLTPTTATITISVLDAPSRSRSNGSRHLGVATSYLDSLSVGDHLQVTVRKNPSSGFRLPSEPETTPMICIAAGSGIAPFRAFLQERAVMMEQDKDRKLAPALLFFGCRAPGIDDLYREQLEEWQARGVVDARWAFSRQSDDTKGCRHVDDRILADREDVVKLWRDGARVYVCGSGALAQSVRSAMVTVLRDEMETTGDGSDNGKAEKWFDEQRNVRYVMDVFD

SEQ ID NO: 34 - protein, 1054 aa, Aspergillus oryzae
  Aspergillus oryzae strain ATCC 42149 cytochrome P450, locus Q2U4F1_ASPOR, CYP505A3
MRQNDNEKQICPIPGPQGLPFLGNILDIDLDNGTMSTLKIAKTYYPIFKFTFAGETSIVINSVALLSELCDETRFHKHVSFGLELLRSGTHDGLFTAYDHEKNWELAHRLLVPAFGPLRIREMFPQMHDIAQQLCLKWQRYGPRRPLNLVDDFTRTTLDTIALCAMGYRFNSFYSEGDFHPFIKSMVRFLKEAETQATLPSFISNLRVRAKRRTQLDIDLMRTVCREIVTERRQTNLDHKNDLLDTMLTSRDSLSGDALSDESIIDNILTFLVAGHETTSGLLSFAVYYLLTTPDAMAKAAHEVDDVVGDQELTIEHLSMLKYLNAILRETLRLMPTAPGFSVTPYKPEIIGGKYEVKPGDSLDVFLAAVHRDPAVYGSDADEFRPERMSDEHFQKLPANSWKPFGNGKRSCIGRAFAWQEALMILALILQSFSLNLVDRGYTLKLKESLTIKPDNLWAYATPRPGRNVLHTRLALQTNSTHPEGLMSLKHETVESQPATILYGSNSGTCEALAHRLAIEMSSKGRFVCKVQPMDAIEHRRLPRGQPVIIITGSYDGRPPENARHFVKWLQSLKGNDLEGIQYAVFGCGLPGHHDWSTTFYKIPTLIDTIMAEHGGARLAPRGSADTAEDDPFAELESWSERSVWPGLEAAFDLVRHNSSDGTGKSTRITIRSPYTLRAAHETAVVHQVRVLTSAETTKKVHVELALPDTINYRPGDHLAILPLNSRQSVQRVLSLFQIGSDTILYMTSSSATSLPTDTPISAHDLLSGYVELNQVATPTSLRSLAAKATDEKTAEYLEALATDRYTTEVRGNHLSLLDILESYSVPSIEIQHYIQMLPLLRPRQYTISSSPRLNRGQASLTVSVMERADVGGPRNCAGVASNYLASCTPGSILRVSLRQANPDFRLPDESCSHPIIMVAAGSGIAPFRAFVQERSVRQKEGIILPPAFLFFGCRRADLDDLYREELDAFEEQGVVTLFRAFSRAQSESHGCKYVQDLLWMERVRVKTLWGQDAKVFVCGSVRMNEGVKAIISKIVSPTPTEELARRYIAETFI

SEQ ID NO: 35 - protein, 1103 aa, Aspergillus oryzae
  Aspergillus oryzae strain ATCC 42149 cytochrome P450, locus Q2UNA2_ASPOR, CYPX
MSTPKAEPVPIPGPRGVPLMGNILDIESEIPLRSLEMMADTYGPIYRLTTFGFSRCMISSHELAAEVFDEERFTKKIMAGLSELRHGIHDGLFTAHMGEENWEIAHRVLMPAFGPLNIQNMFDEMHDIATQLVMKWARQGPKQKIMVTDDFTRLTLDTIALCAMGTRFNSFYSEEMHPFVDAMVGMLKTAGDRSRRPGLVNNLPTTENNKYWEDIDYLRNLCKELVDTRKKNPTDKKDLLNALINGRDPKTGKGMSYDSIIDNMITFLIAGHETTSGSLSFAFYNMLKNPQAYQKAQEEVDRVIGRRRITVEDLQKLPYITAVMRETLRLTPTAPAIAVGPHPTKNHEDPVTLGNGKYVLGKDEPCALLLGKIQRDPKVYGPDAEEFKPERMLDEHFNKLPKHAWKPFGNGMRACIGRPFAWQEALLVIAMLLQNFNFQMDDPSYNIQLKQTLTIKPNHFYMRAALREGLDAVHLGSALSASSSEHADHAAGHGKAGAAKKGADLKPMHVYYGSNTGTCEAFARRLADDATSYGYSAEVESLDSAKDSIPKNGPVVFITASYEGQPPDNAAHFFEWLSALKGDKPLDGVNYAVFGCGHHDWQTTFYRIPKEVNRLVGENGANRLCEIGLADTANADIVTDFDTWGETSFWPAVAAKFGSNTQGSQKSSTFRVEVSSGHRATTLGLQLQEGLVVENTLLTQAGVPAKRTIRFKLPTDTQYKCGDYLAILPVNPSTVVRKVMSRFDLPWDAVLRIEKASPSSSKHISIPMDTQVSAYDLFATYVELSQPASKRDLAVLADAAAVDPETQAELQAIASDPARFAEISQKRISVLDLLLQYPSINLAIGDFVAMLPPMRVRQYSISSSPLVDPTECSITFSVLKAPSLAALTKEDEYLGVASTYLSELRSGERVQLSVRPSHTGFKPPTELSTPMIMACAGSGLAPFRGFVMDRAEKIRGRRSSGSMPEQPAKAILYAGCRTQGKDDIHADELAEWEKIGAVEVRRAYSRPSDGSKGTHVQDLMMEDKKELIDLFESGARIYVCGTPGVGNAVRDSIKSMFLERREEIRRIAKEKGEPVSDDDEETAFEKFLDDMKTKERYTTDIFA

SEQ ID NO: 36 - protein, 1066 aa, Fusarium oxysporum
  bifunctional P-450NADPH-P450 reductase, cytochrome P450foxy, fatty acid omega-hydrolxylase, cytochrome P450 505, NADPH-cytochrome P450 reductase, locus C505_FUSOX, CYP505A1
MAESVPIPEPPGYPLIGNLGEFTSNPLSDLNRLADTYGPIFRLRLGAKAPIFVSSNSLINEVCDEKRFKKTLKSVLSQVREGVHDGLFTAFEDEPNWGKAHRILVPAFGPLSIRGMFPEMHDIATQLCMKFARHGPRTPIDTSDNFTRLALDTLALCAMDFRFYSYYKEELHPFIEAMGDFLTESGNRNRRPPFAPNFLYRAANEKFYGDIALMKSVADEVVAARKASPSDRKDLLAAMLNGVDPQTGEKLSDENITNQLITFLIAGHETTSGTLSFAMYQLLKNPEAYSKVQKEVDEVVGRGPVLVEHLTKLPYISAVLRETLRLNSPITAFGLEAIDDTFLGGKYLVKKGEIVTALLSRGHVDPVVYGNDADKFIPERMLDDEFARLNKEYPNCWKPFGNGKRACIGRPFAWQESLLAMVVLFQNFNFTMTDPNYALEIKQTLTIKPDHFYINATLRHGMTPTELEHVLAGNGATSSSTHNIKAAANLDAKAGSGKPMAIFYGSNSGTCEALANRLASDAPSHGFSATTVGPLDQAKQNLPEDRPVVIVTASYEGQPPSNAAHFIKWMEDLDGNDMEKVSYAVFACGHHDWVETFHRIPKLVDSTLEKRGGTRLVPMGSADAATSDMFSDFEAWEDIVLWPGLKEKYKISDEESGGQKGLLVEVSTPRKTSLRQDVEEALVVAEKTLTKSGPAKKHIEIQLPSAMTYKAGDYLAILPLNPKSTVARVFRRFSLAWDSFLKIQSEGPTTLPTNVAISAFDVFSAYVELSQPATKRNILALAEATEDKDTIQELERLAGDAYQAEISPKRVSVLDLLEKFPAVALPISSYLAMLPPMRVRQYSISSSPFADPSKLTLTYSLLDAPSLSGQGRHVGVATNFLSHLTAGDKLHVSVRASSEAFHLPSDAEKTPIICVAAGTGLAPLRGFIQERAAMLAAGRTLAPALLFFGCRNPEIDDLYAEEFERWEKMGAVDVRRAYSRATDKSEGCKYVQDRVYHDRADVFKVWDQGAKVFICGSREIGKAVEDVCVRLAIEKAQQNGRDVTEEMARAWFERSRNERFATDVFD

SEQ ID NO: 37 - protein, 1115 aa, Fusarium verticillioides
  Fusarium verticillioides (G. moniliformis) Fum6p, CYPX
MSATALFTRRSVSTSNPELRPIPGPKPLPLLGNLFDFDFDNLTKSLGELGKIHGPIYSITFGASTEIMVTSREIAQELCDETRFCKLPGGALDVMKAVVGDGLFTAETSNPKWAIAHRIITPLFGAMRIRGMFDDMKDICEQMCLRWARFGPDEPLNVCDNMTKLTLDTIALCTIDYRFNSFYRENGAAHPFAEAVVDVMTESFDQSNLPDFVNNYVRFRAMAKFKRQAAELRRQTEELIAARRQNPVDRDDLLNAMLSAKDPKTGEGLSPESIVDNLLTFLIAGHETTSSLLSFCFYYLLENPHVLRRVQQEVDTVVGSDTITVDHLSSMPYLEAVLRETLRLRDPGPGFYVKPLKDEVVAGKYAVNKDQPLFIVFDSVHRDQSTYGADADEFRPERMLKDGFDKLPPCAWKPFGNGVRACVGRPFAMQQAILAVAMVLHKFDLVKDESYTLKYHVTMTVRPVGFTMKVRLRQGQRATDLAMGLHRGHSQEASAAASPSRASLKRLSSDVNGDDTDHKSQIAVLYASNSGSCEALAYRLAAEATERGFGIRAVDVVNNAIDRIPVGSPVILITASYNGEPADDAQEFVPWLKSLESGRLNGVKFAVFGNGHRDWANTLFAVPRLIDSELARCGAERVSLMGVSDTCDSSDPFSDFERWIDEKLFPELETPHGPGGVKNGDRAVPRQELQVSLGQPPRITMRKGYVRAIVTEARSLSSPGVPEKRHLELLLPKDFNYKAGDHVYILPRNSPRDVVRALSYFGLGEDTLITIRNTARKLSLGLPLDTPITATDLLGAYVELGRTASLKNLWTLVDAAGHGSRAALLSLTEPERFRAEVQDRHVSILDLLERFPDIDLSLSCFLPMLAQIRPRAYSFSSAPDWKPGHATLTYTVVDFATPATQGINGSSKSKAVGDGTAVVQRQGLASSYLSSLGPGTSLYVSLHRASPYFCLQKSTSLPVIMVGAGTGLAPFRAFLQERRMAAEGAKQRFGPALLFFGCRGPRLDSLYSVELEAYETIGLVQVRRAYSRDPSAQDAQGCKYVTDRLGKCRDEVARLWMDGAQVLVCGGKKMANDVLEVLGPMLLEIDQKRGETTAKTVVEWRARLDKSRYVEEVYV

SEQ ID NO: 38 - protein, 1069 aa, Gibberella zeae
  Gibberella zeae strain PH1 bifuctional P-450NADPH-P450 reductase, fatty acid omega-hyroxylase, P450foxy, locus C505_FUSOX, CYP505A7
MAESVPIPEPPGYPLIGNLGEFKTNPLNDLNRLADTYGPIFRLHLGSKTPTFVSSNAFINEVCDEKRFKKTLKSVLSVVREGVHDGLFTAFEDEPNWGKAHRILIPAFGPLSIRNMFPEMHEIANQLCMKLARHGPHTPVDASDNFTRLALDTLALCAMDFRFNSYYKEELHPFIEAMGDFLLESGNRNRRPAFAPNFLYRAANDKFYADIALMKSVADEVVATRKQNPTDRKDLLAAMLEGVDPQTGEKLSDDNITNQLITFLIAGHETTSGTLSFAMYHLLKNPEAYNKLQKEIDEVIGRDPVTVEHLTKLPYLSAVLRETLRISSPITGFGVEAIEDTFLGGKYLIKKGETVLSVLSRGHVDPVVYGPDAEKFVPERMLDDEFARLNKEFPNCWKPFGNGKRACIGRPFAWQESLLAMALLFQNFNFTQTDPNYELQIKQNLTIKPDNFFFNCTLRHGMTPTDLEGQLAGKGATTSIASHIKAPAASKGAKASNGKPMAIYYGSNSGTCEALANRLASDAAGHGFSASVIGTLDQAKQNLPEDRPVVIVTASYEGQPPSNAAHFIKWMEDLAGNEMEKVSYAVFGCGHHDWVDTFLRIPKLVDTTLEQRGGTRLVPMGSADAATSDMFSDFEAWEDTVLWPSLKEKYNVTDDEASGQRGLLVEVTTPRKTTLRQDVEEALVVSEKTLTKTGPAKKHIEIQLPSGMTYKAGDYLAILPLNPRKTVSRVFRRFSLAWDSFLKIQSDGPTTLPINIAISAFDVFSAYVELSQPATKRNILALSEATEDKATIQELEKLAGDAYQEDVSAKKVSVLDLLEKYPAVALPISSYLAMLPPMRVRQYSISSSPFADPSKLTLTYSLLDAPSLSGQGRHVGVATNFLSQLIAGDKLHISVRASSAAFHLPSDPETTPIICVAAGTGLAPFRGFIQERAAMLAAGRKLAPALLFFGCRDPENDDLYAEELARWEQMGAVDVRRAYSRATDKSEGCKYVQDRIYHDRADVFKVWDQGAKVFICGSREIGKAVEDICVRLAMERSEATQEGKGATEEKAREWFERSRNERFATDVFD

SEQ ID NO: 39 - protein, 1066 aa, Gibberella zeae
  Gibberella zeae strain PH1a hypothetical protein FG07596.1, CYP505C2
MAIKDGGKKSGQIPGPKGLPVLGNLFDLDLSDSLTSLINIGQKYAPIFSLELGGHREVMICSRDLLDELCDETRFHKIVTGGVDKLRPLAGDGLFTAQHGNHDWGIAHRILMPLFGPLKIREMFDDMQDVSEQLCLKWARLGPSATIDVANDFTRLTLDTIALCTMGYRFNSFYSNDKMHPFVDSMVAALIDADKQSMFPDFIGACRVKALSAFRKHAAIMKGTCNELIQERRKNPIEGTDLLTAMMEGKDPKTGEGMSDDLIVQNLITFLIAGHETTSGLLSFAFYYLLENPHTLEKARAEVDEVVGDQALNVDHLTKMPYVNMILRETLRLMPTAPGFFVTPHKDEIIGGKYAVPANESLFCFLHLIHRDPKVWGADAEEFRPERMADEFFEALPKNAWKPFGNGMRGCIGREFAWQEAKLITVMILQNFELSKADPSYKLKIKQSLTIKPDGFNMHAKLRNDRKVSGLFKAPSLSSQQPSLSSRQSINAINAKDLKPISIFYGSNTGTCEALAQKLSADCVASGFMPSKPLPLDMATKNLSKDGPNILLAASYDGRPSDNAEEFTKWAESLKPGELEGVQFAVFGCGHKDWVSTYFKIPKILDKCLADAGAERLVEIGLTDASTGRLYSDFDDWENQKLFTELSKRQGVTPTDDSHLELNVTVIQPQNNDMGGNFKRAEVVENTLLTYPGVSRKHSLLLKLPKDMEYTPGDHVLVLPKNPPQLVEQAMSCFGVDSDTALTISSKRPTFLPTDTPILISSLLSSLVELSQTVSRTSLKRLADFADDDDTKACVERIAGDDYTVEVEEQRMSLLDILRKYPGINMPLSTFLSMLPQMRPRTYSFASAPEWKQGHGMLLFSVVEAEEGTVSRPGGLATNYMAQLRQGDSILVEPRPCRPELRTTMMLPEPKVPIIMIAVGAGLAPFLGYLQKRFLQAQSQRTALPPCTLLFGCRGAKMDDICRAQLDEYSRAGVVSVHRAYSRDPDSQCKYVQGLVTKHSETLAKQWAQGAIVMVCSGKKVSDGVMNVLSPILFAEEKRSGMTGADSVDVWRQNVPKERMILEVFG

SEQ ID NO: 40 - protein, 1130 aa, Magnaporthe oryzae
  Magnaporthe oryzae (M. grisea) strain 70-15 hypothetical protein MGG_01925, CYP505A5
MFFLSSSLAYMAATQSRDWASFGVSLPSTALGRHLQAAMPFLSEENHKSQGTVLIPDAQGPIPFLGSVPLVDPELPSQSLQRLARQYGEIYRFVIPGRQSPILVSTHALVNELCDEKRFKKKVAAALLGLREAIHDGLFTAHNDEPNWGIAHRILMPAFGPMAIKGMFDEMHDVASQMILKWARHGSTTPIMVSDDFTRLTLDTIALCSMGYRFNSFYHDSMHEFIEAMTCWMKESGNKTRRLLPDVFYRTTDKKWHDDAEILRRTADEVLKARKENPSGRKDLLTAMIEGVDPKTGGKLSDSSIIDNLITFLIAGHETTSGMLSFAFYLLLKNPTAYRKAQQEIDDLCGREPITVEHLSKMPYITAVLRETLRLYSTIPAFVVEAIEDTVVGGKYAIPKNHPIFLMIAESHRDPKVYGDDAQEFEPERMLDGQFERRNREFPNSWKPFGNGMRGCIGRAFAWQEALLITAMLLQNFNFVMHDPAYQLSIKENLTLKPDNFYMRAILRHGMSPTELERSISGVAPTGNKTPPRNATRTSSPDPEDGGIPMSIYYGSNSGTCESLAHKLAVDASAQGFKAETVDVLDAANQKLPAGNRGPVVLITASYEGLPPDNAKHFVEWLENLKGGDELVDTSYAVFGCGHQDWTKTFHRIPKLVDEKLAEHGAVRLAPLGLSNAAHGDMFVDFETWEFETLWPALADRYKTGAGRQDAAATDLTAALSQLSVEVSHPRAADLRQDVGEAVVVAARDLTAPGAPPKRHMEIRLPKTGGRVHYSAGDYLAVLPVNPKSTVERAMRRFGLAWDAHVTIRSGGRTTLPTGAPVSAREVLSSYVELTQPATKRGIAVLAGAVTGGPAAEQEQAKAALLDLAGDSYALEVSAKRVGVLDLLERFPACAVPFGTFLALLPPMRVRQYSISSSPLWNDEHATLTYSVLSAPSLADPARTHVGVASSYLAGLGEGDHLHVALRPSHVAFRLPSPETPVVCVCAGSGMAPFRAFAQERAALVGAGRKVAPLLLFFGCREPGVDDLYREELEGWEAKGVLSVRRAYSRRTEQSEGCRYVQDRLLKNRAEVKSLWSQDAKVFVCGSREVAEGVKEAMFKVVAGKEGSSEEVQAWYEEVRNVRYASDIFD

SEQ ID NO: 41 - protein, 1108 aa, Neurospora crassa
  Neurospora crassa strain OR74A bifunctional P-450NADPH-P450 reductase, CYP505A2
MSSDETPQTIPIPGPPGLPLVGNSFDIDTEFPLGSMLNFADQYGEIFRLNFPGRNTVFVTSQALVHELCDEKRFQKTVNSALHEIRHGIHDGLFTARNDEPNWGIAHRILMPAFGPMAIQNMFPEMHEIASQLALKWARHGPNQSIKVTDDFTRLTLDTIALCSMDYRFNSYYHDDMHPFIDAMASFLVESGNRSRRPALPAFMYSKVDRKFYDDIRVLRETAEGVLKSRKEHPSERKDLLTAMLDGVDPKTGGKLSDDSIIDNLITFLIAGHETTSGLLSFAFVQLLKNPETYRKAQKEVDDVCGKGPIKLEHMNKLHYIAAVLRETLRLCPTIPVIGVESKEDTVIGGKYEVSKGQPFALLFAKSHVDPAVYGDTANDFDPERMLDENFERLNKEFPDCWKPFGNGMRACIGRPFAWQEALLVMAVCLQNFNFMPEDPNYTLQYKQTLTTKPKGFYMRAMLRDGMSALDLERRLKGELVAPKPTAQGPVSGQPKKSGEGKPISIYYGSNTGTCETFAQRLASDAEAHGFTATIIDSLDAANQNLPKDRPVVFITASYEGQPPDNAALFVGWLESLTGNELEGVQYAVFGCGHHDWAQTFHRIPKLVDNTVSERGGDRICSLGLADAGKGEMFTEFEQWEDEVFWPAMEEKYEVSRKEDDNEALLQSGLTVNFSKPRSSTLRQDVQEAVVVDAKTITAPGAPPKRHIEVQLSSDSGAYRSGDYLAVLPINPKETVNRVMRRFQLAWDTNITIEASRQTTILPTGVPMPVHDVLGAYVELSQPATKKNILALAEAADNAETKATLRQLAGPEYTEKITSRRVSILDLLEQFPSIPLPFSSFLSLLPPMRVRQYSISSSPLWNPSHVTLTYSLLESPSLSNPDKKHVGVATSYLASLEAGDKLNVSIRPSHKAFHLPVDADKTPLIMIAAGSGLAPFRGFVQERAAQIAAGRSLAPAMLFYGCRHPEQDDLYRDEFDKWESIGAVSVRRAFSRCPESQETKGCKYVGDRLWEDREEVTGLWDRGAKVYVCGSREVGESVKKVVVRIALERQKMIVEAREKGELDSLPEGIVEGLKLKGLTVEDVEVSEERALKWFEGIRNERYATDVFD

SEQ ID NO: 42 - protein, 561 aa, Oryza sativa
  Oryza sativa Japonica group cytochrome P450 family protein, CYP97C
MAAAAAAAVPCVPFLCPPPPPLVSPRLRRGHVRLRLRPPRSSGGGGGGGAGGDEPPITTSWVSPDWLTALSRSVATRLGGGDDSGIPVASAKLDDVRDLLGGALFLPLFKWFREEGPVYRLAAGPRDLVVVSDPAVARHVLRGYGSRYEKGLVAEVSEFLFGSGFAIAEGALWTVRRRSVVPSLHKRFLSVMVDRVFCKCAERLVEKLETSALSGKPVNMEARFSQMTLDVIGLSLFNYNFDSLTSDSPVIDAVYTALKEAELRSTDLLPYWKIDLLCKIVPRQIKAEKAVNIIRNTVEDLITKCKKIVDAENEQIEGEEYVNEADPSILRFLLASREEVTSVQLRDDLLSMLVAGHETTGSVLTWTIYLLSKDPAALRRAQAEVDRVLQGRLPRYEDLKELKYLMRCINESMRLYPHPPVLIRRAIVDDVLPGNYKIKAGQDIMISVYNIHRSPEVWDRADDFIPERFDLEGPVPNETNTEYRFIPFSGGPRKCVGDQFALLEAIVALAVVLQKMDIELVPDQKINMTTGATIHTTNGLYMNVSLRKVDREPDFALSGSR

SEQ ID NO: 43 - protein, 467 aa, Artificial Sequence
  synthetic chimeric heme enzyme C2G9, chimeric cytochrome P450 enzyme C2G9
MKETSPIPQPKTFGPLGNLPLIDKDKPTLSLIKLAEEQGPIFQIHTPAGTTIVVSGHELVKEVCDEERFDKSIEGALEKVRAFSGDGLATSWTHEPNWRKAHNILMPTFSQRAMKDYHEKMVDIAVQLIQKWARLNPNEAVDVPGDMTRLTLDTIGLCGFNYRFNSYYRETPHPFINSMVRALDEAMHQMQRLDVQDKLMVRTKRQFRYDIQTMFSLVDRMIAERKANPDENIKDLLSLMLYAKDPVTGETLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPISKGQPVTVLIPKLHRDQNAWGPDAEDFRPERFEDPSSIPHHAYKPFGNGQRACIGMQFALHEATLVLGMILKYFTLIDHENYELDIKQTLTLKPGDFHISVQSRHQEAIHADVQAAE

SEQ ID NO: 44 - protein, 467 aa, Artificial Sequence
  synthetic chimeric heme enzyme X7, chimeric cytochrome P450 enzyme X7
MKETSPIPQPKTFGPLGNLPLIDKDKPTLSLIKLAEEQGPIFQIHTPAGTTIVVSGHELVKEVCDEERFDKSIEGALEKVRAFSGDGLATSWTHEPNWRKAHNILMPTFSQRAMKDYHEKMVDIATQLIQKWSRLNPNEEIDVADDMTRLTLDTIGLCGFNYRFNSFYRDQPHPFITSMVRALDEAMNKLQRANPDDPAYDENKRQFQEDIKVMNDLVDSIIAERRANGDQDEKDLLARMLNVEDPETGEKLDDENIRFQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQA

SEQ ID NO: 45 - protein, 466 aa, Artificial Sequence
  synthetic chimeric heme enzyme X7-12, chimeric cytochrome P450 enzyme X7-12
MTIKEMPQPKTFGELKNLPLLNTDKPVQALMKIADELGEIFKFEAPGRVTRYLSSQRLIKEACDEERFDKSIEGALEKVRAFSGDGLATSWTHEPNWRKAHNILMPTFSQRAMKDYHEKMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDSIIAERRANGDQDEKDLLARMLNVEDPETGEKLDDENIRFQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQA

SEQ ID NO: 46 - protein, 465 aa, Artificial Sequence
  synthetic chimeric heme enzyme C2E6, chimeric cytochrome P450 enzyme C2E6
MTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDRMIAERKANPDENIKDLLSLMLYAKDPVTGETLDDENIRYQIITFLIAGHETTSGLLSFAIYCLLTHPEKLKKAQEEADRVLTDDTPEYKQIQQLKYIRMVLNETLRLYPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPST

SEQ ID NO: 47 - protein, 467 aa, Artificial Sequence
  synthetic chimeric heme enzyme X7-9, chimeric cytochrome P450 enzyme X7-9
MKQASAIPQPKTYGPLKNLPHLEKEQLSQSLWRIADELGPIFRFDFPGVSSVFVSGHNLVAEVCDEERFDKSIEGALEKVRAFSGDGLATSWTHEPNWRKAHNILMPTFSQRAMKDYHEKMVDIATQLIQKWSRLNPNEEIDVADDMTRLTLDTIGLCGFNYRFNSFYRDQPHPFITSMVRALDEAMNKLQRANPDDPAYDENKRQFQEDIKVMNDLVDSIIAERRANGDQDEKDLLARMLNVEDPETGEKLDDENIRFQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQA

SEQ ID NO: 48 - protein, 467 aa, Artificial Sequence
  synthetic chimeric heme enzyme C2B12, chimeric cytochrome P450 enzyme C2B12
MKQASAIPQPKTYGPLKNLPHLEKEQLSQSLWRIADELGPIFRFDFPGVSSVFVSGHNLVAEVCDEERFDKSIEGALEKVRAFSGDGLATSWTHEPNWRKAHNILMPTFSQRAMKDYHEKMVDIATQLIQKWSRLNPNEEIDVADDMTRLTLDTIGLCGFNYRFNSFYRDQPHPFITSMVRALDEAMNKLQRANPDDPAYDENKRQFQEDIKVMNDLVDRMIAERKANPDENIKDLLSLMLYAKDPVTGETLDDENIRYQIITFLIAGHETTSGLLSFATYFLLKHPDKLKKAYEEVDRVLTDAAPTYKQVLELTYIRMILNESLRLWPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQA

SEQ ID NO: 49 - protein, 467 aa, Artificial Sequence
  synthetic chimeric heme enzyme TSP234, chimeric cytochrome P450 enzyme TSP234
MKETSPIPQPKTFGPLGNLPLIDKDKPTLSLIKLAEEQGPIFQIHTPAGTTIVVSGHELVKEVCDEERFDKSIEGALEKVRAFSGDGLATSWTHEPNWRKAHNILMPTFSQRAMKDYHEKMVDIATQLIQKWSRLNPNEEIDVADDMTRLTLDTIGLCGFNYRFNSFYRDQPHPFITSMVRALDEAMNKLQRANPDDPAYDENKRQFQEDIKVMNDLVDRMIAERKANPDENIKDLLSLMLYAKDPVTGETLDDENIRYQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQA

SEQ ID NO: 50 - protein, 414 aa, Artificial Sequence
  synthetic cytochrome P450cam mutant, CYP101A1 mutant (C357S)
TTETIQSNANLAPLPPHVPEHLVFDFDMYNPSNLSAGVQEAWAVLQESNVPDLVWTRCNGGHWIATRGQLIREAYEDYRHFSSECPFIPREAGEAYDFIPTSMDPPEQRQFRALANQVVGMPVVDKLENRIQELACSLIESLRPQGQCNFTEDYAEPFPIRIFMLLAGLPEEDIPHLKYLTDQMTRPDGSMTFAEAKEALYDYLIPIIEQRRQKPGTDAISIVANGQVNGRPITSDEAKRMCGLLLVGGLDTVVNFLSFSMEFLAKSPEHRQELIERPERIPAACEELLRRFSLVADGRILTSDYEFHGVQLKKGDQILLPQMLSGLDERENACPMHVDFSRQKVSHTTFGHGSHLSLGQHLARREIIVTLKEWLTRIPDFSIAPGAQIQHKSGIVSGVQALPLVWDPATTKAV

SEQ ID NO: 51 - protein, 414 aa, Artificial Sequence
  synthetic cytochrome P450cam mutant, CYP101A1 mutant (T252A)
TTETIQSNANLAPLPPHVPEHLVFDFDMYNPSNLSAGVQEAWAVLQESNVPDLVWTRCNGGHWIATRGQLIREAYEDYRHFSSECPFIPREAGEAYDFIPTSMDPPEQRQFRALANQVVGMPVVDKLENRIQELACSLIESLRPQGQCNFTEDYAEPFPIRIFMLLAGLPEEDIPHLKYLTDQMTRPDGSMTFAEAKEALYDYLIPIIEQRRQKPGTDAISIVANGQVNGRPITSDEAKRMCGLLLVGGLDAVVNFLSFSMEFLAKSPEHRQELIERPERIPAACEELLRRFSLVADGRILTSDYEFHGVQLKKGDQILLPQMLSGLDERENACPMHVDFSRQKVSHTTFGHGSHLCLGQHLARREIIVTLKEWLTRIPDFSIAPGAQIQHKSGIVSGVQALPLVWDPATTKAV

SEQ ID NO: 52 - protein, 414 aa, Artificial Sequence
  synthetic cytochrome P450cam mutant, CYP101A1 mutant (C357A and T252A)
TTETIQSNANLAPLPPHVPEHLVFDFDMYNPSNLSAGVQEAWAVLQESNVPDLVWTRCNGGHWIATRGQLIREAYEDYRHFSSECPFIPREAGEAYDFIPTSMDPPEQRQFRALANQVVGMPVVDKLENRIQELACSLIESLRPQGQCNFTEDYAEPFPIRIFMLLAGLPEEDIPHLKYLTDQMTRPDGSMTFAEAKEALYDYLIPIIEQRRQKPGTDAISIVANGQVNGRPITSDEAKRMCGLLLVGGLDAVVNFLSFSMEFLAKSPEHRQELIERPERIPAACEELLRRFSLVADGRILTSDYEFHGVQLKKGDQILLPQMLSGLDERENACPMHVDFSRQKVSHTTFGHGSHLSLGQHLARREIIVTLKEWLTRIPDFSIAPGAQIQHKSGIVSGVQALPLVWDPATTKAV

SEQ ID NO: 53 - protein, 491 aa, Artificial Sequence
  synthetic cytochrome P450 2B4 mutant, CYP2B4 (C436S)
MEFSLLLLLAFLAGLLLLLFRGHPKAHGRLPPGPSPLPVLGNLLQMDRKGLLRSFLRLREKYGDVFTVYLGSRPVVVLCGTDAIREALVDQAEAFSGRGKIAVVDPIFQGYGVIFANGERWRALRRFSLATMRDFGMGKRSVEERIQEEARCLVEELRKSKGALLDNTLLFHSITSNIICSIVFGKRFDYKDPVFLRLLDLFFQSFSLISSFSSQVFELFPGFLKHFPGTHRQIYRNLQEINTFIGQSVEKHRATLDPSNPRDFIDVYLLRMEKDKSDPSSEFHHQNLILTVLSLFFAGTETTSTTLRYGFLLMLKYPHVTERVQKEIEQVIGSHRPPALDDRAKMPYTDAVIHEIQRLGDLIPFGVPHTVTKDTQFRGYVIPKNTEVFPVLSSALHDPRYFETPNTFNPGHFLDANGALKRNEGFMPFSLGKRISLGEGIARTELFLFFTTILQNFSIASPVPPEDIDLTPRESGVGNVPPSYQIRFLAR

SEQ ID NO: 54 - protein, 491 aa, Artificial Sequence
  synthetic cytochrome P450 2B4 mutant, CYP2B4 (T302A)
MEFSLLLLLAFLAGLLLLLFRGHPKAHGRLPPGPSPLPVLGNLLQMDRKGLLRSFLRLREKYGDVFTVYLGSRPVVVLCGTDAIREALVDQAEAFSGRGKIAVVDPIFQGYGVIFANGERWRALRRFSLATMRDFGMGKRSVEERIQEEARCLVEELRKSKGALLDNTLLFHSITSNIICSIVFGKRFDYKDPVFLRLLDLFFQSFSLISSFSSQVFELFPGFLKHFPGTHRQIYRNLQEINTFIGQSVEKHRATLDPSNPRDFIDVYLLRMEKDKSDPSSEFHHQNLILTVLSLFFAGTEATSTTLRYGFLLMLKYPHVTERVQKEIEQVIGSHRPPALDDRAKMPYTDAVIHEIQRLGDLIPFGVPHTVTKDTQFRGYVIPKNTEVFPVLSSALHDPRYFETPNTFNPGHFLDANGALKRNEGFMPFSLGKRICLGEGIARTELFLFFTTILQNFSIASPVPPEDIDLTPRESGVGNVPPSYQIRFLAR

SEQ ID NO: 55 - protein, 491 aa, Artificial Sequence
  synthetic cytochrome P450 2B4 mutant, CYP2B4 (C436S and T302A)
MEFSLLLLLAFLAGLLLLLFRGHPKAHGRLPPGPSPLPVLGNLLQMDRKGLLRSFLRLREKYGDVFTVYLGSRPVVVLCGTDAIREALVDQAEAFSGRGKIAVVDPIFQGYGVIFANGERWRALRRFSLATMRDFGMGKRSVEERIQEEARCLVEELRKSKGALLDNTLLFHSITSNIICSIVFGKRFDYKDPVFLRLLDLFFQSFSLISSFSSQVFELFPGFLKHFPGTHRQIYRNLQEINTFIGQSVEKHRATLDPSNPRDFIDVYLLRMEKDKSDPSSEFHHQNLILTVLSLFFAGTEATSTTLRYGFLLMLKYPHVTERVQKEIEQVIGSHRPPALDDRAKMPYTDAVIHEIQRLGDLIPFGVPHTVTKDTQFRGYVIPKNTEVFPVLSSALHDPRYFETPNTFNPGHFLDANGALKRNEGFMPFSLGKRISLGEGIARTELFLFFTTILQNFSIASPVPPEDIDLTPRESGVGNVPPSYQIRFLAR

SEQ ID NO: 56 - protein, 463 aa, Artificial Sequence
  synthetic cytochrome P450-BM3 variant WT-AxA (heme)
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRAAIGQQFALHEATLVLGMMLKHFDFEDHTNYELDIKETLSLKPKGFVVKAKSKKIPLGGIPSPST

SEQ ID NO: 57 - protein, 463 aa, Artificial Sequence
  synthetic cytochrome P450-BM3 variant WT-AxD (heme)
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRADIGQQFALHEATLVLGMMLKHFDFEDHTNYELDIKETLSLKPKGFVVKAKSKKIPLGGIPSPST

SEQ ID NO: 58 - protein, 463 aa, Artificial Sequence
  synthetic cytochrome P450-BM3 variant WT-AxH (heme)
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRAHIGQQFALHEATLVLGMMLKHFDFEDHTNYELDIKETLSLKPKGFVVKAKSKKIPLGGIPSPST

SEQ ID NO: 59 - protein, 463 aa, Artificial Sequence
  synthetic cytochrome P450-BM3 variant WT-AxK (heme)
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRAKIGQQFALHEATLVLGMMLKHFDFEDHTNYELDIKETLSLKPKGFVVKAKSKKIPLGGIPSPST

SEQ ID NO: 60 - protein, 463 aa, Artificial Sequence
  synthetic cytochrome P450-BM3 variant WT-AxM (heme)
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRAMIGQQFALHEATLVLGMMLKHFDFEDHTNYELDIKETLSLKPKGFVVKAKSKKIPLGGIPSPST

SEQ ID NO: 61 - protein, 463 aa, Artificial Sequence
  synthetic cytochrome P450-BM3 variant WT-AxN (heme)
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRANIGQQFALHEATLVLGMMLKHFDFEDHTNYELDIKETLSLKPKGFVVKAKSKKIPLGGIPSPST

SEQ ID NO: 62 - protein, 1047 aa, Artificial Sequence
  synthetic cytochrome P450-BM3 variant BM3-CIS-T438S-AxA
TIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQFQEDIKVMNDLVDIIADRKARGEQSDDLLTQMLNGKDPETGEPLDDGNIRYQIITFLIAGHEATSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRAAIGQQFALHEATLVLGMMLKHFDFEDHTNYELDIKETLSLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 63 - protein, 1047 aa, Artificial Sequence
  synthetic cytochrome P450-BM3 variant BM3-CIS-T438S-AxD
TIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQFQEDIKVMNDLVDIIADRKARGEQSDDLLTQMLNGKDPETGEPLDDGNIRYQIITFLIAGHEATSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRADIGQQFALHEATLVLGMMLKHFDFEDHTNYELDIKETLSLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 64 - protein, 1047 aa, Artificial Sequence
  synthetic cytochrome P450-BM3 variant BM3-CIS-T438S-AxM
TIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQFQEDIKVMNDLVDIIADRKARGEQSDDLLTQMLNGKDPETGEPLDDGNIRYQIITFLIAGHEATSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRAMIGQQFALHEATLVLGMMLKHFDFEDHTNYELDIKETLSLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 65 - protein, 1047 aa, Artificial Sequence
  synthetic cytochrome P450-BM3 variant BM3-CIS-T438S-AxY
TIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQFQEDIKVMNDLVDIIADRKARGEQSDDLLTQMLNGKDPETGEPLDDGNIRYQIITFLIAGHEATSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRAYIGQQFALHEATLVLGMMLKHFDFEDHTNYELDIKETLSLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 66 - protein, 1047 aa, Artificial Sequence
  synthetic cytochrome P450-BM3 variant BM3-CIS-T438S-AxT
TIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQFQEDIKVMNDLVDIIADRKARGEQSDDLLTQMLNGKDPETGEPLDDGNIRYQIITFLIAGHEATSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRATIGQQFALHEATLVLGMMLKHFDFEDHTNYELDIKETLSLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 67 - protein, 35 aa, Artificial Sequence
  synthetic cytochrome P450-BM3 sequence of CYP102A1
ENPSAIPQHAFKPFGNGQRACIGQQFALHEATLVL

SEQ ID NO: 68 - protein, 43 aa, Artificial Sequence
  synthetic cytochrome P450-CAM sequence of Pseudomonas putida CYP101A1
ERENACPMHVDFSRQKVSHTTFGHGSHLCLGQHLARREIIVTL

SEQ ID NO: 69 - protein, 60 aa, Artificial Sequence
  synthetic cytochrome P450-BM3 sequence of CYP102A1
GHETTSGLLSFALYFLVKNPHVLQKAAEEAARVLVDPVPSYKQVKQLKYVGMVLNEALRL

SEQ ID NO: 70 - protein, 42 aa, Artificial Sequence
  synthetic cytochrome P450-CAM sequence of Pseudomonas putida CYP101A1
GLDTVVNFLSFSMEFLAKSPEHRQELIERPERIPAACEELLR

SEQ ID NO: 71 - protein, 49 aa, Artificial Sequence
  synthetic cytochrome P450-BM3 sequence of CYP102A1
FRPERFENPSAIPQHAFKPFGNGQRACIGQQFALHEATLVLGMMLKHFD

SEQ ID NO: 72 - protein, 51 aa, Artificial Sequence
  synthetic cytochrome P450 sequence of Oryctolagus cuniculus CYP2B4
FNPGHFLDANGALKRNEGFMPFSLGKRVCLGEGIARTELFLFFTTILQNFS

SEQ ID NO: 73 - protein, 59 aa, Artificial Sequence
  synthetic cytochrome P450-BM3 sequence of CYP102A1
QIITFLIAGHETTSGLLSFALYFLVKNPHVLQKAAEEAARVLVDPVPSYKQVKQLKYVG

SEQ ID NO: 74 - protein, 60 aa, Artificial Sequence
  synthetic cytochrome P450 sequence of Oryctolagus cuniculus CYP2B4
TVLSLFFAGTETTSTTLRYGFLLMLKYPHVTERVQKEIEQVIGSHRPPALDDRAKMPYTD

SEQ ID NO: 75 - protein, 4 aa, Artificial Sequence
  synthetic P450-BM3  (CYP102A1) and Oryctolagus cuniculus (CYP2B4) consensus peptide
ETTS

(19) United States
(12) Patent Application Publication
(10) Pub. No.: US 2015/0267232 A1
(43) Pub. Date: Sep. 24, 2015

(54) IN VIVO AND IN VITRO CARBENE INSERTION AND NITRENE TRANSFER REACTIONS CATALYZED BY HEME ENZYMES

(71) Applicant: California Institute of Technology, Pasadena, CA (US)

(72) Inventors: PEDRO COELHO, Los Angeles, CA (US); Frances H. Arnold, La Canada, CA (US); Jared C. Lewis, Chicago, IL (US); Zhan Wang, San Jose, CA (US)

(73) Assignee: California Institute of Technology, Pasadena, CA (US)

(21) Appl. No.: 14/676,744

(22) Filed: Apr. 1, 2015

Related U.S. Application Data
(63) Continuation of application No. PCT/US2013/063428, filed on Oct. 4, 2013.
(60) Provisional application No. 61/869,518, filed on Aug. 23, 2013.
(60) Provisional application No. 61/838,167, filed on Jun. 21, 2013.
(60) Provisional application No. 61/806,162, filed on Mar. 28, 2013.
(60) Provisional application No. 61/784,917, filed on Mar. 14, 2013.
(60) Provisional application No. 61/740,247, filed on Dec. 20, 2012.
(60) Provisional application No. 61/711,640, filed on Oct. 9, 2012.

(51) Int. Cl. C12P 13/00; C07C 311/16; C12P 17/10; C07D 275/06; C07C 229/18; C12N 9/02; C07C 311/29; C12P 17/14

(52) U.S. Cl. CPC C12P 13/00; C07C 311/29; C07C 311/16; C12P 17/14; C07D 275/06; C07C 229/18; C12P 13/001; C12N 9/0071; C12P 13/002; C12P 17/10; C12Y 114/14001

(57) ABSTRACT

This invention relates to the use of heme-containing enzymes to catalyze carbene and nitrene insertion and transfer reactions with greater selectivity, mild reaction conditions, and convenient production.

### CROSS-REFERENCES TO RELATED APPLICATIONS

The present invention is a continuation of PCT/US2013/063428, filed Oct. 4, 2013, which application claims priority to U.S. Provisional Patent Application No. 61/711,640, filed Oct. 9, 2012; U.S. Provisional Patent Application No. 61/740,247, filed Dec. 20, 2012; U.S. Provisional Patent Application No. 61/784,917, filed Mar. 14, 2013; U.S. Provisional Patent Application No. 61/806,162, filed Mar. 28, 2013; U.S. Provisional Patent Application No. 61/838,167, filed Jun. 21, 2013; and U.S. Provisional Patent Application No. 61/869,518, filed Aug. 23, 2013, each application of which, is hereby incorporated by reference in its entirety for all purposes. In addition, this application is related to PCT Application No. US2013/063577 filed Oct. 4, 2013 (Publication No. WO 2014/058744), which application is hereby incorporated by reference in its entirety for all purposes.

### STATEMENT AS TO RIGHTS TO INVENTIONS MADE UNDER FEDERALLY SPONSORED RESEARCH AND DEVELOPMENT

This invention was made with government support under DE-FG02-06ER15762/T-105789 awarded by the Department of Energy and under 1F32EB015846-01. The government has certain rights in the invention.

### BACKGROUND OF THE INVENTION

Enzymes offer appealing alternatives to traditional chemical catalysts due to their ability to function in aqueous media at ambient temperature and pressure. In addition, the ability of enzymes to orient substrate binding for defined regio- and stereochemical outcomes is highly valuable. This property is exemplified by the cytochrome P450 monooxygenase family of enzymes that catalyze insertion of oxygen atoms into unactivated C—H bonds (P. R. O. d. Montellano, Cytochrome P450: Structure, Mechanism and Biochemistry. Kluwer Academic/Plenum Publishers, New York, ed. 3rd Edition, 2005).

Cytochrome P450s catalyze monooxygenation with high degrees of regio- and stereoselectivity, a property that makes them attractive for use in chemical synthesis. This broad enzyme class is capable of oxygenating a wide variety of organic molecules including aromatic compounds, fatty acids, alkanes and alkenes. Diverse substrate selectivity is a hallmark of this enzyme family and is exemplified in the natural world by their importance in natural product oxidation as well as xenobiotic metabolism (F. P. Guengerich, Chem. Res. Toxicol. 14, 611 (2001)). Limitations to this enzyme class in synthesis include their large size, need for expensive reducing equivalents (e.g., NADPH) and cellular distribution—many cytochrome P450s are membrane bound and therefore difficult to handle (Montellano, Cytochrome P450: Structure, Mechanism and Biochemistry. Kluwer Academic/Plenum Publishers, New York, ed. 3rd Edition, 2005). Several soluble bacterial cytochrome P450s have been isolated, however, that show excellent properties and behavior for chemical synthesis and protein engineering applications.

Natural products and fine chemicals are often highly functionalized with amines and amides, making strategies for the efficient installation of nitrogen atoms of primary importance to organic synthesis. For example, C—N bond formation methods often require preoxidized carbon functional groups and the use of protecting groups, which renders these methods redox- and atom-inefficient (Zalatan, D. & Du Bois, Top. Curr. Chem. 292, 347-378 (2010)). The ability to insert nitrogen directly—via formal nitrene transfers—into unactivated C—H bonds allows for more convenient synthesis of otherwise difficult-to-make molecules (Zalatan, D. & Du Bois, Top. Curr. Chem. 292, 347-378 (2010); Davies, H. M. L. & Manning, J. R., Nature 451, 417-424 (2008)). Significant progress in this direction has been made in the form of organometallic catalysts that can transfer nitrene equivalents to C—H bonds (Ramirez, T. A. et al., Chem. Soc. Rev. 41, 931-942 (2012); Driver, T. G., Org. Biomol. Chem. 8, 3831-3846 (2010)). No enzymes are known to catalyze the oxidative amination of C—H bonds. Although most natural C—N bonds are formed via nucleophilic processes, nature can aminate unactivated C—H bonds via hydroxylation to the alcohol followed by either dehydrogenation to the carbonyl and then reductive amination or direct nucleophilic displacement to give the amine (Tschesche, R. et al., Phytochemistry 15, 1387-1389 (1976); Bennett, R. D. & Heftmann, Phytochemistry 4, 873-879 (1965); Leete, E., Acc. Chem. Res. 4, 100-107 (1971)).

C—H amination is a challenging transformation that allows chemists to rapidly add complexity to a molecule. Notable advances towards transition-metal catalysis of C—H amination have been achieved using rhodium, cobalt, and ruthenium based catalysts (Zalatan, D. & Du Bois, Top. Curr. Chem. 292, 347-378 (2010); Davies, H. M. L. & Manning, J. R., Nature 451, 417-424 (2008)). Transition metal-catalyzed C—H amination proceeds through a nitrenoid intermediate without mechanistic parallel in natural enzymes, but is isoelectronic with formal oxene transfers catalyzed by cytochrome P450 enzymes.

Enzymes offer many advantages over traditional catalysts, such as selectivity, mild reaction conditions, convenient production, and use in whole cells. Cytochrome P450 enzymes are known to be able to carry out monooxygenations of diverse substrates, and exemplify the mild operating conditions that enzymes can afford. Many of the small molecule catalysts developed for C—H amination reaction have been designed in an effort to mimic these enzymes, but with the goal of activating nitrene equivalents rather than the oxene equivalents activated by cytochrome P450 enzymes (Bennett, R. D. & Heftmann, Phytochemistry 4, 873-879 (1965)). Cytochrome P450 enzymes bind to a cofactor consisting of a catalytic transition metal (iron heme) that forms a reactive intermediate known as ‘Compound I’ that is similar in electronic and steric features to metallonitrenoid intermediates used for synthetic C—N bond forming reactions. In addition to C—H amination, P450 variants were investigated to assess their reactivity towards weak C—H, N—H, O—H and Si—for carbene insertion.

There is a need in the art for catalytic processes for achieving carbene and nitrene insertion and transfer reactions with greater selectivity, mild reaction conditions, and convenient production. The present invention satisfies these and other needs.

### BRIEF SUMMARY OF THE INVENTION

The present invention is based on the surprising discovery that engineered heme enzymes such as cytochrome P450_{BM3} enzymes, including a serine-heme-ligated P411 enzyme, efficiently catalyze carbene and nitrene insertion and transfer reactions. Suitable reactions include, but are not limited to, carbene insertion reactions into N—H, C—H, O—H or Si—H bonds, as well as nitrene transfer into C═C and C—H bonds.

In one embodiment, the present invention provides a method for catalyzing a carbene insertion into a N—H bond to produce a product having a new C—N bond, the method comprising:

providing a N—H containing substrate, a diazo carbene precursor and an engineered heme enzyme; and

allowing the reaction to proceed for a time sufficient to form a product having a new C—N bond.

In another embodiment, the present invention provides a method for catalyzing a carbene insertion into a C—H bond to produce a product with a new C—C bond, the method comprising:

providing a C—H containing substrate, a diazo carbene precursor and an engineered heme enzyme; and

allowing the reaction to proceed for a time sufficient to form a product having a new C—C bond.

In yet another embodiment, the present invention provides a method for catalyzing a carbene insertion into a O—H bond to produce a product having a new C—O bond, the method comprising:
providing a O—H containing substrate, a diazo carbene precursor and an engineered heme enzyme; and

allowing the reaction to proceed for a time sufficient to form a product having a new C—O bond.

In another embodiment, the present invention provides a method for catalyzing a carbene insertion into a Si—H bond to produce a product having a new C—Si bond, the method comprising:

providing a Si—H containing substrate, a diazo carbene precursor and an engineered heme enzyme; and

allowing the reaction to proceed for a time sufficient to form a product having a new C—Si bond.

In still yet another embodiment, the present invention provides a method for catalyzing a nitrene insertion reaction into an olefin to produce an aziridine, the method comprising:

providing an olefin substrate, a nitrene precursor and an engineered heme enzyme; and

allowing the reaction to proceed for a time sufficient to produce an aziridine.

In another embodiment, the present invention provides a method for catalyzing a nitrene insertion into a C—H bond to produce a product having a new C—N bond, the method comprising:

providing a C—H containing substrate, a nitrene precursor and an engineered heme enzyme; and

allowing the reaction to proceed for a time sufficient to form a product having a new C—N bond.

In some embodiments, the present invention provides a heme enzyme variant or fragment thereof that can catalyze a carbene insertion into a N—H bond, C—H bond, O—H bond, and/or Si—H bond and/or catalyze a nitrene insertion into a C═C bond and/or C—H bond. In some aspects, the present invention also provides a cell expressing a heme enzyme variant or fragment thereof that can catalyze a carbene insertion into a N—H bond, C—H bond, O—H bond, and/or Si—H bond and/or catalyze a nitrene insertion into a C═C bond and/or C—H bond. In another aspect, the present invention further provides an expression vector comprising a nucleic acid sequence encoding a heme enzyme variant or fragment thereof that can catalyze a carbene insertion into a N—H bond, C—H bond, O—H bond, and/or Si—H bond and/or catalyze a nitrene insertion into a C═C bond and/or C—H bond.

In certain aspects, the present invention provides that wild-type P450_{BM3} and engineered variants therefrom show significant activity in the intramolecular C—H amination of arylsulfonyl azide substrates. To date, no natural enzymes have been described that catalyze a similar C—N bond forming reaction. Described herein is also the discovery that heme enzymes such as variants of P450_{BM3} with at least one and possibly more amino acid mutations catalyze C—H amination reactions efficiently, with increased total turnover numbers and demonstrate highly enantioselective product formation compared to wild type enzymes.

In other aspects, the present invention provides variants of the full-length cytochrome P450_{BM3} that show enhanced stereoselectivity and productivity in C—H bond amination. These enzymes can be produced with comparable convenience to wild-type P450_{BM3}, and their reactions can be driven by either NADPH or alternative reducing agents such as enzymatic electron transfer systems, NADH, or sodium dithionite.

In still other aspects, the present invention provides variants of truncated cytochrome P450_{BM3} containing only the heme-binding domain that show enhanced stereoselectivity and productivity in C—H bond amination. These enzymes can be produced even more readily than wild-type P450_{BM3}, and their reactions can be driven by alternative reducing agents such as enzymatic electron transfer systems, or by sodium dithionite.

In still other aspects, the present invention provides chimeric heme enzymes such as chimeric P450 protein variants comprised of recombined sequences from P450_{BM3} and two distantly related P450s from Bacillus subtillis that are competent C—H amination catalysts using similar conditions to wild type P450_{BM3} and highly active P450_{BM3} variants.

In other aspects, the present invention provides for P450 variants that enhance C—H amination activity at least two- and up to seventy-fold compared to wild-type P450_{BM3}, in vitro. In certain cases, the enzyme is a variant of P450_{BM3}, a variant of the isolated P450_{BM3} heme domain, or a recombinant P450_{BM3} derivative. In certain aspects, mutations that strongly improve C—H amination activity include T268A and C400S. The present invention not only considers enzymes that contain each mutation separately, but both mutations together, in which context a synergistic effect is noted that enhances C—H amination activity.

In still other aspects, the present invention provides that wild-type P450_{BM3}, and full-length and truncated variants therefrom, which are capable of catalyzing enantioselective C—N bond formation. Additionally, certain mutations are found to strongly affect the degree of asymmetric induction observed, which in certain instances, ranges from 1% to 99% such as 16% enantiomeric excess (% ee) to 91% ee.

In still other aspects, the present invention provides that wild-type P450_{BM3} and full-length and truncated variants therefrom are highly active C—H amination catalysts inside living cells. One consequence of this discovery is that bacterial cells (e.g., Escherichia coli) can be used as whole cell catalysts, obviating the requirement for protein extraction and purification. In particular, whole cell catalysts containing P450 enzymes that contain both C400S and T268A mutations are highly active, and show enhanced levels of enantioselectivity relative to purified enzymes.

In still yet other aspects, the invention also provides that engineered P450_{BM3} variants containing metal-substituted porphyrins catalyze intermolecular and intramolecular C—H amination. Mutations described as T268A, C400S, and others are capable of altering regio- and enantioselectivity of enzymes containing metal substituted porphyrins.

In still other aspects, the present invention provides the use of engineered heme enzymes for amination of C—H or C-heteroatom bonds using appropriate nitrene precursors.

In still other aspects, the present invention provides heme enzymes with axial heme serine coordination that catalyze C—H amination of alkyl groups using NAD(P)H as a reducing agent.

In still other aspects, the present invention provides heme enzymes that can effect enantioselective and regioselective C—H amination or heteroatom-H amination. Mutations to the enzyme, including but not limited to T268A, can result in alterations in enantioselectivity.

In still other aspects, the present invention provides engineered heme enzymes that can catalyze enantioselective and regioselective C═C aziridination of olefins.

In still other aspects, the present invention provides non-naturally occurring microbial organisms expressing heme enzymes where the organisms are efficient catalysts of C—H amination using arylsulfonyl azides or other appropriate nitrene precursors.

In still other aspects, the present invention provides enzyme variants comprised of the full-length P450BM3 enzyme, which may contain the mutations C400S and T268A as well as additional amino acid mutations, where such variants are active catalysts of C—H amination. Whole cells using said P450BM3 variants are also active C—H amination catalysts.

In still other aspects, the present invention provides enzyme variants comprised solely of the truncated P450BM3 heme domain that are active catalysts for C—H amination. Whole cells using said heme domains are also active C—H amination catalysts.

In still other aspects, the present invention provides chimeric P450 protein variants which active C—H amination catalysts. Whole cells containing the chimeric enzymes are also active C—H amination catalysts.

In still other aspects, the present invention provides metal-substituted heme enzymes containing protoporphyrin IX or other porphyrin molecules containing metals other than iron, including but not limited to cobalt, rhodium, ruthenium, or manganese, which are active C—H amination catalysts.

In still other aspects, the present invention provides engineered heme enzymes which can be lyophilized, stored and used as a solid or a liquid suspension in chemical reactions.

In still other aspects, the present invention provides engineered heme enzymes which can be used in biphasic reactors where the biocatalyst occurs in the aqueous layer and the substrates and/or products occur in an organic layer.

In still other aspects, the present invention provides the use of analogous mutations to T268A and C400S in other cytochrome P450 enzymes and heme enzymes in order to enhance C—H amination.

These and other aspects, objects and embodiments will become more apparent when read with the detailed description and drawings which follow.

### BRIEF DESCRIPTION OF THE DRAWINGS

FIG. 1 shows that P450BM3 variants display type I binding to arylsulfonyl azides.

FIG. 2 shows absorbance difference spectra for P450_{BM3} variants binding 2-isopropylbenzenesulfonyl azide. Sequence identities are shown on Table 9.

FIG. 3 shows absorbance difference spectra for P450_{BM3} variants binding 2-isopropylbenzenesulfonyl azide. Sequence identities are shown on Table 9.

FIG. 4 shows absorbance difference spectra for P450_{BM3} variants binding 2-isopropylbenzenesulfonyl azide. Sequence identities are shown on Table 9.

FIG. 5 shows absorbance difference spectra for P450_{BM3} variants binding 2-isopropylbenzenesulfonyl azide. Sequence identities are shown on Table 10.

FIG. 6 shows P450 bioconversions with 2-isopropylbenzenesulfonyl azide (1) under anaerobic conditions. Calculated and observed mass values are indicated. NES=negative electrospray, PES=positive electrospray.

FIG. 7 shows P450 reactions with azide 1 in the absence of NADPH. Alcohol 10 and arylsulfonamide 2 (*) are defined in FIG. 6.

FIG. 8 shows P450 reactions with azide 1 in the presence of 0.1 mM NADPH (0.05 eq). Alcohol 10 and arylsulfonamide 2 (*) are defined in FIG. 6.

FIG. 9 shows P450 reactions with azide 1 in the presence of 2 mM NADPH (1 eq). Alcohol 10, arylsulfonamide 2 (*) and dimer 4 (4a and 4b) (#) are defined in FIG. 6.

FIG. 10 shows P450-catalyzed amination of benzylic C—H bonds from arylsulfonyl azides. Products isolated from small-scale (30 mg azide) bioconversions were analyzed by NMR and mass spectrometry.

FIG. 11 show P450 bioconversions with 2,5-diisopropylbenzenesulfonyl azide (5) under anaerobic conditions. NES=negative electrospray, PES=positive electrospray.

FIG. 12 shows P450 reactions with azide 5 in presence of 2 mM NADPH (1 eq). Benzosultam 6, arylsulfonamide 7 (*), and dimer 12 (#) are defined in FIG. 10.

FIG. 13 shows P450 bioconversions with 2,4,6-triisopropylbenzenesulfonyl azide (8) under anaerobic conditions. NES=negative electrospray.

FIG. 14 shows P450 reactions with azide 8 in presence of 2 mM NADPH (1 eq). Benzosultam 9 (a), arylsulfonamide 13 (c), alcohol 14, alkene 15 (b) and dimer 16 (d) are defined in FIG. 12.

FIG. 15 shows P450 reactions with azide 8 in presence of 2 mM NADPH (1 eq). Benzosultam 9 (a), arylsulfonamide 13 (c), alcohol 14, alkene 15 (b) and dimer 16 (d) are defined in FIG. 12.

FIGS. 16A-B shows B1SYN type I binding curves for azides 5 (FIG. 16A) and 8 (FIG. 16B). K_{d} (5)=1.5 μM, K_{d} (8)=19 μM.

FIG. 17 shows C—H and N—H bond insertion by P450 variants in the presence of diazo compounds.

FIG. 18 shows a schematic depicting substrates used to test the dependence of C—H bond strength on amination activity in enzyme- and hemin-catalyzed reactions; 0.1 mol % of P411 catalysts (ABC-T268A and ABC-CIS) and 1 mol % hemin were reacted with 2 mM sulfonyl azide substrates 1, 4, or 6 with 2 mM NADPH, an oxygen depletion system (100 U ml⁻¹ glucose oxidase, 1400 U ml⁻¹ catalase, 25 mM glucose) in 0.1 M KPi pH 8.0 at room temperature for 24 hours.

FIG. 19 shows P450-catalyzed intramolecular C—H amination reactions using a variety of substrates.

FIG. 20 shows P450-catalyzed intermolecular C—H amination reactions using a variety of substrates.

FIG. 21 shows P450-catalyzed intramolecular aziridination reactions using a variety of substrates.

FIG. 22 shows P450-catalyzed intermolecular aziridination reactions using a variety of substrates.

FIG. 23 shows substrates for purified enzyme and whole-cell reactions.

FIGS. 24A-C show a demonstration of enzymatic production of (5). Panel A is an LC-MS 220 nm chromatogram of enzyme reaction mixture containing putative 5, Panel B is a synthetic standard of 5 whose NMR spectra are presented in FIG. 33, and Panel C is a sample containing a mixture of the enzyme reaction and synthetic 5, showing coelution.

FIGS. 25A-D show a demonstration of the enzymatic production of (5). LC runs showing ESI-MS-(−) detection of selected ions (mass window 195.5-196.5) Panels C-D; top panel shows 220 nm trace from enzyme reaction in FIG. 24A.

FIGS. 26A-C show a demonstration of enzymatic production of (7). At top is an LC-MS chromatogram (recorded at 220 nm) of an enzyme reaction mixture containing putative 7; in the middle is a synthetic standard of 7 whose NMR data is presented in FIG. 33; and at bottom is a sample containing a mixture of the enzyme reaction and synthetic 7, showing coelution. Panel A is LC-MS 220 nm chromatogram of enzyme reaction mixture containing putative 7, Panel B is a synthetic standard of 7 whose NMR data is presented in FIG. 34 and Panel C is a sample containing a mixture of the enzyme reaction and synthetic 7, showing coelution.

FIGS. 27A-D show a demonstration of enzymatic production of (7). LC runs from FIG. 25 showing ESI-MS-(−) detection of selected ions (mass window 279.5-280.5); top panel shows 220 nm trace from enzyme reaction. A second isobaric peak with m/z 280 Da can be observed in enzyme reactions. This material was not present in sufficient quantities to permit detailed structural characterization.

FIG. 28 shows ¹H and ¹³C NMR spectra for (1)

FIG. 29 shows ¹H and ¹³C NMR spectra for synthetic (3)

FIG. 30 shows ¹H and ¹³C NMR spectra for enzyme-produced (3)

FIG. 31 shows ¹H and ¹³C NMR spectra of 2,4,6-triethylbenzenesulfonamide (2)

FIG. 32 shows ¹H and ¹³C NMR spectra of 2,4,6-trimethylbenzenesulfonyl azide (4).

FIG. 33 shows ¹H and ¹³C NMR spectra of (5).

FIG. 34 shows ¹H and ¹³C NMR spectra of 2,4,6-trimethylbenzenesulfonyl azide (7)

FIGS. 35A-B show (FIG. 35A) GC-MS trace of reaction of 4.1a, and (FIG. 35B) GC-MS trace of reaction of 4.1b.

FIGS. 36A-C shows examples of carbene C—H insertion by P450s.

FIGS. 37A-B show examples of N—H insertion with H2-5-F10. Products 4.3-6 and 4.9 were analyzed with the following GC method on: 90° C. (hold 2 min), 90-190° C. (6° C./min), 190-230° C. (40° C./min) Products 4.7, 4.8, and 4.10 were analyzed with the following method: 100° C. (hold 1 min), 100-140° C. (6° C./min), 140-260° C. (20° C./min), 260° C. (hold 3 min) Elution times are as follows: 4.3 (9.8 min), 4.4 (10.1 min), 4.5 (10.7 min), 4.6 (11.6 min), 4.7 (9.9 min), 4.8 (10.4 min), 4.9 (14.2 min), and 4.10 (11.3 min) FIG. 37B shows improved yields for reaction of aniline with ethyl 2-diazopropanoate using axial mutant catalysts.

FIG. 38 shows calibration curves for N—H insertion products, with the ratio of the area under the independently-synthesized standard peaks and the area of the product peaks plotted against the concentration for each molecule

FIG. 39 shows calibration curves for N—H insertion products, with the ratio of the area under the independently-synthesized standard peaks and the area of the product peaks plotted against the concentration for each molecule.

### DETAILED DESCRIPTION OF THE INVENTION

### I. Definitions

The following definitions and abbreviations are to be used for the interpretations of the invention. The term “invention” or “present invention” as used herein is a non-limiting term and is not intended to refer to any single embodiment but encompasses all possible embodiments.

As used herein, the terms “comprises,” “comprising,” “includes,” “including,” “has,” “having, “contains,” “containing,” or any other variation thereof, are intended to cover a non-exclusive inclusion. A composition, mixture, process, method, article, or apparatus that comprises a list of elements is not necessarily limited to only those elements but may include other elements no expressly listed or inherent to such composition, mixture, process, method, article, or apparatus. Further, unless expressly stated to the contrary, “or” refers to an inclusive “or” and not to an exclusive “or.”

The term “C—H amination” includes a transfer of a nitrogen atom derived from an appropriate nitrene precursor to saturated carbon atoms with formation of a C—N bond, yielding an amine or amide, or to the transfer of nitrogen atom derived from an appropriate nitrene precursor to unsaturated carbon atoms with formation of two C—N bonds to yield an aziridine.

The term “C—H amination (enzyme) catalyst” or “enzyme with C—H amination activity” includes any and all chemical processes catalyzed by enzymes, by which substrates containing at least one carbon-hydrogen bond can be converted into amine or amide products by using nitrene precursors such as sulfonyl azides, carbonyl azides, aryl azides, azidoformates, phosphoryl azides, azide phosphonates, iminoiodanes, or haloamine derivatives.

The terms “engineered heme enzyme” and “heme enzyme variant” include any heme-containing enzyme comprising at least one amino acid mutation with respect to wild-type and also include any chimeric protein comprising recombined sequences or blocks of amino acids from two, three, or more different heme-containing enzymes that will improve its C—H amination activity or other reactions disclosed herein such as C—H, N—H, O—H and Si—H carbene insertion reactions.

The terms “engineered cytochrome P450” and “cytochrome P450 variant” include any cytochrome P450 enzyme comprising at least one amino acid mutation with respect to wild-type and also include any chimeric protein comprising recombined sequences or blocks of amino acids from two, three, or more different cytochrome P450 enzymes.

As used herein, the term “whole cell catalyst” includes microbial cells expressing heme containing enzymes, where the whole cell displays C—H amination activity and other reactions disclosed herein such as C—H, N—H, O—H and Si—H carbene insertion reactions.

As used herein, the term “carbene equivalent” or “carbene precursor” are intended to mean molecules that can be decomposed in the presence of metal (or enzyme) catalysts to structures that contain at least one divalent carbon with only 6 valence shell electrons and that can be transferred to C═C bonds to form cyclopropanes or to C—H or heteroatom-H bonds to form various carbon ligated products.

As used herein, the terms “carbene transfer” or “formal carbene transfer” are intended to mean chemical transformations where carbene equivalents are added to C═C bonds, carbon-heteroatom double bonds or inserted into C—H or heteroatom-H substrates.

As used herein, the term “nitrene equivalent” or “nitrene precursor” includes molecules that can be decomposed in the presence of metal (or enzyme) catalysts to structures that contain at least one monovalent nitrogen atom with only 6 valence shell electrons and that can be transferred to C—H to form amines, amides, or C═C bonds to form aziridines or to heteroatom-H bonds to form various nitrogen ligated products.

As used herein, the terms “nitrene transfer” or “formal nitrene transfer” includes chemical transformations where nitrene equivalents are added to C—H or C═C bonds, or carbon-heteroatom double bonds.

As used herein, the terms “porphyrin” and “metal-substituted porphyrin” denote any porphyrin that can be bound by a polypeptide with the sequence of CYP102A1 or derivatives therefrom. These porphyrins may contain metals including but not limited to Fe, Mn, Co, Rh, and Ru.

As used herein, the terms “microbial,” “microbial organism” and “microorganism” include any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. Also included are cell cultures of any species that can be cultured for the production of a chemical.

As used herein, the term “non-naturally occurring,” when used in reference to a microbial organism or enzyme activity of the invention, is intended to mean that the microbial organism or enzyme has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary non-naturally occurring microbial organism or enzyme activity includes the C—H amination as well as C—H, N—H, O—H and Si—H carbene insertion reactions.

As used herein, the term “anaerobic”, when used in reference to a reaction, culture or growth condition, is intended to mean that the concentration of oxygen is less than about 25 μM, preferably less than about 5 μM, and even more preferably less than 1 μM. The term is also intended to include sealed chambers of liquid or solid medium maintained with an atmosphere of less than about 1% oxygen. Preferably, anaerobic conditions are achieved by sparging a reaction mixture with an inert gas such as nitrogen or argon.

As used herein, the term “exogenous” is intended to mean that the referenced molecule or the referenced activity is introduced into the host microbial organism. The term as it is used in reference to expression of an encoding nucleic acid refers to the introduction of the encoding nucleic acid in an expressible form into the microbial organism. When used in reference to a biosynthetic activity, the term refers to an activity that is introduced into the host reference organism.

The term “heterologous” as used herein with reference to molecules, and in particular enzymes and polynucleotides, indicates molecules that are expressed in an organism other than the organism from which they originated or are found in nature, independently of the level of expression that can be lower, equal or higher than the level of expression of the molecule in the native microorganism.

On the other hand, the term “native” or “endogenous” as used herein with reference to molecules, and in particular enzymes and polynucleotides, indicates molecules that are expressed in the organism in which they originated or are found in nature, independently of the level of expression that can be lower equal or higher than the level of expression of the molecule in the native microorganism. It is understood that expression of native enzymes or polynucleotides may be modified in recombinant microorganisms.

The term “homolog,” as used herein with respect to an original enzyme or gene of a first family or species, refers to distinct enzymes or genes of a second family or species which are determined by functional, structural or genomic analyses to be an enzyme or gene of the second family or species which corresponds to the original enzyme or gene of the first family or species. Homologs most often have functional, structural, or genomic similarities. Techniques are known by which homologs of an enzyme or gene can readily be cloned using genetic probes and PCR. Identity of cloned sequences as homolog can be confirmed using functional assays and/or by genomic mapping of the genes.

A protein has “homology” or is “homologous” to a second protein if the amino acid sequence encoded by a gene has a similar amino acid sequence to that of the second gene. Alternatively, a protein has homology to a second protein if the two proteins have “similar” amino acid sequences. Thus, the term “homologous proteins” is intended to mean that the two proteins have similar amino acid sequences. In particular embodiments, the homology between two proteins is indicative of its shared ancestry, related by evolution.

The terms “analog” and “analogous” include nucleic acid or protein sequences or protein structures that are related to one another in function only and are not from common descent or do not share a common ancestral sequence. Analogs may differ in sequence but may share a similar structure, due to convergent evolution. For example, two enzymes are analogs or analogous if the enzymes catalyze the same reaction of conversion of a substrate to a product, are unrelated in sequence, and irrespective of whether the two enzymes are related in structure.

As used herein, the term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can refer to alkyl groups having up to 20 carbons atoms, such as, but not limited to heptyl, octyl, nonyl, decyl, etc. Alkyl groups can be optionally substituted with one or more moieties selected from halo, hydroxy, amino, alkylamino, alkoxy, haloalkyl, carboxy, amido, nitro, oxo, and cyano.

As used herein, the term“alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be optionally substituted with one or more moieties selected from halo, hydroxy, amino, alkylamino, alkoxy, haloalkyl, carboxy, amido, nitro, oxo, and cyano.

As used herein, the term “alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be optionally substituted with one or more moieties selected from halo, hydroxy, amino, alkylamino, alkoxy, haloalkyl, carboxy, amido, nitro, oxo, and cyano.

As used herein, the term “aryl” refers to an aromatic carbon ring system having any suitable number of ring atoms and any suitable number of rings. Aryl groups can include any suitable number of carbon ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be optionally substituted with one or more moieties selected from alkyl, halo, hydroxy, amino, alkylamino, alkoxy, haloalkyl, carboxy, amido, nitro, oxo, and cyano.

As used herein, the term “cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, and C₆₋₈. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2]bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. Cycloalkyl groups can be optionally substituted with one or more moieties selected from halo, hydroxy, amino, alkylamino, alkoxy, haloalkyl, carboxy, amido, nitro, oxo, and cyano.

As used herein, the term “heterocyclyl” refers to a saturated ring system having from 3 to 12 ring members and from 1 to 4 heteroatoms selected from N, O and S. Additional heteroatoms including, but not limited to, B, Al, Si and P can also be present in a heterocycloalkyl group. The heteroatoms can be oxidized to form moieties such as, but not limited to, —S(O)— and —S(O)₂—. Heterocyclyl groups can include any number of ring atoms, such as, 3 to 6, 4 to 6, 5 to 6, 4 to 6, or 4 to 7 ring members. Any suitable number of heteroatoms can be included in the heterocyclyl groups, such as 1, 2, 3, or 4, or 1 to 2, 1 to 3, 1 to 4, 2 to 3, 2 to 4, or 3 to 4. Examples of heterocyclyl groups include, but are not limited to, aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. Heterocyclyl groups can be optionally substituted with one or more moieties selected from halo, hydroxy, amino, alkylamino, alkoxy, haloalkyl, carboxy, amido, nitro, oxo, and cyano.

As used herein, the term “heteroaryl” refers to a monocyclic or fused bicyclic or tricyclic aromatic ring assembly containing 5 to 16 ring atoms, where from 1 to 5 of the ring atoms are a heteroatom such as N, O or S. Additional heteroatoms including, but not limited to, B, Al, Si and P can also be present in a heteroaryl group. The heteroatoms can be oxidized to form moieties such as, but not limited to, —S(O)— and —S(O)₂—. Heteroaryl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. Examples of heteroaryl groups include, but are not limited to, pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. Heteroaryl groups can be optionally substituted with one or more moieties selected from alkyl, halo, hydroxy, amino, alkylamino, alkoxy, haloalkyl, carboxy, amido, nitro, oxo, and cyano.

As used herein, the term “alkoxy” refers to an alkyl group having an oxygen atom that connects the alkyl group to the point of attachment: i.e., alkyl-O—. As for alkyl group, alkoxy groups can have any suitable number of carbon atoms, such as C₁₋₆ or C₁₋₄. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. Alkoxy groups can be optionally substituted with one or more moieties selected from halo, hydroxy, amino, alkylamino, alkoxy, haloalkyl, carboxy, amido, nitro, oxo, and cyano.

As used herein, the term “alkylthio” refers to an alkyl group having a sulfur atom that connects the alkyl group to the point of attachment: i.e., alkyl-S—. As for alkyl groups, alkylthio groups can have any suitable number of carbon atoms, such as C₁₋₆ or C₁₋₄. Alkylthio groups include, for example, methoxy, ethoxy, propoxy, iso-propoxy, butoxy, 2-butoxy, iso-butoxy, sec-butoxy, tert-butoxy, pentoxy, hexoxy, etc. Alkylthio groups can be optionally substituted with one or more moieties selected from halo, hydroxy, amino, alkylamino, alkoxy, haloalkyl, carboxy, amido, nitro, oxo, and cyano.

As used herein, the terms “halo” and “halogen” refer to fluorine, chlorine, bromine and iodine.

As used herein, the term “haloalkyl” refers to an alkyl moiety as defined above substituted with at least one halogen atom.

As used herein, the term “alkylsilyl” refers to a moiety —SiR₃, wherein at least one R group is alkyl and the other R groups are H or alkyl. The alkyl groups can be substituted with one more halogen atoms.

As used herein, the term “acyl” refers to a moiety —C(O)R, wherein R is an alkyl group.

As used herein, the term “oxo” refers to an oxygen atom that is double-bonded to a compound (i.e., O═).

As used herein, the term “carboxy” refers to a moiety —C(O)OH. The carboxy moiety can be ionized to form the carboxylate anion.

As used herein, the term “amino” refers to a moiety —NR₃, wherein each R group is H or alkyl.

As used herein, the term “amido” refers to a moiety —NRC(O)R or —C(O)NR₂, wherein each R group is H or alkyl.

### II. Introduction

The present invention is based on the surprising discovery that engineered heme enzymes such as cytochrome P450_{BM3} enzymes, including a serine-heme-ligated P411 enzyme, efficiently catalyze carbene and nitrene insertion and transfer reactions. Suitable reactions include, but are not limited to, carbene insertion reactions into N—H, C—H, O—H or Si—H bonds, as well as nitrene transfer into C═C and C—H bonds. For example, in certain aspects, the present invention provides engineered heme enzymes such as cytochrome P450_{BM3} enzymes, including the serine-heme-ligated ‘P411’, which efficiently catalyze the intramolecular amination of benzylic C—H bonds in arylsulfonyl azides to form benzosultams. Significant enhancements in catalytic activity and enantioselectivity were observed in vivo, using intact bacterial cells expressing the engineered enzymes. The results presented here underscore the utility of natural enzymes in catalyzing new reaction types with the aid of synthetic reagents. The ability to genetically encode catalysts for formal nitrene transfers opens up new biosynthetic pathways to amines and expands the scope of transformations accessible to biocatalysis.

### III. Description of the Embodiments

In one embodiment, the present invention provides a method for catalyzing a carbene insertion into a N—H bond to produce a product having a new C—N bond. The method comprises the steps of:
providing a N—H containing substrate, a diazo carbene precursor and an engineered heme enzyme; and
allowing the reaction to proceed for a time sufficient to form a product having a new C—N bond. Although throughout each of the embodiments described herein, an engineered heme enzyme is preferred, a non-engineered heme enzyme may catalyze a reaction described herein.

In some embodiments, the engineered heme enzyme is a cytochrome P450 enzyme or a variant thereof. In some embodiments, the heme enzyme variant comprises a mutation at the axial position of the heme coordination site. In some instances, the mutation is an amino acid substitution of the naturally occurring residue at this position with Ala, Asp, Arg, Asn, Cys, Glu, Gln, Gly, His, Ile, Lys, Leu, Met, Phe, Pro, Ser, Thr, Trp, Tyr or Val at the axial position. In other instances, the mutation is an amino acid substitution of Cys with Asp or Ser at the axial position.

In some embodiments, the engineered heme enzyme is expressed in a bacterial, archaeal or fungal host organism.

In some embodiments, the cytochrome P450 enzyme is a P450 BM3 enzyme or a variant thereof. In some instances, the P450 BM3 enzyme comprises the amino acid sequence set forth in SEQ ID NO:1 or a variant thereof.

In some embodiments, the P450 enzyme variant comprises a mutation at the axial position of the heme coordination site. In some instances, the mutation is an amino acid substitution of Cys with Ala, Asp, Arg, Asn, Glu, Gln, Gly, His, Ile, Lys, Leu, Met, Phe, Pro, Ser, Thr, Trp, Tyr or Val at the axial position. In other instances, the mutation is an amino acid substitution of Cys with Asp or Ser at the axial position.

In some embodiments, the P450 BM3 enzyme comprises at least one, two, three, four, five, six, seven, eight, nine, ten, eleven, twelve, or all thirteen of the following amino acid substitutions in SEQ ID NO: 1: V78A, F87V, P142S, T175I, A184V, S226R, H236Q, E252G, T268A, A290V, L353V, I366V, and E442K.

In some embodiments, the cytochrome P450 BM3 enzyme variant comprises a T268A mutation and/or a C400X mutation in SEQ ID NO:1, wherein X is any amino acid other than Cys. In another embodiment, the cytochrome P450 BM3 enzyme variant comprises a T438S mutation and/or a C400X mutation in SEQ ID NO:1, wherein X is any amino acid other than Cys.

In some embodiments, the heme enzyme variant comprises a fragment of the cytochrome P450 enzyme or variant thereof. In some embodiments, the heme enzyme variant is a cytochrome P450 BM3 enzyme variant selected from Table 4, Table 5, Table 6 and Table 9.

In one embodiment, the heme enzyme variant for use in the catalysis of a carbene insertion into a N—H bond to produce a product having a new C—N bond is a P450 BM3 variant comprising the following amino acid substitutions to SEQ ID NO:1: V78A, F87V, P142S, T175I, A184V, S226R, H236Q, E252G, T268A, A290V, L353V, I366V, and E442K. In another embodiment, the heme variant optionally comprises the following additional amino acid substitutions to SEQ ID NO:1: L75A, I263A and L437A. In yet another embodiment, the heme variant optionally comprises the additional amino acid substitution C400S to SEQ ID NO:1. In some embodiments, the heme enzyme variant is the H2-5-F10 variant (see, Table 7). In other embodiments, the heme enzyme variant is the P411-CIS variant (see, Table 4).

In another embodiment, the present invention provides a method for catalyzing a carbene insertion into a C—H bond to produce a product with a new C—C bond. The method comprises the steps of:
providing a C—H containing substrate, a diazo carbene precursor and an engineered heme enzyme; and
allowing the reaction to proceed for a time sufficient to form a product having a new C—C bond.

In some embodiments, the engineered heme enzyme is a cytochrome P450 enzyme or a variant thereof.

In some embodiments, the engineered heme enzyme is expressed in a bacterial, archaeal or fungal host organism.

In some embodiments, the cytochrome P450 enzyme is a P450 BM3 enzyme or a variant thereof. In some instances, the P450 BM3 enzyme comprises the amino acid sequence set forth in SEQ ID NO:1 or a variant thereof.

In some embodiments, the P450 enzyme variant comprises a mutation at the axial position of the heme coordination site. In some instances, the mutation is an amino acid substitution of Cys with Ala, Asp, Arg, Asn, Glu, Gln, Gly, His, Ile, Lys, Leu, Met, Phe, Pro, Ser, Thr, Trp, Tyr or Val at the axial position. In other instances, the mutation is an amino acid substitution of Cys with Asp or Ser at the axial position.

In some embodiments, the P450 BM3 enzyme comprises at least one, two, three, four, five, six, seven, eight, nine, ten, eleven, twelve, or all thirteen of the following amino acid substitutions in SEQ ID NO: 1: V78A, F87V, P142S, T175I, A184V, S226R, H236Q, E252G, T268A, A290V, L353V, I366V, and E442K.

In some embodiments, the cytochrome P450 BM3 enzyme variant comprises a T268A mutation and/or a C400X mutation in SEQ ID NO:1, wherein X is any amino acid other than Cys. In another embodiment, the cytochrome P450 BM3 enzyme variant comprises a T438S mutation and/or a C400X mutation in SEQ ID NO:1, wherein X is any amino acid other than Cys.

In some embodiments, the heme enzyme variant comprises a fragment of the cytochrome P450 enzyme or variant thereof. In some embodiments, the heme enzyme variant is a cytochrome P450 BM3 enzyme variant selected from Table 4, Table 5, Table 6 and Table 9. In some embodiments, the enzyme variant is WT-C400D.

In one embodiment, the heme enzyme variant for use in the catalysis of a carbene insertion into a C—H bond to produce a product with a new C—C bond is a P450 BM3 variant comprising the wild-type heme domain of cytochrome P450 BM3 (e.g., amino acids 1-463 of SEQ ID NO:1) and the amino acid substitution C400D.

In another embodiment, the present invention provides a method for catalyzing a nitrene insertion reaction into an olefin to produce an aziridine, the method comprises the steps of:
providing an olefin substrate, a nitrene precursor and an engineered heme enzyme; and
allowing the reaction to proceed for a time sufficient to produce an aziridine.

In some embodiments, the engineered heme enzyme is a cytochrome P450 enzyme or a variant thereof.

In some embodiments, the engineered heme enzyme is expressed in a bacterial, archaeal or fungal host organism.

In some embodiments, the cytochrome P450 enzyme is a P450 BM3 enzyme or a variant thereof. In some instances, the P450 BM3 enzyme comprises the amino acid sequence set forth in SEQ ID NO:1 or a variant thereof.

In some embodiments, the P450 enzyme variant comprises a mutation at the axial position of the heme coordination site. In some instances, the mutation is an amino acid substitution of Cys with Ala, Asp, Arg, Asn, Glu, Gln, Gly, His, Ile, Lys, Leu, Met, Phe, Pro, Ser, Thr, Trp, Tyr or Val at the axial position. In other instances, the mutation is an amino acid substitution of Cys with Asp or Ser at the axial position.

In some embodiments, the P450 BM3 enzyme comprises at least one, two, three, four, five, six, seven, eight, nine, ten, eleven, twelve, or all thirteen of the following amino acid substitutions in SEQ ID NO: 1: V78A, F87V, P142S, T175I, A184V, S226R, H236Q, E252G, T268A, A290V, L353V, I366V, and E442K.

In some embodiments, the cytochrome P450 BM3 enzyme variant comprises a T268A mutation and/or a C400X mutation in SEQ ID NO:1, wherein X is any amino acid other than Cys. In another embodiment, the cytochrome P450 BM3 enzyme variant comprises a T438S mutation and/or a C400X mutation in SEQ ID NO:1, wherein X is any amino acid other than Cys.

In some embodiments, the heme enzyme variant comprises a fragment of the cytochrome P450 enzyme or variant thereof. In some embodiments, the heme enzyme variant is a cytochrome P450 BM3 enzyme variant selected from Table 4, Table 5, Table 6 and Table 9.

In yet another embodiment, the present invention provides a method for catalyzing a nitrene insertion into a C—H bond to produce a product having a new C—N bond. The method comprises the steps of:
providing a C—H containing substrate, a nitrene precursor and an engineered heme enzyme; and
allowing the reaction to proceed for a time sufficient to form a product having a new C—N bond.

In some embodiments, the engineered heme enzyme is a cytochrome P450 enzyme or a variant thereof.

In some embodiments, the engineered heme enzyme is expressed in a bacterial, archaeal or fungal host organism.

In some embodiments, the cytochrome P450 enzyme is a P450 BM3 enzyme or a variant thereof. In some instances, the P450 BM3 enzyme comprises the amino acid sequence set forth in SEQ ID NO:1 or a variant thereof.

In some embodiments, the P450 enzyme variant comprises a mutation at the axial position of the heme coordination site. In some instances, the mutation is an amino acid substitution of Cys with Ala, Asp, Arg, Asn, Glu, Gln, Gly, His, Ile, Lys, Leu, Met, Phe, Pro, Ser, Thr, Trp, Tyr or Val at the axial position. In other instances, the mutation is an amino acid substitution of Cys with Asp or Ser at the axial position.

In some embodiments, the P450 BM3 enzyme comprises at least one, two, three, four, five, six, seven, eight, nine, ten, eleven, twelve, or all thirteen of the following amino acid substitutions in SEQ ID NO: 1: V78A, F87V, P142S, T175I, A184V, S226R, H236Q, E252G, T268A, A290V, L353V, I366V, and E442K.

In some embodiments, the cytochrome P450 BM3 enzyme variant comprises a T268A mutation and/or a C400X mutation in SEQ ID NO:1, wherein X is any amino acid other than Cys. In another embodiment, the cytochrome P450 BM3 enzyme variant comprises a T438S mutation and/or a C400X mutation in SEQ ID NO:1, wherein X is any amino acid other than Cys.

In some embodiments, the heme enzyme variant comprises a fragment of the cytochrome P450 enzyme or variant thereof. In some embodiments, the heme enzyme variant is a cytochrome P450 BM3 enzyme variant selected from Table 4, Table 5, Table 6 and Table 9.

In yet another embodiment, the present invention provides a method for catalyzing a carbene insertion into a O—H bond to produce a product having a new C—O bond. The method comprises the steps of:
providing a O—H containing substrate, a diazo carbene precursor and an engineered heme enzyme; and
allowing the reaction to proceed for a time sufficient to form a product having a new C—O bond.

In some embodiments, the engineered heme enzyme is a cytochrome P450 enzyme or a variant thereof.

In some embodiments, the engineered heme enzyme is expressed in a bacterial, archaeal or fungal host organism.

In some embodiments, the cytochrome P450 enzyme is a P450 BM3 enzyme or a variant thereof. In some instances, the P450 BM3 enzyme comprises the amino acid sequence set forth in SEQ ID NO:1 or a variant thereof.

In some embodiments, the P450 enzyme variant comprises a mutation at the axial position of the heme coordination site. In some instances, the mutation is an amino acid substitution of Cys with Ala, Asp, Arg, Asn, Glu, Gln, Gly, His, Ile, Lys, Leu, Met, Phe, Pro, Ser, Thr, Trp, Tyr or Val at the axial position. In other instances, the mutation is an amino acid substitution of Cys with Asp or Ser at the axial position.

In some embodiments, the P450 BM3 enzyme comprises at least one, two, three, four, five, six, seven, eight, nine, ten, eleven, twelve, or all thirteen of the following amino acid substitutions in SEQ ID NO: 1: V78A, F87V, P142S, T175I, A184V, S226R, H236Q, E252G, T268A, A290V, L353V, I366V, and E442K.

In some embodiments, the cytochrome P450 BM3 enzyme variant comprises a T268A mutation and/or a C400X mutation in SEQ ID NO:1, wherein X is any amino acid other than Cys. In another embodiment, the cytochrome P450 BM3 enzyme variant comprises a T438S mutation and/or a C400X mutation in SEQ ID NO:1, wherein X is any amino acid other than Cys.

In some embodiments, the heme enzyme variant comprises a fragment of the cytochrome P450 enzyme or variant thereof. In some embodiments, the heme enzyme variant is a cytochrome P450 BM3 enzyme variant selected from Table 4, Table 5, Table 6 and Table 9.

In another embodiment, the present invention provides a method for catalyzing a carbene insertion into a Si—H bond to produce a product having a new C—Si bond. The method comprises the steps of:
providing a Si—H containing substrate, a diazo carbene precursor and an engineered heme enzyme; and
allowing the reaction to proceed for a time sufficient to form a product having a new C—Si bond.

In some embodiments, the engineered heme enzyme is a cytochrome P450 enzyme or a variant thereof.

In some embodiments, the engineered heme enzyme is expressed in a bacterial, archaeal or fungal host organism.

In some embodiments, the cytochrome P450 enzyme is a P450 BM3 enzyme or a variant thereof. In some instances, the P450 BM3 enzyme comprises the amino acid sequence set forth in SEQ ID NO:1 or a variant thereof.

In some embodiments, the P450 enzyme variant comprises a mutation at the axial position of the heme coordination site. In some instances, the mutation is an amino acid substitution of Cys with Ala, Asp, Arg, Asn, Glu, Gln, Gly, His, Ile, Lys, Leu, Met, Phe, Pro, Ser, Thr, Trp, Tyr or Val at the axial position. In other instances, the mutation is an amino acid substitution of Cys with Asp or Ser at the axial position.

In some embodiments, the P450 BM3 enzyme comprises at least one, two, three, four, five, six, seven, eight, nine, ten, eleven, twelve, or all thirteen of the following amino acid substitutions in SEQ ID NO: 1: V78A, F87V, P142S, T175I, A184V, S226R, H236Q, E252G, T268A, A290V, L353V, I366V, and E442K.

In some embodiments, the cytochrome P450 BM3 enzyme variant comprises a T268A mutation and/or a C400X mutation in SEQ ID NO:1, wherein X is any amino acid other than Cys. In another embodiment, the cytochrome P450 BM3 enzyme variant comprises a T438S mutation and/or a C400X mutation in SEQ ID NO:1, wherein X is any amino acid other than Cys.

In some embodiments, the heme enzyme variant comprises a fragment of the cytochrome P450 enzyme or variant thereof. In some embodiments, the heme enzyme variant is a cytochrome P450 BM3 enzyme variant selected from Table 4, Table 5, Table 6 and Table 9.

In another embodiment, the present invention provides a heme enzyme variant or fragment thereof that can catalyze a nitrene insertion reaction into an olefin to produce an aziridine.

In some embodiments, the engineered heme enzyme is a cytochrome P450 enzyme or a variant thereof.

In some embodiments, the engineered heme enzyme is expressed in a bacterial, archaeal or fungal host organism.

In some embodiments, the cytochrome P450 enzyme is a P450 BM3 enzyme or a variant thereof. In some instances, the P450 BM3 enzyme comprises the amino acid sequence set forth in SEQ ID NO:1 or a variant thereof.

In some embodiments, the P450 enzyme variant comprises a mutation at the axial position of the heme coordination site. In some instances, the mutation is an amino acid substitution of Cys with Ala, Asp, Arg, Asn, Glu, Gln, Gly, His, Ile, Lys, Leu, Met, Phe, Pro, Ser, Thr, Trp, Tyr or Val at the axial position. In other instances, the mutation is an amino acid substitution of Cys with Asp or Ser at the axial position.

In some embodiments, the P450 BM3 enzyme comprises at least one, two, three, four, five, six, seven, eight, nine, ten, eleven, twelve, or all thirteen of the following amino acid substitutions in SEQ ID NO: 1: V78A, F87V, P142S, T175I, A184V, S226R, H236Q, E252G, T268A, A290V, L353V, I366V, and E442K.

In some embodiments, the cytochrome P450 BM3 enzyme variant comprises a T268A mutation and/or a C400X mutation in SEQ ID NO:1, wherein X is any amino acid other than Cys. In another embodiment, the cytochrome P450 BM3 enzyme variant comprises a T438S mutation and/or a C400X mutation in SEQ ID NO:1, wherein X is any amino acid other than Cys.

In some embodiments, the heme enzyme variant comprises a fragment of the cytochrome P450 enzyme or variant thereof. In some embodiments, the heme enzyme variant is a cytochrome P450 BM3 enzyme variant selected from Table 4, Table 5, Table 6 and Table 9.

In one embodiment, the present invention provides a heme enzyme variant or fragment thereof that can catalyze a carbene insertion into a N—H bond, C—H bond, O—H bond, and/or Si—H bond and/or catalyze a nitrene insertion into a C═C bond and/or C—H bond.

In some embodiments, the heme enzyme variant is isolated and/or purified. In some instances, the heme enzyme variant is in lyophilized form.

In some embodiments, the heme enzyme variant is a cytochrome P450 enzyme or a variant thereof.

In some embodiments, the cytochrome P450 enzyme is a P450 BM3 enzyme or a variant thereof. In some instances, the P450 BM3 enzyme comprises the amino acid sequence set forth in SEQ ID NO:1 or a variant thereof.

In some embodiments, the P450 enzyme variant comprises a mutation at the axial position of the heme coordination site. In some instances, the mutation is an amino acid substitution of Cys with Ala, Asp, Arg, Asn, Glu, Gln, Gly, His, Ile, Lys, Leu, Met, Phe, Pro, Ser, Thr, Trp, Tyr or Val at the axial position. In other instances, the mutation is an amino acid substitution of Cys with Asp or Ser at the axial position.

In some embodiments, the P450 BM3 enzyme comprises at least one, two, three, four, five, six, seven, eight, nine, ten, eleven, twelve, or all thirteen of the following amino acid substitutions in SEQ ID NO: 1: V78A, F87V, P142S, T175I, A184V, S226R, H236Q, E252G, T268A, A290V, L353V, I366V, and E442K.

In some embodiments, the cytochrome P450 BM3 enzyme variant comprises a T268A mutation and/or a C400X mutation in SEQ ID NO:1, wherein X is any amino acid other than Cys. In another embodiment, the cytochrome P450 BM3 enzyme variant comprises a T438S mutation and/or a C400X mutation in SEQ ID NO:1, wherein X is any amino acid other than Cys.

In some embodiments, the heme enzyme variant comprises a fragment of the cytochrome P450 enzyme or variant thereof. In some embodiments, the heme enzyme variant is a cytochrome P450 BM3 enzyme variant selected from Table 4, Table 5, Table 6 and Table 9.

In some embodiments, the heme enzyme variant has a higher total turnover number (TTN) compared to the wild-type sequence.

In one embodiment, provided herein is a cell expressing the heme enzyme variant as described herein. In instances, the cell is a bacterial cell or a yeast cell.

In another embodiment, provided herein is an expression vector comprising a nucleic acid sequence encoding a heme enzyme variant described herein.

In yet another embodiment, provided herein is a cell comprising the expression vector described herein. In some instances, the cell is a bacterial cell or a yeast cell.

### IV. Heme Enzymes

In certain aspects, the present invention provides compositions comprising one or more heme enzymes that catalyze the conversion of an olefinic substrate to products containing one or more cyclopropane functional groups. In particular embodiments, the present invention provides heme enzyme variants comprising at least one or more amino acid mutations therein that catalyze nitrine C—H insertion, intramolecular or intramolecular C—H amination, and/or C═C aziridination, making products described herein with high stereoselectivity. In preferred embodiments, the heme enzyme variants of the present invention have the ability to catalyze carbene insertion and nitrene transfer reactions efficiently, display increased total turnover numbers, and/or demonstrate highly regio- and/or enantioselective product formation compared to the corresponding wild-type enzymes.

The terms “heme enzyme” and “heme protein” are used herein to include any member of a group of proteins containing heme as a prosthetic group. Non-limiting examples of heme enzymes include globins, cytochromes, oxidoreductases, any other protein containing a heme as a prosthetic group, and combinations thereof. Heme-containing globins include, but are not limited to, hemoglobin, myoglobin, and combinations thereof. Heme-containing cytochromes include, but are not limited to, cytochrome P450, cytochrome b, cytochrome c1, cytochrome c, and combinations thereof. Heme-containing oxidoreductases include, but are not limited to, a catalase, an oxidase, an oxygenase, a haloperoxidase, a peroxidase, and combinations thereof.

In certain instances, the heme enzymes are metal-substituted heme enzymes containing protoporphyrin IX or other porphyrin molecules containing metals other than iron, including, but not limited to, cobalt, rhodium, copper, ruthenium, and manganese, which are active cyclopropanation catalysts.

In some embodiments, the heme enzyme is a member of one of the enzyme classes set forth in Table 1. In other embodiments, the heme enzyme is a variant or homolog of a member of one of the enzyme classes set forth in Table 1. In yet other embodiments, the heme enzyme comprises or consists of the heme domain of a member of one of the enzyme classes set forth in Table 1 or a fragment thereof (e.g., a truncated heme domain) that is capable of carrying out the carbene insertion and nitrene transfer reactions described herein.

TABLE 1
Heme enzymes identified by their enzyme classification number
(EC number) and classification name.

| EC Number | Name |
|---|---|
| 1.1.2.3 | L-lactate dehydrogenase |
| 1.1.2.6 | polyvinyl alcohol dehydrogenase (cytochrome) |
| 1.1.2.7 | methanol dehydrogenase (cytochrome c) |
| 1.1.5.5 | alcohol dehydrogenase (quinone) |
| 1.1.5.6 | formate dehydrogenase-N: |
| 1.1.9.1 | alcohol dehydrogenase (azurin): |
| 1.1.99.3 | gluconate 2-dehydrogenase (acceptor) |
| 1.1.99.11 | fructose 5-dehydrogenase |
| 1.1.99.18 | cellobiose dehydrogenase (acceptor) |
| 1.1.99.20 | alkan-1-ol dehydrogenase (acceptor) |
| 1.2.1.70 | glutamyl-tRNA reductase |
| 1.2.3.7 | indole-3-acetaldehyde oxidase |
| 1.2.99.3 | aldehyde dehydrogenase (pyrroloquinoline-quinone) |
| 1.3.1.6 | fumarate reductase (NADH): |
| 1.3.5.1 | succinate dehydrogenase (ubiquinone) |
| 1.3.5.4 | fumarate reductase (menaquinone) |
| 1.3.99.1 | succinate dehydrogenase |
| 1.4.9.1 | methylamine dehydrogenase (amicyanin) |
| 1.4.9.2. | aralkylamine dehydrogenase (azurin) |
| 1.5.1.20 | methylenetetrahydrofolate reductase [NAD(P)H] |
| 1.5.99.6 | spermidine dehydrogenase |
| 1.6.3.1 | NAD(P)H oxidase |
| 1.7.1.1 | nitrate reductase (NADH) |
| 1.7.1.2 | Nitrate reductase [NAD(P)H] |
| 1.7.1.3 | nitrate reductase (NADPH) |
| 1.7.1.4 | nitrite reductase [NAD(P)H] |
| 1.7.1.14 | nitric oxide reductase [NAD(P), nitrous oxide-forming] |
| 1.7.2.1 | nitrite reductase (NO-forming) |
| 1.1.2.2 | nitrite reductase (cytochrome; ammonia-forming) |
| 1.7.2.3 | trimethylamine-N-oxide reductase (cytochrome c) |
| 1.7.2.5 | nitric oxide reductase (cytochrome c) |
| 1.7.2.6 | hydroxylamine dehydrogenase |
| 1.7.3.6 | hydroxylamine oxidase (cytochrome) |
| 1.7.5.1 | nitrate reductase (quinone) |
| 1.7.5.2 | nitric oxide reductase (menaquinol) |
| 1.7.6.1 | nitrite dismutase |
| 1.7.7.1 | ferredoxin-nitrite reductase |
| 1.7.7.2 | ferredoxin-nitrate reductase |
| 1.7.99.4 | nitrate reductase |
| 1.7.99.8 | hydrazine oxidoreductase |
| 1.8.1.2 | sulfite reductase (NADPH) |
| 1.8.2.1 | sulfite dehydrogenase |
| 1.8.2.2 | thiosulfate dehydrogenase |
| 1.8.2.3 | sulfide-cytochrome-c reductase (flavocytochrome c) |
| 1.8.2.4 | dimethyl sulfide:cytochrome c2 reductase |
| 1.8.3.1 | sulfite oxidase |
| 1.8.7.1 | sulfite reductase (ferredoxin) |
| 1.8.98.1 | CoB-CoM heterodisulfide reductase |
| 1.8.99.1 | sulfite reductase |
| 1.8.99.2 | adenyly 1-sulfate reductase |
| 1.8.99.3 | hydrogensulfite reductase |
| 1.9.3.1 | cytochrome-c oxidase |
| 1.9.6.1 | nitrate reductase (cytochrome) |
| 1.10.2.2 | ubiquinol-cytochrome-c reductase |
| 1.10.3.1 | catechol oxidase |
| 1.10.3.B1 | caldariellaquinol oxidase (H+-transporting) |
| 1.10.3.3 | L-ascorbate oxidase |
| 1.10.3.9 | photosystem II |
| 1.10.3.10 | ubiquinol oxidase (H+-transporting) |
| 1.10.3.11 | ubiquinol oxidase |
| 1.10.3.12 | menaquinol oxidase (H+-transporting) |
| 1.10.9.1 | plastoquinol-plastocyanin reductase |
| 1.11.1.5 | cytochrome-c peroxidase |
| 1.11.1.6 | catalase |
| 1.11.1.7 | peroxidase |
| 1.11.1.B2 | chloride peroxidase (vanadium-containing) |
| 1.11.1.B7 | bromide peroxidase (heme-containing) |
| 1.11.1.8 | iodide peroxidase |
| 1.11.1.10 | chloride peroxidase |
| 1.11.1.11 | L-ascorbate peroxidase |
| 1.11.1.13 | manganese peroxidase |
| 1.11.1.14 | lignin peroxidase |
| 1.11.1.16 | versatile peroxidase |
| 1.11.1.19 | dye decolorizing peroxidase |
| 1.11.1.21 | catalase-peroxidase |
| 1.11.2.1 | unspecific peroxygenase |
| 1.11.2.2 | myeloperoxidase |
| 1.11.2.3 | plant seed peroxygenase |
| 1.11.2.4 | fatty-acid peroxygenase |
| 1.12.2.1 | cytochrome-c3 hydrogenase |
| 1.12.5.1 | hydrogen:quinone oxidoreductase |
| 1.12.99.6 | hydrogenase (acceptor) |
| 1.13.11.9 | 2,5-dihydroxypyridine 5,6-dioxygenase |
| 1.13.11.11 | tryptophan 2,3-dioxygenase |
| 1.13.11.49 | chlorite O2-lyase |
| 1.13.11.50 | acetylacetone-cleaving enzyme |
| 1.13.11.52 | indoleamine 2,3-dioxygenase |
| 1.13.11.60 | linoleate 8R-lipoxygenase |
| 1.13.99.3 | tryptophan 2′-dioxygenase |
| 1.14.11.9 | flavanone 3-dioxygenase |
| 1.14.12.17 | nitric oxide dioxygenase |
| 1.14.13.39 | nitric-oxide synthase (NADPH dependent) |
| 1.14.13.17 | cholesterol 7alpha-monooxygenase |
| 1.14.13.41 | tyrosine N-monooxygenase |
| 1.14.13.70 | sterol 14alpha-demethylase |
| 1.14.13.71 | N-methylcoclaurine 3′-monooxygenase |
| 1.14.13.81 | magnesium-protoporphyrin IX monomethyl ester |
| | (oxidative) cyclase |
| 1.14.13.86 | 2-hydroxyisoflavanone synthase |
| 1.14.13.98 | cholesterol 24-hydroxylase |
| 1.14.13.119 | 5-epiaristolochene 1,3-dihydroxylase |
| 1.14.13.126 | vitamin D3 24-hydroxylase |
| 1.14.13.129 | beta-carotene 3-hydroxylase |
| 1.14.13.141 | cholest-4-en-3-one 26-monooxygenase |
| 1.14.13.142 | 3-ketosteroid 9alpha-monooxygenase |
| 1.14.13.151 | linalool 8-monooxygenase |
| 1.14.13.156 | 1,8-cineole 2-endo-monooxygenase |
| 1.14.13.159 | vitamin D 25-hydroxylase |
| 1.14.14.1 | unspecific monooxygenase |
| 1.14.15.1 | camphor 5-monooxygenase |
| 1.14.15.6 | cholesterol monooxygenase (side-chain-cleaving) |
| 1.14.15.8 | steroid 15beta-monooxygenase |
| 1.14.15.9 | spheroidene monooxygenase |
| 1.14.18.1 | tyrosinase |
| 1.14.19.1 | stearoyl-CoA 9-desaturase |
| 1.14.19.3 | linoleoyl-CoA desaturase |
| 1.14.21.7 | biflaviolin synthase |
| 1.14.99.1 | prostaglandin-endoperoxide synthase |
| 1.14.99.3 | heme oxygenase |
| 1.14.99.9 | steroid 17alpha-monooxygenase |
| 1.14.99.10 | steroid 21-monooxygenase |
| 1.14.99.15 | 4-methoxybenzoate monooxygenase (O-demethylating) |
| 1.14.99.45 | carotene epsilon-monooxygenase |
| 1.16.5.1 | ascorbate ferrireductase (transmembrane) |
| 1.16.9.1 | iron:rusticyanin reductase |
| 1.17.1.4 | xanthine dehydrogenase |
| 1.17.2.2 | lupanine 17-hydroxylase (cytochrome c) |
| 1.17.99.1 | 4-methylphenol dehydrogenase (hydroxylating) |
| 1.17.99.2 | ethylbenzene hydroxylase |
| 1.97.1.1 | chlorate reductase |
| 1.97.1.9 | selenate reductase |
| 2.7.7.65 | diguanylate cyclase |
| 2.7.13.3 | histidine kinase |
| 3.1.4.52 | cyclic-guanylate-specific phosphodiesterase |
| 4.2.1.B9 | colneleic acid/etheroleic acid synthase |
| 4.2.1.22 | Cystathionine beta-synthase |
| 4.2.1.92 | hydroperoxide dehydratase |
| 4.2.1.212 | colneleate synthase |
| 4.3.1.26 | chromopyrrolate synthase |
| 4.6.1.2 | guanylate cyclase |
| 4.99.1.3 | sirohydrochlorin cobaltochelatase |
| 4.99.1.5 | aliphatic aldoxime dehydratase |
| 4.99.1.7 | phenylacetaldoxime dehydratase |
| 5.3.99.3 | prostaglandin-E synthase |
| 5.3.99.4 | prostaglandin-I synthase |
| 5.3.99.5 | Thromboxane-A synthase |
| 5.4.4.5 | 9,12-octadecadienoate 8-hydroperoxide 8R-isomerase |
| 5.4.4.6 | 9,12-octadecadienoate 8-hydroperoxide 8S-isomerase |
| 6.6.1.2 | cobaltochelatase |

In particular embodiments, the heme enzyme is a variant or a fragment thereof (e.g., a truncated variant containing the heme domain) comprising at least one mutation such as, e.g., a mutation at the axial position of the heme coordination site. In some instances, the mutation is a substitution of the native residue with Ala, Asp, Arg, Asn, Cys, Glu, Gln, Gly, His, Ile, Lys, Leu, Met, Phe, Pro, Ser, Thr, Trp, Tyr, or Val at the axial position. In certain instances, the mutation is a substitution of Cys with any other amino acid such as Ser at the axial position.

In certain embodiments, the in vitro methods for producing a product described herein comprise providing a heme enzyme, variant, or homolog thereof with a reducing agent such as NADPH or a dithionite salt (e.g., Na₂S₂O₄). In certain other embodiments, the in vivo methods for producing a reaction product provided herein comprise providing whole cells such as E. coli cells expressing a heme enzyme, variant, or homolog thereof.

In some embodiments, the heme enzyme, variant, or homolog thereof is recombinantly expressed and optionally isolated and/or purified for carrying out the in vitro cyclopropanation reactions of the present invention. In other embodiments, the heme enzyme, variant, or homolog thereof is expressed in whole cells such as E. coli cells, and these cells are used for carrying out the in vivo carbene insertion activity and/or nitrene transfer activity of the present invention.

In certain embodiments, the heme enzyme, variant, or homolog thereof comprises or consists of the same number of amino acid residues as the wild-type enzyme (i.e., a full-length polypeptide). In some instances, the heme enzyme, variant, or homolog thereof comprises or consists of an amino acid sequence without the start methionine (e.g., P450 BM3 amino acid sequence set forth in SEQ ID NO:1). In other embodiments, the heme enzyme comprises or consists of a heme domain fused to a reductase domain. In yet other embodiments, the heme enzyme does not contain a reductase domain, e.g., the heme enzyme contains a heme domain only or a fragment thereof such as a truncated heme domain.

In some embodiments, the heme enzyme, variant, or homolog thereof has an enhanced carbene insertion activity and/or nitrene transfer activity of about 1.5, 2, 3, 4, 5, 6, 7, 8, 9, 10, 15, 20, 25, 30, 35, 40, 45, 50, 55, 60, 65, 70, 75, 80, 85, 90, 95, or 100 fold compared to the corresponding wild-type heme enzyme.

In some embodiments, the heme enzyme comprises a heme domain fused to a reductase domain. In other embodiments, the heme enzyme does not comprise a reductase domain, e.g., a heme domain only or a fragment thereof.

In particular embodiments, the heme enzyme comprises a cyctochrome P450 enzyme. Cytochrome P450 enzymes constitute a large superfamily of heme-thiolate proteins involved in the metabolism of a wide variety of both exogenous and endogenous compounds.

Usually, they act as the terminal oxidase in multicomponent electron transfer chains, such as P450-containing monooxygenase systems. Members of the cytochrome P450 enzyme family catalyze myriad oxidative transformations, including, e.g., hydroxylation, epoxidation, oxidative ring coupling, heteratom release, and heteroatom oxygenation (E. M. Isin et al., Biochim. Biophys. Acta 1770, 314 (2007)). The active site of these enzymes contains an Fe^{III}-protoporphyrin IX cofactor (heme) ligated proximally by a conserved cysteine thiolate (M. T. Green, Current Opinion in Chemical Biology 13, 84 (2009)). The remaining axial iron coordination site is occupied by a water molecule in the resting enzyme, but during native catalysis, this site is capable of binding molecular oxygen. In the presence of an electron source, typically provided by NADH or NADPH from an adjacent fused reductase domain or an accessory cytochrome P450 reductase enzyme, the heme center of cytochrome P450 activates molecular oxygen, generating a high valent iron(IV)-oxo porphyrin cation radical species intermediate and a molecule of water.

One skilled in the art will appreciate that the cytochrome P450 superfamily of enzymes has been compiled in various databases, including, but not limited to, the cytochrome P450 homepage (available at http://drnelson.uthsc.edu/CytochromeP450.html; see also, D. R. Nelson, Hum. Genomics 4, 59 (2009)), the cytochrome P450 enzyme engineering database (available at http://www.cyped.uni-stuttgart.de/cgi-bin/CYPED5/index.pl; see also, D. Sirim et al., BMC Biochem 10, 27 (2009)), and the SuperCyp database (available at http://bioinformatics.charite.de/supercyp/; see also, S. Preissner et al., Nucleic Acids Res. 38, D237 (2010)), the disclosures of which are incorporated herein by reference in their entirety for all purposes.

In certain embodiments, the cytochrome P450 enzymes of the invention are members of one of the classes shown in Table 2 (see, http://www.icgeb.org/˜p450srv/P450enzymes.html, the disclosure of which is incorporated herein by reference in its entirety for all purposes).

TABLE 2
Heme enzymes identified by their enzyme classification number
(EC number) and classification name.

| EC | Recommended name | Family/gene |
|---|---|---|
| 1.3.3.9 | secologanin synthase | CYP72A1 |
| 1.14.13.11 | trans-cinnamate 4-monooxygenase | CYP73 |
| 1.14.13.12 | benzoate 4-monooxygenase | CYP53 |
| 1.14.13.13 | calcidiol 1-monooxygenase | CYP27 |
| 1.14.13.15 | cholestanetriol 26-monooxygenase | CYP27 |
| 1.14.13.17 | cholesterol 7α-monooxygenase | CYP7 |
| 1.14.13.21 | flavonoid 3′-monooxygenase | CYP75 |
| 1.14.13.28 | 3,9-dihydroxypterocarpan 6a- | CYP93A1 |
| | monooxygenase | |
| 1.14.13.30 | leukotriene-B₄ 20-monooxygenase | CYP4F |
| 1.14.13.37 | methyltetrahydroprotoberberine 14- | CYP93A1 |
| | monooxygenase | |
| 1.14.13.41 | tyrosine N-monooxygenase | CYP79 |
| 1.14.13.42 | hydroxyphenylacetonitrile 2- | — |
| | monooxygenase | |
| 1.14.13.47 | (−)-limonene 3-monooxygenase | — |
| 1.14.13.48 | (−)-limonene 6-monooxygenase | — |
| 1.14.13.49 | (−)-limonene 7-monooxygenase | — |
| 1.14.13.52 | isoflavone 3′-hydroxylase | — |
| 1.14.13.53 | isoflavone 2′-hydroxylase | — |
| 1.14.13.55 | protopine 6-monooxygenase | — |
| 1.14.13.56 | dihydrosanguinarine 10-monooxygenase | — |
| 1.14.13.57 | dihydrochelirubine 12-monooxygenase | — |
| 1.14.13.60 | 27-hydroxycholesterol 7α-monooxygenase | — |
| 1.14.13.70 | sterol 14-demethylase | CYP51 |
| 1.14.13.71 | N-methylcoclaurine 3′-monooxygenase | CYP80B1 |
| 1.14.13.73 | tabersonine 16-hydroxylase | CYP71D12 |
| 1.14.13.74 | 7-deoxyloganin 7-hydroxylase | — |
| 1.14.13.75 | vinorine hydroxylase | — |
| 1.14.13.76 | taxane 10β-hydroxylase | CYP725A1 |
| 1.14.13.77 | taxane 13α-hydroxylase | CYP725A2 |
| 1.14.13.78 | ent-kaurene oxidase | CYP701 |
| 1.14.13.79 | ent-kaurenoic acid oxidase | CYP88A |
| 1.14.14.1 | unspecific monooxygenase | multiple |
| 1.14.15.1 | camphor 5-monooxygenase | CYP101 |
| 1.14.15.3 | alkane 1-monooxygenase | CYP4A |
| 1.14.15.4 | steroid 11β-monooxygenase | CYP11B |
| 1.14.15.5 | corticosterone 18-monooxygenase | CYP11B |
| 1.14.15.6 | cholesterol monooxygenase (side-chain- | CYP11A |
| | cleaving) | |
| 1.14.21.1 | (S)-stylopine synthase | — |
| 1.14.21.2 | (S)-cheilanthifoline synthase | — |
| 1.14.21.3 | berbamunine synthase | CYP80 |
| 1.14.21.4 | salutaridine synthase | — |
| 1.14.21.5 | (S)-canadine synthase | — |
| 1.14.99.9 | steroid 17α-monooxygenase | CYP17 |
| 1.14.99.10 | steroid 21-monooxygenase | CYP21 |
| 1.14.99.22 | ecdysone 20-monooxygenase | — |
| 1.14.99.28 | linalool 8-monooxygenase | CYP111 |
| 4.2.1.92 | hydroperoxide dehydratase | CYP74 |
| 5.3.99.4 | prostaglandin-I synthase | CYP8 |
| 5.3.99.5 | thromboxane-A synthase | CYP5 |

Table 3 below lists additional cyctochrome P450 enzymes that are suitable for use in the cyclopropanation reactions of the present invention. The accession numbers in Table 3 are incorporated herein by reference in their entirety for all purposes. The cytochrome P450 gene and/or protein sequences disclosed in the following patent documents are hereby incorporated by reference in their entirety for all purposes: WO 2013/076258; CN 103160521; CN 103223219; KR 2013081394; JP 5222410; WO 2013/073775; WO 2013/054890; WO 2013/048898; WO 2013/031975; WO 2013/064411; U.S. Pat. No. 8,361,769; WO 2012/150326, CN 102747053; CN 102747052; JP 2012170409; WO 2013/115484; CN 103223219; KR 2013081394; CN 103194461; JP 5222410; WO 2013/086499; WO 2013/076258; WO 2013/073775; WO 2013/064411; WO 2013/054890; WO 2013/031975; U.S. Pat. No. 8,361,769; WO 2012/156976; WO 2012/150326; CN 102747053; CN 102747052; US 20120258938; JP 2012170409; CN 102399796; JP 2012055274; WO 2012/029914; WO 2012/028709; WO 2011/154523; JP 2011234631; WO 2011/121456; EP 2366782; WO 2011/105241; CN 102154234; WO 2011/093185; WO 2011/093187; WO 2011/093186; DE 102010000168; CN 102115757; CN 102093984; CN 102080069; JP 2011103864; WO 2011/042143; WO 2011/038313; JP 2011055721; WO 2011/025203; JP 2011024534; WO 2011/008231; WO 2011/008232; WO 2011/005786; IN 2009DE01216; DE 102009025996; WO 2010/134096; JP 2010233523; JP 2010220609; WO 2010/095721; WO 2010/064764; US 20100136595; JP 2010051174; WO 2010/024437; WO 2010/011882; WO 2009/108388; US 20090209010; US 20090124515; WO 2009/041470; KR 2009028942; WO 2009/039487; WO 2009/020231; JP 2009005687; CN 101333520; CN 101333521; US 20080248545; JP 2008237110; CN 101275141; WO 2008/118545; WO 2008/115844; CN 101255408; CN 101250506; CN 101250505; WO 2008/098198; WO 2008/096695; WO 2008/071673; WO 2008/073498; WO 2008/065370; WO 2008/067070; JP 2008127301; JP 2008054644; KR 794395; EP 1881066; WO 2007/147827; CN 101078014; JP 2007300852; WO 2007/048235; WO 2007/044688; WO 2007/032540; CN 1900286; CN 1900285; JP 2006340611; WO 2006/126723; KR 2006029792; KR 2006029795; WO 2006/105082; WO 2006/076094; US 2006/0156430; WO 2006/065126; JP 2006129836; CN 1746293; WO 2006/029398; JP 2006034215; JP 2006034214; WO 2006/009334; WO 2005/111216; WO 2005/080572; US 2005/0150002; WO 2005/061699; WO 2005/052152; WO 2005/038033; WO 2005/038018; WO 2005/030944; JP 2005065618; WO 2005/017106; WO 2005/017105; US 20050037411; WO 2005/010166; JP 2005021106; JP 2005021104; JP 2005021105; WO 2004/113527; CN 1472323; JP 2004261121; WO 2004/013339; WO 2004/011648; DE 10234126; WO 2004/003190; WO 2003/087381; WO 2003/078577; US 20030170627; US 20030166176; US 20030150025; WO 2003/057830; WO 2003/052050; CN 1358756; US 20030092658; US 20030078404; US 20030066103; WO 2003/014341; US 20030022334; WO 2003/008563; EP 1270722; US 20020187538; WO 2002/092801; WO 2002/088341; US 20020160950; WO 2002/083868; US 20020142379; WO 2002/072758; WO 2002/064765; US 20020076777; US 20020076774; US 20020076774; WO 2002/046386; WO 2002/044213; US 20020061566; CN 1315335; WO 2002/034922; WO 2002/033057; WO 2002/029018; WO 2002/018558; JP 2002058490; US 20020022254; WO 2002/008269; WO 2001/098461; WO 2001/081585; WO 2001/051622; WO 2001/034780; CN 1271005; WO 2001/011071; WO 2001/007630; WO 2001/007574; WO 2000/078973; U.S. Pat. No. 6,130,077; JP 2000152788; WO 2000/031273; WO 2000/020566; WO 2000/000585; DE 19826821; JP 11235174; U.S. Pat. No. 5,939,318; WO 99/19493; WO 99/18224; U.S. Pat. No. 5,886,157; WO 99/08812; U.S. Pat. No. 5,869,283; JP 10262665; WO 98/40470; EP 776974; DE 19507546; GB 2294692; U.S. Pat. No. 5,516,674; JP 07147975; WO 94/29434; JP 06205685; JP 05292959; JP 04144680; DD 298820; EP 477961; SU 1693043; JP 01047375; EP 281245; JP 62104583; JP 63044888; JP 62236485; JP 62104582; and JP 62019084.

TABLE 3
Additional cytochrome P450 enzymes of the present invention.

| | | | SEQ |
|---|---|---|---|
| Species | Cyp No. | Accession No. | ID NO |
| Bacillus megaterium | 102A1 | AAA87602 | 1 |
| Bacillus megaterium | 102A1 | ADA57069 | 2 |
| Bacillus megaterium | 102A1 | ADA57068 | 3 |
| Bacillus megaterium | 102A1 | ADA57062 | 4 |
| Bacillus megaterium | 102A1 | ADA57061 | 5 |
| Bacillus megaterium | 102A1 | ADA57059 | 6 |
| Bacillus megaterium | 102A1 | ADA57058 | 7 |
| Bacillus megaterium | 102A1 | ADA57055 | 8 |
| Bacillus megaterium | 102A1 | ACZ37122 | 9 |
| Bacillus megaterium | 102A1 | ADA57057 | 10 |
| Bacillus megaterium | 102A1 | ADA57056 | 11 |
| Mycobacterium sp. HXN-1500 | 153A6 | CAH04396 | 12 |
| Tetrahymena thermophile | 5013C2 | ABY59989 | 13 |
| Nonomuraea dietziae | | AGE14547.1 | 14 |
| Homo sapiens | 2R1 | NP_078790 | 15 |
| Macca mulatta | 2R1 | NP_001180887.1 | 16 |
| Canis familiaris | 2R1 | XP_854533 | 17 |
| Mus musculus | 2R1 | AAI08963 | 18 |
| Bacillus halodurans C-125 | 152A6 | NP_242623 | 19 |
| Streptomyces parvus | aryC | AFM80022 | 20 |
| Pseudomonas putida | 101A1 | P00183 | 21 |
| Homo sapiens | 2D7 | AAO49806 | 22 |
| Rattus norvegicus | C27 | AAB02287 | 23 |
| Oryctolagus cuniculus | 2B4 | AAA65840 | 24 |
| Bacillus subtilis | 102A2 | O08394 | 25 |
| Bacillus subtilis | 102A3 | O08336 | 26 |
| B. megaterium DSM 32 | 102A1 | P14779 | 27 |
| B. cereus ATCC14579 | 102A5 | AAP10153 | 28 |
| B. licheniformis ATTC1458 | 102A7 | YP 079990 | 29 |
| B. thuringiensis serovar | X | YP 037304 | 30 |
| konkukian | | | |
| str.97-27 | | | |
| R. metallidurans CH34 | 102E1 | YP 585608 | 31 |
| A. fumigatus Af293 | 505X | EAL92660 | 32 |
| A. nidulans FGSC A4 | 505A8 | EAA58234 | 33 |
| A. oryzae ATCC42149 | 505A3 | Q2U4F1 | 34 |
| A. oryzae ATCC42149 | X | Q2UNA2 | 35 |
| F. oxysporum | 505A1 | Q9Y8G7 | 36 |
| G. moniliformis | X | AAG27132 | 37 |
| G. zeae PH1 | 505A7 | EAA67736 | 38 |
| G. zeae PH1 | 505C2 | EAA77183 | 39 |
| M. grisea 70-15 syn | 505A5 | XP 365223 | 40 |
| N. crassa OR74 A | 505A2 | XP 961848 | 41 |
| Oryza sativa* | 97A | | |
| Oryza sativa* | 97B | | |
| Oryza sativa | 97C | ABB47954 | 42 |

The start methionine (“M”) may be present or absent from these sequences.
*See, M. Z. Lv et al., Plant Cell Physiol., 53(6): 987-1002 (2012).

In certain embodiments, the present invention provides amino acid substitutions that efficiently remove monooxygenation activity from cytochrome P450 enzymes. This system permits selective enzyme-driven cyclopropanation chemistry without competing side reactions mediated by native P450 catalysis. The invention also provides P450-mediated catalysis that is competent for cyclopropanation chemistry but not able to carry out traditional P450-mediated monooxygenation reactions as ‘orthogonal’ P450 catalysis and respective enzyme variants as ‘orthogonal’ P450s. In some instances, orthogonal P450 variants comprise a single amino acid mutation at the axial position of the heme coordination site (e.g., a C400S mutation in the P450 BM3 enzyme) that alters the proximal heme coordination environment. Accordingly, the present invention also provides P450 variants that contain an axial heme mutation in combination with one or more additional mutations described herein to provide orthogonal P450 variants that show enriched diastereoselective and/or enantioselective product distributions. The present invention further provides a compatible reducing agent for orthogonal P450 cyclopropanation catalysis that includes, but is not limited to, NAD(P)H or sodium dithionite.

In particular embodiments, the cytochrome P450 enzyme is one of the P450 enzymes or enzyme classes set forth in Table 2 or 3. In some embodiments, the cytochrome P450 enzyme is a variant or homolog of one of the P450 enzymes or enzyme classes set forth in Table 2 or 3. In preferred embodiments, the P450 enzyme variant comprises a mutation at the conserved cysteine (Cys or C) residue of the corresponding wild-type sequence that serves as the heme axial ligand to which the iron in protoporphyrin IX is attached. As non-limiting examples, axial mutants of any of the P450 enzymes set forth in Table 2 or 3 can comprise a mutation at the axial position (“AxX”) of the heme coordination site, wherein “X” is selected from Ala, Asp, Arg, Asn, Glu, Gln, Gly, His, Ile, Lys, Leu, Met, Phe, Pro, Ser, Thr, Trp, Tyr, and Val.

In certain embodiments, the conserved cysteine residue in a cytochrome P450 enzyme of interest that serves as the heme axial ligand and is attached to the iron in protoporphyrin IX can be identified by locating the segment of the DNA sequence in the corresponding cytochrome P450 gene which encodes the conserved cysteine residue. In some instances, this DNA segment is identified through detailed mutagenesis studies in a conserved region of the protein (see, e.g., Shimizu et al., Biochemistry 27, 4138-4141, 1988). In other instances, the conserved cysteine is identified through crystallographic study (see, e.g., Poulos et al., J. Mol. Biol 195:687-700, 1987).

In situations where detailed mutagenesis studies and crystallographic data are not available for a cytochrome P450 enzyme of interest, the axial ligand may be identified through phylogenetic study. Due to the similarities in amino acid sequence between P450 enzymes, standard protein alignment algorithms may show a phylogenetic similarity between a P450 enzyme for which crystallographic or mutagenesis data exist and a new P450 enzyme for which such data do not exist. Thus, the polypeptide sequences of the present invention for which the heme axial ligand is known can be used as a “query sequence” to perform a search against a specific new cytochrome P450 enzyme of interest or a database comprising cytochrome P450 sequences to identify the heme axial ligand. Such analyses can be performed using the BLAST programs (see, e.g., Altschul et al., J Mol Biol. 215(3):403-10(1990)). Software for performing BLAST analyses publicly available through the National Center for Biotechnology Information. BLASTP is used for amino acid sequences.

Exemplary parameters for performing amino acid sequence alignments to identify the heme axial ligand in a P450 enzyme of interest using the BLASTP algorithm include E value=10, word size=3, Matrix=Blosum62, Gap opening=11, gap extension=1, and conditional compositional score matrix adjustment. Those skilled in the art will know what modifications can be made to the above parameters, e.g., to either increase or decrease the stringency of the comparison and/or to determine the relatedness of two or more sequences.

In preferred embodiments, the cytochrome P450 enzyme is a cytochrome P450 BM3 enzyme or a variant, homolog, or fragment thereof. The bacterial cytochrome P450 BM3 from Bacillus megaterium is a water soluble, long-chain fatty acid monooxygenase. The native P450 BM3 protein is comprised of a single polypeptide chain of 1048 amino acids and can be divided into 2 functional subdomains (see, L. O. Narhi et al., J. Biol. Chem. 261, 7160 (1986)). An N-terminal domain, amino acid residues 1-472, contains the heme-bound active site and is the location for monoxygenation catalysis. The remaining C-terminal amino acids encompass a reductase domain that provides the necessary electron equivalents from NADPH to reduce the heme cofactor and drive catalysis. The presence of a fused reductase domain in P450 BM3 creates a self-sufficient monooxygenase, obviating the need for exogenous accessory proteins for oxygen activation (see, id.). It has been shown that the N-terminal heme domain can be isolated as an individual, well-folded, soluble protein that retains activity in the presence of hydrogen peroxide as a terminal oxidant under appropriate conditions (P. C. Cirino et al., Angew. Chem., Int. Ed. 42, 3299 (2003)).

In preferred embodiments, the cytochrome P450 enzyme is a cytochrome P450 BM3 or a variant or homolog thereof. In certain instances, the cytochrome P450 BM3 enzyme comprises or consists of the amino acid sequence set forth in SEQ ID NO:1. In certain other instances, the cytochrome P450 BM3 enzyme is a natural variant thereof as described, e.g., in J. Y. Kang et al., AMB Express 1:1 (2011), wherein the natural variants are divergent in amino acid sequence from the wild-type cytochrome P450 BM3 enzyme sequence (SEQ ID NO:1) by up to about 5% (e.g., SEQ ID NOS:2-11).

In particular embodiments, the P450 BM3 enzyme variant comprises or consists of the heme domain of the wild-type P450 BM3 enzyme sequence (e.g., amino acids 1-463 of SEQ ID NO:1) and optionally at least one mutation as described herein. In other embodiments, the P450 BM3 enzyme variant comprises or consists of a fragment of the heme domain of the wild-type P450 BM3 enzyme sequence (SEQ ID NO:1), wherein the fragment is capable of carrying out the cyclopropanation reactions of the present invention.

In certain embodiments, the P450 BM3 enzyme variant comprises a mutation at the axial position (“AxX”) of the heme coordination site, wherein “X” is selected from Ala, Asp, Arg, Asn, Glu, Gln, Gly, His, Ile, Lys, Leu, Met, Phe, Pro, Ser, Thr, Trp, Tyr, and Val. The conserved cysteine (Cys or C) residue in the wild-type P450 BM3 enzyme is located at position 400 in SEQ ID NO:1. As used herein, the terms “AxX” and “C400X” refer to the presence of an amino acid substitution “X” located at the axial position (i.e., residue 400) of the wild-type P450 BM3 enzyme (i.e., SEQ ID NO:1). In some instances, X is Ser (S). In other instances, X is Ala (A), Asp (D), His (H), Lys (K), Asn (N), Met (M), Thr (T), or Tyr (Y). In some embodiments, the P450 BM3 enzyme variant comprises or consists of the heme domain of the wild-type P450 BM3 enzyme sequence (e.g., amino acids 1-463 of SEQ ID NO:1) or a fragment thereof and an AxX mutation (i.e., “WT-AxX heme”).

In other embodiments, the P450 BM3 enzyme variant comprises at least one or more (e.g., at least two, three, four, five, six, seven, eight, nine, ten, eleven, twelve, or all thirteen) of the following amino acid substitutions in SEQ ID NO:1: V78A, F87V, P142S, T175I, A184V, S226R, H236Q, E252G, T268A, A290V, L353V, I366V, and E442K. In certain instances, the P450 BM3 enzyme variant comprises a T268A mutation alone or in combination with one or more additional mutations such as a C400X mutation (e.g., C400S) in SEQ ID NO:1. In other instances, the P450 BM3 enzyme variant comprises all thirteen of the amino acid substitutions (“BM3-CIS”) in combination with a C400X mutation (e.g., C400S) in SEQ ID NO:1. In some instances, the P450 BM3 enzyme variant comprises or consists of the heme domain of the BM3-CIS enzyme sequence (e.g., amino acids 1-463 of SEQ ID NO:1 comprising all thirteen of the amino acid substitutions) or a fragment thereof and an “AxX” mutation (i.e., “BM3-CIS-AxX heme”).

In some embodiments, the P450 BM3 enzyme variant further comprises at least one or more (e.g., at least two, or all three) of the following amino acid substitutions in SEQ ID NO:1: I263A, A328G, and a T438 mutation. In certain instances, the T438 mutation is T438A, T438S, or T438P. In some instances, the P450 BM3 enzyme variant comprises a T438 mutation such as T438A, T438S, or T438P alone or in combination with one or more additional mutations such as a C400X mutation (e.g., C400S) in SEQ ID NO:1 or a heme domain or fragment thereof. In other instances, the P450 BM3 enzyme variant comprises a T438 mutation such as T438A, T438S, or T438P in a BM3-CIS backbone alone or in combination with a C400X mutation (e.g., C400S) in SEQ ID NO:1 (i.e., “BM3-CIS-T438S-AxX”). In yet other instances, the P450 BM3 enzyme variant comprises or consists of the heme domain of the BM3-CIS enzyme sequence or a fragment thereof in combination with a T438 mutation and an “AxX” mutation (e.g., “BM3-CIS-T438S-AxX heme”).

In other embodiments, the P450 BM3 enzyme variant further comprises from one to five (e.g., one, two, three, four, or five) active site alanine substitutions in the active site of SEQ ID NO:1. In certain instances, the active site alanine substitutions are selected from the group consisting of L75A, M177A, L181A, I263A, L437A, and a combination thereof.

Table 4 below provides non-limiting examples of cytochrome P450 BM3 variants of the present invention.

TABLE 4
Exemplary cytochrome P450 BM3 enzyme variants of the present invention.

| P450_{BM3} variants | Mutations compared to wild-type P450_{BM3} (SEQ ID NO: 1) |
|---|---|
| P450_{BM3} (WT-BM3; SEQ ID NO: 1) | None |
| P450_{BM3}-C400A (WT-C400A) | C400A |
| P450_{BM3}-T268A (BM3-T268) | T268A |
| P411_{BM3} (ABC) | C400S |
| P411_{BM3}-T268A (ABC-T268A) | T268A, C400S |
| P411_{BM3}-T438S (ABC-T438A) | T438S, C400S |
| 9-10A | R47C, V78A, K94I, P142S, T175I, A184V, F205C, S226R, |
| | H236Q, E252G, R255S, A290V, L353V |
| B1SYN | 9-10A + C47S, N70Y, A78L, F87A, I174N, I94K, V184T, |
| | I263M, G315S, A330V |
| 9-10A TS | V78A, P142S, T175I, A184V, S226R, H236Q, E252G, A290V, |
| | L353V, I366V, E442K |
| 9-10A-TS-F87V | 9-10A TS + F87V |
| H2A10 | 9-10A TS + F87V, L75A, L181A, T268A |
| H2-5-F10 | 9-10A TS + F87V, L75A, I263A, T268A, L437A |
| H2-4-D4 | 9-10A TS + F87V, L75A, M177A, L181A, T268A, L437A |
| BM3-CIS (P450_{BM3}-CIS; C3C) | 9-10A TS + F87V, T268A |
| BM3-CIS-I263A | BM3-CIS + I263A |
| BM3-CIS-A328G | BM3-CIS + A328G |
| BM3-CIS-T438S | BM3-CIS + T438S |
| BM3-CIS-C400S (P411_{BM3}-CIS; ABC-CIS) | BM3-CIS + C400S |
| BM3-CIS-C400S-A268T (P411_{BM3}-CIS; | BM3-CIS + C400S + A268T (9-10A TS + F87V, C400S) |
| ABC-CIS-A268T) | |
| BM3-CIS-C400D (BM3-CIS-AxD) | BM3-CIS + C400D |
| BM3-CIS-C400Y (BM3-CIS-AxY) | BM3-CIS + C400Y |
| BM3-CIS-C400K (BM3-CIS-AxK) | BM3-CIS + C400K |
| BM3-CIS-C400H (BM3-CIS-AxH) | BM3-CIS + C400H |
| BM3-CIS-C400M (BM3-CIS-AxM) | BM3-CIS + C400M |
| WT-BM3 (heme) | WT heme domain (amino acids 1-463 of SEQ ID NO: 1) |
| WT-AxA (heme) | WT heme domain (amino acids 1-463 of SEQ ID NO: 1) + C400A |
| WT-AxD (heme) | WT heme domain (amino acids 1-463 of SEQ ID NO: 1) + C400D |
| WT-AxH (heme) | WT heme domain (amino acids 1-463 of SEQ ID NO: 1) + C400H |
| WT-AxK (heme) | WT heme domain (amino acids 1-463 of SEQ ID NO: 1) + C400K |
| WT-AxM (heme) | WT heme domain (amino acids 1-463 of SEQ ID NO: 1) + C400M |
| WT-AxN (heme) | WT heme domain (amino acids 1-463 of SEQ ID NO: 1) + C400N |
| WT-AxS (heme) | WT heme domain (amino acids 1-463 of SEQ ID NO: 1) + C400S |
| WT-AxY (heme) | WT heme domain (amino acids 1-463 of SEQ ID NO: 1) + C400Y |
| BM3-CIS-T438S-AxA | BM3-CIS-T438S + C400A |
| BM3-CIS-T438S-AxD | BM3-CIS-T438S + C400D |
| BM3-CIS-T438S-AxM | BM3-CIS-T438S + C400M |
| BM3-CIS-T438S-AxY | BM3-CIS-T438S + C400Y |
| BM3-CIS-T438S-AxT | BM3-CIS-T438S + C400T |
| 7-11D | R47C, V78A, K94I, P142S, T175I, A184V, F205C, S226R, |
| | H236Q, E252G, R255S, A290V, L353V, A82F, A328V |

One skilled in the art will understand that any of the mutations listed in Table 4 can be introduced into any cytochrome P450 enzyme of interest by locating the segment of the DNA sequence in the corresponding cytochrome P450 gene which encodes the conserved amino acid residue as described above for identifying the conserved cysteine residue in a cytochrome P450 enzyme of interest that serves as the heme axial ligand. In certain instances, this DNA segment is identified through detailed mutagenesis studies in a conserved region of the protein (see, e.g., Shimizu et al., Biochemistry 27, 4138-4141, 1988). In other instances, the conserved amino acid residue is identified through crystallographic study (see, e.g., Poulos et al., J. Mol. Biol 195:687-700, 1987). In yet other instances, protein sequence alignment algorithms can be used to identify the conserved amino acid residue.

In further embodiments, the P450 BM3 enzyme variant comprises at least one or more (e.g., at least about 2, 3, 4, 5, 6, 7, 8, 9, 10, 11, 12, 13, 14, 15, 16, 17, 18, 19, 20, 21, or 22) of the following amino acid substitutions in SEQ ID NO:1: R47C, L52I, I58V, L75R, F81 (e.g., F81L, F81W), A82 (e.g., A82S, A82F, A82G, A82T, etc.), F87A, K94I, I94K, H100R, S106R, F107L, A135S, F1621, A197V, F205C, N239H, R255S, S274T, L324I, A328V, V340M, and K434E. In particular embodiments, the P450 BM3 enzyme variant comprises any one or a plurality of these mutations alone or in combination with one or more additional mutations such as those described above, e.g., an “AxX” mutation and/or at least one or more mutations including V78A, F87V, P142S, T175I, A184V, S226R, H236Q, E252G, T268A, A290V, L353V, I366V, and E442K.

Table 5 below provides non-limiting examples of cytochrome P450 BM3 variants of the present invention. Each P450 BM3 variant comprises one or more of the listed mutations (Variant Nos. 1-31), wherein a “+” indicates the presence of that particular mutation in the variant. Any of the variants listed in Table 4 can further comprise an I263A and/or an A328G mutation and/or at least one, two, three, four, or five of the following alanine substitutions, in any combination, in the P450 BM3 enzyme active site: L75A, M177A, L181A, I263A, and L437A. In particular embodiments, the P450 BM3 variant comprises or consists of the heme domain of any one of Variant Nos. 1-31 listed in Table 5 or a fragment thereof, wherein the fragment is capable of carrying out the cyclopropanation reactions of the present invention.

TABLE 5
Exemplary cytochrome P450 BM3 enzyme variants of the present invention.

| | P450_{BM3} variant | | | | | | | | | | | | | | | |
|---|---|---|---|---|---|---|---|---|---|---|---|---|---|---|---|---|
| Mutation | 1 | 2 | 3 | 4 | 5 | 6 | 7 | 8 | 9 | 10 | 11 | 12 | 13 | 14 | 15 | 16 |
| C400X | + | | | | | + | + | + | + | | | | | | | + |
| T268A | | + | | | | + | | | | + | + | + | | | | + |
| F87V | | | + | | | | + | | | + | | | + | + | | + |
| 9-10A-TS | | | | + | | | | + | | | + | | + | | + | |
| T438Z | | | | | + | | | | + | | | + | | + | + | |

| | P450_{BM3} variant | | | | | | | | | | | | | | |
|---|---|---|---|---|---|---|---|---|---|---|---|---|---|---|---|
| Mutation | 17 | 18 | 19 | 20 | 21 | 22 | 23 | 24 | 25 | 26 | 27 | 28 | 29 | 30 | 31 |
| C400X | + | + | + | + | + | | | | | + | + | + | + | | + |
| T268A | + | + | | | | + | + | + | | + | + | + | | + | + |
| F87V | | | + | + | | + | + | | + | + | + | | + | + | + |
| 9-10A-TS | + | | + | | + | + | | + | + | + | | + | + | + | + |
| T438Z | | + | | + | + | | + | + | + | | + | + | + | + | + |

Mutations relative to the wild-type P450_{BM3} amino acid sequence (SEQ ID NO: 1); “X” is selected from Ala, Asp, Arg, Asn, Glu, Gln, Gly, His, Ile, Lys, Leu, Met, Phe, Pro, Ser, Thr, Trp, Tyr, and Val; “Z” is selected from Ala, Ser, and Pro; “9-10A-TS” includes the following amino acid substitutions in SEQ ID NO: 1: V78A, P142S, T175I, A184V, S226R, H236Q, E252G, A290V, L353V, I366V, and E442K.

One skilled in the art will understand that any of the mutations listed in Table 5 can be introduced into any cytochrome P450 enzyme of interest by locating the segment of the DNA sequence in the corresponding cytochrome P450 gene which encodes the conserved amino acid residue as described above for identifying the conserved cysteine residue in a cytochrome P450 enzyme of interest that serves as the heme axial ligand. In certain instances, this DNA segment is identified through detailed mutagenesis studies in a conserved region of the protein (see, e.g., Shimizu et al., Biochemistry 27, 4138-4141, 1988). In other instances, the conserved amino acid residue is identified through crystallographic study (see, e.g., Poulos et al., J. Mol. Biol 195:687-700, 1987). In yet other instances, protein sequence alignment algorithms can be used to identify the conserved amino acid residue. For example, BLAST alignment with the P450 BM3 amino acid sequence as the query sequence can be used to identify the heme axial ligand site and/or the equivalent T268 residue in other cytochrome P450 enzymes.

In other aspects, the present invention provides chimeric heme enzymes such as, e.g., chimeric P450 proteins comprised of recombined sequences from P450 BM3 and at least one, two, or more distantly related P450 enzymes from Bacillus subtillis or any other organism that are competent cyclopropanation catalysts using similar conditions to wild-type P450 BM3 and highly active P450 BM3 variants. As a non-limiting example, site-directed recombination of three bacterial cytochrome P450s can be performed with sequence crossover sites selected to minimize the number of disrupted contacts within the protein structure. In some embodiments, seven crossover sites can be chosen, resulting in eight sequence blocks. One skilled in the art will understand that the number of crossover sites can be chosen to produce the desired number of sequence blocks, e.g., 1, 2, 3, 4, 5, 6, 7, 8, or 9 crossover sites for 2, 3, 4, 5, 6, 7, 8, 9, or 10 sequence blocks, respectively. In other embodiments, the numbering used for the chimeric P450 refers to the identity of the parent sequence at each block. For example, “12312312” refers to a sequence containing block 1 from P450 #1, block 2 from P450 #2, block 3 from P450 #3, block 4 from P450 #1, block 5 from P450 #2, and so on. A chimeric library useful for generating the chimeric heme enzymes of the invention can be constructed as described in, e.g., Otey et al., PLoS Biology, 4(5):e112 (2006), following the SISDC method (see, Hiraga et al., J. Mol. Biol., 330:287-96 (2003)) using the type IIb restriction endonuclease BsaXI, ligating the full-length library into the pCWori vector and transforming into the catalase-deficient E. coli strain SN0037 (see, Nakagawa et al., Biosci. Biotechnol. Biochem., 60:415-420 (1996)); the disclosures of these references are hereby incorporated by reference in their entirety for all purposes.

As a non-limiting example, chimeric P450 proteins comprising recombined sequences or blocks of amino acids from CYP102A1 (Accession No. J04832), CYP102A2 (Accession No. CAB12544), and CYP102A3 (Accession No. U93874) can be constructed. In certain instances, the CYP102A1 parent sequence is assigned “1”, the CYP102A2 parent sequence is assigned “2”, and the CYP102A3 is parent sequence assigned “3”. In some instances, each parent sequence is divided into eight sequence blocks containing the following amino acids (aa): block 1: aa 1-64; block 2: aa 65-122; block 3: aa 123-166; block 4: aa 167-216; block 5: aa 217-268; block 6: aa 269-328; block 7: aa 329-404; and block 8: aa 405-end. Thus, in this example, there are eight blocks of amino acids and three fragments are possible at each block. For instance, “12312312” refers to a chimeric P450 protein of the invention containing block 1 (aa 1-64) from CYP102A1, block 2 (aa 65-122) from CYP102A2, block 3 (aa 123-166) from CYP102A3, block 4 (aa 167-216) from CYP102A1, block 5 (aa 217-268) from CYP102A2, and so on. See, e.g., Otey et al., PLoS Biology, 4(5):e112 (2006). Non-limiting examples of chimeric P450 proteins include those set forth in Table 6 (C2G9, X7, X7-12, C2E6, X7-9, C2B12, TSP234). In some embodiments, the chimeric heme enzymes of the invention can comprise at least one or more of the mutations described herein.

TABLE 6
Exemplary preferred chimeric cytochrome P450 enzymes of the invention.

| | Heme domain | |
|---|---|---|
| Chimeric P450s | block sequence | SEQ ID NO |
| C2G9 | 22223132 | 43 |
| X7 | 22312333 | 44 |
| X7-12 | 12112333 | 45 |
| C2E6 | 11113311 | 46 |
| X7-9 | 32312333 | 47 |
| C2B12 | 32313233 | 48 |
| TSP234 | 22313333 | 49 |

An enzyme's total turnover number (or TTN) refers to the maximum number of molecules of a substrate that the enzyme can convert before becoming inactivated. In general, the TTN for the heme enzymes of the invention range from about 1 to about 100,000 or higher. For example, the TTN can be from about 1 to about 1,000, or from about 1,000 to about 10,000, or from about 10,000 to about 100,000, or from about 50,000 to about 100,000, or at least about 100,000. In particular embodiments, the TTN can be from about 100 to about 10,000, or from about 10,000 to about 50,000, or from about 5,000 to about 10,000, or from about 1,000 to about 5,000, or from about 100 to about 1,000, or from about 250 to about 1,000, or from about 100 to about 500, or at least about 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000, or more. In certain embodiments, the variant or chimeric heme enzymes of the present invention have higher TTNs compared to the wild-type sequences. In some instances, the variant or chimeric heme enzymes have TTNs greater than about 100 (e.g., at least about 100, 150, 200, 250, 300, 325, 350, 400, 450, 500, or more) in carrying out in vitro cyclopropanation reactions. In other instances, the variant or chimeric heme enzymes have TTNs greater than about 1000 (e.g., at least about 1000, 2500, 5000, 10,000, 25,000, 50,000, 75,000, 100,000, or more) in carrying out in vivo whole cell reactions.

When whole cells expressing a heme enzyme are used to carry out a cyclopropanation reaction, the turnover can be expressed as the amount of substrate that is converted to product by a given amount of cellular material. In general, in vivo cyclopropanation reactions exhibit turnovers from at least about 0.01 to at least about 1 mmol·g_{cdw}⁻¹, wherein g_{cdw} is the mass of cell dry weight in grams. For example, the turnover can be from about 0.01 to about 0.1 mmol·g_{cdw}⁻¹, or from about 0.1 to about 1 mmol·g_{cdw}⁻¹, or greater than 1 mmol·g_{cdw}⁻¹. The turnover can be about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or about 1 mmol·g_{cdw}⁻¹.

In certain embodiments, mutations can be introduced into the target gene using standard cloning techniques (e.g., site-directed mutagenesis) or by gene synthesis to produce the heme enzymes (e.g., cytochrome P450 variants) of the present invention. The mutated gene can be expressed in a host cell (e.g., bacterial cell) using an expression vector under the control of an inducible promoter or by means of chromosomal integration under the control of a constitutive promoter. Cyclopropanation activity can be screened in vivo or in vitro by following product formation by GC or HPLC as described herein.

The expression vector comprising a nucleic acid sequence that encodes a heme enzyme variant of the present invention can be a viral vector, a plasmid, a phage, a phagemid, a cosmid, a fosmid, a bacteriophage (e.g., a bacteriophage P1-derived vector (PAC)), a baculovirus vector, a yeast plasmid, or an artificial chromosome (e.g., bacterial artificial chromosome (BAC), a yeast artificial chromosome (YAC), a mammalian artificial chromosome (MAC), or a human artificial chromosome (HAC)). Expression vectors can include chromosomal, non-chromosomal, and synthetic DNA sequences. Equivalent expression vectors to those described herein are known in the art and will be apparent to the ordinarily skilled artisan.

The expression vector can include a nucleic acid sequence encoding a heme enzyme variant that is operably linked to a promoter, wherein the promoter comprises a viral, bacterial, archaeal, fungal, insect, or mammalian promoter. In certain embodiments, the promoter is a constitutive promoter. In some embodiments, the promoter is an inducible promoter. In other embodiments, the promoter is a tissue-specific promoter or an environmentally regulated or a developmentally regulated promoter.

Non-limiting expression vectors for use in bacterial host cells include pCWori, pET vectors such as pET22 (EMD Millipore), pBR322 (ATCC37017), pQE™ vectors (Qiagen), pBluescript™ vectors (Stratagene), pNH vectors, lambda-ZAP vectors (Stratagene); ptrc99a, pKK223-3, pDR540, pRIT2T (Pharmacia), pRSET, pCR-TOPO vectors, pET vectors, pSyn_{—}1 vectors, pChlamy_{—}1 vectors (Life Technologies, Carlsbad, Calif.), pGEM1 (Promega, Madison, Wis.), and pMAL (New England Biolabs, Ipswich, Mass.). Non-limiting examples of expression vectors for use in eukaryotic host cells include pXT1, pSG5 (Stratagene), pSVK3, pBPV, pMSG, pSVLSV40 (Pharmacia), pcDNA3.3, pcDNA4/TO, pcDNA6/TR, pLenti6/TR, pMT vectors (Life Technologies), pKLAC1 vectors, pKLAC2 vectors (New England Biolabs), pQE™ vectors (Qiagen), BacPak baculoviral vectors, pAdeno-X™ adenoviral vectors (Clontech), and pBABE retroviral vectors. Any other vector may be used as long as it is replicable and viable in the host cell.

The host cell can be a bacterial cell, an archaeal cell, a fungal cell, a yeast cell, an insect cell, or a mammalian cell.

Suitable bacterial host cells include, but are not limited to, BL21 E. coli, DE3 strain E. coli, E. coli M15, DH5α, DH10β, HB101, T7 Express Competent E. coli (NEB), B. subtilis cells, Pseudomonas fluorescens cells, and cyanobacterial cells such as Chlamydomonas reinhardtii cells and Synechococcus elongates cells. Non-limiting examples of archaeal host cells include Pyrococcus furiosus, Metallosphera sedula, Thermococcus litoralis, Methanobacterium thermoautotrophicum, Methanococcus jannaschii, Pyrococcus abyssi, Sulfolobus solfataricus, Pyrococcus woesei, Sulfolobus shibatae, and variants thereof. Fungal host cells include, but are not limited to, yeast cells from the genera Saccharomyces (e.g., S. cerevisiae), Pichia (P. Pastoris), Kluyveromyces (e.g., K. lactis), Hansenula and Yarrowia, and filamentous fungal cells from the genera Aspergillus, Trichoderma, and Myceliophthora. Suitable insect host cells include, but are not limited to, Sf9 cells from Spodoptera frugiperda, Sf21 cells from Spodoptera frugiperda, Hi-Five cells, BTI-TN-5B1-4 Trichophusia ni cells, and Schneider 2 (S2) cells and Schneider 3 (S3) cells from Drosophila melanogaster. Non-limiting examples of mammalian host cells include HEK293 cells, HeLa cells, CHO cells, COS cells, Jurkat cells, NS0 hybridoma cells, baby hamster kidney (BHK) cells, MDCK cells, NIH-3T3 fibroblast cells, and any other immortalized cell line derived from a mammalian cell.

In certain embodiments, the present invention provides heme enzymes such as the P450 variants described herein that are active cyclopropanation catalysts inside living cells. As a non-limiting example, bacterial cells (e.g., E. coli) can be used as whole cell catalysts for the in vivo cyclopropanation reactions of the present invention. In some embodiments, whole cell catalysts containing P450 enzymes with the equivalent C400X mutation are found to significantly enhance the total turnover number (TTN) compared to in vitro reactions using isolated P450 enzymes.

In particular embodiments, cytochrome P450 BM3 variants with at least one or more amino acid mutations such as, e.g., C400X (e.g., C400S) and/or T268A amino acid substitutions catalyze nitrine C—H insertion, intramolecular or intramolecular C—H amination, and/or C═C aziridination reactions efficiently, displaying increased total turnover numbers and demonstrating highly regio- and/or enantioselective product formation compared to the wild-type enzyme.

### V. Compounds

In order to generate certain of the compounds below (see sections A, B, E and F), a diazo carbene precursor is useful in the methods described. In certain instances, the structure of the diazo carbene precursor has the following formula:

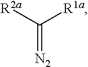

wherein R^{1a} is independently selected from H, optionally substituted C₁₋₁₈ alkyl, optionally substituted C₆₋₁₀ aryl, optionally substituted 6- to 10-membered heteroaryl, halo, cyano, C(O)OR^{1b}, C(O)N(R^{7a})₂, C(O)R⁸, C(O)C(O)OR^{8a}, and Si(R^{8a})₃; and R^{2a} is independently selected from H, optionally substituted C₁₋₁₈ alkyl, optionally substituted C₆₋₁₀ aryl, optionally substituted 6- to 10-membered heteroaryl, halo, cyano, C(O)OR^{2b}, C(O)N(R^{7a})₂, C(O)R^{8a}, C(O)C(O)OR^{8a}, and Si(R⁸a)₃. R^{1b} and R^{2b} are independently selected from H, optionally substituted C₁₋₁₈ alkyl and -L-R^{C}.

When the moiety -L-R^{C} is present, L is selected from a bond, —C(R¹)₂—, and —NR^{L}—C(R^{L})₂—. Each R^{L} is independently selected from H, C₁₋₆alkyl, halo, —CN, and —SO₂, and each R^{C} is selected from optionally substituted C₆₋₁₀ aryl, optionally substituted 6- to 10-membered heteroraryl, and optionally substituted 6- to 10-membered heterocyclyl.

Each R^{7a} and R^{8a} is independently selected from H, optionally substituted C₁₋₁₂ alkyl, optionally substituted C₂₋₁₂ alkenyl, and optionally substituted C₆₋₁₀ aryl.

Any diazo carbene precursor can be added to the reaction as a reagent itself, or the diazo carbene precursor can be prepared in situ.

In some embodiments, the diazo carbene precursor is selected from an α-diazoester, an α-diazoamide, an α-diazonitrile, an α-diazoketone, an α-diazoaldehyde, and an α-diazosilane. In certain embodiments, the diazo reagent has a formula selected from:

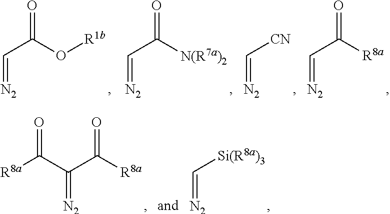

wherein R^{1b} is selected from H and optionally substituted C₁-C₆ alkyl; and each R^{7a} and R^{8a} is independently selected from H, optionally substituted C₁₋₁₂ alkyl, optionally substituted C₂₋₁₂ alkenyl, and optionally substituted C₆₋₁₀ aryl.

In some embodiments, the diazo carbene precursor is selected from the group consisting of diazomethane, ethyl diazoacetate, and (trimethylsilyl)diazomethane.

In some embodiments, the diazo reagent is an α-diazoester. In some embodiments, the diazo carbene precursor has the formula:

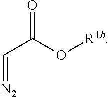

In certain instances, the following reaction is an example of the enzyme catalyzed reaction of the present invention:

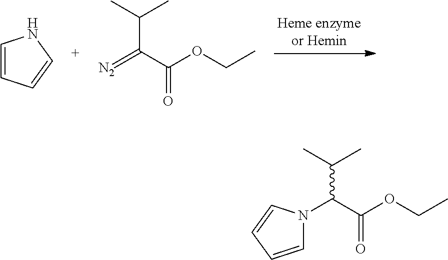

The present invention is based on the surprising discovery that engineered heme enzymes such as cytochrome P450_{BM3} enzymes, including a serine-heme-ligated P411 enzyme, efficiently catalyze carbene and nitrene insertion and transfer reactions. Suitable reactions include, but are not limited to, carbene insertion reactions into N—H, C—H, O—H or Si—H bonds, as well as nitrene transfer into C═C and C—H bonds. Carbenes are highly electron deficient species as carbene carbons have only 6 electrons in the valence shell and thus are highly electrophilic. In certain instances, the present invention provides methods for carbenes insertion reactions into N—H bonds and C—H bonds. In certain other aspects, the present invention also provides methods and systems for heme-containing enzyme to catalyze nitrogen insertion into C═C bonds, also known as aziridination and C—H bonds.

In certain aspects, the methods herein produce a plurality of products, such as products having an Z or E configuration. The plurality of products having a Z:E configuration have a ratio of from 1:99 to 99:1. In certain instances, the products have a % ee_{Z} of at least −90% to at least 90%. In certain instances, the reaction is at least 10% to 100% stereoselective such as 30% to at least 90% diasteroselective.

A. Carbene Insertion into N—H

In certain aspects, the present invention provides methods and systems for heme-containing enzymes to catalyze a carbene insertion into a nitrogen-hydrogen bond. In certain instances, the enzyme catalyzed reaction interposes a carbene into an existing N—H bond.

In one embodiment, the present invention provides a method for catalyzing a carbene insertion into a N—H bond to produce a product having a new C—N bond, the method comprising:

providing a N—H containing substrate, a diazo carbene precursor and an engineered heme enzyme; and

allowing the reaction to proceed for a time sufficient to form a product having a new C—N bond.

In certain instances the N—H containing substrate is an aryl amine such as an endocyclic nitrogen or a secondary exocyclic amine. Alternatively, the N—H containing substrate is an aliphatic amine such as a secondary aliphatic amine like a C₁₋₁₂ alkylamine or C₁₋₁₂ dialkylamine. In other embodiments, the present invention provides a product of the methods herein. NH containing substrates include, but are not limited to, optionally substituted pyrrole, optionally substituted imidazole, optionally substituted pyrazole, optionally substituted indole, optionally substituted indazole, optionally substituted carbazole, optionally substituted carboline, optionally substituted perimidine, optionally substituted phenothiazine, optionally substituted phenoxazine, optionally substituted pyrrolidione, optionally substituted pyrroline, optionally substituted imidazolidine, optionally substituted imidazoline, optionally substituted pyrazolidine, optionally substituted pyrazoline, optionally substituted piperidine, optionally substituted piperazine, optionally substituted indoline, optionally substituted isoindoline, optionally substituted morpholine and optionally substituted phenylamine (analine).

In certain instances, the diazo carbene precursor is an aryl diazo carbene precursor. Alternatively, the diazo carbene precursor is an aliphatic diazo carbene precursor.

In certain instances, the product is a compound of Formula Ia:

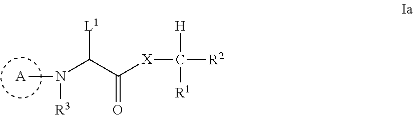

wherein: the dotted circle A is an optionally substituted aryl group, wherein the nitrogen represents an endocyclic nitrogen atom which is part of ring A or an exocyclic nitrogen atom bonded to a ring atom of A;

R¹ is a member selected from the group consisting of hydrogen, an optionally substituted alkyl, and cyano;

R² is a member selected from the group consisting of hydrogen, an optionally substituted alkyl, an optionally substituted aryl, an optionally substituted heteroaryl, and an optionally substituted heterocyclyl;

R³ is a member selected from the group consisting of hydrogen and an optionally substituted alkyl,

X is a heteroatom selected form the group consisting of S, O and NR, wherein R is hydrogen or optionally substituted alkyl; and

L¹ is an optionally substituted alkyl or hydrogen.

In certain instances, R² is an optionally substituted aryl group such as an optionally substituted phenoxybenzyl.

In certain instances, A is an optionally substituted aryl group and the nitrogen is exocyclic.

In certain instances, L¹ is an isopropyl group.

In certain instances, A is an analinyl group optionally substituted with 1 to 5 substituents, which may be the same or different, selected from the group consisting of a halogen atom, an alkyl, haloalkyl, phenyl, alkoxy, haloalkoxy, cycloalkoxy, phenoxy, alkenyl, haloalkenyl, alkynyl, haloalkynyl, alkoxyalkyl, alkenyloxy, haloalkenyloxy, alkynyloxy, haloalkynyloxy, alkylthio, haloalkylthio, alkylsulfoxyl, acyl, alkoxyalkoxy, alkenylthio, alkoxycarbonyl, haloalkoxycarbonyl, alkynyloxycarbonyl, alkenyloxycarbonyl, nitro, and haloalkenylthio.

In certain instances, the compound is a member selected from the group consisting of cyano(3-phenoxyphenyl)methyl 2-((2-fluoro-4-(trifluoromethyl)phenyl)amino)-3-methylbutanoate; cyano(3-fluoro-5-phenoxyphenyl)methyl 2-((2-chloro-4-(trifluoromethyl)phenyl)amino)-3-methylbutanoate; cyano(4-fluoro-3-phenoxyphenyl)methyl 2-((2-chloro-4-(trifluoromethyl)phenyl)amino)-3-methylbutanoate; cyano(2-fluoro-5-phenoxyphenyl)methyl 2-((2-chloro-4-(trifluoromethyl)phenyl)amino)-3-methylbutanoate; cyano(3-phenoxyphenyl)methyl 2-((2-fluoro-4-((trifluoromethyl)thio)phenyl)amino)-3-methylbutanoate; and (2,5-dioxo-3-(prop-2-yn-1-yl)imidazolidin-1-yl)methyl 3-methyl-2-((4-(trifluoromethyl)phenyl)amino)butanoate.

In certain instances, A is an optionally substituted aryl group and the nitrogen is endocylic.

In certain instances, A is an optionally substituted pyrroyl group optionally substituted with 1 to 4 substituents, which may be the same or different, selected from the group consisting of a halogen atom, an alkyl, haloalkyl, phenyl, alkoxy, haloalkoxy, cycloalkoxy, phenoxy, alkenyl, haloalkenyl, alkynyl, haloalkynyl, alkoxyalkyl, alkenyloxy, haloalkenyloxy, alkynyloxy, haloalkynyloxy, alkylthio, haloalkylthio, alkylsulfoxyl, acyl, alkoxyalkoxy, alkenylthio, alkoxycarbonyl, haloalkoxycarbonyl, alkynyloxycarbonyl, alkenyloxycarbonyl, nitro, and haloalkenylthio.

In certain instances, R² has the formula:

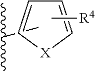

wherein X is a member selected from the group consisting of O, S and NR, wherein R is hydrogen or optionally substituted alkyl; and

R⁴ is a member selected from the group consisting an alkyl, haloalkyl, alkoxy, haloalkoxy, cycloalkoxy, phenoxy, alkenyl, haloalkenyl, alkynyl, haloalkynyl, alkoxyalkyl, alkenyloxy, haloalkenyloxy, alkynyloxy, haloalkynyloxy, phenyl, phenyoxy, thiophenyl, benzyl and furyl.

In certain instances, the compound is a member selected from the group consisting of 3-phenoxybenzyl 3-methyl-2-(1H-pyrrol-1-yl)butanoate, cyano(3-phenoxyphenyl)methyl 3-methyl-2-(1H-pyrrol-1-yl)butanoate.

In certain instances, R² is an optionally substituted benzylpyrrolyl.

In certain instances, the compound is (3-benzyl-1H-pyrrol-1-yl)methyl 2-((2-chloro-4-(trifluoromethyl)phenyl)amino)-3-methylbutanoate.

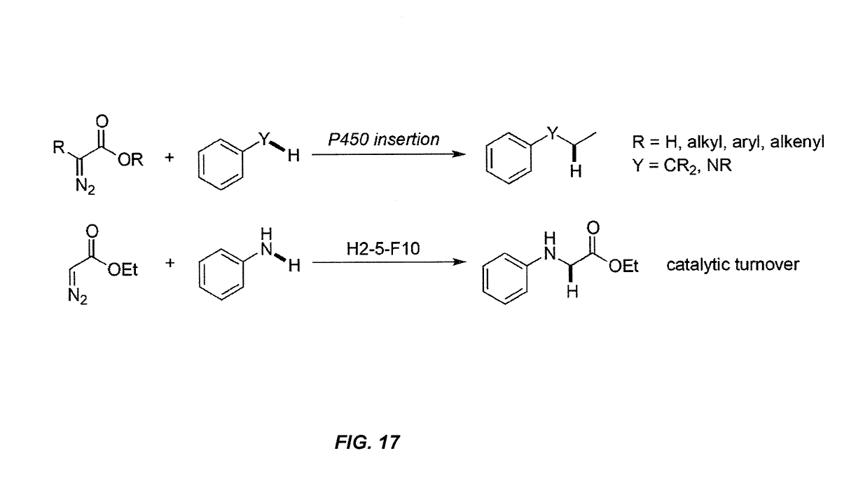

In certain aspects, FIG. 17 shows C—H and N—H bond insertion by P450 variants in the presence of diazo compounds. Further compounds are set forth below:

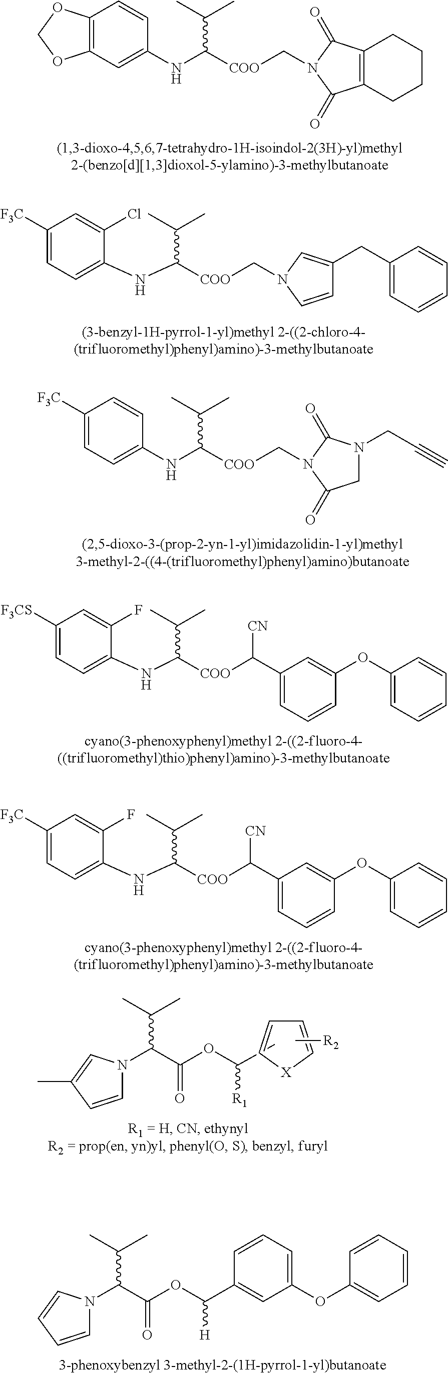

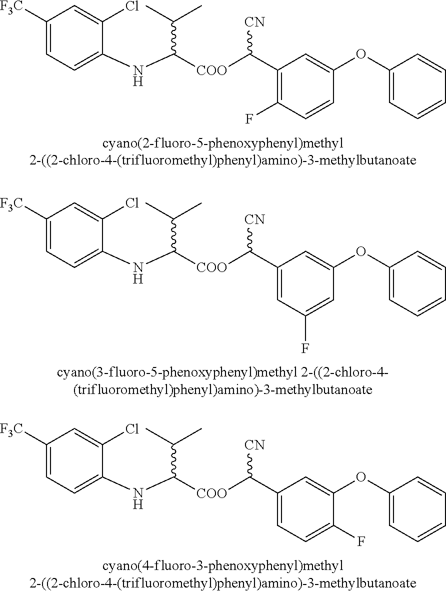

B. Carbene Insertion into C—H

In certain aspects, the present invention provides methods and systems for heme-containing enzymes to catalyze a carbene insertion into a carbon-hydrogen bond. In certain instances, the enzyme catalyzed reaction interposes a carbene i.e., H₂C: into an existing —C—H bond, to produce, for example —C—CH₃. The present methods and systems enable intermolecular insertions, intramolecular insertions and/or a combination thereof.

In certain aspects, for example in intermolecular CH insertion reactions, the methods described herein are synthetically very useful due to the high degree of selectivity.

In certain aspects, such as in intramolecular carbene C—H insertion reactions, the carbon that stabilizes a positive charge will be most reactive. As such, tertiary carbons are more reactive than secondary carbons, which are more reactive than primary carbons due to the electron density in the C—H bond. In certain instances, steric or conformational aspects will outweigh the electronic effects.

In one embodiment, the present invention provides a method for catalyzing a carbene insertion into a C—H bond to produce a product with a new C—C bond. The method comprises:

providing a C—H containing substrate, a diazo carbene precursor and an engineered heme enzyme; and

allowing the reaction to proceed for a time sufficient to form a product having a new C—C bond. In other embodiments, the present invention provides a product of the methods herein.

In certain aspects, the C—H containing substrate is an aryl molecule. Alternatively, the C—H containing substrate is an aliphatic molecule such as an optionally substituted alkane or optionally substituted heterocycle.

In certain aspects, the C—H containing aryl molecule is an optionally substituted arylalkane or optionally substituted heteroarylalkane.

In certain aspects, the diazo carbene precursor is an aryl diazo carbene precursor. Alternatively, the diazo carbene precursor is an aliphatic diazo carbene precursor.

In certain aspects, the product having a new C—C bond is a compound of Formula II:

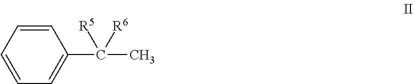

wherein R⁵ and R⁶ may be the same are different, wherein each is a member selected from the group consisting of hydrogen, an alkyl, haloalkyl, alkoxy, haloalkoxy, alkenyl, haloalkenyl and optionally substituted aryl.

In certain aspects, the product having a new C—C bond is a compound of Formula IIa:

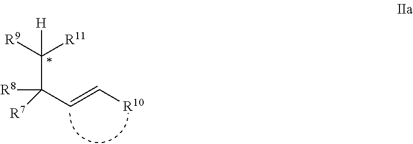

wherein each R⁷, R⁸, and R⁹, may be the same or different, and is a member selected from the group consisting of hydrogen, optionally substituted alkyl, optionally substituted aryl and wherein the carbon designated with a * can be either E or Z configuration;
R¹⁰ represents a number of atoms making a 5 or 6-membered aryl, heteroaryl, heterocyclyl or cycloalkyl ring; and
R¹¹ is a member selected from the group consisting of hydrogen, carbonyl, nitrile or amide.

In certain aspects, the product having a new C—C bond is a compound of Formula IIb

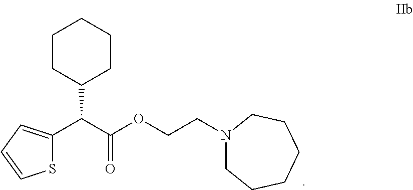 [SMILES: O=C(OCCN1CCCCCC1)[C@H](c1cccs1)C1CCCCC1]

In certain aspects, the product having a new C—C bond is a compound of Formula IIc:

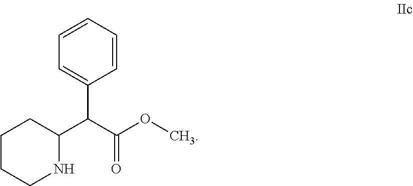 [SMILES: COC(=O)C(c1ccccc1)C1CCCCN1]

In certain aspects, the product having a new C—C bond is a compound of Formula IId:

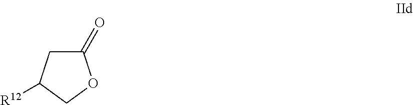

wherein R¹² is a member selected from the group consisting of optionally substituted aryl and optionally substituted O-aryl.

FIG. 17 shows C—H and N—H bond insertion by P450 variants in the presence of diazo compounds.

C. Nitrene Transfer into C═C

In certain aspects, the present invention provides methods and systems for heme-containing enzyme to catalyze nitrogen insertion into C═C bonds, also known as aziridination. The aziridination reactions can be intermolecular, intramolecular and/or a combination thereof. These heme containing enzymes catalyze aziridination reactions, via nitrene insertion, which in certain instances, allows the direct transformation of a C═C into an aziridine. Aziridines are organic compounds containing the aziridine functional group, a three-membered heterocycle with one amine group (—NH—) and two methylene groups (—CH₂). Although in certain exemplary embodiments the inventive reactions produce an aziridine, the products are not limited to a 3 membered ring. The reactions proceed with high regio, chemo, and/or diastereoselectivity as a result of using a heme containing enzyme. In certain instances, a nitrene inserts into a carbon-carbon double bond yielding a secondary amine or amide.

In one embodiment, the present invention provides a method for catalyzing a nitrene insertion reaction into an olefin to produce an aziridine, the method comprising:

providing an olefin substrate, a nitrene precursor and an engineered heme enzyme; and

allowing the reaction to proceed for a time sufficient to produce an aziridine. In other embodiments, the present invention provides a product of the methods herein. In one aspect, the olefin substrate and the nitrene precursor are the same molecule.

In certain aspects, the nitrene precursor contains an azide functional group.

In one aspect, the nitrene precursor has the formula IIIa

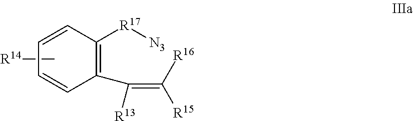

In certain aspects, the aziridine is a compound of formula III:

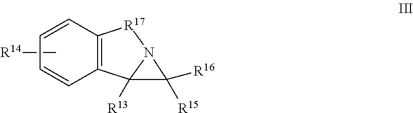

wherein R¹³ is a member selected from the group consisting of hydrogen, alkyl, haloalkyl and optionally substituted aryl;

R¹⁴ is a member selected from the group consisting of hydrogen, halogen, alkyl, haloalkyl, optionally substituted aryl, alkoxy, alkylthio, and optionally substituted amino;

R¹⁵ and R¹⁶ may be the same or different and are selected from the group consisting of hydrogen, halogen, alkyl, haloalkyl, optionally substituted aryl, alkoxy, alkylthio, and optionally substituted amino; and

R¹⁷ is a member selected from the group consisting of C═O, C═S, SO₂ and PO₂OR¹⁸, wherein R¹⁸ is a member selected from the group consisting of hydrogen, alkyl, haloalkyl and optionally substituted aryl.

In certain aspects, the olefin substrate and the nitrene precursor are different molecules.

In one aspect, the nitrene precursor and olefin substrate enzymatically react as follows:

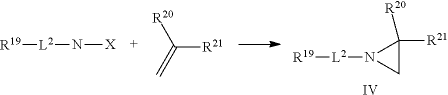

In certain aspects, the nitrene precursor contains a leaving group. Suitable leaving groups X include, but are not limited to, OTs (tosylates), OMs (mesylates), halogen, N₂, H₂ and ITs (N-tosylimine).

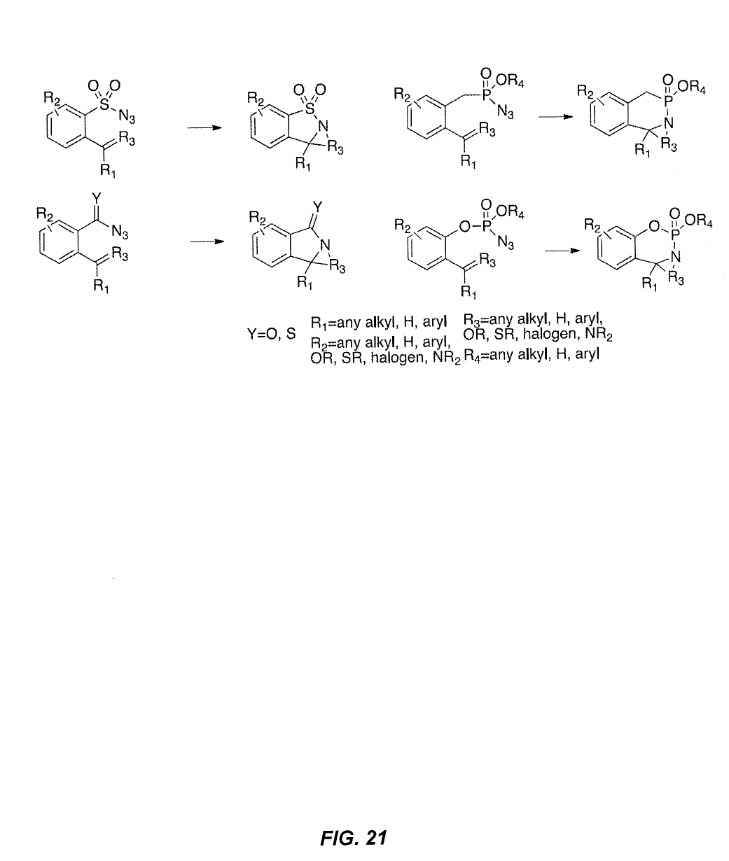

In certain aspects, FIG. 21 illustrates some of the substrate scope of P450-catalyzed intramolecular aziridination.

In certain aspects, the aziridine is a compound of formula IV:

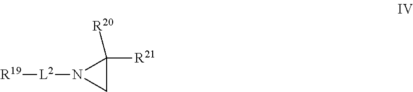

wherein R¹⁹ is a member selected from the group consisting of optionally substituted aryl, optionally substituted heteroaryl, and optionally substituted alkyl;

L² is a member selected from the group consisting of C═O, C═S, SO₂ and PO₂OR¹⁸, wherein R¹⁸ is a member selected from the group consisting of hydrogen, alkyl, haloalkyl and optionally substituted aryl; and

R²⁰ and R²¹ may be the same or different and are selected from the group consisting of hydrogen, halogen, alkyl, haloalkyl, optionally substituted aryl, alkoxy, alkylthio, and optionally substituted amino.

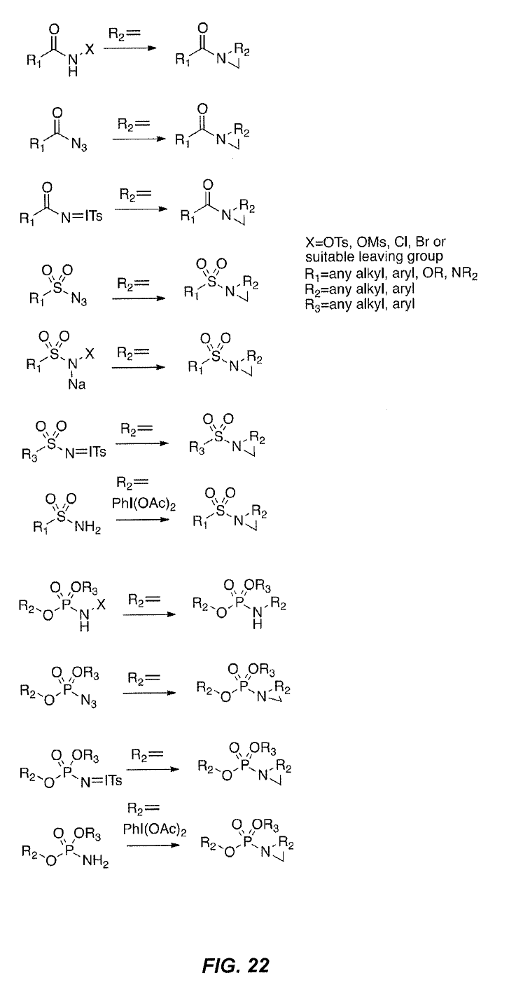

In certain aspects, FIG. 22 illustrates some of the substrate scope of P450-catalyzed intermolecular aziridination.

D. Nitrene Transfer into C—H

In certain aspects, the present invention provides methods and systems for heme-containing enzymes to catalyze nitrogen insertion into C—H bonds, also known as C—H amination. The C—H amination reactions can be intermolecular, intramolecular and a combination thereof. These heme containing enzymes catalyze C—H amination via nitrene insertion, which allows the direct transformation of a C—H into a C—N bond. The reactions proceed with high regio, chemo, and/or diastereoselectivity as a result of using a heme containing enzyme. In certain instances, a nitrene inserts into a carbon-hydrogen covalent bond yielding a secondary amine

In one embodiment, the present invention provides a method for catalyzing a nitrene insertion into a C—H bond to produce a product having a new C—N bond. The method comprises:

providing a C—H containing substrate, a nitrene precursor and an engineered heme enzyme; and

allowing the reaction to proceed for a time sufficient to form a product having a new C—N bond. In other embodiments, the present invention provides a product of the methods herein.

In certain aspects, the C—H containing substrate and the nitrene precursor are the same molecule.

In certain aspects, the nitrene precursor contains an azide functional group.

In certain aspects, the nitrene precursor is a compound of formula Va:

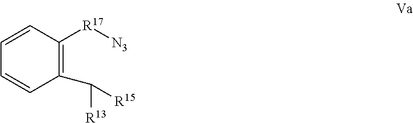

In certain aspects, the product is a compound of formula V:

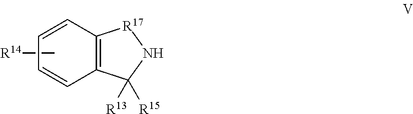

wherein R¹³ is a member selected from the group consisting of hydrogen, alkyl, haloalkyl and optionally substituted aryl;

R¹⁴ is a member selected from the group consisting of hydrogen, halogen, alkyl, haloalkyl, optionally substituted aryl, alkoxy, alkylthio, and optionally substituted amino;

R¹⁵ is a member selected from the group consisting of hydrogen, halogen, alkyl, haloalkyl, optionally substituted aryl, alkoxy, alkylthio, and optionally substituted amino; and

R¹⁷ is a member selected from the group consisting of C═O, C═S, SO₂ and PO₂OR¹⁸, wherein R¹⁸ is a member selected from the group consisting of hydrogen, alkyl, haloalkyl and optionally substituted aryl.

In certain aspects, wherein the C—H containing substrate and the nitrene precursor are different molecules.

In one aspect, the C—H containing substrate and the nitrene precursor undergo the following reaction:

R¹⁹-L²-N—X+R²⁰—H→R¹⁹-L²-NH—R²⁰ VI

wherein the nitrene precursor contains a leaving group X. Suitable leaving groups for X include, but are not limited to, OTs (tosylates), OMs (mesylates), halogen, N₂, H₂ and ITs (N-tosylimine).

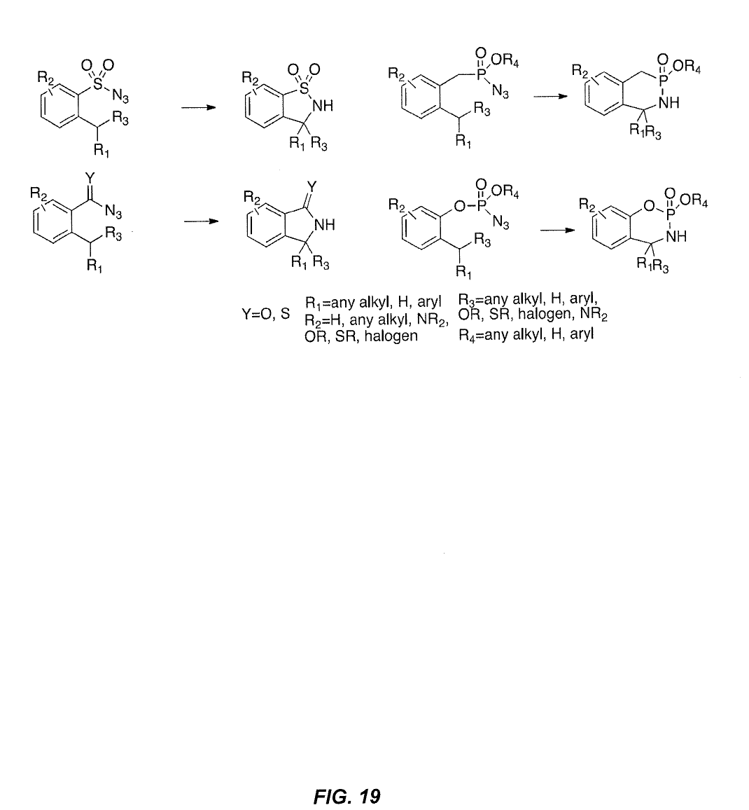

In certain aspects, FIG. 19 illustrates substrate scope of P450-catalyzed intramolecular C—H amination.

In certain aspects, the product is a compound of formula VI:

R¹⁹-L²-NH—R²⁰ VI

wherein: R¹⁹ is a member selected from the group consisting of optionally substituted aryl, an optionally substituted heteroaryl, and optionally substituted alkyl; L² is a member selected from the group consisting of C═O, C═S, SO₂ and PO₂OR¹⁸, wherein

R¹⁸ is a member selected from the group consisting of hydrogen, alkyl, haloalkyl and optionally substituted aryl; and

R²⁰ is selected from the group consisting of hydrogen, halogen, alkyl, haloalkyl, optionally substituted aryl, alkoxy, alkylthio, and optionally substituted amino.

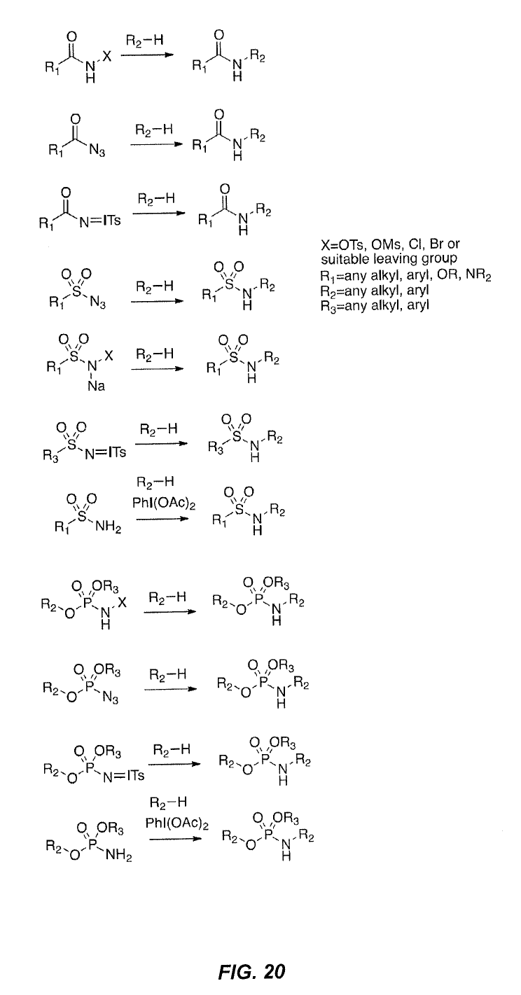

In certain aspects, FIG. 20 illustrates some of the substrate scope of P450-catalyzed intermolecular C—H amination.

In one embodiment, the present invention provides the synthesis of tirofiban as set forth below:

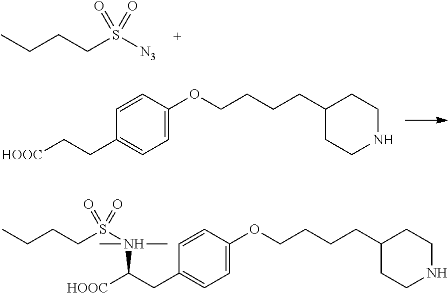

E. Carbene Insertion into O—H

In one embodiment, the present invention provides a method for catalyzing a carbene insertion into a O—H bond to produce a product having a new C—O bond. The method comprises:

providing a O—H containing substrate, a diazo carbene precursor and an engineered heme enzyme; and

allowing the reaction to proceed for a time sufficient to form a product having a new C—O bond. In other embodiments, the present invention provides a product of the methods herein.

In certain instances, the O—H containing substrate can be an aliphatic alcohol or aromatic alcohol. Suitable alcohols include, but are not limited to, optionally substituted alkanols, optionally substituted arylalkanols, optionally substituted heterocyclylalkanols and optionally substituted heteroarylalkanols.

In certain aspects, the product is a compound of Formula VII:

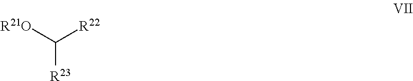

wherein R²¹, R²² and R²³ are each independently, hydrogen, optionally substituted alkyl, optionally substituted aryl and optionally substituted heteroaryl.

In certain aspects, the present invention provides synthesis methods and a product as set forth below:

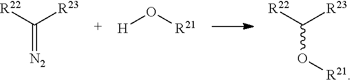

In certain aspects, the present invention provide a synthesis process for duloxetine and the product per se as follows:

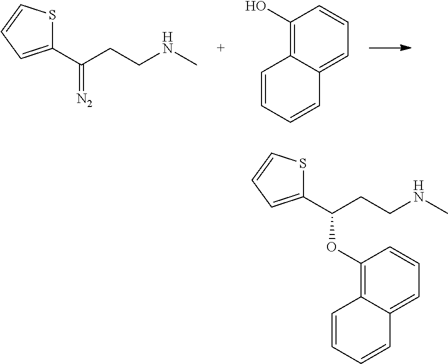

F. Carbene Insertion into Si—H

In one embodiment, the present invention provides a method for catalyzing a carbene insertion into a Si—H bond to produce a product having a new C—Si bond. The method comprises:

providing a Si—H containing substrate, a diazo carbene precursor and an engineered heme enzyme; and

allowing the reaction to proceed for a time sufficient to form a product having a new C—Si bond. In other embodiments, the present invention provides a product of the methods herein.

Various silanes are suitable for the present invention. These silanes include for example, primary, secondary and tertiary silanes. The silanes can be aliphatic silanes or aromatic silanes. Suitable silanes include, but are not limited to, optionally substituted alkylsilanes, optionally substituted arylsilanes, optionally substituted heterocyclylsilanes and optionally substituted heteroarylsilanes.

In certain aspects, product is a compound of Formula VIII:

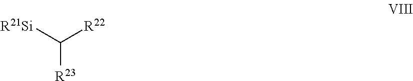

wherein R²¹, R²² and R²³ are each independently hydrogen, optionally substituted alkyl, optionally substituted aryl and optionally substituted heteroaryl.

### VI. Reaction Conditions

The methods of the invention include forming reaction mixtures that contain the heme enzymes described herein. The heme enzymes can be, for example, purified prior to addition to a reaction mixture or secreted by a cell present in the reaction mixture. The reaction mixture can contain a cell lysate including the enzyme, as well as other proteins and other cellular materials. Alternatively, a heme enzyme can catalyze the reaction within a cell expressing the heme enzyme. Any suitable amount of heme enzyme can be used in the methods of the invention. In general, the reaction mixtures contain from about 0.01 mol % to about 10 mol % heme enzyme with respect to the diazo reagent and/or substrate. The reaction mixtures can contain, for example, from about 0.01 mol % to about 0.1 mol % heme enzyme, or from about 0.1 mol % to about 1 mol % heme enzyme, or from about 1 mol % to about 10 mol % heme enzyme. The reaction mixtures can contain from about 0.05 mol % to about 5 mol % heme enzyme, or from about 0.05 mol % to about 0.5 mol % heme enzyme. The reaction mixtures can contain about 0.1, 0.2, 0.3, 0.4, 0.5, 0.6, 0.7, 0.8, 0.9, or about 1 mol % heme enzyme.

The concentration of olefinic substrate and diazo reagent are typically in the range of from about 100 μM to about 1 M. The concentration can be, for example, from about 100 μM to about 1 mM, or about from 1 mM to about 100 mM, or from about 100 mM to about 500 mM, or from about 500 mM to 1 M. The concentration can be from about 500 μM to about 500 mM, 500 μM to about 50 mM, or from about 1 mM to about 50 mM, or from about 15 mM to about 45 mM, or from about 15 mM to about 30 mM. The concentration of olefinic substrate or diazo reagent can be, for example, about 100, 200, 300, 400, 500, 600, 700, 800, or 900 μM. The concentration of olefinic substrate or diazo reagent can be about 1, 5, 10, 15, 20, 25, 30, 35, 40, 45, 50, 55, 60, 65, 70, 75, 80, 85, 90, 95, 100, 150, 200, 250, 300, 350, 400, 450, or 500 mM.

Reaction mixtures can contain additional components. As non-limiting examples, the reaction mixtures can contain buffers (e.g., 2-(N-morpholino)ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, and sodium borate), cosolvents (e.g., dimethylsulfoxide, dimethylformamide, ethanol, methanol, isopropanol, glycerol, tetrahydrofuran, acetone, acetonitrile, and acetic acid), salts (e.g., NaCl, KCl, CaCl₂, and salts of Mn²⁺ and Mg²⁺), denaturants (e.g., urea and guandinium hydrochloride), detergents (e.g., sodium dodecylsulfate and Triton-X 100), chelators (e.g., ethylene glycol-bis(2-aminoethylether)-N,N,N′,N′-tetraacetic acid (EGTA), 2-({2-[Bis(carboxymethyl)amino]ethyl}(carboxymethyl)amino)acetic acid (EDTA), and 1,2-bis(o-aminophenoxy)ethane-N,N,N′,N′-tetraacetic acid (BAPTA)), sugars (e.g., glucose, sucrose, and the like), and reducing agents (e.g., sodium dithionite, NADPH, dithiothreitol (DTT), β-mercaptoethanol (BME), and tris(2-carboxyethyl)phosphine (TCEP)). Buffers, cosolvents, salts, denaturants, detergents, chelators, sugars, and reducing agents can be used at any suitable concentration, which can be readily determined by one of skill in the art. In general, buffers, cosolvents, salts, denaturants, detergents, chelators, sugars, and reducing agents, if present, are included in reaction mixtures at concentrations ranging from about 1 μM to about 1 M. For example, a buffer, a cosolvent, a salt, a denaturant, a detergent, a chelator, a sugar, or a reducing agent can be included in a reaction mixture at a concentration of about 1 μM, or about 10 μM, or about 100 μM, or about 1 mM, or about 10 mM, or about 25 mM, or about 50 mM, or about 100 mM, or about 250 mM, or about 500 mM, or about 1 M. In some embodiments, a reducing agent is used in a sub-stoichiometric amount with respect to the olefin substrate and the diazo reagent. Cosolvents, in particular, can be included in the reaction mixtures in amounts ranging from about 1% v/v to about 75% v/v, or higher. A cosolvent can be included in the reaction mixture, for example, in an amount of about 5, 10, 20, 30, 40, or 50% (v/v).

Reactions are conducted under conditions sufficient to catalyze the formation of the desired products. The reactions can be conducted at any suitable temperature. In general, the reactions are conducted at a temperature of from about 4° C. to about 40° C. The reactions can be conducted, for example, at about 25° C. or about 37° C. The reactions can be conducted at any suitable pH. In general, the reactions are conducted at a pH of from about 6 to about 10. The reactions can be conducted, for example, at a pH of from about 6.5 to about 9. The reactions can be conducted for any suitable length of time. In general, the reaction mixtures are incubated under suitable conditions for anywhere between about 1 minute and several hours. The reactions can be conducted, for example, for about 1 minute, or about 5 minutes, or about 10 minutes, or about 30 minutes, or about 1 hour, or about 2 hours, or about 4 hours, or about 8 hours, or about 12 hours, or about 24 hours, or about 48 hours, or about 72 hours. Reactions can be conducted under aerobic conditions or anaerobic conditions. Reactions can be conducted under an inert atmosphere, such as a nitrogen atmosphere or argon atmosphere. In some embodiments, a solvent is added to the reaction mixture. In some embodiments, the solvent forms a second phase, and the cyclopropanation occurs in the aqueous phase. In some embodiments, the heme enzyme is located in the aqueous layer whereas the substrates and/or products occur in an organic layer. Other reaction conditions may be employed in the methods of the invention, depending on the identity of a particular heme enzyme, olefinic substrate, or diazo reagent.

Reactions can be conducted in vivo with intact cells expressing a heme enzyme of the invention. The in vivo reactions can be conducted with any of the host cells used for expression of the heme enzymes, as described herein. A suspension of cells can be formed in a suitable medium supplemented with nutrients (such as mineral micronutrients, glucose and other fuel sources, and the like). Carbene insertion and/or nitrene transfer yields from reactions in vivo can be controlled, in part, by controlling the cell density in the reaction mixtures. Cellular suspensions exhibiting optical densities ranging from about 0.1 to about 50 at 600 nm can be used for carbene insertion and/or nitrene transfer reactions. Other densities can be useful, depending on the cell type, specific heme enzymes, or other factors.

The methods of the invention can be assessed in terms of the diastereoselectivity and/or enantioselectivity of cyclopropanation reaction—that is, the extent to which the reaction produces a particular isomer, whether a diastereomer or enantiomer. A perfectly selective reaction produces a single isomer, such that the isomer constitutes 100% of the product. As another non-limiting example, a reaction producing a particular enantiomer constituting 90% of the total product can be said to be 90% enantioselective. A reaction producing a particular diastereomer constituting 30% of the total product, meanwhile, can be said to be 30% diastereoselective.

In general, the methods of the invention include reactions that are from about 1% to about 99% diastereoselective. The reactions are from about 1% to about 99% enantioselective. The reaction can be, for example, from about 10% to about 90% diastereoselective, or from about 20% to about 80% diastereoselective, or from about 40% to about 60% diastereoselective, or from about 1% to about 25% diastereoselective, or from about 25% to about 50% diastereoselective, or from about 50% to about 75% diastereoselective. The reaction can be about 10%, 15%, 20%, 25%, 30%, 35%, 40%, 45%, 50%, 55%, 60%, 65%, 70%, 75%, 80%, 85%, 90%, or about 95% diastereoselective. The reaction can be from about 10% to about 90% enantioselective, from about 20% to about 80% enantioselective, or from about 40% to about 60% enantioselective, or from about 1% to about 25% enantioselective, or from about 25% to about 50% enantioselective, or from about 50% to about 75% enantioselective. The reaction can be about 10%, 15%, 20%, 25%, 30%, 35%, 40%, 45%, 50%, 55%, 60%, 65%, 70%, 75%, 80%, 85%, 90%, or about 95% enantioselective. Accordingly some embodiments of the invention provide methods wherein the reaction is at least 30% to at least 90% diastereoselective. In some embodiments, the reaction is at least 30% to at least 90% enantioselective.

One of skill in the art will appreciate that stereochemical configuration of certain of the products herein will be determined in part by the orientation of the product of the enzymatic step. Certain of the products herein will be “cis” compounds or “Z” compounds. Other products will be “trans” compounds or “E” compounds.

In certain instances, two cis isomers and two trans isomers can arise from the reaction of an olefinic substrate with a diazo reagent. The two cis isomers are enantiomers with respect to one another, in that the structures are non-superimposable mirror images of each other. Similarly, the two trans isomers are enantiomers. One of skill in the art will appreciate that the absolute stereochemistry of a product—that is, whether a given chiral center exhibits the right-handed “R” configuration or the left-handed “S” configuration-will depend on factors including the structures of the particular substrate and diazo reagent used in the reaction, as well as the identity of the enzyme. The relative stereochemistry—that is, whether a product exhibits a cis or trans configuration—as well as for the distribution of product mixtures will also depend on such factors.

In certain instances, the product mixtures have cis:trans ratios ranging from about 1:99 to about 99:1. The cis:trans ratio can be, for example, from about 1:99 to about 1:75, or from about 1:75 to about 1:50, or from about 1:50 to about 1:25, or from about 99:1 to about 75:1, or from about 75:1 to about 50:1, or from about 50:1 to about 25:1. The cis:trans ratio can be from about 1:80 to about 1:20, or from about 1:60 to about 1:40, or from about 80:1 to about 20:1 or from about 60:1 to about 40:1. The cis:trans ratio can be about 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, or about 1:95. The cis:trans ratio can be about 5:1, 10:1, 15:1, 20:1, 25:1, 30:1, 35:1, 40:1, 45:1, 50:1, 55:1, 60:1, 65:1, 70:1, 75:1, 80:1, 85:1, 90:1, or about 95:1.

The distribution of a product mixture can be assessed in terms of the enantiomeric excess, or “% ee,” of the mixture. The enantiomeric excess refers to the difference in the mole fractions of two enantiomers in a mixture. In certain instances, as a non-limiting example, for instance, the enantiomeric excess of the “E” or trans (R,R) and (S,S) enantiomers can be calculated using the formula: % ee_{E}=[(χ_{R,R}−χ_{S,S})/(χ_{R,R}+χ_{S,S})]×100%, wherein χ is the mole fraction for a given enantiomer. The enantiomeric excess of the “Z” or cis enantiomers (% ee_{Z}) can be calculated in the same manner.

In certain instances, product mixtures exhibit % ee values ranging from about 1% to about 99%, or from about −1% to about −99%. The closer a given % ee value is to 99% (or −99%), the purer the reaction mixture is. The % ee can be, for example, from about −90% to about 90%, or from about −80% to about 80%, or from about −70% to about 70%, or from about −60% to about 60%, or from about −40% to about 40%, or from about −20% to about 20%. The % ee can be from about 1% to about 99%, or from about 20% to about 80%, or from about 40% to about 60%, or from about 1% to about 25%, or from about 25% to about 50%, or from about 50% to about 75%. The % ee can be from about −1% to about −99%, or from about −20% to about −80%, or from about −40% to about −60%, or from about −1% to about −25%, or from about −25% to about −50%, or from about −50% to about −75%. The % ee can be about −99%, −95%, −90%, −85%, −80%, −75%, −70%, −65%, −60%, −55%, −50%, −45%, −40%, −35%, −30%, −25%, −20%, −15%, −10%, 10%, 15%, 20%, 25%, 30%, 35%, 40%, 45%, 50%, 55%, 60%, 65%, 70%, 75%, 80%, 85%, 90%, or about 95%. Any of these values can be % ee_{E} values or % ee_{Z} values.

Accordingly, some embodiments of the invention provide methods for producing a plurality of products having a % ee_{Z} of from about −90% to about 90%. In some embodiments, the % ee_{Z} is at least 90%. In some embodiments, the % ee_{Z} is at least −99%. In some embodiments, the % ee_{E} is from about −90% to about 90%. In some embodiments, the % ee_{E} is at least 90%. In some embodiments, the % ee_{E} is at least −99%.

### VII. Examples

The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1

### C—H Nitrene Insertions Catalyzed by P450 Variants

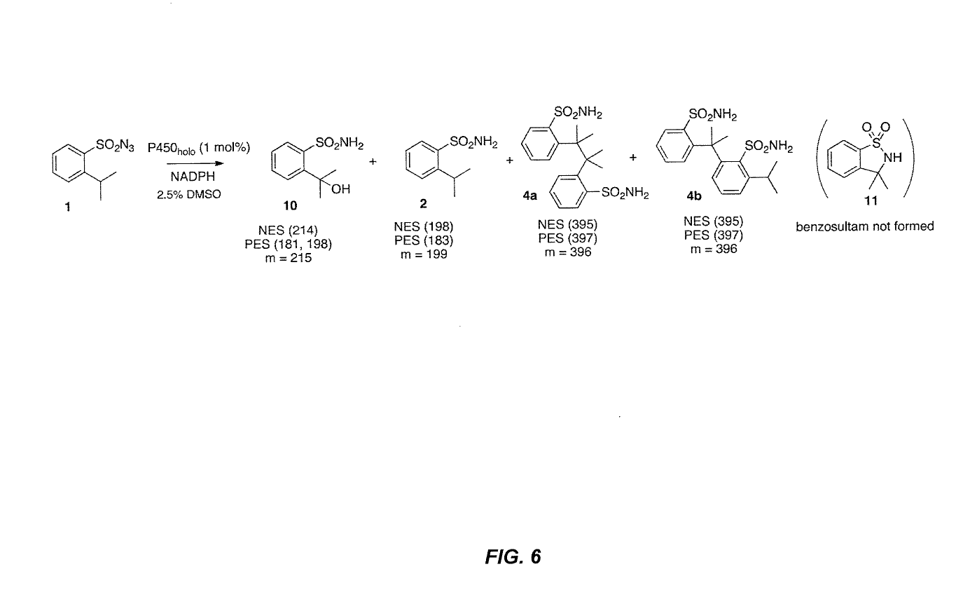

In this Example, we investigated whether heme containing enzymes could promote C—H nitrene insertions. We chose to utilize arylsulfonyl azides as nitrene precursors due to their ease of synthesis, their solubility in P450-compatible co-solvents and their previously demonstrated activation by metallo-porphyrins (J. V. Ruppel et al., Org. Lett. 9, 4889 (2007)). We screened P450s for binding to 2-isopropylbenzenesulfonyl azide (1, FIG. 6) and assessed selected ones for reaction under anaerobic conditions in the presence of NADPH (0, 0.05 and 1 equiv). The desired benzosultam 11, however, was not formed in any of these reactions.

FIG. 6 shows P450 bioconversions with 2-isopropylbenzenesulfonyl azide (1) under anaerobic conditions. NES=negative electrospray, PES=positive electrospray.

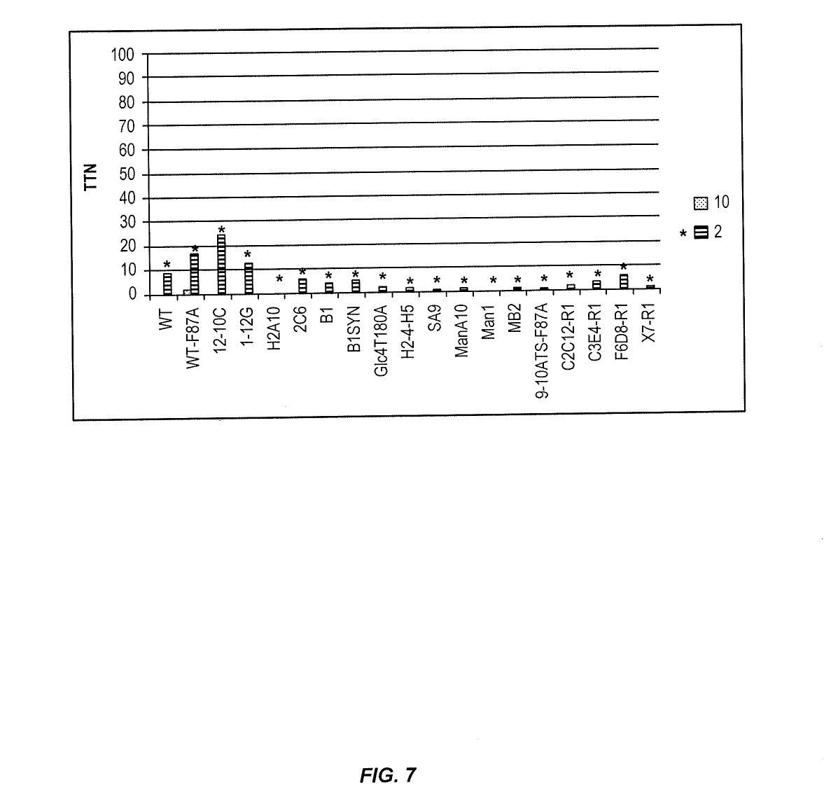

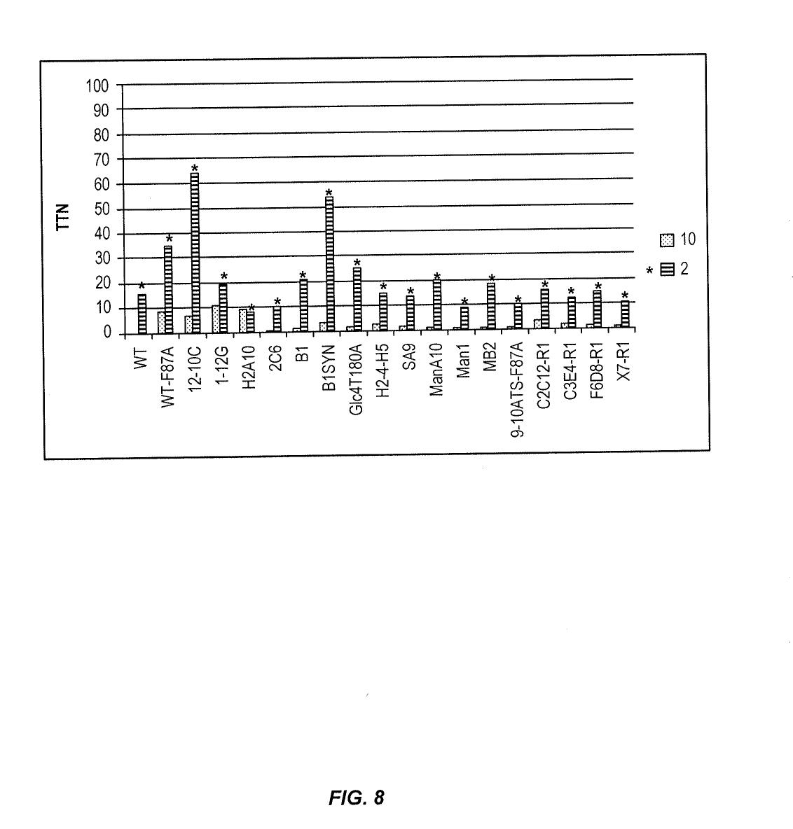

FIG. 7 shows P450 reactions with azide 1 in the absence of NADPH. Alcohol 10 and arylsulfonamide 2 (*) are defined in FIG. 6. Benzosultam was not formed in these reactions.

FIG. 8 shows P450 reactions with azide 1 in presence of 0.1 mM NADPH (0.05 eq). Alcohol 10 and arylsulfonamide 2 (*) are defined in FIG. 6. Benzosultam was not formed in these reactions.

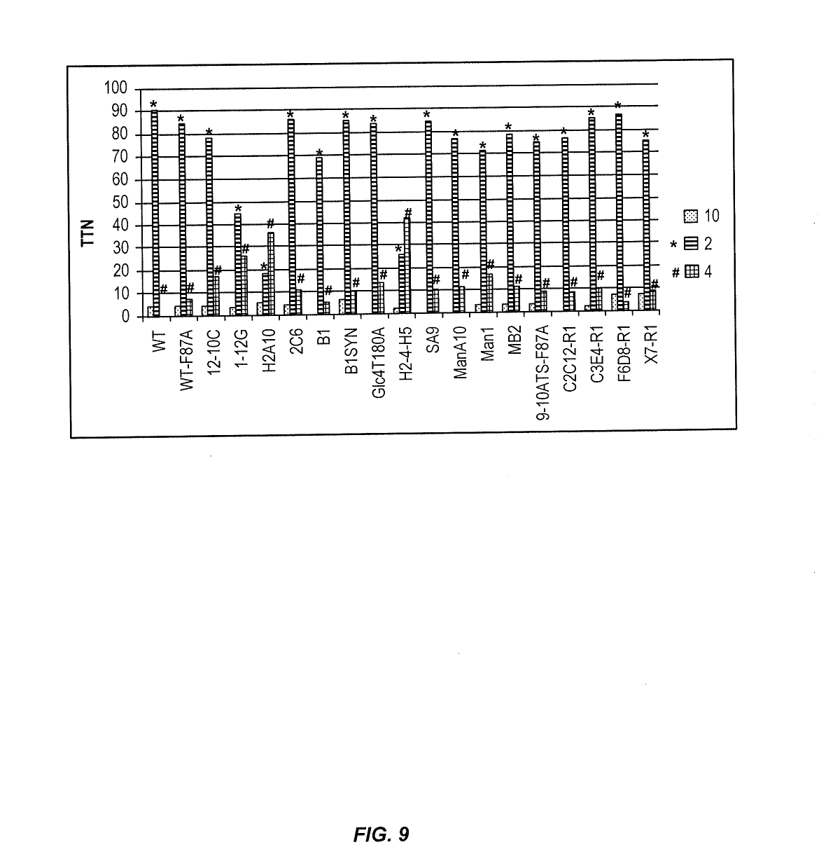

FIG. 9 shows P450 reactions with azide 1 in presence of 2 mM NADPH (1 eq). Alcohol 10, arylsulfonamide 2 and dimer 4 are defined in FIG. 6. The reaction was scaled with H2A10 to identify competing by-products by NMR and mass spectrometry. Benzosultam was not formed in these reactions.

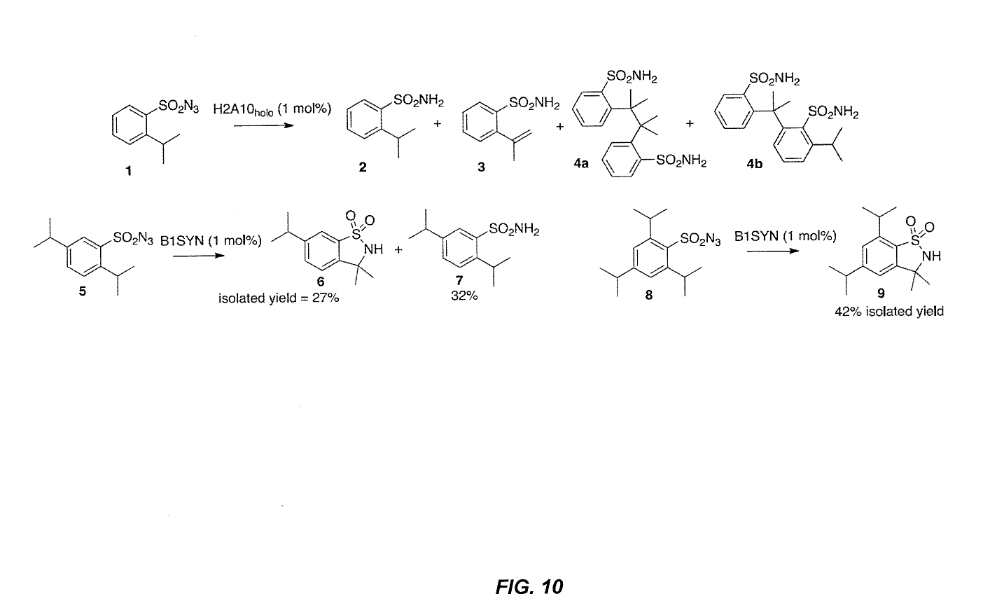

FIG. 10 shows P450-catalyzed amination of benzylic C—H bonds from arylsulfonyl azides. Products isolated from small-scale (30 mg azide) bioconversions were analyzed by NMR and mass spectrometry. Due to the unexpected dimerizations (4a and 4b), we reasoned that intramolecular C—H amination might be favored in more bulky multi-substituted arylsulfonyl azides. P450s chosen based on their ability to bind 1 could in fact catalyze benzosultam formation from azides 5 and 8. B1SYN (23 mutations from P450_{BM3}) binds azides 5 and 8 with micromolar affinity (K_{d} [5]=1.5 μM, K_{d} [8]=19 μM) and catalyzes up to 42 TTN of C—H amination to form the desired benzosultams (C. J. C. Whitehouse et al., Chem. Soc. Rev. 41, 1218 (2012) and E. W. Svastits et al., J. Am. Chem. Soc. 107, 6427 (1985)).

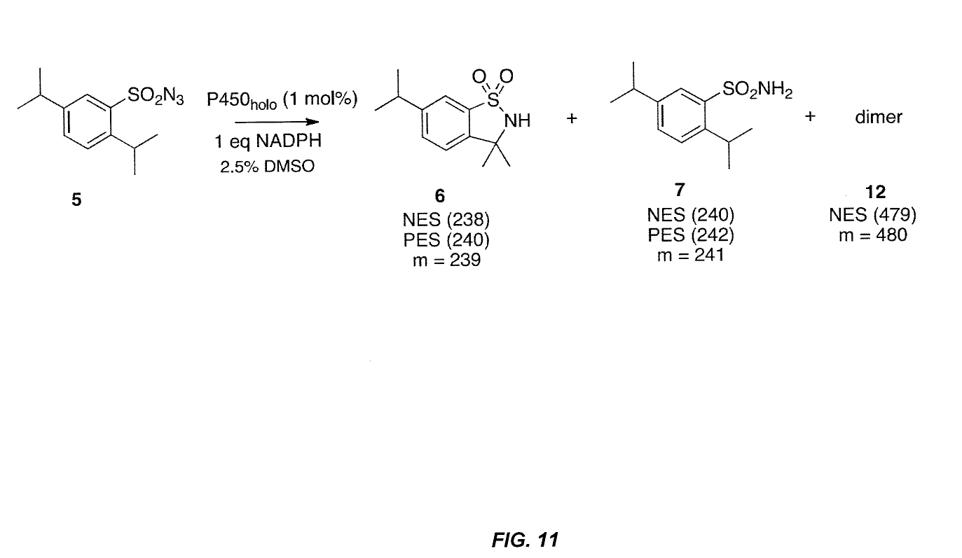

FIG. 11 shows shows P450 bioconversions with 2,5-disopropylbenzenesulfonyl azide 5 under anaerobic conditions. NES=negative electrospray, PES=positive electrospray.

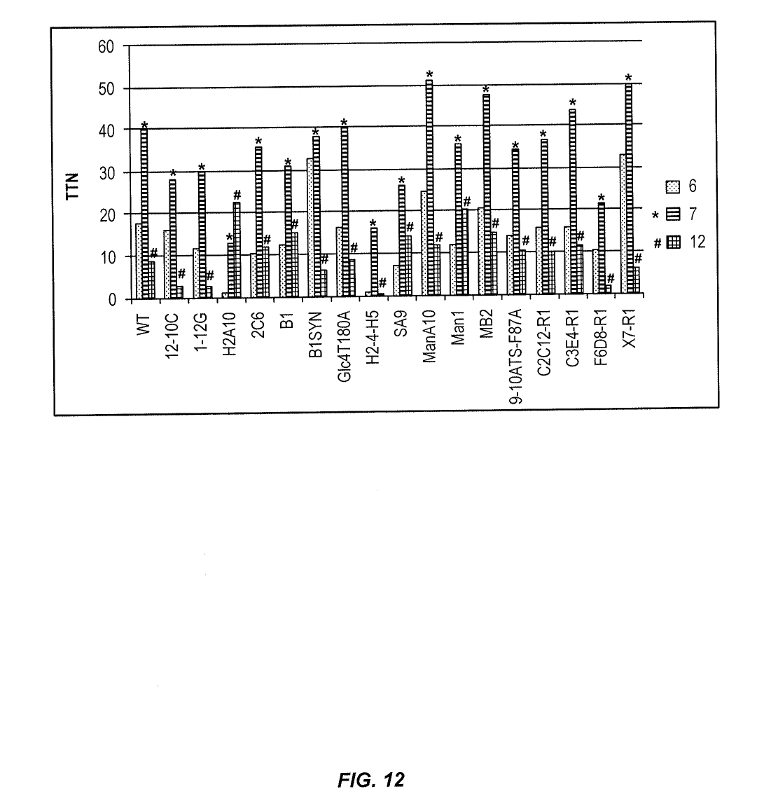

FIG. 12 shows P450 reactions with azide 8 in presence of 2 mM NADPH (1 eq). Benzosultam 6, arylsulfonamide 7 (*), and dimer 12 (#) are defined in FIG. 10. P450 variants catalyzed increased total turnover numbers (TTN) of C—H amination to form benzosultams from 2,5-diisopropylbenzenesulfonyl azide 5. For instance, B1SYN (e.g., P450_{BM3} with 23 amino acid substitutions) catalyzed over 30 TTN for form the benzosultam.

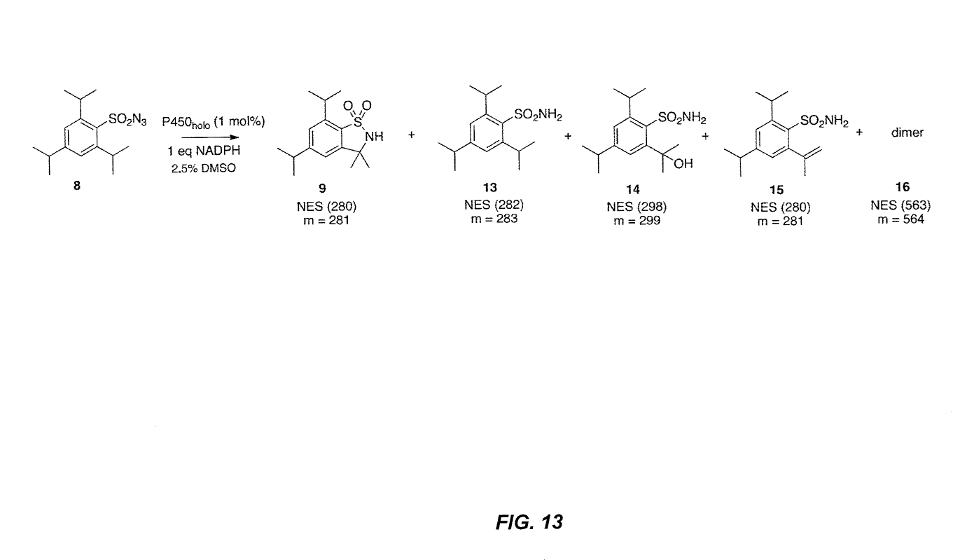

FIG. 13 shows P450 bioconversions with 2,4,6-triisopropylbenzenesulfonyl azide 8 under anaerobic conditions. NES=negative electrospray, PES=positive electrospray.

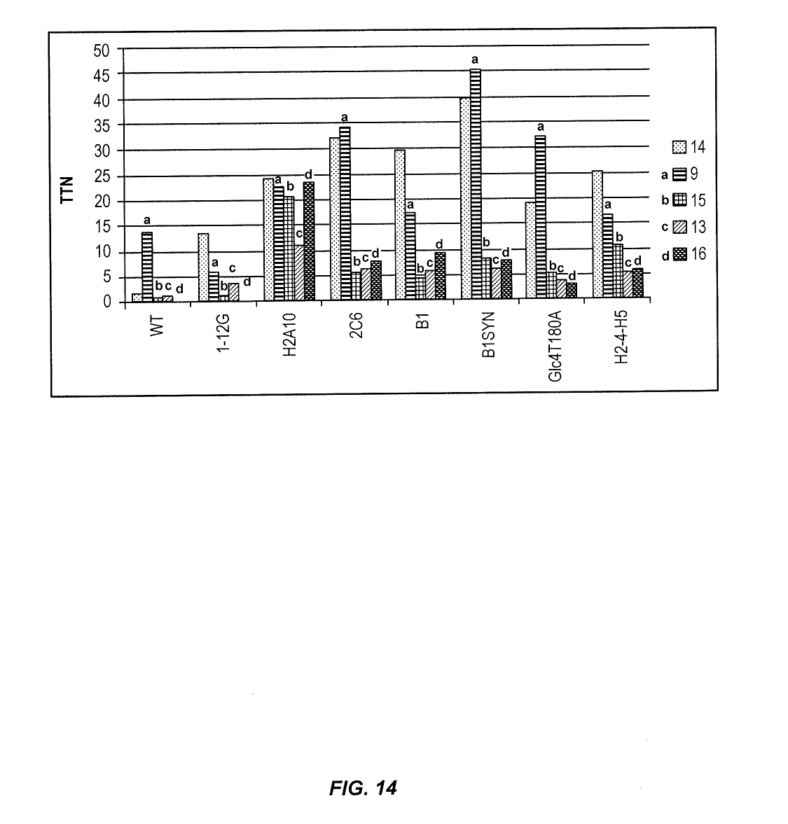

FIG. 14 shows P450 reactions with azide 8 in presence of 2 mM NADPH (1 eq). Benzosultam 9 (a), arylsulfonamide 13 (c), alcohol 14 (b), alkene 15 and dimer 16 (d) are defined in FIG. 13. P450 variants catalyzed increased total turnover numbers (TTN) of C—H amination to form benzosultams from 2,4,6-triisopropylbenzenesulfonyl azide 8. B1SYN (e.g., P450_{BM3} with 23 amino acid substitutions) catalyzed about 45 TTN for form the benzosultam.

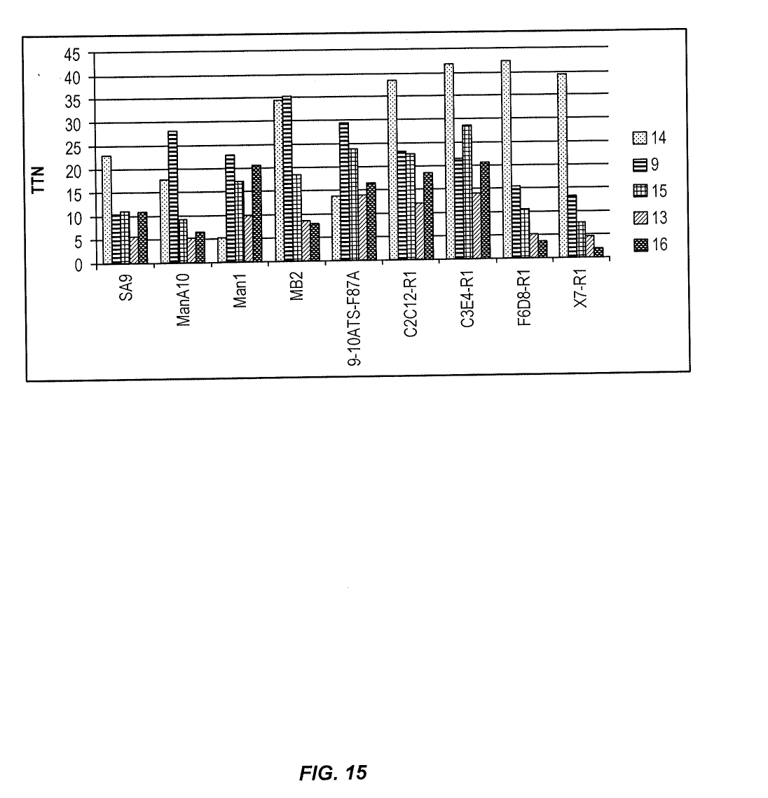

FIG. 15 shows P450 reactions with azide 8 in presence of 2 mM NADPH (1 eq). Benzosultam 9 (a), arylsulfonamide 13 (c), alcohol 14, alkene 15 (b) and dimer 16 (d) are defined in FIG. 13.

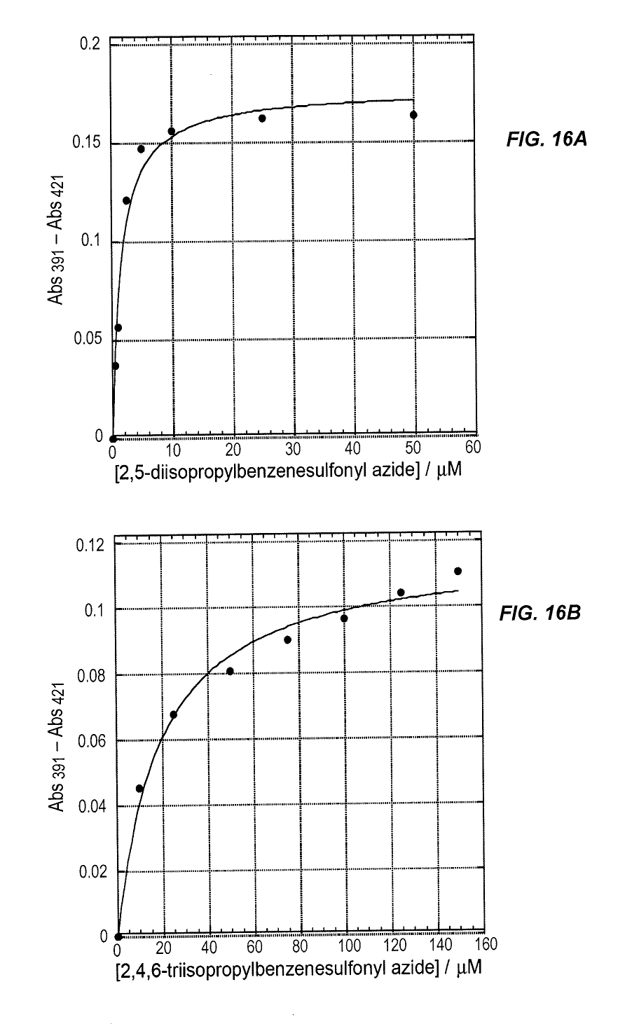

FIG. 16 shows B1SYN type I binding curves for azides 5 (A) and 8 (B). Kₐ (5)=1.5 μM, K_{d} (8)=19 μM.

Interestingly, free hemin was only an effective amination catalyst for azide 8 and not 5 (Table 7 and Table 8, FIG. 10), suggesting that the enzyme serves primarily to impose a conformation on the substrate that is favorable for nitrene C—H insertion.

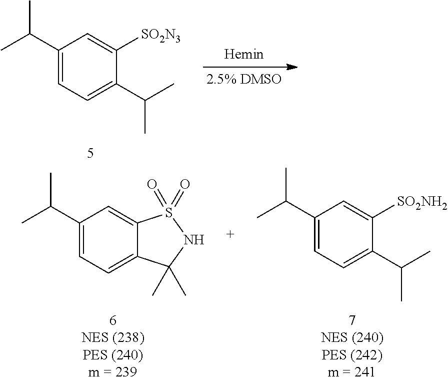

TABLE 7
Hemin reactions with azide 5 under anaerobic conditions.

| | | | TTN | |
|---|---|---|---|---|
| Cat. loading | | TTN | arylsulfonamide | |
| (mol %) | Reductant | benzosultam 6 | 7 (% yield) | |
| 1 | None | 0 | 0 | |
| 1 | 2 mM Na₂S₂O₄ | 0 | 5 | (5) |
| 1 | 10 mM Na₂S₂O₄ | 0 | 58 | (58) |
| 1 | 2 mM NADPH | 0 | 1 | (1) |
| 10 | 2 mM Na₂S₂O₄ | 0 | 1 | (10) |
| 10 | 10 mM Na₂S₂O₄ | 0 | 6 | (60) |

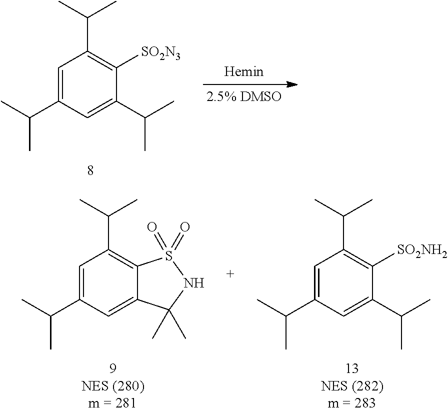

TABLE 8
Hemin reactions with azide 8 under anaerobic conditions.

| Cat. loading | | TTN benzosultam | | TTN arylsulfonamide | |
|---|---|---|---|---|---|
| (mol %) | Reductant | 9 (% yield) | | 13 (% yield) | |
| 1 | None | 0 | | 0 | |
| 1 | 2 mM Na₂S₂O₄ | 4 | (4) | 1 | (1) |
| 1 | 10 mM | 61 | (61) | 23 | (23) |
| | Na₂S₂O₄ | | | | |
| 1 | 2 mM NADPH | 0 | | 1 | (1) |
| 10 | 2 mM Na₂S₂O₄ | 0 | | 0 | |
| 10 | 10 mM | 4 | (40) | 3 | (30) |
| | Na₂S₂O₄ | | | | |

Intramolecular C—H Amination from Arylsulfonyl Azides

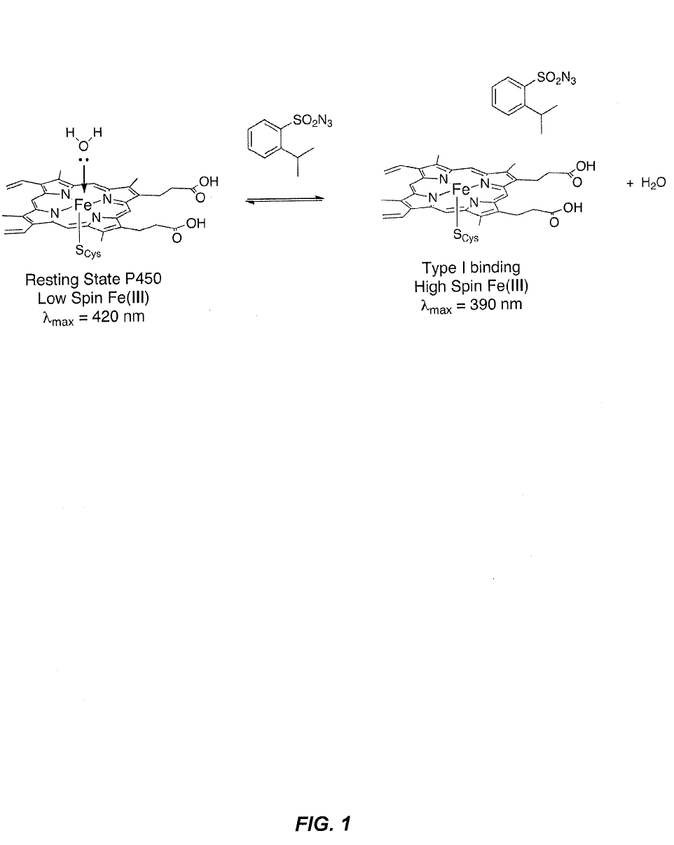

Arylsulfonyl azide binding screen. Cell lysate of the previously described compilation plate (Table 9 and Table 10) was scanned from 500-350 nm in a plate reader (Tecan M1000 UV/Vis) in the absence and presence of 100 μM 2-isopropylbenzenesulfonyl azide. Selected absorbance difference spectra that displayed Type I binding to the azide. FIG. 1 shows that P450BM3 variants display Type I binding to arylsulfonyl azides.

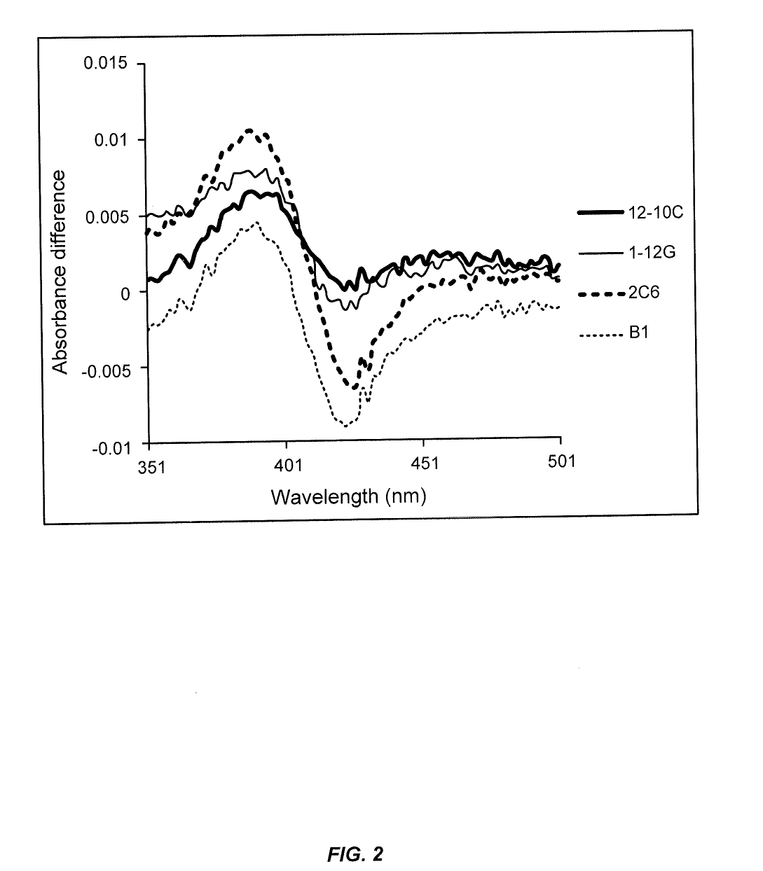

FIG. 2 shows an absorbance difference spectra for P450_{BM3} variants binding 2-isopropylbenzenesulfonyl azide. Sequence identities are shown on Table 9.

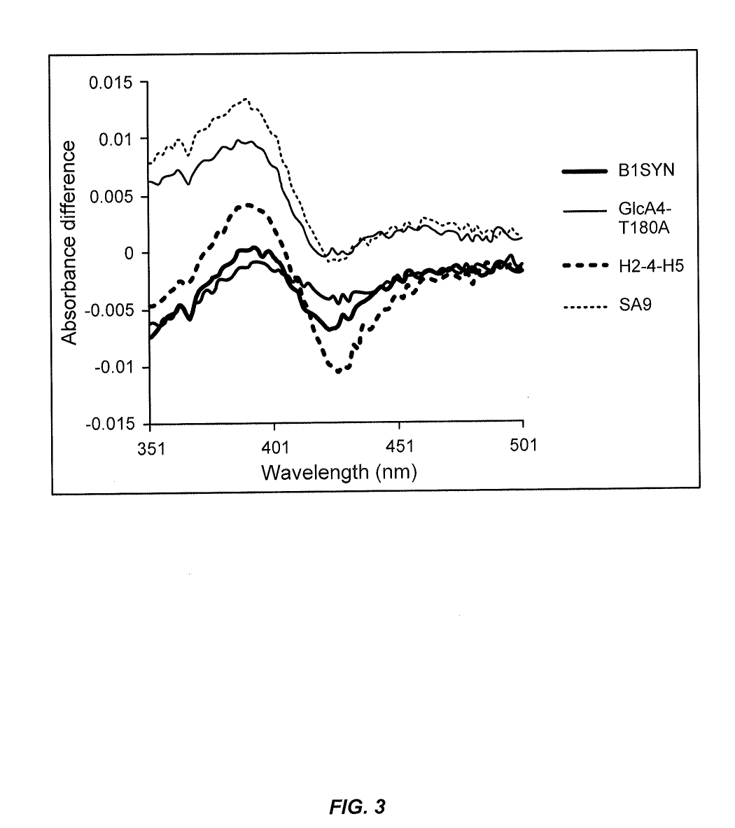

FIG. 3 shows an absorbance difference spectra for P450_{BM3} variants binding 2-isopropylbenzenesulfonyl azide. Sequence identities are shown on Table 9.

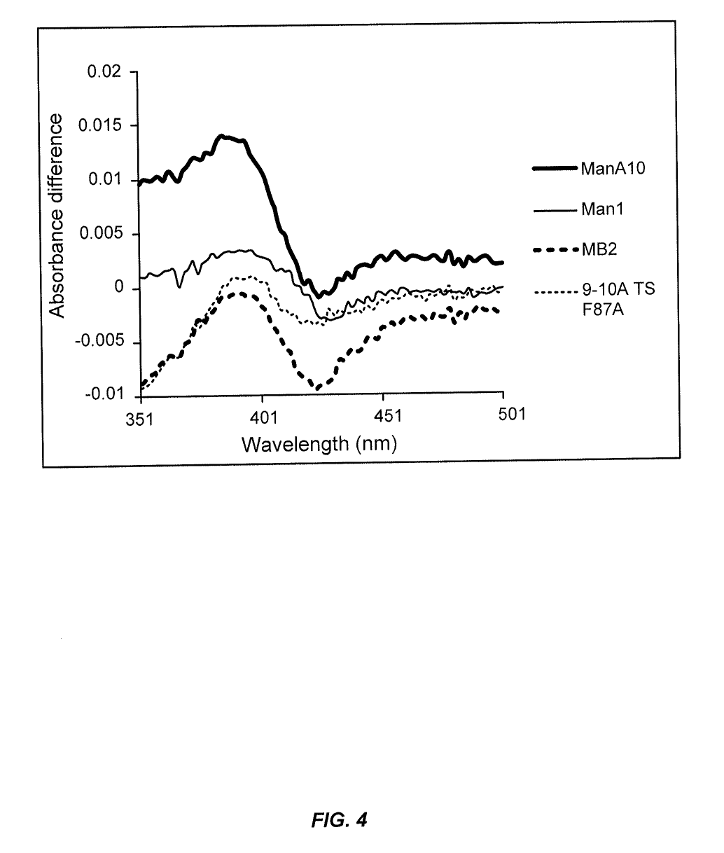

FIG. 4 shows an absorbance difference spectra for P450_{BM3} variants binding 2-isopropylbenzenesulfonyl azide. Sequence identities are shown on Table 9.

TABLE 9
Raw data from P450_{BM3} compilation plate screen.

| | Mutations compared to wild-type | Absolute | | |
|---|---|---|---|---|
| P450_{BM3} variants | P450_{BM3} (SEQ ID NO: 1) | activity^{a} | de^{b} | ee (cis)^{c} |
| CYP102A3 (SEQ ID NO: 30) | N/A | 0.004053 | −74 | −8 |
| CYP102A2 (SEQ ID NO: 29) | N/A | 0.002963 | −76 | −36 |
| P450_{BM3} (CYP102A1; SEQ ID | None | 0.002240 | −81 | 7 |
| NO: 1) | | | | |
| WT F87A | F87A | 0.001704 | −28 | 57 |
| WT T88L | T88L | 0.004522 | −78 | 23 |
| WT A328V | A328V | 0.000830 | −100 | N/A |
| J⁴ | V78A, T175I, A184V, F205C, S226R, | 0.001334 | −100 | N/A |
| | H236Q, E252G, R255S, A290V, L353V | | | |
| 139-3⁵ | V78A, H138Y, T175I, V178I, A184V, | 0.001386 | −86 | 0 |
| | F205C, S226R, H236Q, E252G, R255S, | | | |
| | A290V, L353V | | | |
| 9-10A⁴ | R47C, V78A, K94I, P142S, T175I, | 0.004292 | −74 | −20 |
| | A184V, F205C, S226R, H236Q, E252G, | | | |
| | R255S, A290V, L353V | | | |
| 9-10A L75W¹ | 9-10A + L75W | 0.005191 | −83 | −8 |
| 9-10A L75I¹ | 9-10A + L75I | 0.002267 | −85 | −3 |
| 9-10A A78F¹ | 9-10A + A78F | 0.002008 | −82 | −35 |
| 9-10A A78S¹ | 9-10A + A78S | 0.005098 | −81 | −6 |
| 9-10A A82G¹ | 9-10A + A82G | 0.002245 | −76 | −7 |
| 9-10A A82F¹ | 9-10A + A82F | N/A | N/A | N/A |
| 9-10A A82C¹ | 9-10A + A82C | 0.002487 | −74 | 16 |
| 9-10A A82I¹ | 9-10A + A82I | 0.001031 | −100 | N/A |
| 9-10A A82S¹ | 9-10A + A82S | 0.001483 | −82 | 14 |
| 9-10A A82L⁴ | 9-10A + A82L | 0.000591 | −100 | N/A |
| 9-10A F87A¹ | 9-10A + F87A | 0.001701 | −61 | −10 |
| 9-10A F87V¹ | 9-10A + F87V | 0.000000 | N/A | N/A |
| 9-10A F87I¹ | 9-10A + F87I | 0.000983 | −100 | N/A |
| 9-10A F87L¹ | 9-10A + F87L | 0.000710 | −100 | N/A |
| 9-10A T88C¹ | 9-10A + T88C | 0.002516 | −77 | 3 |
| 9-10A T260S¹ | 9-10A + T260S | 0.004259 | −82 | −6 |
| 9-10A T260N¹ | 9-10A + T260N | 0.003882 | −77 | 15 |
| 9-10A T260L¹ | 9-10A + T260L | 0.006173 | −77 | −2 |
| 9-10A A328V¹ | 9-10A + A328V | 0.006471 | −68 | −8 |
| 9-10A A328M¹ | 9-10A + A328M | 0.005180 | −82 | 6 |
| 9-10A A328F¹ | 9-10A + A328F | 0.002009 | −63 | −32 |
| 49-1A | R47C, V78T, A82G, F87V, K94I, | 0.001874 | −75 | −32 |
| | P142S, T175I, A184V, F205C, S226R, | | | |
| | H236Q, E252G, R255S, A290V, A328L, | | | |
| | L353V | | | |
| 35-7F | R47C, V78F, A82S, K94I, P142S, | 0.004514 | −73 | −52 |
| | T175I, A184V, F205C, S226R, H236Q, | | | |
| | E252G, R255S, A290V, A328L, L353V | | | |
| 53-5H¹ | 9-10A + A78F, A82S, A328F | 0.002840 | −80 | 2 |
| 7-11D | 9-10A + A82F, A328V | 0.036840 | −24 | −28 |
| 49-9B | R47C, V78A, A82G, F87V, K94I, | 0.000000 | N/A | N/A |
| | P142S, T175I, A184V, F205C, S226R, | | | |
| | H236Q, E252G, R255S, A290V, A328L, | | | |
| | L353V | | | |
| 41-5B | R47C, V78F, A82G, K94I, P142S, | 0.008391 | −77 | −17 |
| | T175I, A184V, F205C, S226R, H236Q, | | | |
| | E252G, R255S, A290V, A328V, L353V | | | |
| 13-7C¹ | 9-10A + A78T, A328L | 0.005493 | −73 | −43 |
| 12-10C | R47C, V78A, A82G, F87V, K94I, | 0.004566 | −73 | −21 |
| | P142S, T175I, A184V, F205C, S226R, | | | |
| | H236Q, E252G, R255S, A290V, | | | |
| | A328V, L353V | | | |
| 77-9H¹ | 9-10A + A78T, A82G, A328L | 0.003053 | −73 | −34 |
| 11-8E | R47C, V78A, F87V, K94I, P142S, | 0.001453 | −77 | 15 |
| | T175I, A184V, F205C, S226R, H236Q, | | | |
| | E252G, R255S, A290V, A328L, L353V | | | |
| 1-12G⁴ | 9-10A + A82L, A328V | 0.003884 | −70 | −19 |
| 29-3E | R47C, V78A, A82F, K94I, P142S, | 0.003425 | −80 | 15 |
| | T175I, A184V, F205C, S226R, H236Q, | | | |
| | E252G, R255S, A290V, A328F, L353V | | | |
| 29-10E | R47C, V78F, A82G, K94I, P142S, | 0.001935 | −70 | 16 |
| | T175I, A184V, F205C, S226R, H236Q, | | | |
| | E252G, R255S, A290V, A328F, L353V | | | |
| 68-8F¹ | 9-10A A78F, A82G, A328L | 0.004127 | −72 | −32 |
| 35E11⁶ | R47C, V78F, A82S, K94I, P142S, T175I, | 0.003600 | −71 | −14 |
| | A184V, F205C, S226R, H236Q, E252G, | | | |
| | R255S, A290V, A328F, L353V, E464G, | | | |
| | I710T | | | |
| 19A12⁶ | 35E11 + L52I, L188P, I366V | 0.006909 | −70 | −27 |
| ETS8⁶ | 35E11 + L52I, I366V | 0.003966 | −79 | −19 |
| (11-3)⁶ | 35E11 + L52I, A74S, L188P, | 0.005633 | −76 | −39 |
| | I366V | | | |
| (7-7)⁶ | 35E11 + L52I, A74E, S82G, A184V, | 0.010499 | −77 | −9 |
| | L188P, I366V | | | |
| H2A10 | 9-10A TS + F87V, L75A, L181A, | 0.066422 | −8 | −94 |
| | T268A | | | |
| SL2-6F8 | R47C, L52I, V78F, A82S, K94I, | 0.000778 | −100 | N/A |
| | P142S, T175I, A184V, F205C, S226R, | | | |
| | H236Q, E252G, R255S, A290V, A328L, | | | |
| | K349N, L353V, I366V, E464G, I710T | | | |
| A12SL-17-4 | R47C, L52I, A74E, V78F, A82S, K94I, | 0.010935 | −80 | 6 |
| | P142S, T175I, A184V, L188P, F205C, | | | |
| | S226R, H236Q, E252G, R255S, A290V, | | | |
| | A328F, L353V, I366V, E464G, I710T | | | |
| H2-2-A1² | 9-10A TS + F87V, L75A, L181A, | 0.003042 | −75 | −11 |
| | L437A | | | |
| A12RM-2-8 | R47C, L52I, A74E, V78F, A82S, K94I, | 0.007705 | −77 | −13 |
| | P142S, T175I, A184S, L188P, F205C, | | | |
| | S226R, H236Q, E252G, R255S, A290V, | | | |
| | A328F, L353V, I366V, E464G, I710T | | | |
| H2-5-F10 | 9-10A TS F87V, L75A, I263A, T268A, | 0.141237 | −46 | −56 |
| | L437A | | | |
| 13C9R1 | L52I, I58V, L75R, F87A, H100R, | 0.001980 | −100 | N/A |
| | S106R, F107L, A135S, A184V, N239H, | | | |
| | S274T, L324I, V340M, I366V, K434E, | | | |
| | E442K, V446I | | | |
| 22A3 | 13C9R1 + F162I E434K K442E, I446V | 0.004053 | −70 | 4 |
| 2C6³ | 9-10A + A78L, F87A, V184T, G315S, | 0.004257 | −78 | −15 |
| | A330V | | | |
| 9C7³ | 9-10A + C47R, A78L, F87G, I94K, | 0.007258 | −79 | −5 |
| | A180V, V184T, G315S, A330V, Y345C | | | |
| B1³ | 9-10A + C47R, A78L, F87A, I94K, | 0.002246 | −61 | −14 |
| | V184T, I263M, G315S, A330V | | | |
| B1SYN³ | 9-10A + C47S, N70Y, A78L, F87A, | 0.002705 | −76 | −23 |
| | I174N, I94K, V184T, I263M, G315S, | | | |
| | A330V | | | |
| H2-4-D4 | 9-10A TS + F87V, L75A, M177A, | 0.052439 | 57 | −84 |
| | L181A, T268A, L437A | | | |
| E12 A87V³ | 9-10A + C47R, A78L, F87V, I94K, | 0.001990 | −65 | −52 |
| | A111V, V141I, A180V, V184T, G315S, | | | |
| | A330V | | | |
| GlcA4 T180A | 9-10A + C47R, F81W, A82S, F87A, | 0.004925 | −78 | 12 |
| | I94K | | | |
| H2-8-C7² | 9-10A TS + F87V, L75A, L181A | 0.000808 | −100 | N/A |
| CH-F8 | 9-10A + L51A, C47A, F87V, I94K, | 0.001126 | −100 | N/A |
| | L181A, C205F, S254R, I366V, L437A, | | | |
| | E442K | | | |
| H2-4-H5² | 9-10A TS + F87V, L75A, M177A, | 0.001229 | −100 | N/A |
| | L181A | | | |
| SA9 | 9-10A + C47R, F81W, A82I, F87A, | 0.004170 | −81 | 11 |
| | I94K, A180T, A197V | | | |
| ManA10 | 9-10A + C47R, F81S, A82V, F87A, | 0.006340 | −82 | 14 |
| | I94K, A180T, A197V | | | |
| Man1 | 9-10A + C47R, F81L, A82T, F87A, | 0.003053 | −73 | 21 |
| | I94K | | | |
| MB2 | 9-10A + C47R, F81W, A82I, F87A, | 0.003282 | −77 | 10 |
| | I94K | | | |
| HA62 | 9-10A + C47R, F81A, A82L, F87A, | 0.003375 | −81 | −5 |
| | I94K | | | |
| 9-10A TS | V78A, P142S, T175I, A184V, S226R, | 0.001920 | −75 | −54 |
| | H236Q, E252G, A290V, L353V, I366V, | | | |
| | E442K | | | |
| 9-10A TS F87A | 9-10A TS + F87A | 0.001546 | −60 | 5 |
| 25F7 | 9-10A + C47R, A74F, A78S, F87A, | 0.001829 | −81 | 43 |
| | I282K, C205F, S255R | | | |
| 24C4 | 9-10A + C47R, A74I, A78L, F87A, | 0.000783 | −100 | N/A |
| | I94K, C205F, S255R | | | |
| 5A1 | 9-10A + M30T, C47R, A74F, A78S, | 0.002471 | −80 | 15 |
| | I94K, C205F, S255R, Q310L, I366V, | | | |
| | E442K | | | |
| 8B3 | 9-10A + M30T, C47R, A74F, A78S, | 0.001315 | −100 | N/A |
| | I94K, C205F, C255R, L310Q, Q323L, | | | |
| | I366V, N381K, R398H, E441K | | | |

Determined by GC analysis on a chiral β-CDX column.
^{a}Reported as the sum of the area of the cyclopropane peaks over the area of the internal standard.
^{b}Diastereomeric excess = ([cis] − [trans])/([cis] + [trans]).
^{c}(R,S)-(S,R).).
¹P. Meinhold et al., Adv. Synth. Catal. 348, 763 (2006).
²J. C. Lewis et al., Chembiochem: a European journal of chemical biology 11, 2502 (2010).
³J. C. Lewis et al., Proceedings of the National Academy of Sciences of the United States of America 106, 16550 (2009).
⁴M. W. Peters et al., J. Am. Chem. Soc. 125, 13442 (2003).
⁵A. Glieder et al., Nat. Biotechnol. 20, 1135 (2002).
⁶R. Fasan et al., Angew. Chem., Int. Ed. 46, 8414 (2007).

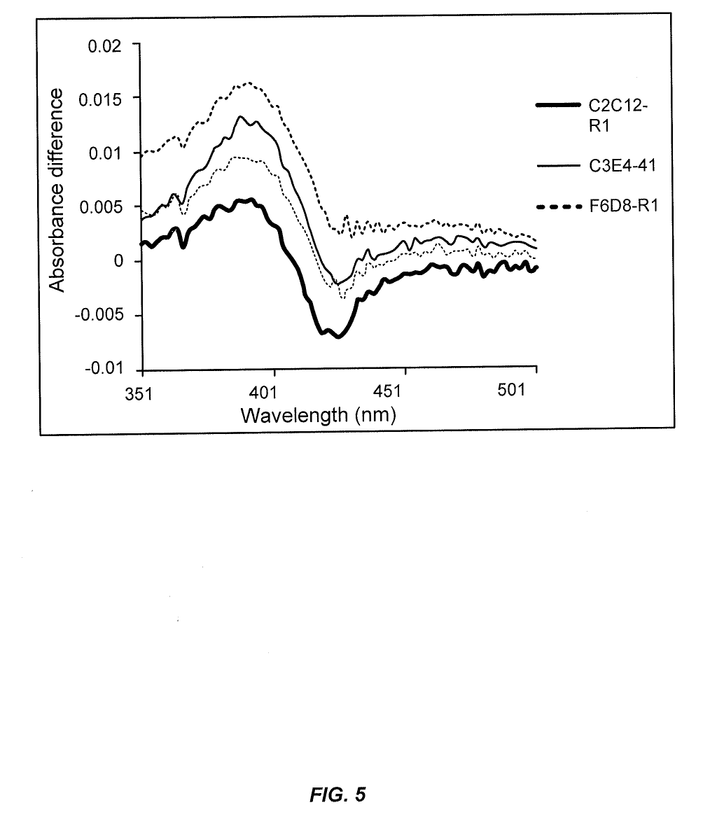

FIG. 5 Absorbance difference spectra for P450_{BM3} variants binding 2-isopropylbenzenesulfonyl azide. Sequence identities are shown on Table 10.

TABLE 10
Raw GC screening data for the chimeric P450s in the compilation plate.

| | Chimeric P450s | | | |
|---|---|---|---|---|
| | (heme domain | | | |
| | block | Absolute | | |
| P450 variant | sequence)¹ | activity^{a} | de^{b} | ee (cis)^{c} |
| CYP102A1(P450_{BM3}) + | 11111111 | 0.001704 | −28 | 56 |
| F87A¹ | | | | |
| CYP102A2 + F88A¹ | 22222222 | N/A | N/A | N/A |
| CYP102A3 + F88A¹ | 33333333 | N/A | N/A | N/A |
| 5R1² | 32312231 | 0.008625 | 58 | 19 |
| 9R1² | 12112333 | 0.0042707 | 58 | 24 |
| 12R1² | 12112333 | 0.0701514 | 32 | −49 |
| C1D11R1² | 21113312 | 0.007138 | 51 | 9 |
| C2B12R1² | 32313233 | 0.005914 | 38 | −5 |
| C2C12R1² | 21313111 | 0.006226 | 28 | 9 |
| C2E6R1² | 11113311 | 0.008731 | 25 | 6 |
| C2G9R1² | 22213132 | 0.007975 | 15 | 31 |
| C3D10R1² | 22132231 | 0.004898 | −16 | −2 |
| C3E4R1² | 21313311 | 0.007893 | 14 | 17 |
| F3H12R1² | 21333233 | 0.005586 | −56 | −17 |
| F6D8R1² | 22313233 | 0.008088 | −76 | −6 |
| C3B5R1² | 23132233 | 0.014722 | −81 | 4 |
| X7R1² | 22312333 | 0.017305 | −4 | −34 |

^{a}Reported as the sum of the area of the cyclopropane peaks over the area of the internal standard.
^{b}Diastereomeric excess = ([cis] − [trans])/([cis] + [trans]).
^{c}(R,S)-(S,R).
¹C. R. Otey et al., PLoS Biol. 4, 789 (2006).
²M. Landwehr et al., Chem. Biol. 14, 269 (2007).

Small-Scale Amination Reactions Under Anaerobic Conditions.

Reaction conditions were as described herein and analyzed by reverse-phase LC-MS (Agilent 1100 series LC-MSD), acetonitrile-water, using a C18 column (Peeke Scientific, Kromasil 100 5 μm, 50×4.6 mm ID). Acetonitrile gradient for 2-isopropylbenzenesulfonyl azide reactions: 10-22% (8 min), 22-60% (10 min), 60% (2 min), at 1.5 mL min⁻¹. Retention times: alcohol 10 (6.3 min), sulfonamide 2 (10.6 min), dimer 4 (12.3 min), azide 1 (17.6 min). Acetonitrile gradient for 2,5-diisopropylbenzenesulfonyl azide and 2,4,6-triisopropylbenzenesulfonyl azide reactions: 30-50% (10 min), 50-90% (8 min), 90% (2 min), at 1.5 mL min⁻¹ Retention times: benzosultam 6 (5.4 min), sulfonamide 7 (7.2 min), dimer 12 (8.6 min), azide 5 (14.4 min); alcohol 14 (8.3 min), benzosultam 9 (9.8 min), olefin 15 (10.8 min), sulfonamide 13 (11.8 min), dimer 16 (15.4 min), azide 8 (16.5 min) (see FIGS. 6, 10, 11 and 13).

Preparative-Scale Bioconversions.

These reactions were conducted anaerobically as described herein.

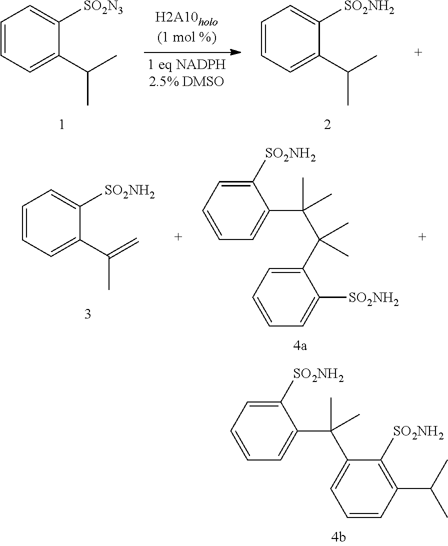

H2A10 scale-up with 2-isopropylbenzenesulfonyl azide (1). Preparation used 48 mg of azide 1 and 2 μmol H2A10ₕₒₗₒ (0.01 equiv). The products were purified by reverse phase HPLC to give 6 mg of arylsulfonamide 2 (15%), 2 mg of olefin 3 (5%), 11 mg of dimer 4a (25%) and 4 mg of dimer 4b (5%).

2-isopropylbenzenesulfonamide (2). ¹H NMR (500 MHz, DMSO): δ 7.82 (1H, d, J=8.14), 7.54 (2H, m), 7.45 (2H, br s), 7.31 (1H, ddd, J=2.16, 6.03, 8.16), 3.84 (1H, sep, J=6.71), 1.20 (6H, d, J=6.71). 13C NMR (125 MHz, DMSO): δ 147.04, 141.35, 132.11, 127.63, 126.76, 125.64, 28.54, 23.87. Expected m/z for C9H13NO2NaS⁺ 222.0559. Observed m/z 222.0552.

2-(prop-1-en-2-yl)benzenesulfonamide (3). ¹H NMR (500 MHz, DMSO): δ 7.89 (1H, dd, J=8.08, 1.23), 7.53 (1H, m), 7.44 (1H, m), 7.26 (1H, dd, J=7.60, 1.34), 7.22 (2H, s), 5.20 (1H, ap p, J=1.60), 4.86 (1H, m), 2.05 (3H, br s). ¹³C NMR (HMBC/HSQC 500 MHz, DMSO): δ 141.83, 141.39, 131.27, 129.76, 126.89, 126.70, 115.79, 24.86. Expected m/z for C9H12NO2S⁺ 198.0583. Observed m/z 198.19.

2,2′-(2,3-dimethylbutane-2,3-diyl)dibenzenesulfonamide (4a). ¹H NMR (500 MHz, DMSO): δ 8.16 (1H, dd, J=8.22, 1.45), 7.53 (2H, s), 7.33 (1H, ddd, J=8.18, 6.74, 1.26), 7.25 (1H, m), 7.18 (1H, d, J=8.06), 1.59 (6H, s). ¹³C NMR (HMBC/HSQC 500 MHz, DMSO): δ 145.11, 144.81, 134.17, 128.95, 128.26, 125.77, 48.79, 29.78. Expected m/z for C18H25N2O4S2⁺ =397.1250. Observed m/z 397.0147.

TABLE 11
Summary of NMR data for dimer 4b.

| position | ¹³C, type | ¹H (J in Hz) | HMBC | COSY |
|---|---|---|---|---|
| 1 | 140.45, C | — | — | — |
| 2 | 143.51, C | — | — | — |
| 3 | 27.97, CH | 3.77, sep (6.77) | 1, 2, 4, 5 | 4 |
| 4 | 23.72, CH3 | 1.17, d (6.72) | 2, 3 | 3 |
| 5 | 126.72, CH | 7.38, d (8.22) | 1, 3, 8 | 6 |
| 6 | 129.67, CH | 7.18, d (8.23) | 2, 7, 9 | 5, 7 |
| 7 | 124.17, CH | 7.63, m | 1, 2, 6, 9 | 7 |
| 8 | 148.33, C | — | — | — |
| 9 | 44.56, C | — | — | — |
| 10 | 31.82, CH3 | 1.80, s | 8, 11, 12 | — |
| 11 | 145.77, C | — | — | — |
| 12 | 129.96, CH | 7.57, m | 9, 14, 16 | — |
| 13 | 131.47, CH | 7.56, m | — | — |
| 14 | 126.59, CH | 7.46, m | 12, 16 | 15 |
| 15 | 129.74, CH | 8.08, d (7.97) | 11, 13 | 14 |
| 16 | 143.15, C | — | — | — |
| NH | | 6.83, br s | — | — |
| NH | | 7.30, br s | — | — |

Expected m/z for C₁₈H₂₅N₂O₄S₂⁺ 397.1250. Observed m/z 397.1245.

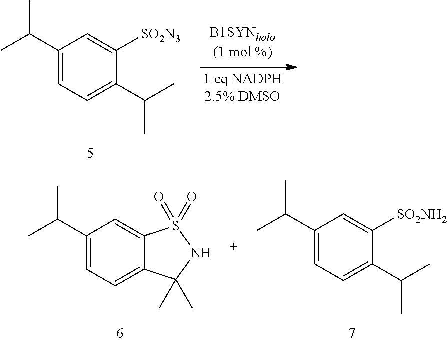

B1SYN Scale-Up with 2,5-Diisopropylbenzenesulfonyl Azide (5).

Preparation used 24 mg of azide 5 and 0.9 μmol B1SYNₕₒₗₒ (0.01 equiv). The products were purified by reverse phase HPLC to give 6 mg of benzosultam 6 (27%) and 7 mg of arylsulfonamide 7 (32%).

Enzymatically produced diisopropyl benzosultam (6). ¹H NMR (600 MHz, CDCl₃): δ 7.59 (1H, s), 7.48 (1H, d, J=8.03), 7.29 (1H, d, J=8.11), 4.50 (1H, s), 3.01 (1H, sep, J=6.95), 1.64 (6H, s), 1.28 (6H, d, J=6.98). ¹³C NMR (150 MHz, CDCl₃): δ 150.83, 143.69, 135.34, 132.39, 122.74, 118.70, 60.78, 34.17, 29.92, 23.92. Expected m/z for C₁₂H₁₈NO₂S⁺ 240.1053. Observed m/z 240.1059. NMR spectra of enzymatically produced diisopropylbenzosultam were identical with those of a synthetic standard produced according to Ruppel et al (J. V. Ruppel et al., Org. Lett. 9, 4889 (2007)). Moreover, the identity of the benzosultam could be further supported by the observation of HMBC correlations from the amide proton to the geminal dimethyl groups.

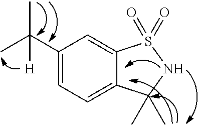 [SMILES: CC(C)c1ccc2c(c1)S(=O)(=O)NC2(C)C]

2,5-diisopropylbenzenesulfonamide (7). ¹H NMR (500 MHz, CDCl₃): δ 7.86 (1H, d, J=1.7 Hz), 7.41 (2H, m), 4.81 (br s, 2H), 3.76 (1H, sep, J=6.80 Hz), 2.94 (1H, sep, J=6.97 Hz), 1.30 (6H, J=6.80 Hz), 1.26 (6H, d, J=6.96 Hz). ¹³C NMR (125 MHz, CDCl₃): δ 146.93, 145.34, 138.67, 131.43, 128.03, 126.16, 33.85, 29.53, 24.21, 23.92. Expected m/z for C₁₂H₂₀NO₂S⁺ 242.1209. Observed m/z 242.1210.

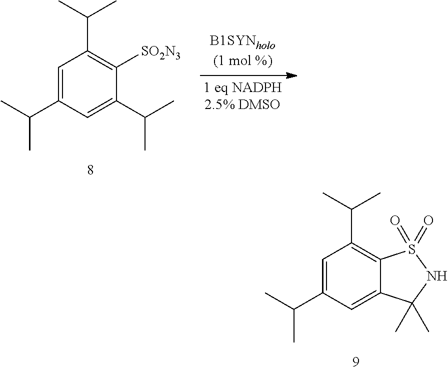

B1SYN Scale-Up with 2,5-Diisopropylbenzenesulfonyl Azide (5).

Preparation used 13 mg of azide 5 and 0.4 μmol B1SYNₕₒₗₒ (0.01 equiv). The product was purified by reverse phase HPLC to give 5 mg of benzosultam 9 (42%).

Enzymatically produced triisopropyl benzosultam (9). ¹H NMR (500 MHz, CDCl₃): δ 7.22 (1H, d, J=1.37), 6.98 (1H, d, J=1.38), 4.47 (1H, s), 3.61 (1H, sep, J=6.80), 2.98 (1H, sep, J=7.01), 1.63 (6H, s), 1.35 (6H, d, 6.81), 1.27 (6H, d, 6.92). ¹³C NMR (125 MHz, CDCl₃): δ 155.7, 146.8, 145.5, 131.0, 124.5, 117.9, 59.9, 34.8, 30.0, 29.6, 24.05, 23.72. Expected m/z for C₁₅H₂₄NO₂S⁺ 282.1522. Observed m/z 282.1528.

### Synthesis of 2-isopropylbenzenesulfonyl azide (1)

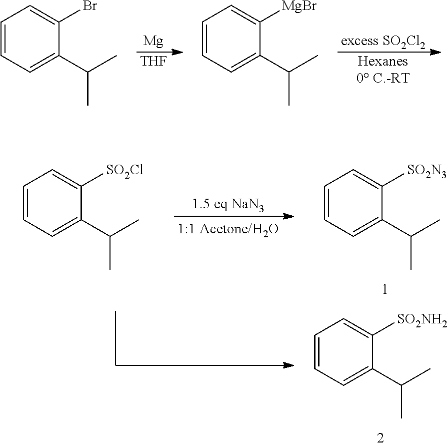

### 2-isopropylbenzenesulfonyl chloride

Freshly polished magnesium turnings (0.488 g, 20.1 mmol) were suspended in dry THF (16 ml) and stirred vigorously. An aliquot of a solution of 2-bromocumene (2.00 g, 10 mmol) in 8 ml dry THF was added and the reaction was initiated by heating to a brief boil. The remainder of the starting material was slowly added to maintain reaction. After three hours, the reaction was cooled with an ice bath and the solution was transferred under nitrogen via Teflon tubing to a solution of SO₂Cl₂ (4.0 ml, 50 mmol) in dry hexanes (25 ml) also at 0° C. and left overnight. The reaction was slowly poured over ice cold water (50 ml) and extracted with DCM 4 times. The organic layer was dried (Na₂SO₄), filtered and concentrated in vacuo. The crude product was purified by chromatography (SiO₂, 10% ether/hexanes) to afford the sulfonyl chloride (1.302 g, 60%). ¹H NMR (CDCl₃, 300 MHz): δ=8.00 (m, 1H), 7.73-7.55 (m, 2H), 7.40-7.31 (m, 1H), 4.22-4.08 (m, 1H), 1.36 (d, J=6.8 Hz, 6H).

### 2-isopropylbenzenesulfonyl azide (1)

The chloride (0.800 g, 3.7 mmol) was dissolved in acetone (9.5 ml) and cooled with an ice bath. A cold solution of sodium azide (0.358 g, 5.5 mmol) in water (9.5 ml) was added dropwise and left to react overnight. The reaction mixture was extracted with DCM, dried, filtered and solvent was evaporated in vacuo. Flash chromatography (SiO₂, 10% ether/hexanes) gave the sulfonyl azide 1 (0.666 g, 80%). ¹H NMR (CDCl₃, 300 MHz): δ=8.04 (dd, J=8.1, 1.3 Hz, 1H), 7.70-7.56 (m, 2H), 7.38 (m, 1H), 3.82-3.67 (m, 1H), 1.36-1.27 (d, J=6.8 Hz, 6H). HRMS (EI⁺): Calcd. for C₉H₁₁SO₂N₃ (M⁺) m/z 225.0572. found 225.0581.

### 2-isopropylbenzenesulfonyl amide (2)

The chloride (0.241 g, 1.11 mmol) was dissolved in chloroform (9 ml) and cooled with an ice bath Ammonium hydroxide (30%, 0.35 mL, 5.6 mmol) was added dropwise and left to react overnight. The reaction mixture was extracted with DCM, dried, filtered and solvent was evaporated in vacuo. Flash chromatography (SiO₂, 10% ether/hexanes) gave the sulfonyl amide 7 (0.68 g, 62%). ¹H NMR (CDCl₃, 300 MHz): δ=8.02 (d, J=9.3 Hz, 1H), 7.60-7.47 (m, 2H), 7.35-7.27 (m, 1H), 4.78 (s, 2H), 3.87-3.74 (m, 1H), 1.32 (d, J=6.8 Hz, 6H). HRMS (EI⁺): Calcd. for C₉H₁₃SO₂N (M⁺) m/z 199.0667. found 199.0627.

### 2,5-diisopropylbenzenesulfonyl amide (7)

Same procedure as used above for 2. 7 was obtained in 75% yield (0.345 g). ¹H NMR (CDCl₃, 300 MHz): δ=7.92-7.80 (m, 1H), 7.46-7.36 (m, 2H), 4.75 (s, 2H), 3.85-3.68 (m, 1H), 3.02-2.85 (m, 1H), 1.31 (d, J=6.8 Hz, 6H), 1.26 (d, J=6.9 Hz, 6H).

### Example 2

### C—H and N—H Bond Insertion by P450 Variants in the Presence of Diazo Compounds

Metal carbenoids formed via diazo transfer are known to participate in C—H and heteroatom-H insertion reactions (H. M. L. Davies and J. R. Manning, Nature (London, United Kingdom) 451, 417 (2008); S.-F. Zhu and Q.-L. Zhou, Accounts of Chemical Research 45, 1365 (2012)). The engineered P450 variants described herein demonstrate reactivity towards weak C—H and N—H bonds. We have examined the reaction of a few existing enzymes with aniline in the presence of ethyl diazopropionate, and found that the reaction proceeds catalytically even with P450 variants that are unoptimized for the aniline substrate. FIG. 17 shows C—H and N—H bond insertion by P450 variants in the presence of diazo compounds.

### Example 3

### In Vivo and In Vitro C—H Amination and C═C Aziridination Catalyzed by Heme Enzymes

Organometallic Catalysts for C—

H amination. Traditional approaches to C—H amination have employed organic scaffolds such as porphyrins, salens, corrins, among others to bind and tune the reactivity of a metal (Fe, Co, Ru, Rh, Mn, among others) that mediates the C—H nitrogen insertion reaction. Typical precursors are iminoiodanes that are either formed in situ or are added as the preformed reagents to the reaction mixture, or organoazide reagents such as alkyl, aryl, phorphoryl or sulfonyl azides. Still other precursors that have been successfully used include haloamine derivatives such as chloramine-T and bromamine-T, as well as N-tosyloxycarbamates and N-mesyloxycarbamates.

Several highly active catalysts have been described that rely on in situ-formed iminoiodane substrates and rhodium-based organometallic complexes. Other successful catalyst designs include metal-porphyrin-based catalysts that are highly active on azide substrates, and are capable of mediating inter- or intramolecular C—H amination reactions. Still other catalysts that employ palladium, silver, or gold metals with organic ligand scaffolds have been used with success in inter- and intra-molecular amination reactions.

Cytochrome P450s.

Cytochrome P450 enzymes are a diverse and broadly distributed class of monooxygenases. These enzymes are present in all domains of life, and catalyze many important reactions in cellular detoxification and secondary metabolism. Conserved features of this enzyme class include a conserved protein fold, and a conserved cysteine residue that coordinates the iron atom bound by the porphyrin cofactor. Additionally, most P450 enzymes, when treated with carbon monoxide under reducing conditions, give an intense absorption band at 450 nm.

P450 enzymes also share a common mechanism. The resting state of the enzyme is iron(III), at which time the metal is coordinated by the porphyrin, cysteine, and a water molecule (See, FIG. 1). Substrate binding displaces the coordinating water molecule, resulting in an increase in reduction potential, which triggers reduction by a separate fused reductase domain. Reduction of the iron-center to iron(II) triggers very fast oxygen binding. Donation of an additional electron and two protonations of the iron-peroxo intermediate results in loss of distal oxygen atom, generating a Fe(V)-oxene species or a Fe(IV)═O cation radical species, which reacts by insertion into alkyl C—H or alkenyl C═C bonds, yielding alcohols or epoxides, respectively. Hydroxylation by cytochrome P450s is analogous to C—H amination mediated by metalloporphyrin complexes, and other metal-based catalysts.

The P450 family of enzymes is involved in myriad oxidative transformations that are crucial to the production of natural products in many organisms. Some of the reactions mediated by P450 enzymes include hydroxylation, epoxidation, phenolic ring coupling, radical rearrangements, heteroatom oxidation, and demethylation.

Advantages of P450s include the ability to activate recalcitrant C—H bonds within diverse scaffolds, a broad substrate selectivity, and the ability to regioselectively target C—H bonds for hydroxylation. Some limitations to their use include the requirement for expensive cofactors (such as NADPH), and their problematic expression in bacterial hosts. However, several soluble bacterial cytochrome P450 enzymes exist that are more readily expressed than eukaryotic isoforms.

Cytochrome P450BM3 (CYP102A1).

This cytochrome P450 was the third P450 enzyme isolated from Bacillus megaterium. Unlike previously characterized P450 enzymes, P450_{BM3} contained a heme domain typical of P450s that was fused to a normally separate reductase domain. The fused reductase domain has two tightly-bound flavin cofactors. Electrons donated from transiently bound NADPH are passed from one flavin cofactor to the second, and from there to the iron center of the heme domain. P450BM3 has been the subject of many engineering and biochemical studies, and has been shown to be able to carry out regio- and enantioselective hydroxylation and epoxidation of diverse substrates. The wild-type enzyme is composed of 1048 amino acids, and has two subdomains. The first subdomain (residues 1-472) binds to the heme cofactor and is the site of oxidation reactions, while the latter subdomain binds to the two flavin cofactors and contains the NADPH binding site. Although the presence of the fused reductase domain is advantageous for many applications, the heme domain can be expressed separately and tends to give higher yields of expressed protein. Isolated heme domains can catalyze monooxygenation reactions if provided with hydrogen peroxide. Wild-type BM3 is perhaps the fastest P450 enzyme ever characterized, and shows specificity for fatty acids, such as palmitic and arachidonic acids.

Cytochrome P450BM3 Engineering.

Given its robust nature, P450_{BM3} has been the subject of many engineering studies. In particular, directed evolution, a process in which rounds of mutation and selection are performed iteratively, has been strikingly successful at altering the substrate selectivity for hydroxylation, as well as altering the reactivity of the enzyme to catalyze epoxidation of alkenes. Directed evolution has also been applied to P450_{BM3} for the purposes of enhancing its thermostability and solvent tolerance.

Notable examples of directed evolution of P450_{BM3} include alteration of its native selectivity for long-chain fatty acids to prefer small, gaseous alkanes such as propane, as well as a library of P450 enzymes that can hydroxylate large substrates. Additionally, P450s have been generated that metabolize approved drugs in a fashion identical with human liver enzymes. Many engineering studies have also shown that the regioselectivity and enantioselectivity of oxidation reactions catalyzed by P450BM3 can be systematically modified via mutagenesis.

The above examples attest to the usefulness of an enzyme-based oxidation catalyst whose activity can be readily modified by directed evolution. Prior to the advent of the present invention, no enzymes were known to catalyze the oxidative amination of C—H bonds to yield amines or amides, although these transformations are isoelectronic with oxidation reactions. Additionally, no enzymes were known to carry-out the intermolecular aziridination of olefins.

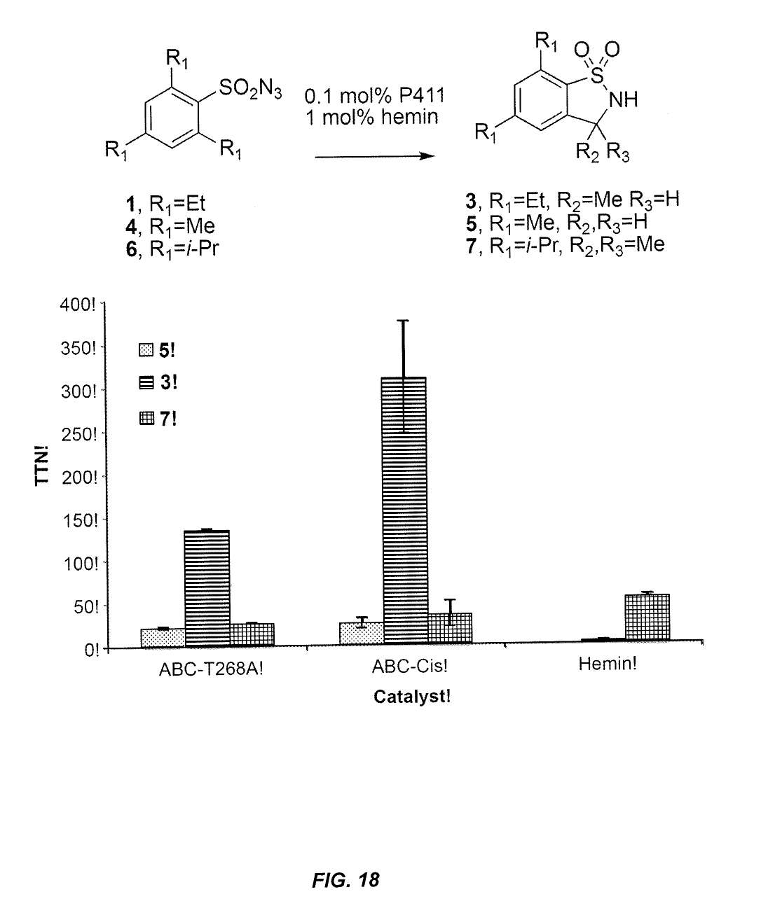

Metal-porphyrin C—H amination catalysts have been described (Breslow, R. & Gellman, S. H., J. Chem. Soc. Chem. Commun., 1400-1401 (1982); Fantauzzi, S. et al., Dalton Trans., 5434-5443 (2009)), as have trace levels of intramolecular amination catalyzed by mammalian cytochrome P450s from iminoiodanes (Svastis, E. W. et al., J. Am. Chem. Soc. 107, 6427-6428 (1985)), a transformation which is isoelectronic to the well-established P450-catalyzed transfers of ‘oxenes’ from iodosylbenzene (Groves, J. T. et al., J. Am. Chem. Soc. 101, 1032-1033 (1979)). We chose to use arylsulfonylazides rather than iminoiodanes as nitrene precursors due to their ease of synthesis, greater solubility in protein-compatible cosolvents, and superior atom efficiency. In initial experiments, we tested a panel of 20 purified cytochrome P450_{BM3} (BM3) variants, including wild-type BM3 and others that had shown monooxygenation and cyclopropanation activity at a catalyst loading of 0.5 mol % for reaction with arylsulfonylazide 1 under anaerobic conditions in the presence of NADPH in aqueous media (phosphate buffer, 2.5% DMSO). Most reactions gave sulfonamide 2 as the major product, though all of the tested enzymes, including wild-type, yielded small amounts of the C—H amination product, 3 (see, FIG. 18 for structures).

By far the most active enzyme in C—H amination was the serine-heme ligated “P411” cyclopropanation catalyst, BM3-CIS-C400S (henceforth ABC-CIS, 14 mutations from wild-type) (Coelho, P. S. et al., Highly efficient carbene transfer to olefins catalyzed in vivo. Submitted (2013)), which supported over 140 total turnovers (TTNs) (73% yield of 3 by HPLC). Variant BM3-CIS, which lacks the C400S mutation at the axial heme ligand, was significantly less active (9 TTN), suggesting that serine-heme ligation enhances BM3-catalyzed C—H amination, as it does for cyclopropanation. The BM3-C400S single mutant (henceforth ‘ABC’) was also tested; its activity (49 TTN), though markedly improved relative to BM3 (4 TTN), was modest compared to ABC-CIS.

We hypothesized that one or several mutations in BM3-CIS beyond C400S helped to support high C—H amination activity. BM3-T268A also exhibited significant activity with azide 1 (28 TTN). The T268A mutation is present in BM3-CIS, and has been reported to enhance cyclopropanation catalysis (Coelho, P. S. et al., Science 339, 307-310 (2013)). To clarify the roles of the T268A and C400S mutations in BM3-catalyzed amination, we performed further experiments at 0.1 mol % catalyst loading with the BM3-T268A and BM3-C400S (ABC) single mutants as well as the T268A/C400S double mutant (ABC-T268A) in reaction with sulfonyl azide 1 (Table 12).

TABLE 12

| Comparison of activities and enantioselectivies | | |
|---|---|---|
| of purified P450 and P411 variants | | |
| with azide 1 at 0.1 mol % catalyst loading | | |
| giving sulfonamide 2 and benzosultam 3. | | |
| 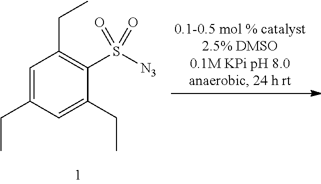 [SMILES: CCc1cc(CC)c(S(=O)(=O)N=[N+]=[N-])c(CC)c1] | | |
| 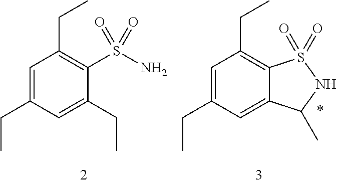 [SMILES: CCc1cc(CC)c(S(N)(=O)=O)c(CC)c1 ; CCc1cc(CC)c2c(c1)C(C)NS2(=O)=O] | | |
| In vitro catalyst | TTN | % ee* |
| BM3 (WT) | 2.1 | nd |
| BM3-T268A | 15 | 36 |
| ABC | 32 | 20 |
| ABC-T268A | 120 | 58 |
| ABC-CIS | 310 | 67 |
| ABC-CIS-A268T | 82 | 47 |

Activities are presented in TTN. Reactions conditions were as follows: 2 μM catalyst, 2 mM azide 1, 2 mM NADPH, oxygen depletion system (100 U mL⁻¹ glucose oxidase, 1400 U ml⁻¹ catalase, 25 mM glucose) in 0.1 M KPi pH 8.0 with 2.5% (v/v) DMSO. Yields and enantioselectivies determined by HPLC analysis. * (S−R)/(S+R). nd=not determined.

We found that the T268A and C400S mutations combined to yield a highly active enzyme (120 TTN for ABC-T268A double mutant versus 313 TTN for ABC-CIS, Table 12), indicating that the T268A and C400S mutations were primary contributors to the high activity of ABC-CIS. In fact, reverting the T268A mutation in ABC-CIS markedly reduced activity (82 TTN).

Control experiments revealed that the enzyme-catalyzed reaction was inhibited by carbon monoxide, air, and heat denaturation of the enzyme, supporting the enzyme-bound heme as the site of catalysis (Table 13).

TABLE 13
Controls experiments for variant ABC-T268A. Conditions as decribed
herein.

| Conditions | TTN | % activity^{a} | % ee* |
|---|---|---|---|
| Complete system (CS) | 110 | — | 38 |
| CS-NADPH + Na₂S₂O₄ | 130 | 120 | 44 |
| CS + CO | 5 | 4.4 | — |
| Boiled P450ₕₒₗₒ | 33 | 30 | 1 |
| CS aerobic | 10 | 9.1 | 60 |
| CS-P450 | 0 | 0 | — |
| CS heme-Na₂S₂O₄ + | 4 | 3.6 | — |
| NADPH | | | |
| CS heme | 160 | 145 | 91 |
| CS heme + CO | 0 | 0 | — |
| Boiled P450ₕₑₘₑ | 10 | 9.1 | 3 |
| CS heme aerobic | 0 | 0 | — |
| CS-P450 + Hemin | 0 | 0 | — |

^{a}Percent residual activity (CS = 100%). % ee = (S − R)/(S + R). Complete system (CS) includes 10 mM styrene, 20 mm EDA, 20 mM Na₂SO₄, 20 μM P450 (H2A10) under anaerobic conditions.

Hemin also was capable of catalyzing this reaction when reduced with dithionite (Table 13). FIG. 18 shows a schematic depicting substrates used to test the dependence of C—H bond strength on amination activity in enzyme- and hemin-catalyzed reactions; 0.1 mol % of P411 catalysts (ABC-T268A and ABC-CIS) and 1 mol % hemin were reacted with 2 mM sulfonyl azide substrates 1, 4, or 6 with 2 mM NADPH, an oxygen depletion system (100 U ml⁻¹ glucose oxidase, 1400 U ml⁻¹ catalase, 25 mM glucose) in 0.1 M KPi pH 8.0 at room temperature for 24 hours.

TABLE 14
Substrate selectivity of ABC catalysts versus free hemin.

| | Product | | |
|---|---|---|---|
| Catalyst | (3) | (5) | (7) |
| ABC-T268A | 135 | 22 | 27 |
| ABC-CIS | 310 | 26 | 36 |
| Hemin | 3.1 | nd | 55 |

Small scale reactions containing either 0.1% loading of ABC-CIS or ABC-T268A or 1% loading of hemin with azides 1, 4, or 6 according to standard procedures.

However, whereas enzyme reactions with prochiral substrate 1 resulted in asymmetric induction (Table 14), reaction with hemin unsurprisingly yielded only racemic 3, which indicates that BM3-catalyzed amination occurs within the chiral environment of the enzyme active site. Addition of substoichiometric amounts of NADPH or dithionite was sufficient for activity (Table 15), supporting the hypothesis that ferrous-heme is the azide-reactive state, akin to P450-catalyzed cyclopropanation (Coelho, P. S. et al., Science 339, 307-310 (2013)).

TABLE 15
Dependence of holoenzyme activity on NADPH concentration.

| [NADPH] (mM) | [sultam] (mM) | TTN |
|---|---|---|
| 2 | 0.322 | 161 |
| 0.1 | 0.486 | 243 |
| 0.02 | 0.164 | 82 |
| 0.01 | 0.053 | 27 |
| 0 | 0 | 0 |

Small-scale reactions (400 μL) were assembled and worked up as described above. NADPH concentration was systematically varied within the concentration range of sultam product formation to assess stoichiometry of iron reduction in the enzyme-catalyzed reactions. The ABC-CIS enzyme was used at 0.1 mol % loading (2 μM) relative to substrate (80 mM 1, 10 μL) 0.1 M KPi pH 8.0, 2.5% DMSO co-solvent. Although dithionite could support catalysis, its effect was comparable with that of NADPH for both cysteine and serine-ligated enzymes BM3-T268A and ABC-T268A (Table 16), suggesting that reduction to ferrous heme was not limiting.

TABLE 16
Comparison of NADPH and dithionite in reaction of BM3-T268A and
ABC-T268A with azide 1.

Reductant

| Catalyst | Dithionite | NADPH |
|---|---|---|
| BM3-T268A | 25 | 22 |
| ABC-T268A | 110 | 110 |

Small scale reactions containing either NADPH (2 mM) or dithionite (2 mM) as reductant, enzymes were used at 0.1 mol % loading (2 μM) relative to substrate (80 mM 1, 10 μL) 0.1 M KPi pH 8.0, 2.5% DMSO co-solvent.

To examine the effect of C—H bond strength on amination activity, we reacted ABC-CIS and ABC-T268A with the trimethyl and triisopropyl analogs of 1 (substrates 4 and 6, respectively). In reactions with either analog, the desired benzosultam products were obtained, though the productivity was lower with both trimethyl and triisopropyl substrates (FIG. 18, Table 17) Free hemin activity was inversely correlated with the C—H bond strength of the substrates, showing no measurable activity on substrate 4, minimal activity on substrate 1 (3 TTN), and the highest activity on substrate 6 (55 TTN). The different pattern of reactivity observed with the enzyme reactions suggests that factors such as steric effects and active site structure are important influences in enzymatic amination.

Given the additional expenses and time-costs associated with the use of using purified enzymes as catalysts, we next investigated whether ABC and BM3 catalysts expressed in intact E. coli cells could efficiently catalyze amination reactions when provided with azide substrate. Remarkably, both the ABC-T268A and ABC-CIS enzymes were highly active on 1, catalyzing hundreds of turnovers (245 TTN, 89% ee ABC-T268A, 680 TTN, 60% ee ABC-CIS) under anaerobic conditions with added glucose. Lyophilized cells containing ABC-CIS support catalysis, with productivity that was similar to freshly-prepared cell suspensions (750 TTN, 61% ee). Enantioselectivity was comparable or enhanced for whole-cell catalysts relative to purified enzymes (Table 17).

TABLE 17
Comparison of C—H amination activities of intact
E. coli cells expressing P450 and P411 variants.

| | Cell | | Yield | Yield | | |
|---|---|---|---|---|---|---|
| In vivo | density | [P450] | sulfon- | sultam | | |
| catalyst | (g_{cdw}/L) | (μM) | amide (%) | (%) | TTN | % ee* |
| pCWori | 11 | nd | 92 | 0.8 | n/a | nd |
| BM3 | 8.5 | 6.6 | 33 | 0.5 | 5.1 | nd |
| BM3-T268A | 9.5 | 5.8 | 51 | 7.8 | 26 | 84 |
| ABC | 8.8 | 4.3 | 80 | 6.7 | 29 | 16 |
| ABC-T268A | 9.4 | 2.2 | 45 | 30 | 250 | 89 |
| ABC-CIS | 9.1 | 1.4 | 50 | 46 | 680 | 60 |

*(S − R)/(S + R).
nd = not determined.

Reaction conditions were as follows: 2 mM azide 1, 25 mM glucose, E. coli BL21(DE3) cells in M9-N minimal medium (OD₆₀₀=30), 2.5% DMSO, oxygen depletion system (100 U ml⁻¹ glucose oxidase, 1400 U ml⁻¹ catalase) reacted for 24 hours under anaerobic conditions at 298 K. Yields determined by HPLC quantification.

The previously characterized T438S mutation in ABC-CIS strongly enhanced enantioselectivity (430 TTN, 86% ee) (Coelho, P. S. et al., Science 339, 307-310 (2013); Huang, W. C. et al., Metallomics 3, 410-416 (2011)). Optimization of expression conditions increased the productivity of whole-cell C—H amination catalysts, enabling conversions of nearly 70% in small scale reactions (Table 18).

TABLE 18
Effect of growth media and expression strain on productivity with azide 1.

| | Cell density | [P450] | Yield | Yield |
|---|---|---|---|---|
| Media | (g_{cdw}/L) | μM | sulfonamide (%) | sultam (%) |
| M9Y-ALA | 10.5 | 0.45 | 35 | 14 |
| TB_{P} | 10.5 | 0.89 | 33 | 29 |
| FB | 10.7 | 4.9 | 46 | 48 |
| Hyperbroth | 12.7 | 11 | 26 | 66 |
| M9Y + ALA | 10.5 | 4.1 | 39 | 43 |
| C* | 8.3 | 2.6 | 32 | 41 |

Inspired by the simplicity of employing whole cells as amination catalysts, we performed a preparative scale reaction (50 mg) using anaerobic resting cells expressing the ABC-CIS-T438S catalyst, affording sultam 3 (77% conversion, 69% isolated yield, 87% ee).

The beneficial effect of the T268A and C400S mutations for C—H amination is striking in that both residues play key roles in P450-catalyzed monooxygenation (Meunier et al., Chem. Rev. 104, 3947-3980 (2004); Whitehouse et al., Chem. Soc. Rev. 41, 1218-1260 (2012)). While important for protonation of iron-peroxo intermediates that occur during dioxygen activation, T268 may sterically hinder bulkier azide substrates in C—H amination. Consistent with a steric role, the T268A mutation enhances the stereos electivity of C—H amination, and in styrene cyclopropanation it strongly impacts diastereo and enantioselectivity (Coelho, P. S. et al., Science 339, 307-310 (2013)). For cyclopropanation, the C400S mutation is not necessary to drive in vitro reactions, and its strong effect in vivo can be attributed to the higher reduction potential of the serine-ligated heme, facilitating reduction by NADPH (Coelho, P. S. et al., Highly efficient carbene transfer to olefins catalyzed in vivo. Submitted (2013)). In contrast, here we find that the C400S mutation gives high levels of in vitro activity (Table 12). This effect persists even when dithionite is used as a reductant (Table 16), suggesting that the C400S mutation does not simply facilitate NADPH-drive reduction to the active ferrous state, but rather exerts a strong effect on subsequent steps of the reaction.

FIG. 19 illustrates substrate scope of P450-catalyzed intramolecular C—H amination.

FIG. 20 illustrates substrate scope of P450-catalyzed intermolecular C—H amination.

FIG. 21 illustrates substrate scope of P450-catalyzed intramolecular aziridination.

FIG. 22 illustrates substrate scope of P450-catalyzed intermolecular aziridination.

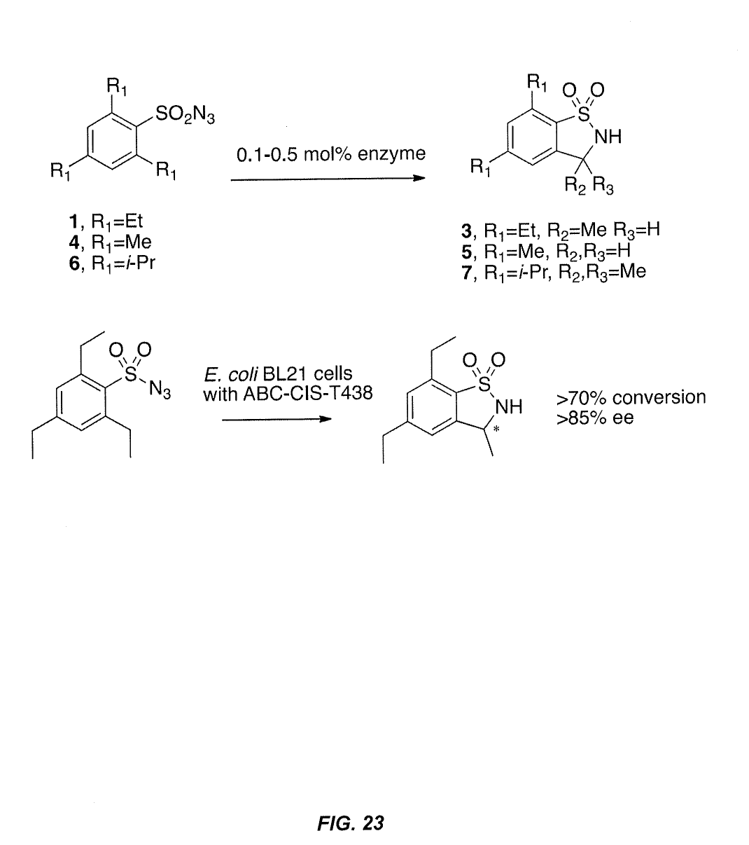

FIG. 23 illustrates substrates for purified enzyme and whole-cell reactions.

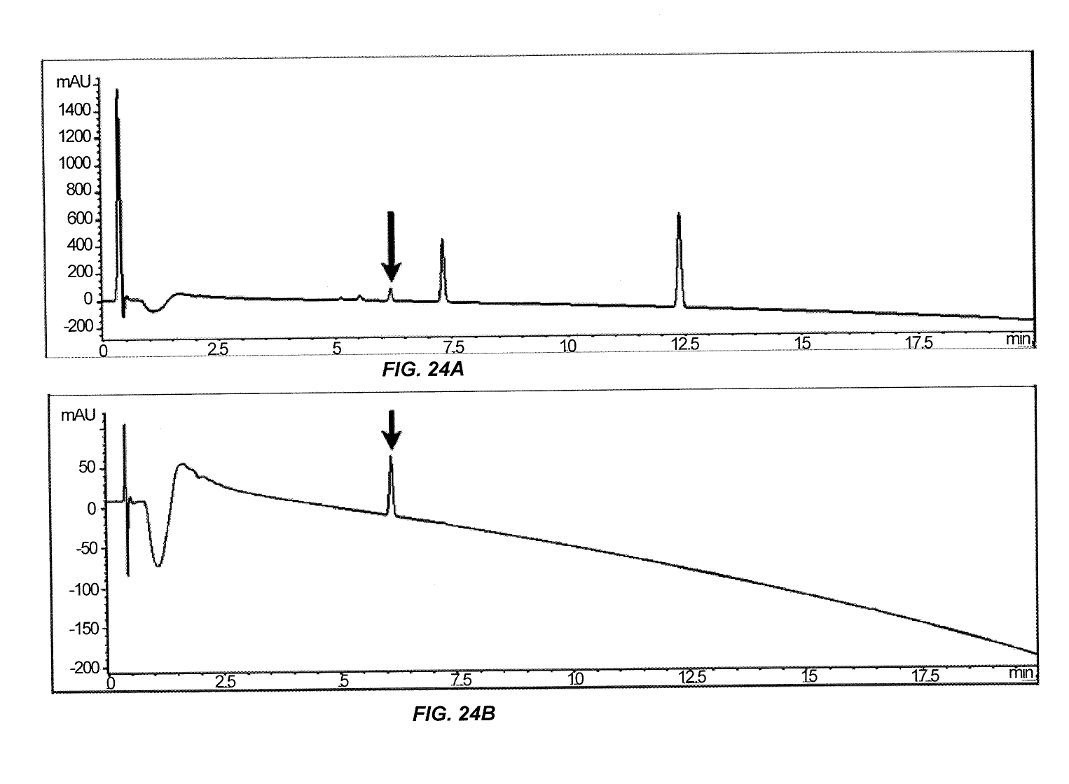

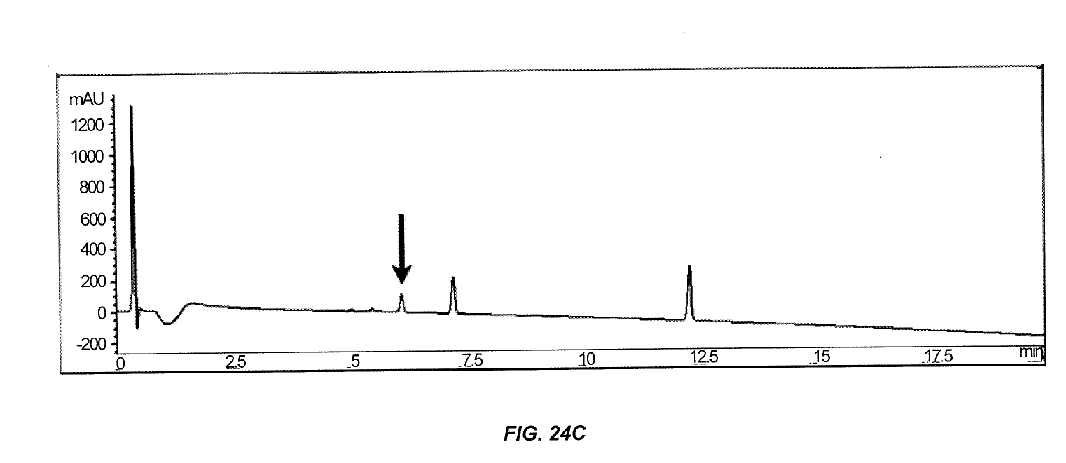

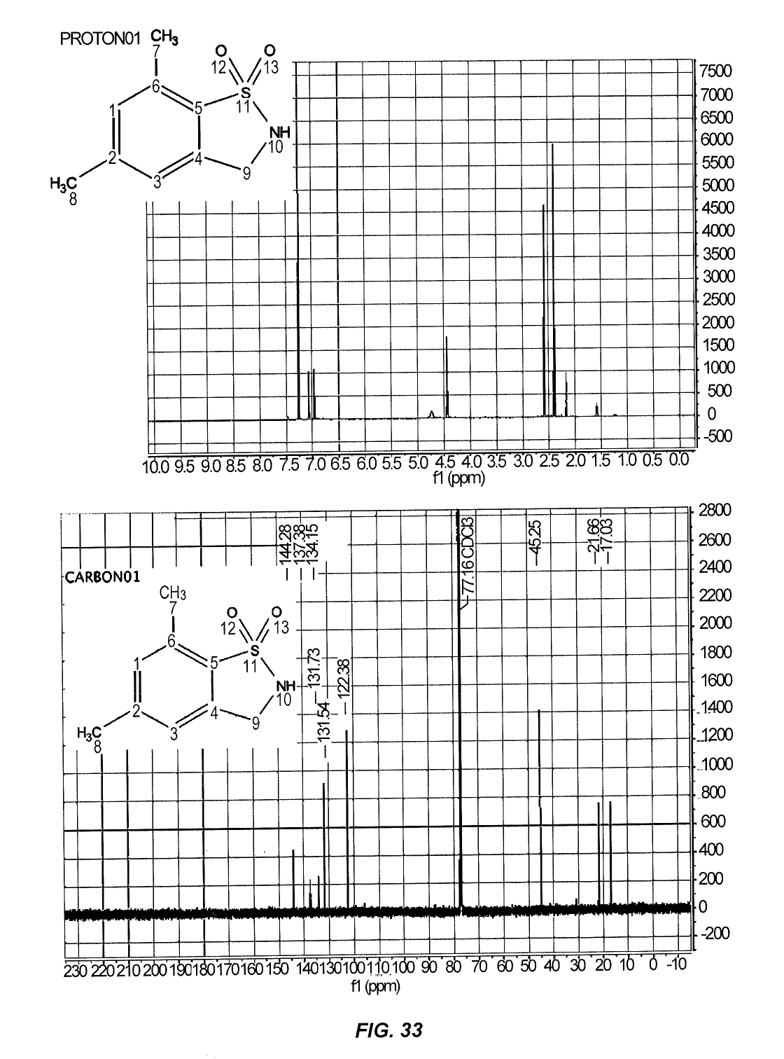

FIG. 24A-C show a demonstration of enzymatic production of (5). Panel A is an LC-MS 220 nm chromatogram of enzyme reaction mixture containing putative 5, Panel B is a synthetic standard of 5 whose NMR spectra are presented in FIG. 33, and Panel C is a sample containing a mixture of the enzyme reaction and synthetic 5, showing coelution.

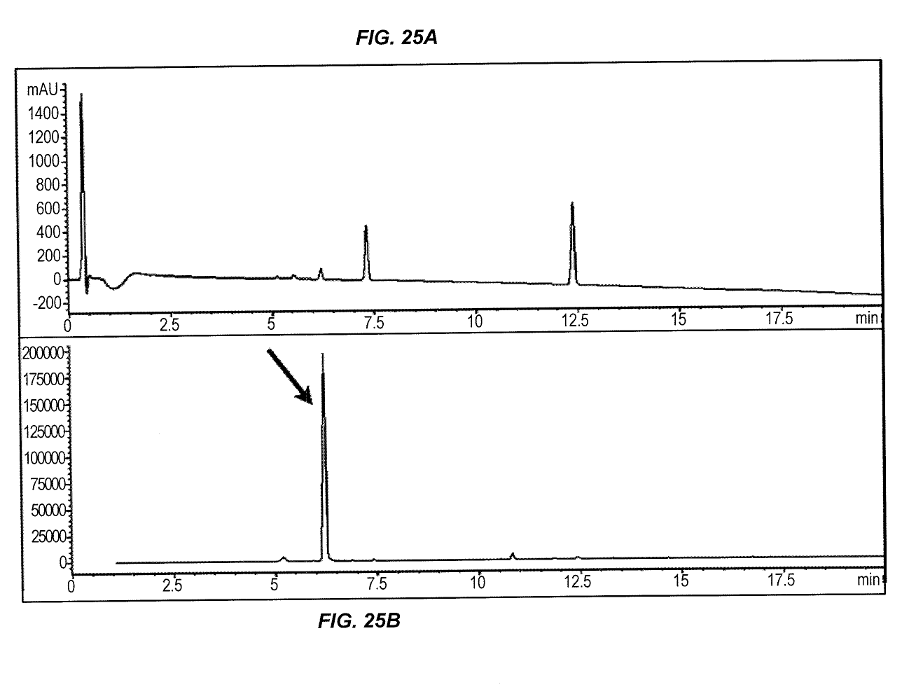

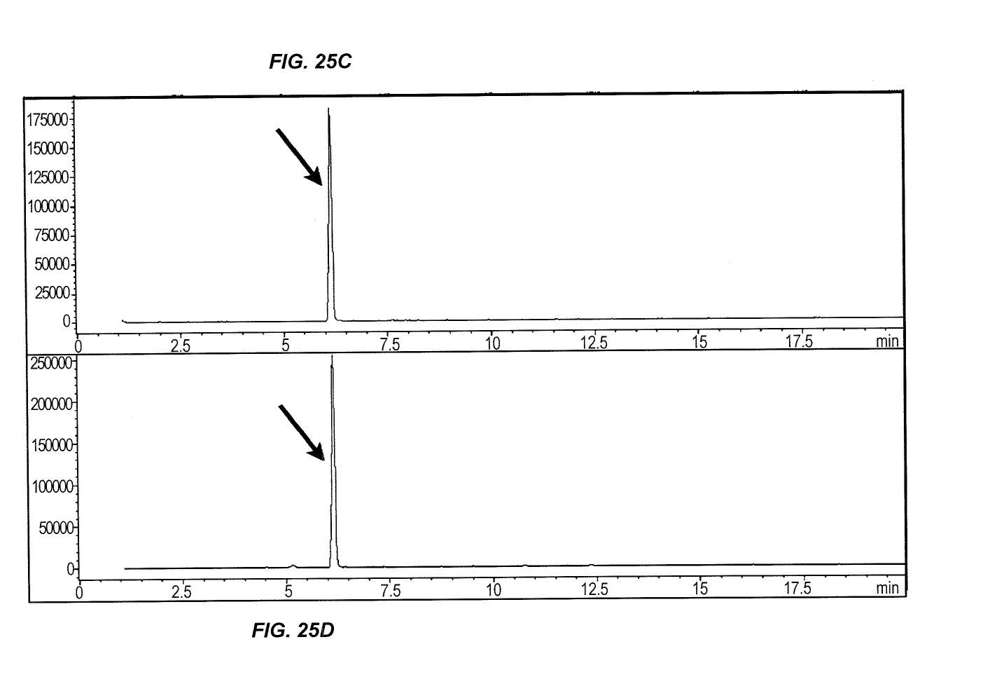

FIGS. 25A-D show a demonstration of enzymatic production of (5). LC runs showing ESI-MS-(−) detection of selected ions (mass window 195.5-196.5) Panels C-D; top panel shows 220 nm trace from enzyme reaction in FIG. 24A.

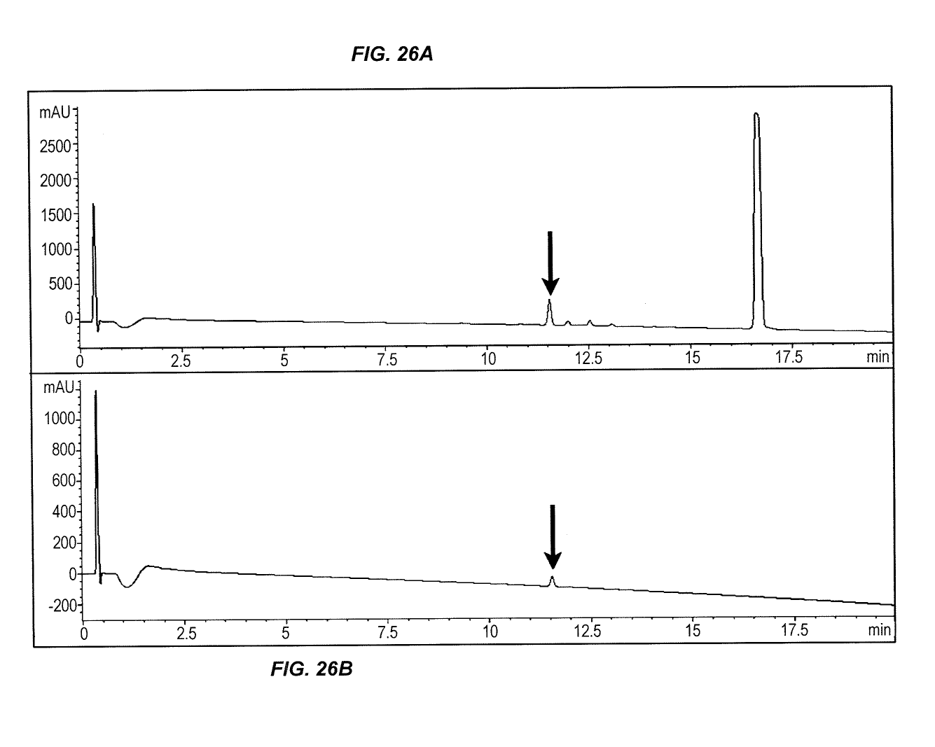

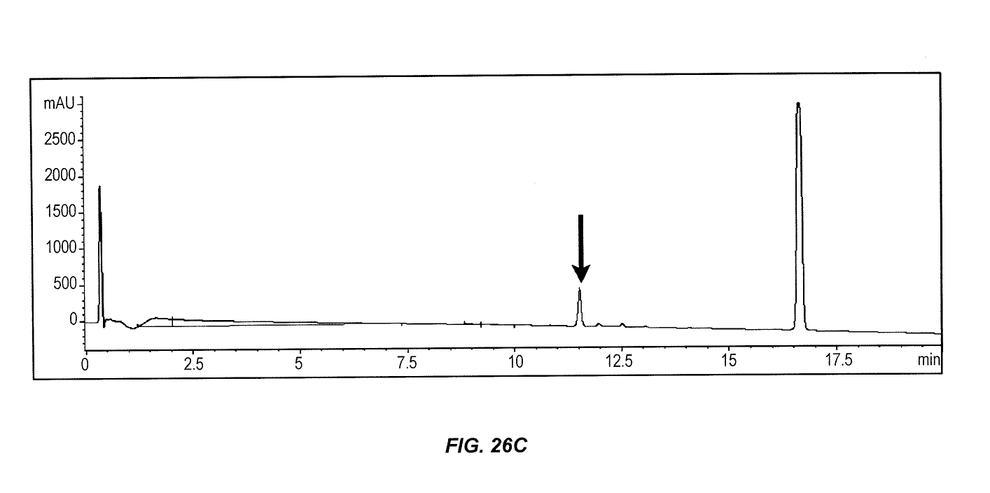

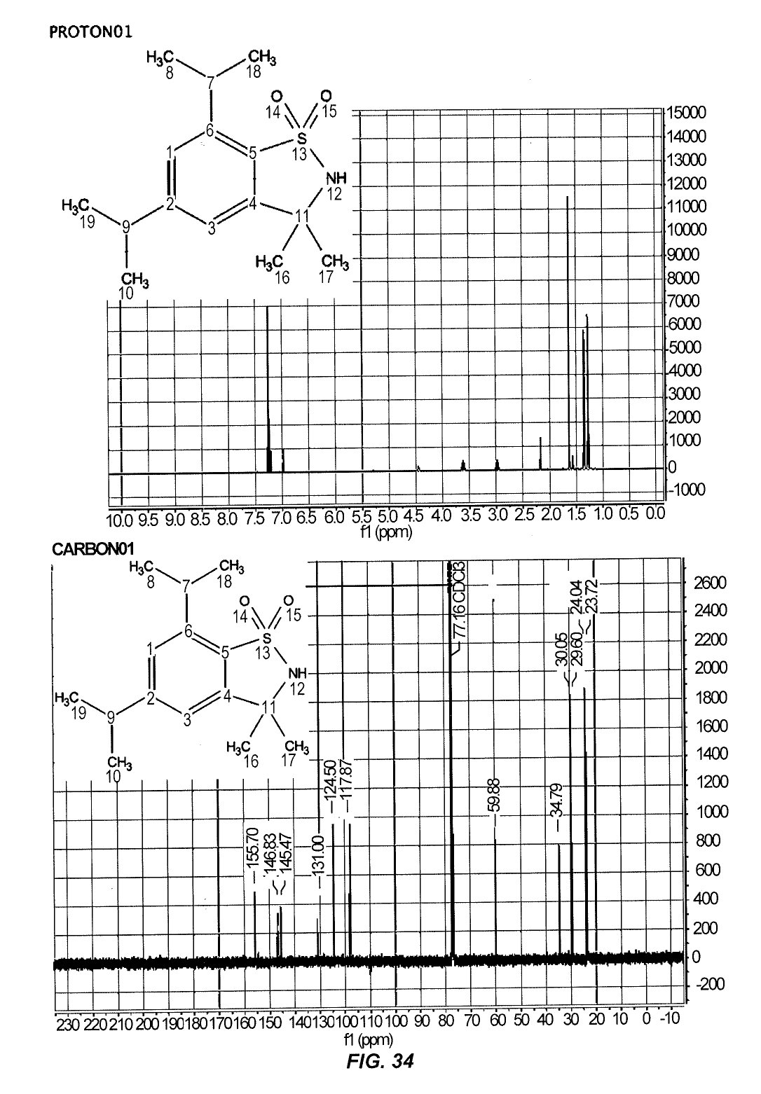

FIGS. 26A-C show a demonstration of enzymatic production of (7). Panel A is LC-MS 220 nm chromatogram of enzyme reaction mixture containing putative 7, Panel B is a synthetic standard of 7 whose NMR data is presented in FIG. 34 and Panel C is a sample containing a mixture of the enzyme reaction and synthetic 7, showing coelution.

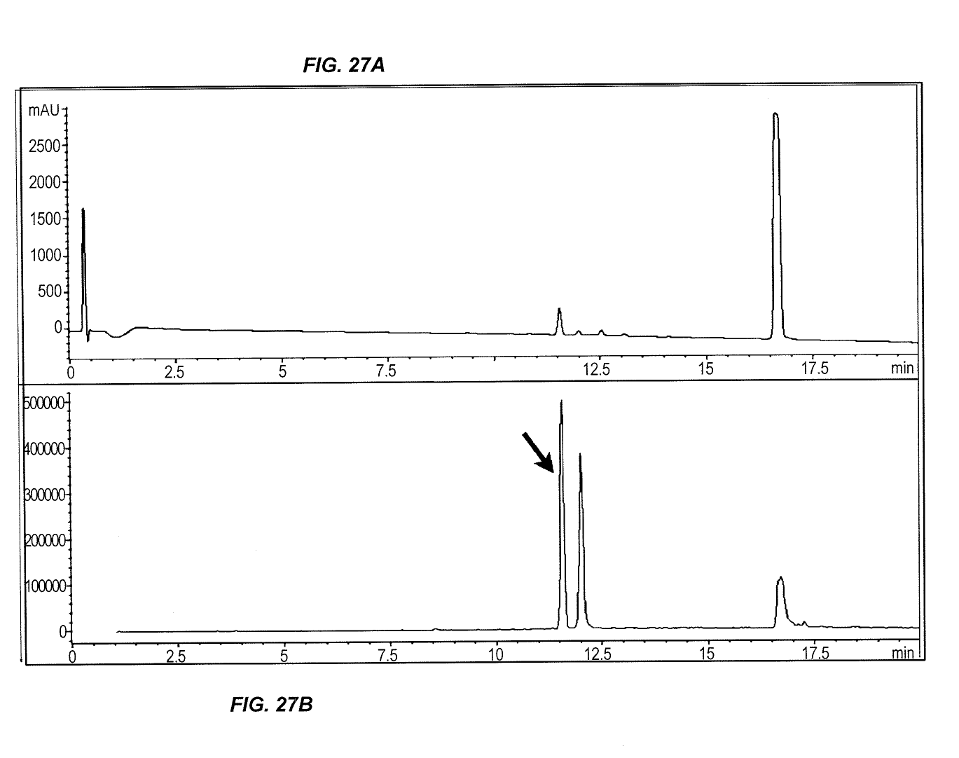

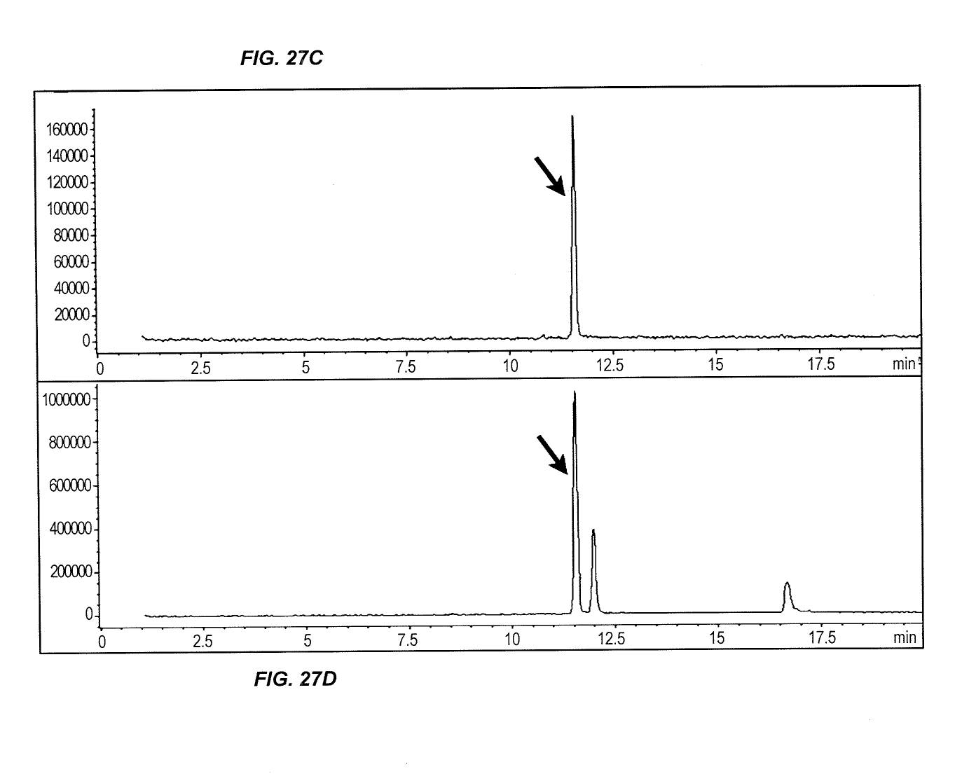

FIGS. 27A-D show a demonstration of enzymatic production of (7). LC runs showing ESI-MS-(−) detection of selected ions (mass window 279.5-280.5) in panels C-D. Panel A shows 220 nm trace from enzyme reaction in FIG. 26A. A second isobaric peak with m/z 280 Da can be observed in enzyme reactions. This material was not present in sufficient quantities to permit detailed structural characterization.

Many enzyme-catalyzed reactions such as ketoreduction, monooxygenation, and transamination are increasingly useful in organic synthesis, and biocatalytic applications of these and other naturally-occurring reaction types will continue to develop. However, it is no longer necessary to limit biocatalysis to reactions that have natural antecedents (Coelho, P. S. et al., Science 339, 307-310 (2013); Hyster, T. K. et al., Science 338, 500-503 (2012); Köhler, V. et al., Nat. Chem. 5, 93-99 (2013)). Rather, the scope of biocatalysis can be expanded by directing natural enzymes to imitate the artificial by accessing the chemistry enabled by synthetic reagents.

### Methods

Enzymes used as purified catalysts were expressed as previously described (Lewis, J. C. et al., Proc. Natl. Acad. Sci. U.S.A. 106, 16550-16555 (2009)), and were purified by anion-exchange chromatography (for holoenzymes) or Ni-NTA chromatography (for isolated heme domains). Concentrations of P450 or P411 enzymes were determined as previously reported (Omura, T. & Sato, R., J. Biol. Chem. 239, 2370-2378 (1964); Vatsis, K. P. et al., J. Inorg. Biochem. 91, 542-553 (2002)). Small-scale reactions (400 μL) were conducted in 2 mL crimp vials containing degassed buffer (0.1 M potassium phosphate pH 8.0), enzyme (0.1-0.5% catalyst loading) and oxygen depletion system (10× stock solution containing 14,000 U/mL catalase, 1,000 U/mL glucose oxidase dissolved in 0.1 M KPi pH 8.0). Enzyme (P450 or P411) solution and oxygen depletion mixture were added to the vial with a small stir bar before crimp-sealing. Degassed solutions of glucose (250 mM, 40 μL), NADPH (40 mM, 40 μL) and phosphate buffer (0.1 M, pH 8.0, up to 390 μL) were added by syringe, followed by substrate (80 mM in DMSO, 10 μL). Reactions were stirred at room temperature for 24 h under positive argon pressure.

Whole cell reactions used E. coli BL21(DE3) cells containing P450 or P411 catalysts, which were expressed and prepared as described elsewhere (Coelho, P. S. et al., Highly efficient carbene transfer to olefins catalyzed in vivo. Submitted (2013)). Following expression, cells were resuspended to an OD600 of 30 in M9 salts lacking NH₄Cl (M9-N), and then degassed by sparging with argon in a sealed 6 mL crimp vial for at least 0.5 h. Separately, glucose (250 mM dissolved in 1×M9-N, 40 μL, or multiples thereof) was degassed by sparging with argon for at least five minutes. The oxygen quenching mixture was added to sealed 2 mL crimp vials containing stir bars and the headspace of the vials was purged with argon for five minutes at which time glucose, and then cells were added by syringe. Substrate (80 mM arylsulfonyl azide, 10 uL in DMSO) was added via syringe, and the reactions were stirred at room temperature for 24 h under positive argon pressure.

The above concept has been demonstrated for a single P450 enzyme, CYP102A1, from Bacillus megaterium, and for chimeras of the B. megaterium enzyme with other, related P450s from B. subtilis. Those of skill in the art, however, will recognize that other P450s from other organisms can be engineered to carry out C—H amination, and that those catalysts in turn can be employed in whole-cell reactions such as those described above. In particular, we expect that the equivalent mutations to T268A and C400S, when made in other P450 or heme-containing enzymes, will enable catalysis of C—H amination. One of skill in the art knows how to identify the equivalent residue to C400 in other P450s, based on sequence alignments, an example of which is given below. Methods known in the art, such as site-directed mutagenesis or gene synthesis, can be used to alter these residues to alanine (for T268) and to serine (for C400) in any P450. The resulting enzyme serves as a catalyst for C—H amination. We expect that this mutation in a purified protein or whole cell catalyst will improve the activity over the parent enzyme that does not include this mutation.

To illustrate the above, we provide below a BLAST alignment of the amino acid sequence of P450_{BM3} (CYP102A1) to other P450s, such as the one from Pseudomonas putida (CYP101A1, P450_{CAM}; SEQ ID NO:21) or the mammalian enzyme from Oryctolagus cuniculus (CYP2B4; SEQ ID NO:24), enables identification of the proximal cysteine residue or of the equivalent T268 (marked in bold and highlighted), as shown below:

| | | | |
|---|---|---|---|
| CYP102A1 | 380 | ENPSAIPQH--------AFKPFGNGQRACIGQQFALHEATLVL | 414 |
| | | E +A P H + FG+G C+GQ A E + L | |
| CYP101A1 | 329 | ERENACPMHVDFSRQKVSHTTFGHGSHLCLGQHLARREIIVTL | 371 |
| CYP102A1 | 265 | GHETTSGLLSFALYFLVKNPHVLQKAAEEAARVLVDPVPSYKQVKQLKYVGMVLNEALRL | 324 |
| | | G +T LSF++ FL K+P Q+ E R +P+ E LR | |
| CYP101A1 | 249 | GLDTVVNFLSFSMEFLAKSPEHRQELIERPER-----IPA------------ACEELLR- | 290 |
| CYP102A1 | 374 | FRPERF--ENPSAIPQHAFKPFGNGQRACIGQQFALHEATLVLGMMLKHFD | 422 |
| | | F P F N + F PF G+R C+G+ A E L +L++F | |
| CYP2B4 | 408 | FNPGHFLDANGALKRNEGFMPFSLGKRVCLGEGIARTELFLFFTTILQNFS | 458 |
| CYP102A1 | 257 | QIITFLIAGHETTSGLLSFALYFLVKNPHVLQKAAEEAARVL-VDPVPSYKQVKQLKYVG | 315 |
| | | +++ AG ETTS L + ++K PHV ++ +E +V+ P+ ++ Y | |
| CYP2B4 | 291 | TVLSLFFAGTETTSTTLRYGFLLMLKYPHVTERVQKEIEQVIGSHRPPALDDRAKMPYTD | 350 |

Therefore, the mutations C357S and T252A in CYP101A1 or C436S and T302A in CYP2B4 can enhance the C—H amination activity in these enzymes. For instance, the CYP101A1 variants with the single C357S mutation (SEQ ID NO: 50), the single T252A mutation (SEQ ID NO: 51), and the C357S and T252A mutations (SEQ ID NO: 52) can increase C—H amination. The CYP2B4 variants with the single C436S mutation (SEQ ID NO: 53), the single T302A mutation (SEQ ID NO: 54), and the C436S and T302A mutations (SEQ ID NO: 55) can also increase C—H amination.

The mutation can be introduced into the target gene by using standard cloning or by gene synthesis. The mutated gene can be expressed in the appropriate microbial host under the control of an inducible promoter or by means of chromosomal integration under the control of a constitutive promoter. C—H amination activity can be screened in vivo or in vitro by following product formation by HPLC or LC-MS.

As demonstrated above, the C—H amination catalysts reported herein function very well in whole-cells, and therefore can be used as part of a multigene pathway, wherein the nitrene precursor would be added exogenously or generated in situ.

The portability of the C400S and T268A mutations allow us to generate large libraries of P450-based C—H amination catalysts. These catalysts react with a wide-variety of nitrene precursors, and thereby provide access to a plethora of nitrogen functionalized molecules. These precursors include, but are not limited to, aryl azides, sulfonyl azides, phosphoryl azides, carbonyl azides, azidoformates, as well as non-azide nitrene precursors such as iminoiodanes, chloramines, bromamines, N-sulfonyloxy compounds, and amines (oxidized in situ for example by high valent metals such as lead(IV)acetate to give nitrenes). These nitrene precursors can then be expected to react intra- or intermolecularly with C—H bonds or C-heteroatom bonds to form nitrogen ligated products.

### Additional Methods

General.

Unless otherwise noted, all chemicals and reagents for chemical reactions were obtained from commercial suppliers (Sigma-Aldrich, Acros) and used without further purification. Silica gel chromatography purifications were carried out using AMD Silica Gel 60, 230-400 mesh. ¹H and ¹³C NMR spectra were recorded on either a Varian Inova 500 MHz (500 MHz and 125 MHz, respectively) in CDCl₃, and are internally referenced to residual solvent peak. Optical rotation values were measured on a Jasco J-2000 polarimeter. Reactions were monitored using thin layer chromatography (Merck 60 silica gel plates) using an UV-lamp for visualization or stains where indicated.

Analytical high-performance liquid chromatography (HPLC) was carried out using an Agilent 1200 series, an UV detector, and a Kromasil 100 C18 column (Peeke Scientific, 4.6×50 mm, 5 μm). Semi-preparative HPLC was performed using an Agilent XDB-C18 (9.4×250 mm, 5 μm). Analytical chiral HPLC used a Chiralpak AD-H column (Daicel, 4.6×150), while preparative chiral HPLC used a Chiralpak AD-H column (Daicel, 21×250 mm, 5 μm). Azides 1 and 4, and benzosultam standards 3, 5, and 7 (FIG. 18) were prepared as reported (Ruppel, J. V. et al., Org. Lett. 9, 4889-4892 (2007)). The precursor to azide 7 is commercially available (Sigma, 723045-1G). These standards were used in co-injection experiments to determine the authenticity of P450-catalyzed benzosultams. Enzymatically produced benzosultam 3 was prepared as described in section VI and characterized by NMR (¹H and ¹³C).

pCWori was used as a cloning and expression vector for all enzymes described in this study. Site-directed mutagenesis of ABC-CIS to yield ABC-CIS-A268T via overlap extension PCR, followed by digestion of vector and PCR products with BamHI and SacI, gel purified and ligated using T4 ligase (NEB, Quickligase).

Determination of P450/P411 Concentration.

Concentration of P450 or P411 enzymes was determined from ferrous carbon monoxide binding difference spectra using previously reported extinction coefficients for cysteine-ligated (ε=91,000 M⁻¹ cm⁻¹) and serine-ligated enzymes (ε=103,000 M⁻¹ cm⁻¹) (Omura, T. & Sato, R., J. Biol. Chem. 239, 2370-2378 (1964); Vatsis, K. P. et al., J. Inorg. Biochem. 91, 542-553 (2002)).

Protein Expression and Purification.

Enzymes used in purified protein experiments were expressed from E. coli cultures transformed with P450 or P411 variants. BL21(DE3) was used for expression of ABC-CIS, while DH5α was used as an expression host for all other enzymes. Expression and purification was performed as described (see, Lewis, J. C. et al., Proc. Natl. Acad. Sci. U.S.A. 106, 16550-16555 (2009)), with the exception that the agitation rate was lowered to 180 RPM for P411 after induction. Following expression, cells were pelleted and frozen at −20° C. For purification, frozen cells were resuspended in lysis buffer (25 mM tris pH 7.5, 4 mL/g of cell wet weight), and disrupted by sonication (2×1 min, output control 5, 50% duty cycle; Sonicator, Heat Systems—Ultrasonic, Inc.). To pellet insoluble material, lysates were centrifuged at 24,000×g for 0.5 h at 4° C. Cleared lysates were then purified on a Q Sepharose column (5 mL HiTrap™ Q HP, GE Healthcare, Piscataway, N.J.) using an AKTAxpress purifier FPLC system (GE healthcare). P450 or P411 enzymes were then eluted on a linear gradient from 100% buffer A (25 mM tris pH 8.0), 0% buffer B (25 mM tris pH 8.0, 1 M NaCl) to 50% buffer A/50% buffer B over 10 column volumes (P450/P411 enzymes elute at around 0.35 M NaCl). Fractions containing P450 or P411 enzymes were then pooled, concentrated, and subjected to three exchanges of phosphate buffer (0.1 M KPi pH 8.0) to remove excess salt. Enzyme concentrations were determined by CO binding difference spectra as described above. Concentrated proteins were aliquoted, flash-frozen on powdered dry ice, and stored at −20° C. until later use.

Typical Procedure for Small-Scale Amination Bioconversions Under Anaerobic Conditions Using Purified Enzymes.

Small-scale reactions (400 μL) were conducted in 2 mL crimp vials (Agilent Technologies, San Diego, Calif.) containing buffer (0.1 M potassium phosphate pH 8.0), enzyme (0.1-0.5% catalyst loading) and oxygen depletion system (10× stock solution containing 14,000 U/mL catalase, 1,000 U/mL glucose oxidase dissolved in 0.1 M KPi pH 8.0). Enzyme (P450 or P411) solution and oxygen depletion mixture were added to the vial with a small stir bar before crimp-sealing. Portions of phosphate buffer (190 μL, 0.1 M, pH=8.0), glucose (40 μL, 250 mM) and NADPH (40 μL, 20 mM), or multiples thereof, were combined in a larger crimp sealed vial and degassed by sparging with argon for at least 5 min. In the meantime, the headspace of the sealed 2 mL reaction vial with the P450 solution was made anaerobic by flushing argon over the protein solution (with no bubbling). The buffer/reductant/glucose solution (270 μL) was syringed into the reaction vial with continuous argon purge of the vial headspace. An arylsulfonyl azide solution in DMSO (10 μL, 80 mM) was added to the reaction vial via a glass syringe, and the reaction was left stirring for 24 h at room temperature under positive argon pressure. Final concentrations of the reagents were typically: 2 mM arylsulfonyl azide, 2 mM NADPH, 25 mM glucose, 2 or 10 μM P450. Reactions were quenched by adding 30 μL 3 M HCl under argon. To the vials were then added acetonitrile (430 uL) and internal standard (o-toluenesulfonamide 10 mM in 50% acetonitrile 50% water, 1 mM final concentration). This mixture was then transferred to a microcentrifuge tube, and centrifuged at 17,000×g for 10 minutes. A portion (20 μL) of the supernatant was then analyzed by HPLC. For LC-MS analysis, the quenched reaction mixture was extracted twice with ethyl acetate (2×350 μL), dried under a light argon stream and resuspended in 50% water-acetonitrile (100 μL). For chiral HPLC the reactions were extracted as above with ethyl acetate, dried and resuspended in DMSO (100 μL) and then C18 purified as described above. The C18 purified material was dried, and resuspended in acetonitrile, and then injected onto the chiral HPLC system for analysis. Sultam formation was quantified comparison of integrated peak areas of internal standard and sultam at 220 nm to a calibration curve made using synthetically produced sultam and internal standard.

Typical Procedure for Small-Scale Amination Bioconversions Under Anaerobic Conditions Using Whole Cells.

E. coli BL21(DE3) cells containing P450 or P411 catalysts were expressed and prepared as described elsewhere Coelho, P. S. et al., Highly efficient carbene transfer to olefins catalyzed in vivo. Submitted (2013)). Following expression, cells were resuspended to an OD600 of 30 in M9 salts lacking NH₄Cl (M9-N), and then degassed by sparging with argon in a sealed 6 mL crimp vial for at least 0.5 h. Separately, glucose (250 mM dissolved in 1×M9-N, 40 μL, or multiples thereof) was degassed by sparging with argon for at least five minutes. The oxygen quenching mixture was added to sealed 2 mL crimp vials containing stir bars and the headspace of the vials was purged with argon for five minutes at which time glucose, and then cells were added by syringe. Substrate (80 mM arylsulfonyl azide, 10 μL in DMSO) was added via syringe, and the reactions were stirred at room temperature for 24 h under positive argon pressure. Reactions were quenched in a manner identical to reactions containing purified enzymes as described above. For chiral HPLC, the reactions were extracted and purified in an identical manner as for reactions that employed purified enzymes. Lyophilized intact cells containing sucrose as a cryoprotectant were prepared as described elsewhere (Coelho, P. S. et al., Highly efficient carbene transfer to olefins catalyzed in vivo. Submitted (2013)). The resulting cell powder, containing expressed ABC-CIS (26 mg) were added along with a stir bar to a 2 mL crimp vial and then sealed. The headspace of the vial was degassed, oxygen quenching system (40 μL) was added via syringe, followed by degassed glucose (250 mM, 40 μL), M9-N (310 μL), and finally substrate (80 mM 1, 10 μL). Lyophilized cell reactions were stirred for 24 h at room temperature, then quenched and analyzed as described above.

Optimization Experiments for Whole Cell Reactions.

Media conditions, reaction buffer, and E. coli strains were varied. With the exception of media condition experiments, optimization experiments were performed according to the standard procedure described above using M9Y. Variable media condition experiments were performed according to the standard procedure except that the seed culture was grown in LB rather than M9Y, and the expression culture was grown in the alternative medium. Tested media conditions TB+power-mix, C-*, and FB were selected based on previously published work concerning P450 expression (Schulz, F., Monooxygenases: Experiments to turn a class of enzymes into a toolbox for biocatalysis Ph.D thesis, Ruhr-University Bochum, (2007), Pflug, S. et al., J. Biotechnol. 129, 481-488 (2007)). Hyperbroth was purchased from Athena Environmental Sciences (Baltimore, Md.) and used according to the manufacturer's instructions.

Compilation Plate Screening.

Purified enzymes were screened for activity in small scale reactions (400 μL) under anaerobic conditions as described above. Reactions were carried out using 0.5% mol of enzyme (10 μM) with respect to the triethylsulfonyl azide substrate 1 (2 mM). Standard reaction conditions described above were employed. Diethyl benzosultam product 3 (FIG. 18) was analyzed and quantified by reversed-phase HPLC (see above).

### Controls to Confirm the Enzymatic Amination Activity of Variant ABC-T268A.

Small-scale reactions (400 μL total volume) were set up and worked up. Control reactions were performed with both the holoenzyme (BM3 with covalently linked reductase domain) and the isolated heme domain. Reactions denoted by complete system (CS) indicate holo enzyme with reaction conditions as displayed in the scheme below. Reactions of the complete system with heme domain (CS heme) included 2 mM Na₂S₂O₄ rather than NADPH unless otherwise indicated in the table. For carbon monoxide (CO) inhibition and heat-denatured enzyme controls were performed as previously described (Coelho, P. S. et al., Science 339, 307-310 (2013)). Buffer for the CO controls was supplemented with 2 mM Na₂S₂O₄ in both holo and heme domain experiments. For the hemin experiment, 0.8 μL of a 1 mM solution in 50% DMSO-H₂O, such that the final concentration in the reaction was 2 μM to afford a direct comparison with the enzyme reactions. TTNs and enantioselectivity were determined as described above.

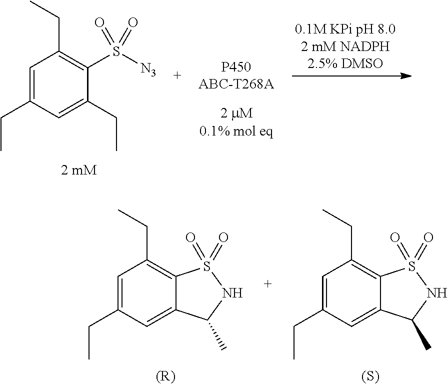 [SMILES: CCc1cc(CC)c(S(=O)(=O)N=[N+]=[N-])c(CC)c1 ; CCc1cc(CC)c2c(c1)[C@@H](C)NS2(=O)=O ; CCc1cc(CC)c2c(c1)[C@H](C)NS2(=O)=O ; "(R)" ; "(S)"]

Preparative-Scale Bioconversions.

The preparative reaction was scaled up proportionally from small-scale reactions. E. coli BL21 cells containing ABC-CIS (OD600 30, 90 mL in M9-N) were sparged with argon for 45 minutes in a round bottom flask (250 mL) containing a stir bar. Separately, glucose solution (250 mM, 11.6 mL) was sparged with argon in a conical flask. The oxygen quenching mixture (10×, 11.6 mL) was degassed in a conical flask that was placed under high vacuum until slight foaming occurred (1-2 s) and then back-filled with argon; this sequence was repeated several times. Sparged glucose solution was then added to the anaerobic cell suspension via syringe, followed by the oxygen quenching system. Finally, substrate 1 (80 mM, 2.9 mL DMSO) was added dropwise via syringe, and the reaction was stirred for 24 h at room temperature. To quench the reaction, dilute HCl (3 M, 8.7 mL) and acetonitrile (125 mL) were added. Cell debris was pelleted by centrifugation (8000×g, 10 minutes), and the supernatant was then extracted with ethyl acetate (2×250 mL). Solvent removal in vacuo left a brown oil (1 g), which was purified on silica gel via a stepwise elution (hexanes, 90/10 hexanes/ethyl acetate, 80/20 hexanes/ethyl acetate, 70/30 hexanes/ethyl acetate, ethyl acetate). Fractions containing 3 (as judged by TLC developed in 90/10 hexanes/ethyl acetate and stained with Cl₂/O-tolidine) were pooled and solvent was removed in vacuo. The resulting material was further purified on silica gel via an isocratic elution (50/50 hexanes/ether) affording 3 (38.6 mg, 69% yield), which was established by chiral HPLC as well as ¹H and ¹³C NMR.

Synthesis of Substrates and Standards.

Synthesis of azide substrates and benzosultam standards was accomplished as previously described, their spectra matched those previously reported (Ruppel, J. V. et al., Org. Lett. 9, 4889-4892 (2007)).

2,4,6-triethylbenzenesulfonyl azide (1) ¹H NMR (500 MHz, CDCl₃): δ 7.07 (2H, s), 3.06 (4H, q, J=7.39 Hz), 2.66 (2H, q, J=7.59 Hz), 1.29 (6H, t, J=7.41 Hz), 1.26 (3H, t, J=7.65 Hz); ¹³C NMR (125 MHz, CDCl₃): δ 150.9, 146.5, 132.5, 129.8, 28.7, 28.5, 17.0, 15.0.

FIG. 28 shows ¹H and ¹³C NMR spectra for synthetic (1).

Synthetic (3)¹H NMR (500 MHz, CDCl₃): δ 7.13 (1H, s), 6.98 (1H, s), 4.69 (m, 1H) 4.62 (s, 1H), 2.99 (2H, q, J=7.57 Hz), 2.71 (2H, q, J=7.62 Hz), 1.59 (3H, d, J=6.69 Hz), 1.35 (3H, t, J=7.57 Hz), 1.26 (3H, t, J=7.67 Hz); ¹³C NMR (125 MHz, CDCl₃): δ 150.8, 142.6, 140.5, 131.5, 128.9, 120.4, 52.8, 29.2, 24.5, 21.8, 15.6, 14.8.

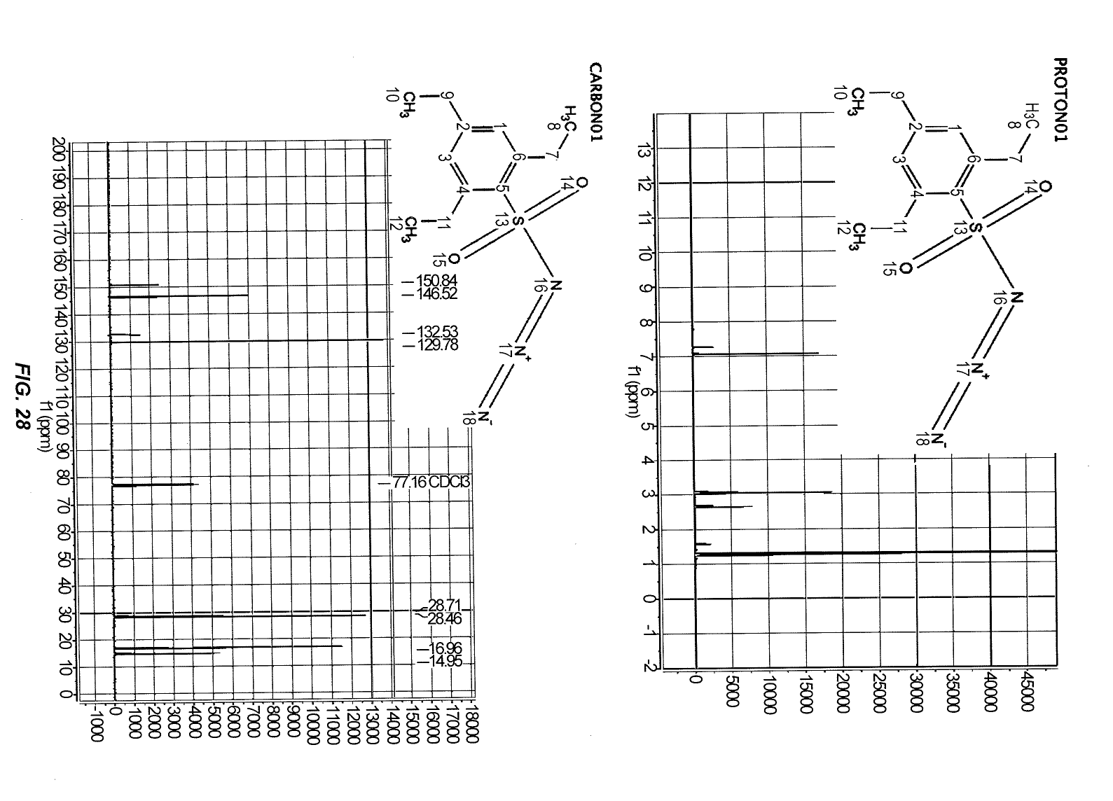

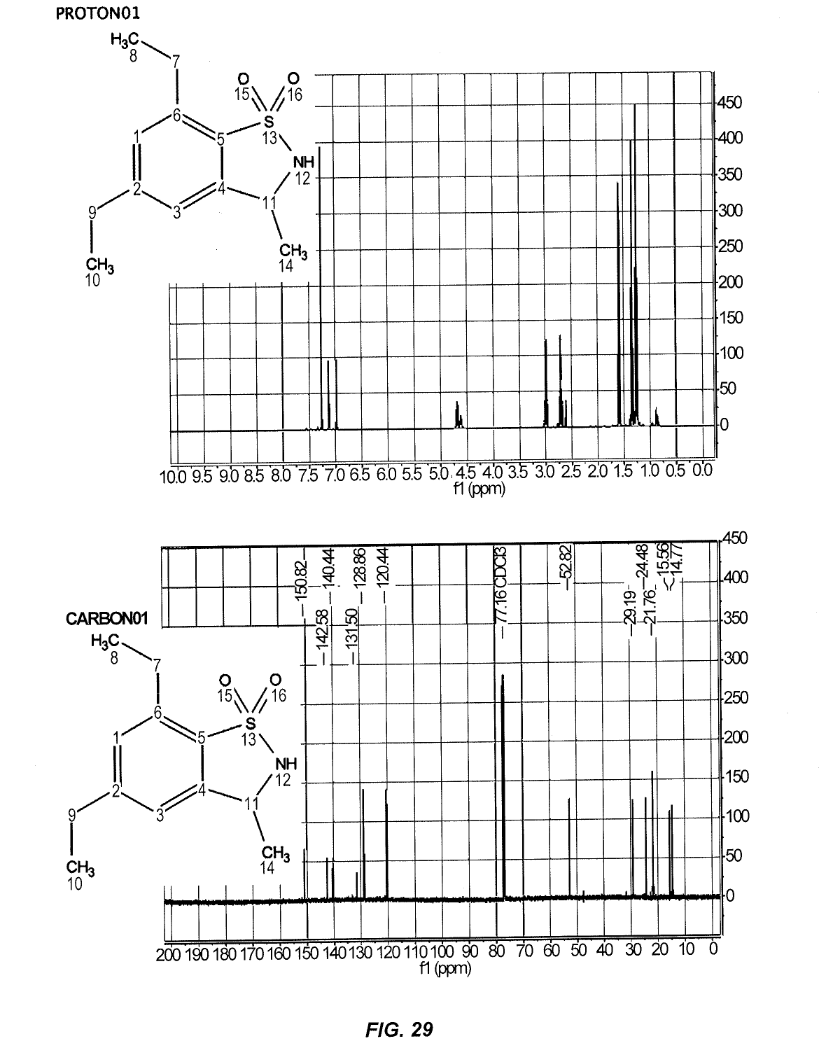

FIG. 29 shows ¹H and ¹³C NMR spectra for synthetic (3).

Enzyme synthesized (3)¹H NMR (600 MHz, CDCl₃): δ 7.13 (1H, s), 6.98 (1H, s), 4.69 (1H, m), 4.56 (1H, br) 3.00 (2H, q, J=7.64 Hz), 2.71 (2H, q, J=7.65 Hz), 1.59 (3H, t, J=6.68 Hz), 1.35 (3H, t, J=7.56 Hz), 1.26 (3H, t, J=7.61 Hz); ¹³C NMR (125 MHz, CDCl₃): 150.8, 142.6, 140.4, 131.5, 128.8, 120.4, 52.8, 29.2, 24.5, 21.8, 15.6, 14.8.

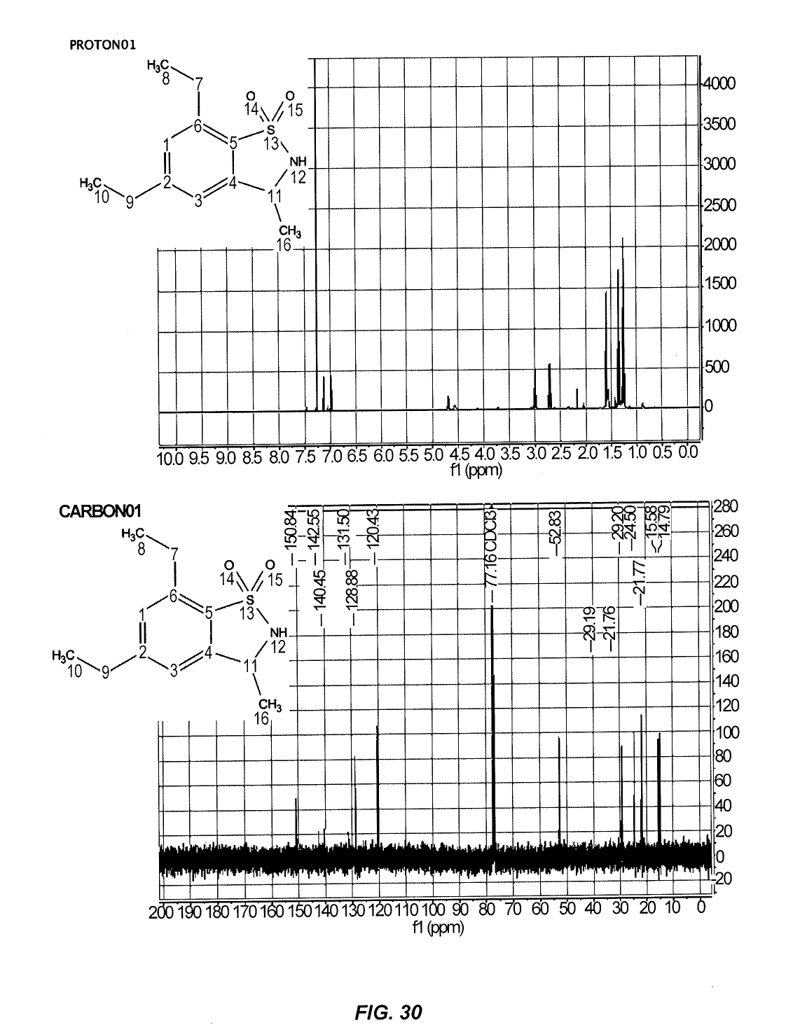

FIG. 30 shows ¹H and ¹³C NMR spectra for enzyme-produced (3).

2,4,6-triethylbenzenesulfonamide (2) was synthesized by sodium borohydride reduction of 1. ¹H NMR (500 MHz, CDCl₃): δ 7.01 (2H, s), 4.80 (br), 3.07 (4H, q, J=7.25 Hz), 2.63 (2H, q, J=7.66 Hz), 1.29 (6H, t, J=7.43 Hz), 1.24 (3H, t, J=7.76); ¹³C NMR (125 MHz, CDCl₃): δ 148.7, 144.8, 135.5, 129.4, 28.6, 28.5, 16.9, 15.2. Expected m/z for C₁₂H₁₉NO₂S⁺241.1136. found 241.1146.

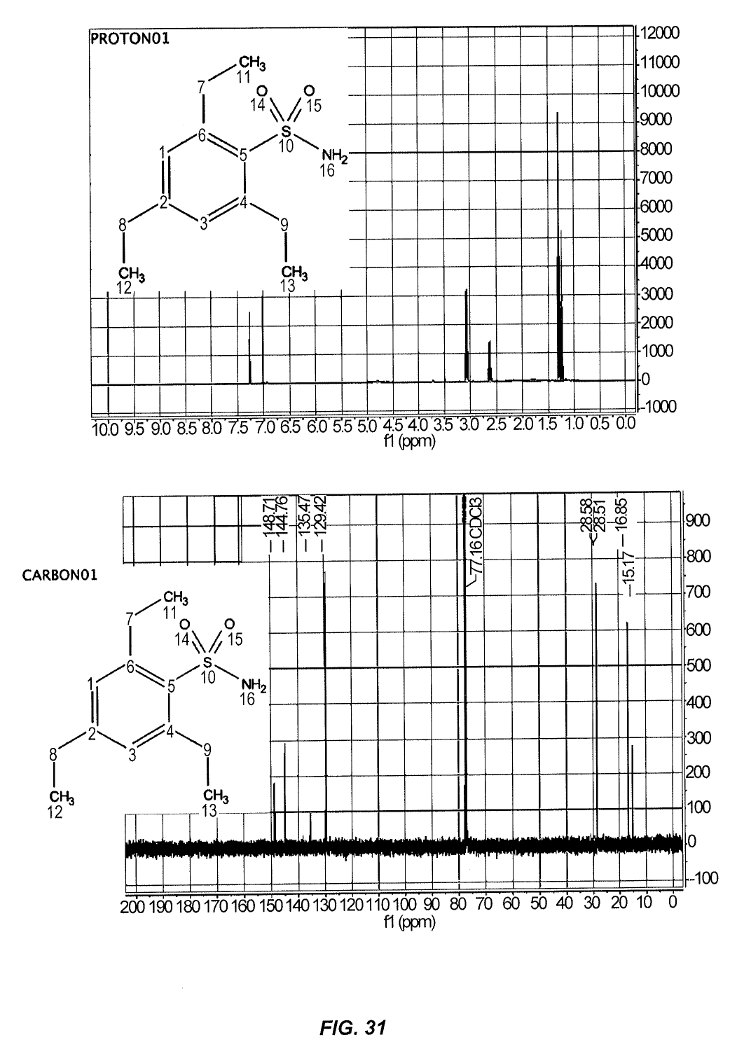

FIG. 31 shows ¹H and ¹³C NMR spectra of 2,4,6-triethylbenzenesulfonamide (2).

2,4,6-trimethylbenzenesulfonyl azide (4)¹H NMR (500 MHz, CDCl₃): δ 7.02 (2H, s), 2.66 (6H, s), 2.34 (3H, s); ¹³C NMR (125 MHz, CDCl₃): δ 144.7, 140.1, 133.4, 132.3, 22.9, 21.3.

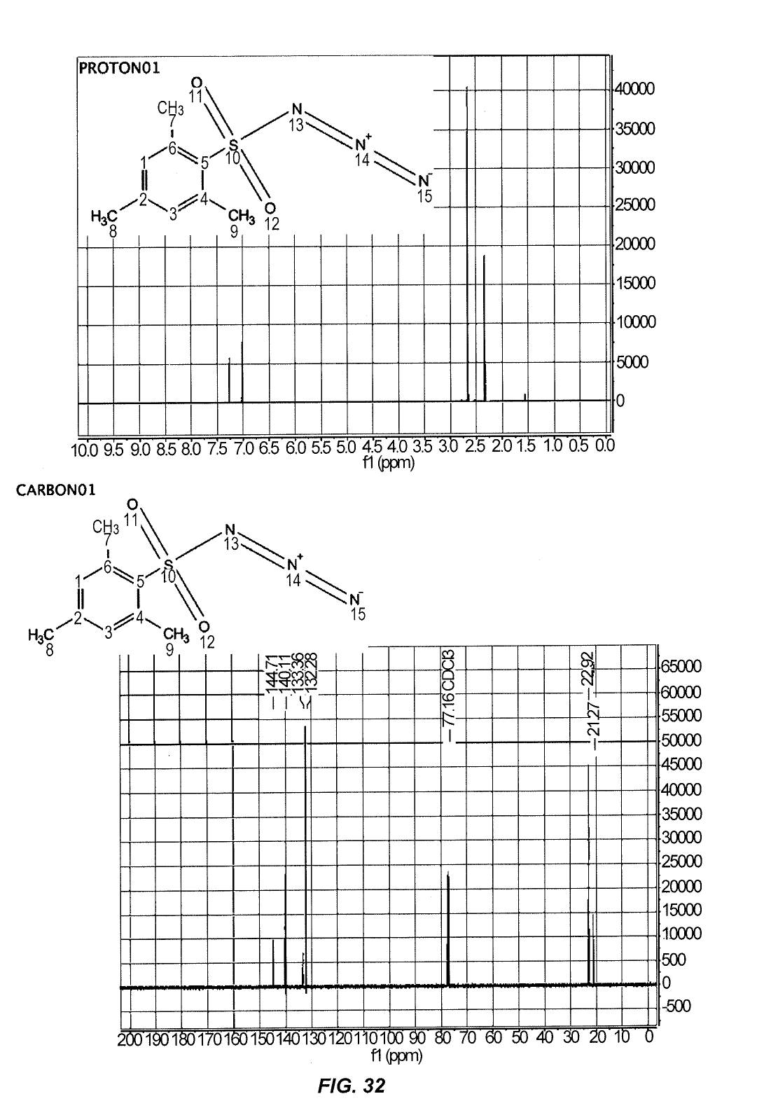

FIG. 32 shows ¹H and ¹³C NMR spectra of 2,4,6-trimethylbenzenesulfonyl azide (4).

(5)¹H NMR (500 MHz, CDCl₃): δ 7.06 (1H, s), 6.96 (1H, s), 4.73 (1H, br), 4.43 (2H, d, J=5.41), 2.59 (3H, s), 2.39 (3H, s); ¹³C NMR (125 MHz, CDCl₃): δ 144.3, 137.4, 134.2, 131.7, 131.5, 122.4, 45.2, 21.7, 17.0.

FIG. 33 shows ¹H and ¹³C NMR spectra of (5).

(7)¹H NMR (500 MHz, CDCl₃): δ 7.22 (1H, d, J=1.11 Hz), 6.98 (1H, d, J=1.32 Hz), 4.45 (br, 1H), 3.61 (1H, sep, J=6.85 Hz), 2.98 (1H, sep, J=6.88 Hz), 1.63 (6H, s), 1.35 (6H, d, J=6.90), 1.27 (6H, d, J=6.92); ¹³C NMR (125 MHz, CDCl₃): δ 155.7, 146.8, 145.5, 131.0, 124.5, 117.9, 59.9, 34.8, 30.0, 29.6, 24.0, 23.7.

FIG. 34 shows ¹H and ¹³C NMR spectra of 2,4,6-trimethylbenzenesulfonyl azide (7)

Assignment of Absolute Configuration.

Absolute configuration of triethylsultam 3 was assigned by comparison to other compounds described in the literature Oppolzer, W. et al., Tetrahedron lett. 31, 4117-4120 (1990); Ichinose, M. et al., Angew. Chem. Int. Ed. Engl. 50, 9884-9887 (2011)). In particular, sultam 3 and monoethyl derivative 8 (shown below) were previously synthesized in enantiopure form using a BINOL-iridium catalyst, which preferentially synthesized both (−)-3 α_{D}²⁰ −21.3 (c=1.01, CHCl₃) and (−)-8 [α_{D}²⁴]−26.9 (c=1.00, CHCl₃) (Ichinose, M. et al., Angew. Chem. Int. Ed. Engl. 50, 9884-9887 (2011)). The absolute configuration of 8, and the optical rotation values for its enantiomers has been previously reported (R)-8 [α_{D}²⁰]+31.0 (c=0.6, CHCl₃) and (S)-8 [α_{D}²⁰]−30 (c=1.21, CHCl₃) (Oppolzer, W. et al., Tetrahedron lett. 31, 4117-4120 (1990)). By analogy, the absolute configuration of the previously reported (−)-3 can be assigned as (S). Preparative chiral HPLC to separate the enantiomers of the racemic standard of 3 allowed isolation of the earlier-eluting enantiomer (which was the enzymatically preferred product). Measurement of the optical rotation of this material [α_{D}²⁵]−20.7 (c=1.1, CHCl₃) revealed it to be (S)-3.

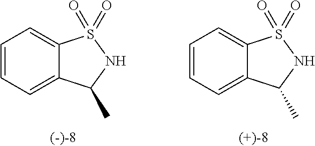

The examples set forth above are provided to give those of ordinary skill in the art a complete disclosure and description of how to make and use the embodiments of the devices, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

### Example 4

### In Vitro and In Vivo C—H, N—H, O—H and Si—H Insertion Catalyzed by Heme Proteins

### C—H Insertion

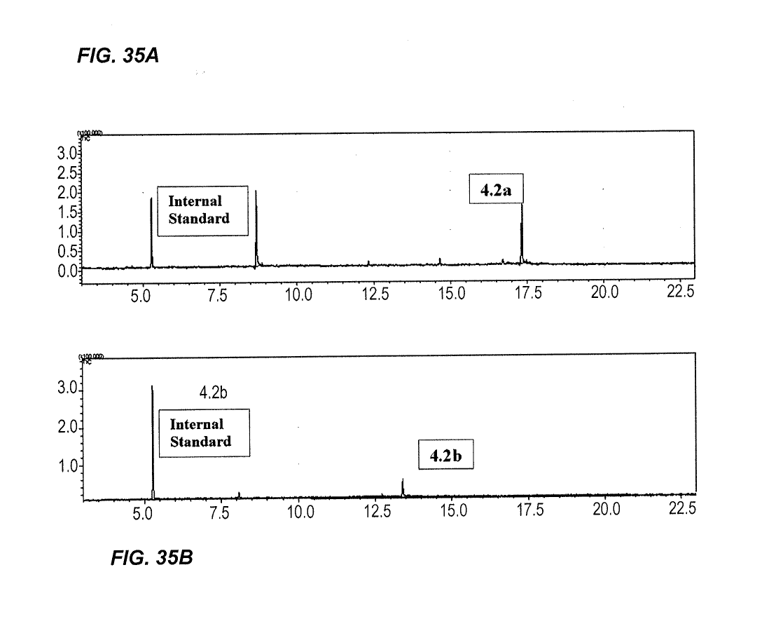

We have found that variants of P450 BM3 heme domain with mutations at the C400 axial site are competent catalysts for intramolecular C—H insertion under anaerobic conditions. These proteins are denoted by “AxX” where X is the amino acid at the axial position (i. e. position 400 in wild type BM3, denoted “WT-BM3 (heme domain)”. When substrates 4.1a and 4.1b (10 mM) were combined with BM3 heme domain variants (10 μM) and Na₂S₂O₄ (10 mM), we observed lactone formation via C—H bond insertion with variant WT-AxD (heme domain) after 16 h at room temperature (Table 17). The product was extracted with ethyl acetate or cyclohexane, was identified by GC-mass spectrometry (FIG. 35), and compared to an authentic sample of product 4.2b synthesized independently from hydroxy-γ-butyrolactone. Yield of product 4.22b was established after calibration with phenethyl alcohol as an internal standard. The reaction is not catalyzed by the WT-BM3 heme domain or hemin at 5% catalyst loading.

A representative procedure for P450-catalyzed C—H insertion is as follows: A vial containing WT-AxD (20 μL, 200 μM) was sealed with a teflon cap and the headspace of this vial was purged with argon for 5 min. Concurrently, a solution of sodium dithionite (11 mM in phosphate buffer pH=8) was degassed for a minimum of 5 min and 360 μL of this solution was transferred to the vial containing protein. The gas lines were disconnected from the vials. The substrate 4.1a or 4.1b was then added as a solution (20 μL, 200 mM). Final concentrations of all components were: 10 uM enzyme, 10 mM substrate, and 10 mM dithionite. The reaction vials were then placed in a tray on a plate shaker and left to shake at 40 rpm for 12 h then 20 μL phenethyl alcohol (20 mM) was added as an internal standard.

The mixture was extracted with 1 mL cyclohexane and analyzed by GC-MS or GC. GC-MS was performed on a Shimadzu TQ8030 GC-MS with ion count detector and J&W HP-5 column (30 m×0.32 mm, 0.25 μm film) using the following method: 90° C. (hold 2 min), 90-190° C. (20° C./min), and 190-230 (40° C./min)

We have previously shown that carbene cyclopropanation catalyzed by P450s can be performed by whole cells expressing these enzymes (Coelho, P. et al. Nat. Chem. Biol. 2013, 9, 485-487). Thus, the class of carbene transformation described herein can also be catalyzed by intact E. coli expressing the active variant of P450 under anaerobic conditions.

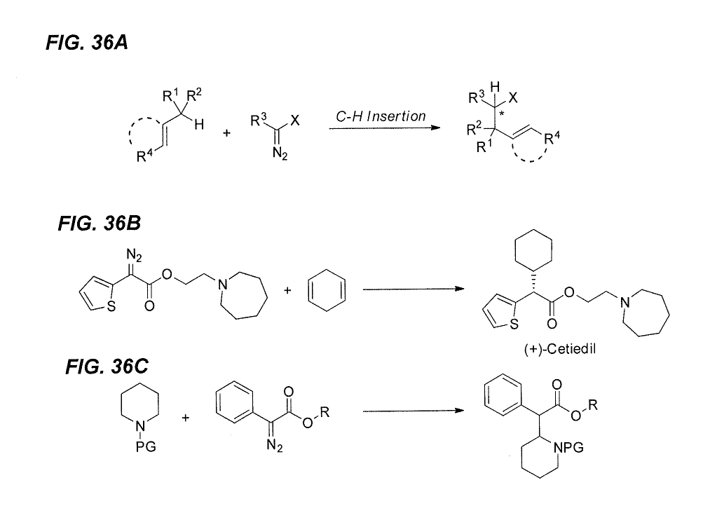

Additionally, P450 carbene insertion into C—H bonds can be used to construct carbon stereocenters including, but not limited to, the general form shown above (FIG. 36A), where X=carbonyl, nitrile, or amide and R=alkyl, aryl or H. Variants of P450 optimized for carbene cyclopropanation have shown high diastereo- and enantioselectivity and optimization of active mutants for carbene C—H insertion will yield enantioenriched products as well. The intramolecular transformation can be used to make chiral lactones, lactams and other rings of various sizes, and the intermolecular transformation can be used to make pharmaceutical targets such as (+)-cetiedil as outlined in FIG. 36B (Davies, H. L. et al. Tetrahedron Leu. 2002. 43, 4981-4983) and Ritalin as outlined in FIG. 36C (H. M. L. Davies, et al. Nature. 2008, 451, 417-424).

TABLE 19

| Intramolecular carbene C—H insertion. | | | |
|---|---|---|---|
| Reactions are performed with 10 mM 4.1, | | | |
| 10 uM P450 or 5% hemin, and 10 mM Na₂S₂O₄. | | | |
| 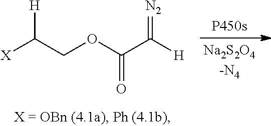 | | | |
| 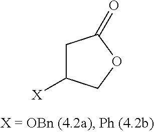 | | | |
| Substrate | Product | Catalyst | Yield % |
| 4.1a | 4.2a | 5% hemin | 0 |
| 4.1a | 4.2a | WT-BM3 heme | 0 |
| 4.1a | 4.2a | WT-AxA | trace |
| 4.1a | 4.2a | WT-AxD | 14% |
| 4.1a | 4.2a | WT-AxH | 0 |
| 4.1a | 4.2a | WT-AxK | 0 |
| 4.1a | 4.2a | WT-AxM | 0 |
| 4.1a | 4.2a | WT-AxN | 0 |
| 4.1a | 4.2a | WT-AxY | trace |
| 4.1b | 4.2b | WT-AxD | 4% |

### N—H Insertion

We have found a variety of P450 variants to be general catalysts for carbenoid N—H insertion. We combined aniline with EDA in the presence of a reductant (Na₂S₂O₄) under argon atmosphere and tested the mixture with seven P450_{BM3} variants previously identified as competent catalysts for cyclopropanation (Coelho, P. et al., Science. 2013, 339, 307-310). In choosing this set of P450s, we hypothesized that cyclopropanation activity could correlate with ability to generate the iron-carbenoid intermediate that is also necessary for N—H insertion. Whereas wild type BM3 (WT-BM3 (SEQ ID NO:1), Table 20, entry 1) provided only trace amounts of the desired product (4.3), a few variants gave 4.3 in good yields after 12 h at room temperature. In particular, variant H2-5-F10 which is derived from a thermostable P450_{BM3} lineage (Lewis, J. C. et al. ChemBioChem, 2010, 11, 2502-2505) and contains 15 mutation from WT-BM3 (SEQ ID NO:1), formed 4.3 in 47% yield and 473 turnovers (TTN) using 0.1% protein relative to EDA (entry 7). No double insertion product was observed, as determined by GC-MS and ¹H NMR of the products in milligram-scale reactions. In contrast, when 1 mol % of the isolated hemin prosthetic group was used as catalyst, the single and double insertion products were produced in a 3.5 to 1 ratio (with a total product yield of 51%).

A representative procedure for P450-catalyzed N—H insertion is as follows. To an unsealed crimp vial, 60 μL of a P450 solution (67 or 133 μM) was added and the vial was sealed. A 12.5 mM solution of sodium dithionite in phosphate buffer (0.1 M, pH=8.0) was degassed by bubbling with argon in a 6 mL crimp-sealed vial. The headspace of the 2 mL vials containing P450 solution were flushed with argon (no bubbling). The buffer/dithionite solution (360 μL) was then added to each reaction vial via syringe, and the gas lines were disconnected from the vials. 10 μL of an 800 mM stock of aniline was added via a glass syringe, followed by 10 μL of a 340 mM stock of EDA (both stocks in MeOH). The reaction vials were then placed in a tray on a plate shaker and left to shake at 40 rpm. The final concentrations of the reagents were typically: 20 mM aniline, 8.5 mM EDA, 10 mM Na₂S₂O₄, and 10 or 20 μM P450. After 12 h at 25° C., the vials were removed from the shaker and uncapped and 1 mL of cyclohexane was added, followed by 20 μL of a 20 mM solution of phenethyl alcohol (internal standard). The mixture was transferred to a 1.5 mL Eppendorf tube and vortexed and centrifuged (13,000×g, 1 min.). The organic layer was dried over sodium sulfate if necessary then analyzed by GC, with comparison to an authentically prepared sample that has been verified by proton NMR (300 mHz, Varian, CDCl₃).

When CO was bubbled gently through the protein solution before the addition of EDA and aniline, no product formation was observed (Table 20, entry 9), presumably due to complexation of CO to the iron center. Additionally, variants BM3-CIS, H2-4-D4, and H2-5-F10, (Table 20, entries 1, 6, and 8, respectively) differ by only 1-2 active site amino acids from variant H2-A-10″ yet all four exhibit a range of activity (24-47% yield). This demonstrates that slight changes in sequence and presumably the geometry around the protein active site lead to substantial differences in activity.

TABLE 20
N—H insertion with Cys-ligated P450 variants.

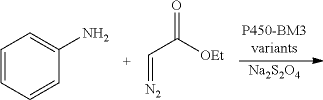

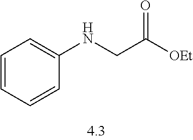 [SMILES: CCOC(=O)CNc1ccccc1]

| Entry | Catalyst^{a,b} | % Yield^{c} | TTn |
|---|---|---|---|
| 1 | BM3-WT | 1.7 | 17 |
| 2 | WT-T268A | 16 | 160 |
| 3 | BM3-CIS | 36 | 363 |
| 4 | P411-CIS | 14 | 136 |
| 5 | P411-T268A | 9.5 | 95 |
| 6 | H2-4-D4 | 34 | 340 |
| 7 | H2-A-10 | 24 | 238 |
| 8 | H2-5-F10 | 47 | 473 |
| 9 | H2-5-F10 + CO | 0 | 0 |

^{a}Reactions were carried out with protein (10 uM), ethyl diazo acetate (8.5 mM), aniline (20 mM) and Na₂S₂O₄ (10 mM) in phosphate buffer (pH 8) and allowed to shake at room temperature for 12 h.
^{b}See Table 15 and Provisional Application No. 61/711,640, filed Oct. 9, 2012 for amino acid differences from BM3-WT for each mutant.
^{c}Yields were determined by GC calibrated for 4.3.

TABLE 21
Amino acid variations from WT-BM3 (SEQ ID NO: 1) for each variant
discussed above.

| | Amino acid sequence with respect to wild type |
|---|---|
| Enzyme | P450_{BM3} (SEQ ID NO: 1) |
| WT-BM3 | None |
| WT-T268A | T268A |
| P411-T268A | T268A, C400S |
| 9-10A TS | V78A, P142S, T175I, A184V, S226R, H236Q, |
| | E252G, A290V, L353V, I366V, E442K |
| BM3-CIS | 9-10 A TS + F87V, T268A |
| P411-CIS | 9-10 A TS + F87V, T268A, C400S |
| H2A10 | 9-10 A TS + F87V, L75A, L181A, T268A |
| H2-4-D4 | 9-10 A TS + F87V, L75A, M177A, L181A, |
| | T268A, L437A, |
| H2-5-F10 | 9-10A TS + F87V, L75A, I263A, T268A, |
| | L437A |

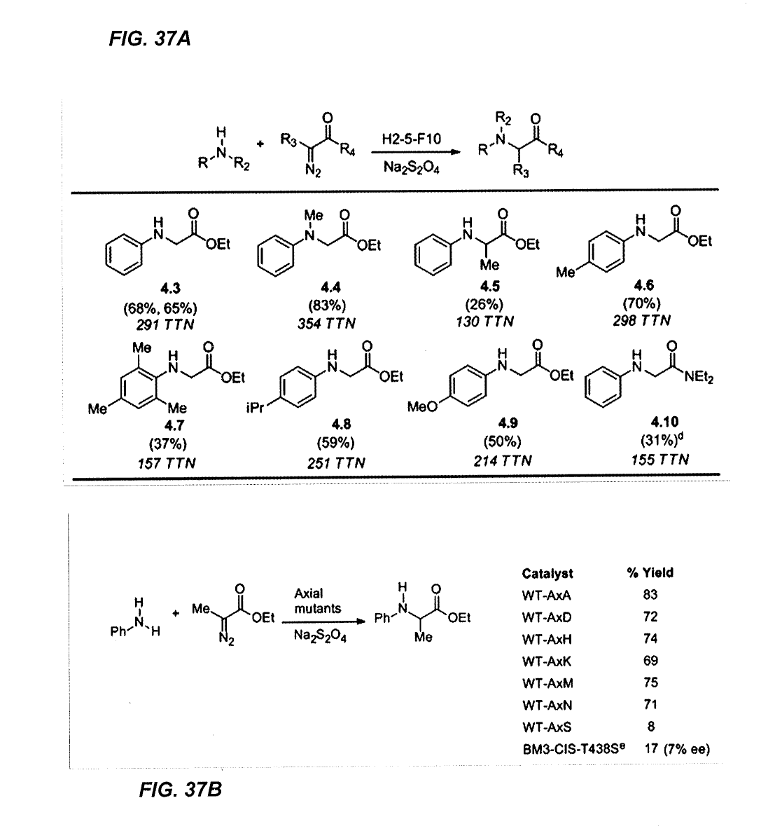

We examined a variety of substrates for N—H insertion using P450 variant H2-5-F10 and found that this catalyst is fairly general and can catalyze N—H insertion with both primary and secondary amines (FIG. 37A). Substitution is well-tolerated on the aniline partner on both the ortho and para positions. Reactions were analyzed by a Shimadzu GC with FID detector and J&W HP-5 column (30 m×0.32 mm, 0.25 μm film) and calibrated to an independently prepared sample of each product using phenethyl alcohol as internal standard. Reaction of aniline and EDA was also performed in milligram scale without any organic cosolvent to provide 4.3 in 65% isolated yield.

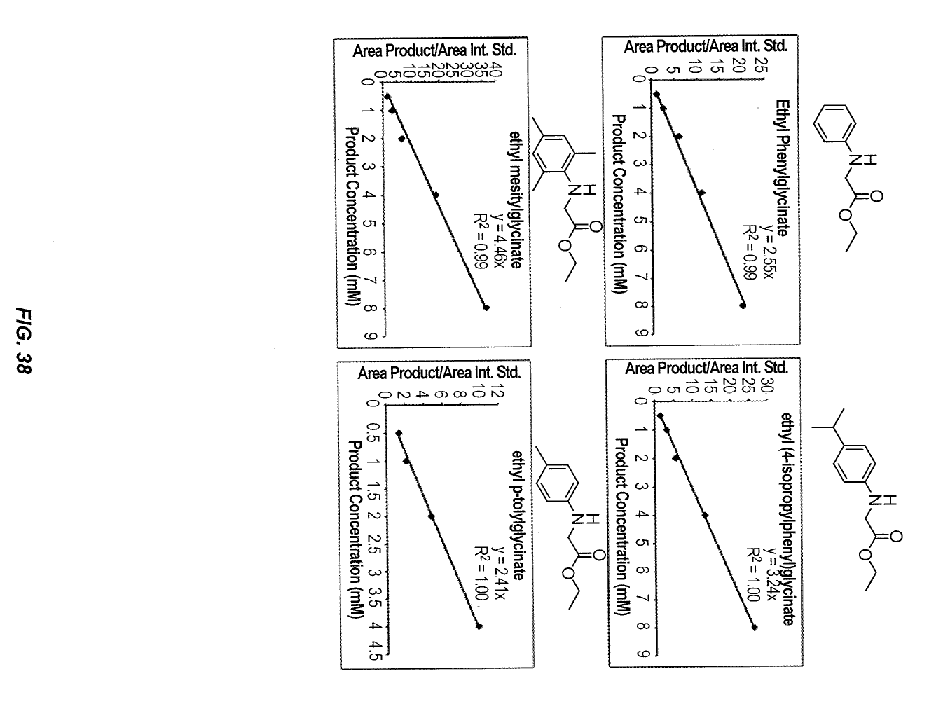

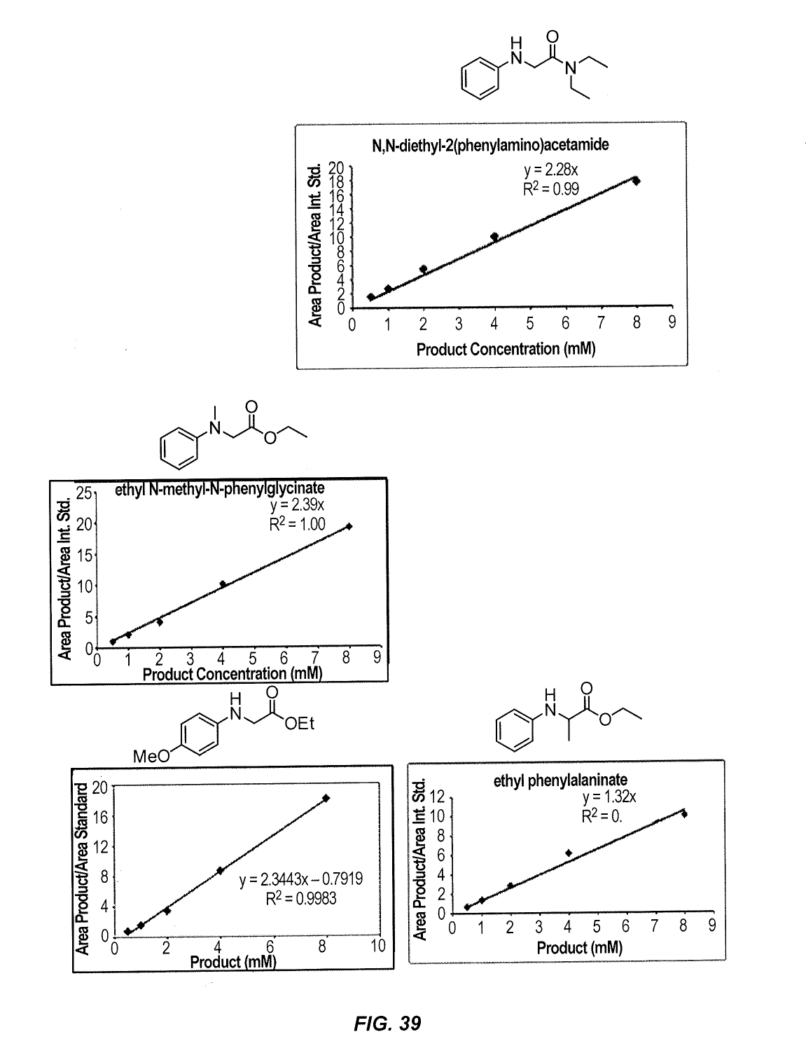

Calibration curves were plotted as follows. Yields of N—H insertion products were determined using calibration curves made with independently synthesized standards that have been verified by proton NMR, with comparison to known literature data (Baumann, L. K. et al. Organometallics. 2007, 26, 3995-4002; Anding, B. J. et al. Organometallics. 2013, 32, 2599-2607). Two stock solutions of product were made at 160 mM and 40 mM. To four or five samples containing 380 μL of buffer, product was added from either of the stock solutions such at a final concentration of 0.5-8.0 mM in 400 μL was obtained. 20 μL of internal standard was added to each eppendorf tube, then 1 mL of cyclohexane was added and the tubes were vortexed and centrifuged (13,000 ×g, 30 seconds). The organic layer was then analyzed by GC. The ratio of the areas under the internal standard and product peaks were plotted against the concentration for each solution. Calibration curves for each product are shown in FIG. 38 and FIG. 39.

The reactions shown in FIG. 37A were carried out with protein (20 μM), ethyl diazo acetate (8.5 mM), aniline (20 mM) and Na₂S₂O₄ (10 mM) in phosphate buffer (pH 8) and allowed to shake at room temperature for 12 h. Yields are reported in parenthesis and TTN are presented in italics. Yields were determined by GC calibrated for each product. An isolated yield of 65% for compound 4.3 was determined for the milligram scale reaction run as follows: 25 mM EDA, 25 mM aniline, 25 μM protein, and 25 mM Na₂S₂O₄. The reaction producing compounds 4.5 and 4.10 were carried out with protein (20 μM), ethyl diazo acetate (10.0 mM), aniline (20 mM) and Na₂S₂O₄ (10 mM).

While substituted diazo compounds can also be used for insertion, the yield from N—H insertion of ethyl 2-diazopropanoate into aniline is only 26% (product 4.5). To improve efficiency of this reaction, we examined the reaction of ethyl 2-diazopropanoate with a variety of axial mutants. In particular, the axial mutant WT-AxA provided the desired product in 83% yield, compared to 8% yield and 1.7% yield with axial mutant WT-AxS and WT-BM3 heme domain, respectively (FIG. 37B). The reaction can also be performed with whole cells expressing BM3-CIS-T438S to provide 4.5 in 17% yield and 7% enantioselectivity (FIG. 37B), demonstrating the first enantioselective N—H insertion catalyzed by a protein.

The reactions shown in FIG. 37B were carried out with 10 μM catalyst loading, 20 mM aniline, 10 mM Na₂S₂O₄ and 10 mM ethyl 2-diazopropanoate as described above. For the final entry in FIG. 37B, whole cells expressing BM3-CIS-T438S at 2.5 μM were used. See, U.S. Provisional Application No. 61/711,640 for the procedure for catalysis with whole cells.

Diazo amides are also competent substrates for this reaction, and alpha-amino amides like 4.10 (FIG. 37A) can be produced in moderate yields. This transformation is particularly valuable because diazo amides can give rise to important building block compounds like diamines Additionally, this transformation can be used for the synthesis of lidocaine, which we were able to prepare using diethyl amine and 4.11 in the presence of H2-5-F10 (Scheme 1). Production of lidocaine was observed by GC-MS using the method 100° C. (hold 1 min), 100-140° C. (6° C./min), 140-260° C. (20° C./min), 260° C. (hold 3 min)

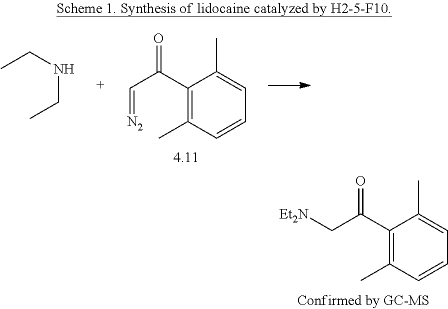

### O—H and Si—H Insertion

As we have demonstrated that P450 can catalyze the insertion of diazo compounds into C—H and N—H bonds, catalytic O—H and Si—H insertion may also be achieved as shown in Scheme 2. O—H insertion is used to construct C—O bonds from diazo compounds. As we have demonstrated that heme protein catalyzed cyclopropanation and N—H insertion is stereoselective and efficient and can be performed with whole cells expressing the heme proteins, enantioselective O—H insertion can also be achieved both in vivo and in vitro. Enantioselective O—H insertion is useful for building chiral C—O stereocenters, including but not limited to the C—O stereocenter found in duloxetine. Both aryl O—H and alkyl O—H bonds are used for this insertion reaction. Si—H insertion reactions yield silane products that have many applications as materials, polymers, and substrates for Hiyama cross coupling.

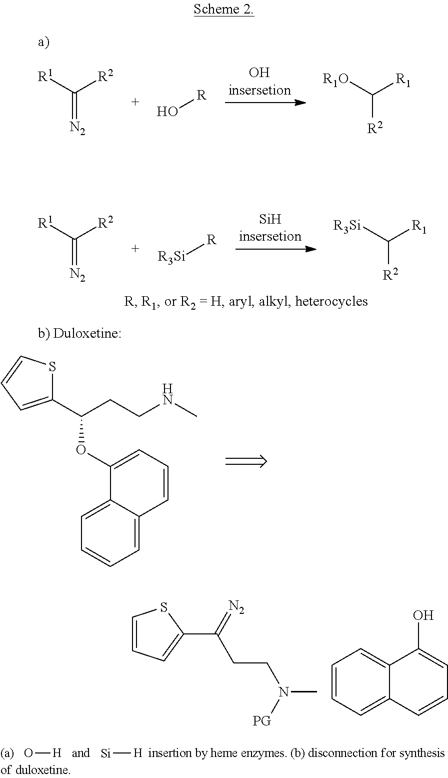

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

Informal Sequence Listing

SEQ ID NO: 1
CYP102A1
Cytochrome P450 (BM3)
Bacillus megaterium
GenBank Accession No. AAA87602
>gi|142798|gb|AAA87602.1|cytochrome P-450: NADPH-P-450 reductase precursor
[Bacillus megaterium]
TIKEMPQPK TFGELKNLPL LNTDKPVQAL MKIADELGEI FKFEAPGRVT RYLSSQRLIK
EACDESRFDK NLSQALKFVR DFAGDGLFTS WTHEKNWKKA HNILLPSFSQ QAMKGYHAMM
VDIAVQLVQK WERLNADEHI EVPEDMTRLT LDTIGLCGFN YRFNSFYRDQ PHPFITSMVR
ALDEAMNKLQ RANPDDPAYD ENKRQFQEDI KVMNDLVDKI IADRKASGEQ SDDLLTHMLN
GKDPETGEPL DDENIRYQII TFLIAGHETT SGLLSFALYF LVKNPHVLQK AAEEAARVLV
DPVPSYKQVK QLKYVGMVLN EALRLWPTAP AFSLYAKEDT VLGGEYPLEK GDELMVLIPQ
LHRDKTIWGD DVEEFRPERF ENPSAIPQHA FKPFGNGQRA CIGQQFALHE ATLVLGMMLK
HFDFEDHTNY ELDIKETLTL KPEGFVVKAK SKKIPLGGIP SPSTEQSAKK VRKKAENAHN
TPLLVLYGSN MGTAEGTARD LADIAMSKGF APQVATLDSH AGNLPREGAV LIVTASYNGH
PPDNAKQFVD WLDQASADEV KGVRYSVFGC GDKNWATTYQ KVPAFIDETL AAKGAENIAD
RGEADASDDF EGTYEEWREH MWSDVAAYFN LDIENSEDNK STLSLQFVDS AADMPLAKMH
GAFSTNVVAS KELQQPGSAR STRHLEIELP KEASYQEGDH LGVIPRNYEG IVNRVTARFG
LDASQQIRLE AEEEKLAHLP LAKTVSVEEL LQYVELQDPV TRTQLRAMAA KTVCPPHKVE
LEALLEKQAY KEQVLAKRLT MLELLEKYPA CEMKFSEFIA LLPSIRPRYY SISSSPRVDE
KQASITVSVV SGEAWSGYGE YKGIASNYLA ELQEGDTITC FISTPQSEFT LPKDPETPLI
MVGPGTGVAP FRGFVQARKQ LKEQGQSLGE AHLYFGCRSP HEDYLYQEEL ENAQSEGIIT
LHTAFSRMPN QPKTYVQHVM EQDGKKLIEL LDQGAHFYIC GDGSQMAPAV EATLMKSYAD
VHQVSEADAR LWLQQLEEKG RYAKDVWAG
SEQ ID NO: 2
CYP102A1
B. megaterium
>gi|281191140|gb|ADA57069.1|NADPH-cytochrome P450 reductase 102A1V9
[Bacillus megaterium]
MTIKEMPQPKTFGELKNLPLLNTDKPIQTLMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNTDEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPREGAVLIVTASYNGHPPDNAKEFVDWLDQASADEV
KGVRYSVFGCGDKNWATTYQKVPAFIDETLAAKGAENIAERGEADASDDFEGTYEEWREHMWSDLAAYFN
LDIENSEENASTLSLQFVDSAADMPLAKMHRAFSANVVASKELQKPGSARSTRHLEIELPKEASYQEGDH
LGVIPRNYEGIVNRVATRFGLDASQQIRLEAEEEKLAHLPLGKTVSVEELLQYVELQDPVTRTQLRAMAA
KTVCPPHKVELEVLLEKQAYKEQVLAKRLTMLELLEKYPACEMEFSEFIALLPSMRPRYYSISSSPRVDE
KQASITVSVVSGEAWSGYGEYKGIASNYLANLQEGDTITCFVSTPQSGFTLPKGPETPLIMVGPGTGVAP
FRGFVQARKQLKEQGQSLGEAHLYFGCRSPHEDYLYQKELENAQNEGIITLHTAFSRVPNQPKTYVQHVM
EQDGKKLIELLDQGAHFYICGDGSQMAPDVEATLMKSYAEVHQVSEADARLWLQQLEEKGRYAKDVWAG
SEQ ID NO: 3
CYP102A1
B. megaterium
>gi|281191138|gb|ADA57068.1|NADPH-cytochrome P450 reductase 102A1V10
[Bacillus megaterium]
MTIKEMPQPKTFGELKNLPLLNTDKPIQTLMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNTDEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPREGAVLIVTASYNGHPPDNAKEFVDWLDQASADEV
KGVRYSVFGCGDKNWATTYQKVPAFIDETFAAKGAENIAERGEADASDDFEGTYEEWREHMWSDLAAYFN
LDIENSEENASTLSLQFVDSAADMPLAKMHRAFSANVVASKELQKPGSARSTRHLEIELPKEASYQEGDH
LGVIPRNYEGIVNRVATRFGLDASQQIRLEAEEEKLAHLPLGKTVSVEELLQYVELQDPVTRTQLRAMAA
KTVCPPHKVELEVLLEKQAYKEQVLAKRLTMLELLEKYPACEMEFSEFIALLPSMRPRYYSISSSPRVDE
KQASITVSVVSGEAWSGYGEYKGIASNYLANLQEGDTITCFVSTPQSGFTLPKGPETPLIMVGPGTGVAP
FRGFVQARKQLKEQGQSLGEAHLYFGCRSPHEDYLYQKELENAQNEGIITLHTAFSRVPNQPKTYVQHVM
EQDGKKLIELLDQGAHFYICGDGSQMAPDVEATLMKSYAEVHQVSEADARLWLQQLEEKGRYAKDVWAG
SEQ ID NO: 4
CYP102A1
B. megaterium
>gi|281191126|gb|ADA57062.1|NADPH-cytochrome P450 reductase 102A1V4
[Bacillus megaterium]
MTIKEMPQPKTFGELKNLPLLNTDKPIQTLMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNTDEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEATRVLVDPVPSYKQVKQLKYVGMVLNEALRLWPTAPAFSLYAKEDTVLGGEYPLEK
GDELMVLIPQLHRDKTIWGEDVEEFRPERFENPSAIPQHAFKPFGNGQRACIGQQFALHEATLVLGMMLK
HFDFEDHTNYELDIKETLTLKPEGFVVKAKSKKIPLGGIPSPSTEQSAKKVRKKVENAHNTPLLVLYGSN
MGTAEGTARDLADIAMSKGFAPQVATLDSHAGNLPREGAVLIVTASYNGHPPDNAKQFVDWLDQASADDV
KGVRYSVFGCGDKNWATTYQKVPAFIDETLAAKGAENIADRGEADASDDFEGTYEEWREHMWSDVAAYFN
LDIENSEDNKSTLSLQFVDSAADMPLAKMHGAFSANVVASKELQQPGSERSTRHLEIALPKEASYQEGDH
LGVIPRNYEGIVNRVTARFGLDASQQIRLEAEEEKLAHLPLGKTVSVEELLQYVELQDPVTRTQLRAMAA
KTVCPPHKVELEALLEKQAYKEQVLAKRLTMLELLEKYPACEMEFSEFIALLPSIRPRYYSISSSPRVDE
KQASITVSVVSGEAWSGYGEYKGIASNYLANLQEGDTITCFVSTPQSGFTLPKDSETPLIMVGPGTGVAP
FRSFVQARKQLKEQGQSLGEAHLYFGCRSPHEDYLYQEELENAQNEGIITLHTAFSRVPNQPKTYVQHVM
EQDGKKLIELLDQGAHFYICGDGSQMAPDVEATLMKSYADVYEVSEADARLWLQQLEEKGRYAKDVWAG
SEQ ID NO: 5
CYP102A1
B. megaterium
>gi|281191124|gb|ADA57061.1|NADPH-cytochrome P450 reductase 102A1V8
[Bacillus megaterium]
MTIKEMPQPKTFGELKNLPLLNTDKPIQTLMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNTDEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPRVATLDSHAGNLPREGAVLIVTASYNGHPPDNAKEFVDWLDQASADEV
KGVRYSVFGCGDKNWATTYQKVPAFIDETLAAKGAENIAERGEADASDDFEGTYEEWREHMWSDLAAYFN
LDIENSEENASTLSLQFVDSAADMPLAKMHRAFSANVVASKELQKPGSARSTRHLEIELPKEASYQEGDH
LGVIPRNYEGIVNRVATRFGLDASQQIRLEAEEEKLAHLPLGKTVSVEELLQYVELQDPVTRTQLRAMAA
KTVCPPHKVELEVLLEKQAYKEQVLAKRLTMLELLEKYPACEMEFSEFIALLPSMRPRYYSISSSPRVDE
KQASITVSVVSGEAWSGYGEYKGIASNYLANLQEGDTITCFVSTPQSGFTLPKGPETPLIMVGPGTGVAP
FRGFVQARKQLKEQGQSLGEAHLYFGCRSPHEDYLYQKELENAQNEGIITLHTAFSRVPNQPKTYVQHVM
EQDGKKLIELLDQGAHFYICGDGSQMAPDVEATLMKSYAEVHQVSEADARLWLQQLEEKGRYAKDVWAG
SEQ ID NO: 6
CYP102A1
B. megaterium
>gi|281191120|gb|ADA57059.1|NADPH-cytochrome P450 reductase 102A1V3
[Bacillus megaterium]
MTIKEMPQPKTFGELKNLPLLNTDKPVQALMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLVQKWERLNADEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKKIPLGGIPSPSTEQSAKKVRKKVENAHNTPLLVLYGSN
MGTAEGTARDLADIAMSKGFAPQVATLDSHAGNLPREGAVLIVTASYNGHPPDNAKQFVDWLDQASADDV
KGVRYSVFGCGDKNWATTYQKVPAFIDETLAAKGAENIADRGEADASDDFEGTYEEWREHMWSDVAAYFN
LDIENSEDNKSTLSLQFVDSAADMPLAKMHGAFSANVVASKELQQLGSERSTRHLEIALPKEASYQEGDH
LGVIPRNYEGIVNRVTARFGLDASQQIRLEAEEEKLAHLPLGKTVSVEELLQYVELQDPVTRTQLRAMAA
KTVCPPHKVELEALLEKQAYKEQVLAKRLTMLELLEKYPACEMEFSEFIALLPSISPRYYSISSSPHVDE
KQASITVSVVSGEAWSGYGEYKGIASNYLANLQEGDTITCFVSTPQSGFTLPKDSETPLIMVGPGTGVAP
FRGFVQARKQLKEQGQSLGEAHLYFGCRSPHEDYLYQEELENAQNEGIITLHTAFSRVPNQPKTYVQHVM
ERDGKKLIELLDQGAHFYICGDGSQMAPDVEATLMKSYADVYEVSEADARLWLQQLEEKGRYAKDVWAG
SEQ ID NO: 7
CYP102A1
B. megaterium
>gi|281191118|gb|ADA57058.1|NADPH-cytochrome P450 reductase 102A1V7
[Bacillus megaterium]
MTIKEMPQPKTFGELKNLPLLNTDKPIQTLMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNTDEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPPEGAVLIVTASYNGHPPDNAKEFVDWLDQASADEV
KGVRYSVFGCGDKNWATTYQKVPAFIDETLAAKGAENIAERGEADASDDFEGTYEEWREHMWSDLAAYFN
LDIENSEENASTLSLQFVDSAADMPLAKMHRAFSANVVASKELQKPGSARSTRHLEIELPKEASYQEGDH
LGVIPRNYEGIVNRVATRFGLDASQQIRLEAEEEKLAHLPLGKTVSVEELLQYVELQDPVTRTQLRAMAA
KTVCPPHKVELEVLLEKQAYKEQVLAKRLTMLELLEKYPACEMEFSEFIALLPSMRPRYYSISSSPRVDE
KQASITVSVVSGEAWSGYGEYKGIASNYLANLQEGDTITCFVSTPQSGFTLPKGPETPLIMVGPGTGVAP
FRGFVQARKQLKEQGQSLGEAHLYFGCRSPHEDYLYQKELENAQNEGIITLHTAFSRVPNEPKTYVQHVM
EQDGKKLIELLDQGAHFYICGDGSQMAPDVEATLMKSYAEVHQVSEADARLWLQQLEEKGRYAKDVWAG
SEQ ID NO: 8
CYP102A1
B. megaterium
>gi|281191112|gb|ADA57055.1|NADPH-cytochrome P450 reductase 102A1V2
[Bacillus megaterium]
MTIKEMPQPKTFGELKNLPLLNTDKPIQTLMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNTDEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEATRVLVDPVPSYKQVKQLKYVGMVLNEALRLWPTAPAFSLYAKEDTVLGGEYPLEK
GDELMVLIPQLHRDKTIWGEDVEEFRPERFENPSAIPQHAFKPFGNGQRACIGQQFALHEATLVLGMMLK
HFDFEDHTNYELDIKETLTLKPEGFVVKAKSKKIPLGGIPSPSTEQSAKKVRKKVENAHNTPLLVLYGSN
MGTAEGTARDLADIAMSKGFAPQVATLDSHAGNLPREGAVLIVTASYNGHPPDNAKQFVDWLDQASADDV
KGVRYSVFGCGDKNWATTYQKVPAFIDETLAAKGAENIADRGEADASDDFEGTYEEWREHMWSDVAAYFN
LDIENSEDNKSTLSLQFVDSAADMPLAKMHGAFSANVVASKELQQLGSERSTRHLEIALPKEASYQEGDH
LGVIPRNYEGIVNRVTARFGLDASQQIRLEAEEEKLAHLPLGKTVSVEELLQYVELQDPVTRTQLRAMAA
KTVCPPHKVELEALLEKQAYKEQVLAKRLTMLELLEKYPACEMEFSEFIALLPSISPRYYSISSSPHVDE
KQASITVSVVSGEAWSGYGEYKGIASNYLANLQEGDTITCFVSTPQSGFTLPKDSETPLIMVGPGTGVAP
FRGFVQARKQLKEQGQSLGEAHLYFGCRSPHEDYLYQEELENAQNEGIITLHTAFSRVPNQPKTYVQHVM
ERDGKKLIELLDQGAHFYICGDGSQMAPDVEATLMKSYADVYEVSEADARLWLQQLEEKGRYAKDVWAG
SEQ ID NO: 9
CYP102A1
B. megaterium
>gi|269315992|gb|ACZ37122.1|cytochrome P450: NADPH P450 reductase
[Bacillus megaterium]
MTIKEMPQPKTFGELKNLPLLNTDKPIQTLMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNTDEHI
EVPEDMTRLTLDTIGLCGFNYRFNSFYRDQPHPFITSMVRALDEAMNKLQRANPDDPAYDENKRQFQEDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPREGAVLIVTASYNGHPPDNAKEFVDWLDQASADEV
KGVRYSVFGCGDKNWATTYQKVPAFIDETLAAKGAENIAERGEADASDDFEGTYEEWREHMWSDLAAYFN
LDIENSEENASTLSLQFVDSAADMPLAKMHRAFSANVVASKELQKPGSARSTRHLEIELPKEASYQEGDH
LGVIPRNYEGIVNRVATRFGLDASQQIRLEAEEEKLAHLPLGKTVSVEELLQYVELQDPVTRTQLRAMAA
KTVCPPHKVELEVLLEKQAYKEQVLAKRLTMLELLEKYPACEMEFSEFIALLPSMRPRYYSISSSPRVDE
KQASITVSVVSGEAWSGYGEYKGIASNYLANLQEGDTITCFVSTPQSGFTLPKGPETPLIMVGPGTGVAP
FRGFVQARKQLKEQGQSLGEAHLYFGCRSPHEDYLYQKELENAQNEGIITLHTAFSRVPNQPKTYVQHVM
EQDGKKLIELLDQGAHFYICGDGSQMAPDVEATLMKSYAEVHQVSEADARLWLQQLEEKGRYAKDVWAG
SEQ ID NO: 10
CYP102A1
B. megaterium
>gi|281191116|gb|ADA57057.1|NADPH-cytochrome P450 reductase 102A1V6
[Bacillus megaterium]
MTIKEMPQPKTFGELKNLPLLNTDKPVQALMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNADEHI
EVPEDMTRLTLDTIGLCGFNYRFNSFYRDQPHPFITSMVRALDEAMNKLQRANPDDPAYDENKRQFQDDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPREGAVLIVTASYNGHPPDNAKQFVDWLDQASADEV
KGVRYSVFGCGDKNWATTYQKVPAFIDETLSAKGAENIAERGEADASDDFEGTYEEWREHMWSDLAAYFN
LNIENSEDNASTLSLQFVDSAADMPLAKMHGAFSANVVASKELQQPGSARSTRHLEIELPKEASYQEGDH
LGVIPRNYEGIVNRVTTRFGLDASQQIRLEAEEEKLAHLPLGKTVSVEELLQYVELQDPVTRTQLRAMAA
KTVCPPHKVELEALLEKQAYKEQVLTKRLTMLELLEKYPACEMEFSEFIALLPSMRPRYYSISSSPRVDE
KQASITVSVVSGEAWSGYGEYKGIASNYLAELQEGDTITCFVSTPQSGFTLPKDPETPLIMVGPGTGVAP
FRGFVQARKQLKEQGQSLGEAHLYFGCRSPHEDYLYQEELENAQNEGIITLHTAFSRVPNQPKTYVQHVV
EQDGKKLIELLDQGAHFYICGDGSQMAPDVEATLMKSYAEVHKVSEADARLWLQQLEEKSRYAKDVWAG
SEQ ID NO: 11
CYP102A1
B. megaterium
>gi|281191114|gb|ADA57056.1|NADPH-cytochrome P450 reductase 102A1V5
[Bacillus megaterium]
MTIKEMPQPKTFGELKNLPLLNTDKPVQALMKIADELGEIFKFEAPGRVTRYLSSQRLIKEACDESRFDK
NLSQALKFVRDFAGDGLFTSWTHEKNWKKAHNILLPSFSQQAMKGYHAMMVDIAVQLIQKWERLNADEHI
EVPEDMTRLTLDTIGLCGFNYRFNSFYRDQPHPFITSMVRALDEAMNKLQRANPDDPAYDENKRQFQDDI
KVMNDLVDKIIADRKASGEQSDDLLTHMLNGKDPETGEPLDDENIRYQIITFLIAGHETTSGLLSFALYF
LVKNPHVLQKAAEEAARVLVDPVPSYKQVKQLKYVGMVLNEALRLWPTAPAFSLYAKEDTVLGGEYPLEK
GDELMVLIPQLHRDKTIWGDDVEEFRPERFENPSAIPQHAFKPFGNGQRACIGQQFALHEATLVLGMMLK
HFDFEDHTNYELDIKETLTLKPEGFVVKAKSKQIPLGGIPSPSREQSAKKERKTVENAHNTPLLVLYGSN
MGTAEGTARDLADIAMSKGFAPQVATLDSHAGNLPREGAVLIVTASYNGHPPDNAKQFVDWLDQASADEV
KGVRYSVFGCGDKNWATTYQKVPAFIDETLSAKGAENIAERGEADASDDFEGTYEEWREHMWSDLAAYFN
LNIENSEDNASTLSLQFVDSAADMPLAKMHGAFSANVVASKELQQPGSARSTRHLEIELPKEASYQEGDH
LGVIPRNYEGIVNRVTTRFGLDASQQIRLEAEEEKLAHLPLGKTVSVEELLQYVELQDPVTRTQLRAMAA
KTVCPPHKVELEALLEKQAYKEQVLTKRLTMLELLEKYPACEMEFSEFIALLPSMRPRYYSISSSPRVDE
KQASITVSVVSGEAWSGYGEYKGIASNYLAELQEGDTITCFVSTPQSGFTLPKDPETPLIMVGPGTGVAP
FRGFVQARKQLKEQGQSLGEAHLYFGCRSPHEDYLYQEELENAQNEGIITLHTAFSRVPNQPKTYVQHVV
EQDGKKLIELLDQGAHFYICGDGSQMAPDVEATLMKSYAEVHKVSEADARLWLQQLEEKSRYAKDVWAG
SEQ ID NO: 12
CYP153A6
Mycobacterium sp. HXN-1500
GenBank Accession No.: CAH04396
>gi|51997117|emb|CAH04396.1|cytochrome P450 alkane hydroxylase
[Mycobacterium sp. HXN-1500]

| | |
|---|---|
| 1 | MTEMTVAASD ATNAAYGMAL EDIDVSNPVL FRDNTWHPYF KRLREEDPVH YCKSSMFGPY |
| 61 | WSVTKYRDIM AVETNPKVFS SEAKSGGITI MDDNAAASLP MFIAMDPPKH DVQRKTVSPI |
| 121 | VAPENLATME SVIRQRTADL LDGLPINEEF DWVHRVSIEL TTKMLATLFD FPWDDRAKLT |
| 181 | RWSDVTTALP GGGIIDSEEQ RMAELMECAT YFTELWNQRV NAEPKNDLIS MMAHSESTRH |
| 241 | MAPEEYLGNI VLLIVGGNDT TRNSMTGGVL ALNEFPDEYR KLSANPALIS SMVSEIIRWQ |
| 301 | TPLSHMRRTA LEDIEFGGKH IRQGDKVVMW YVSGNRDPEA IDNPDTFIID RAKPRQHLSF |
| 361 | GFGIHRCVGN RLAELQLNIL WEEILKRWPD PLQIQVLQEP TRVLSPFVKG YESLPVRINA |

SEQ ID NO: 13
CYP5013C2
Tetrahymena thermophile
GenBank Accession No.: ABY59989
>gi|164519863|gb|ABY59989.1|cytochrome P450 monooxygenase CYP5013C2
[Tetrahymena thermophila]

| | |
|---|---|
| 1 | MIFELILIAV ALFAYFKIAK PYFSYLKYRK YGKGFYYPIL GEMIEQEQDL KQHADADYSV |
| 61 | HHALDKDPDQ KLFVTNLGTK VKLRLIEPEI IKDFFSKSQY YQKDQTFIQN ITRFLKNGIV |
| 121 | FSEGNTWKES RKLFSPAFHY EYIQKLTPLI NDITDTIFNL AVKNQELKNF DPIAQIQEIT |
| 181 | GRVIIASFFG EVIEGEKFQG LTIIQCLSHI INTLGNQTYS IMYFLFGSKY FELGVTEEHR |
| 241 | KFNKFIAEFN KYLLQKIDQQ IEIMSNELQT KGYIQNPCIL AQLISTHKID EITRNQLFQD |
| 301 | FKTFYIAGMD TTGHLLGMTI YYVSQNKDIY TKLQSEIDSN TDQSAHGLIK NLPYLNAVIK |
| 361 | ETLRYYGPGN ILFDRIAIKD HELAGIPIKK GTIVTPYAMS MQRNSKYYQD PHKYNPSRWL |
| 421 | EKQSSDLHPD ANIPFSAGQR KCIGEQLALL EARIILNKFI KMFDFTCPQD YKLMMNYKFL |
| 481 | SEPVNPLPLQ LTLRKQ |

SEQ ID NO: 14
Nonomuraea dietziae
GenBank Accession No.: AGE14547
>gi|445067389|gb|AGE14547.1|cytochrome P450 hydroxylase sb8
[Nonomuraea dietziae]
VNIDLVDQDHYATFGPPHEQMRWLREHAPVYWHEGEPGFWAVTRHEDVVHVSRHSDLFSSARRLALFNEMPEEQR
ELQRMMMLNQDPPEHTRRRSLVNRGFTPRTIRALEQHIRDICDDLLDQCSGEGDFVTDLAAPLPLYVICELLGAP
VADRDKIFAWSNRMIGAQDPDYAASPEEGGAAAMEVYAYASELAAQRRAAPRDDIVTKLLQSDENGESLTENEFE
LFVLLLVVAGNETTRNAASGGMLTLFEHPDQWDRLVADPSLAATAADEIVRWVSPVNLFRRTATADLTLGGQQVK
ADDKVVVFYSSANRDASVFSDPEVFDIGRSPNPHIGFGGGGAHFCLGNHLAKLELRVLFEQLARRFPRMRQTGEA
RRLRSNFINGIKTLPVTLG
SEQ ID NO: 15
CYP2R1
Homo sapiens
GenBank Accession No.: NP_078790
>gi|45267826|ref|NP_078790.2|vitamin D 25-hydroxylase [Homo sapiens]

| | |
|---|---|
| 1 | MWKLWRAEEG AAALGGALFL LLFALGVRQL LKQRRPMGFP PGPPGLPFIG NIYSLAASSE |
| 61 | LPHVYMRKQS QVYGEIFSLD LGGISTVVLN GYDVVKECLV HQSEIFADRP CLPLFMKMTK |
| 121 | MGGLLNSRYG RGWVDHRRLA VNSFRYFGYG QKSFESKILE ETKFFNDAIE TYKGRPFDFK |
| 181 | QLITNAVSNI TNLIIFGERF TYEDTDFQHM IELFSENVEL AASASVFLYN AFPWIGILPF |
| 241 | GKHQQLFRNA AVVYDFLSRL IEKASVNRKP QLPQHFVDAY LDEMDQGKND PSSTFSKENL |
| 301 | IFSVGELIIA GTETTTNVLR WAILFMALYP NIQGQVQKEI DLIMGPNGKP SWDDKCKMPY |
| 361 | TEAVLHEVLR FCNIVPLGIF HATSEDAVVR GYSIPKGTTV ITNLYSVHFD EKYWRDPEVF |
| 421 | HPERFLDSSG YFAKKEALVP FSLGRRHCLG EHLARMEMFL FFTALLQRFH LHFPHELVPD |
| 481 | LKPRLGMTLQ PQPYLICAER R |

SEQ ID NO: 16
CYP2R1
Macca mulatta
GenBank Accession No.: NP 001180887
>gi|302565346|ref|NP_001180887.1|vitamin D 25-hydroxylase [Macaca mulatta]

| | |
|---|---|
| 1 | MWKLWGGEEG AAALGGALFL LLFALGVRQL LKLRRPMGFP PGPPGLPFIG NIYSLAASAE |
| 61 | LPHVYMRKQS QVYGEIFSLD LGGISTVVLN GYDVVKECLV HQSGIFADRP CLPLFMKMTK |
| 121 | MGGLLNSRYG QGWVEHRRLA VNSFRYFGYG QKSFESKILE ETKFFTDAIE TYKGRPFDFK |
| 181 | QLITSAVSNI TNLIIFGERF TYEDTDFQHM IELFSENVEL AASASVFLYN AFPWIGILPF |
| 241 | GKHQQLFRNA SVVYDFLSRL IEKASVNRKP QLPQHFVDAY FDEMDQGKND PSSTFSKENL |
| 301 | IFSVGELIIA GTETTTNVLR WAILFMALYP NIQGQVQKEI DLIMGPNGKP SWDDKFKMPY |
| 361 | TEAVLHEVLR FCNIVPLGIF HATSEDAVVR GYSIPKGTTV ITNLYSVHFD EKYWRDPEVF |
| 421 | HPERFLDSSG YFAKKEALVP FSLGRRHCLG EQLARMEMFL FFTALLQRFH LHFPHELVPD |
| 481 | LKPRLGMTLQ PQPYLICAER R |

SEQ ID NO: 17
CYP2R1
Canis familiaris
GenBank Accession No.: XP_854533
>gi|73988871|ref|XP_854533.1|PREDICTED: vitamin D 25-hydroxylase
[Canis lupus familiaris]

| | |
|---|---|
| 1 | MRGPPGAEAC AAGLGAALLL LLFVLGVRQL LKQRRPAGFP PGPSGLPFIG NIYSLAASGE |
| 61 | LAHVYMRKQS RVYGEIFSLD LGGISAVVLN GYDVVKECLV HQSEIFADRP CLPLFMKMTK |
| 121 | MGGLLNSRYG RGWVDHRKLA VNSFRCFGYG QKSFESKILE ETNFFIDAIE TYKGRPFDLK |
| 181 | QLITNAVSNI TNLIIFGERF TYEDTDFQHM IELFSENVEL AASASVFLYN AFPWIGIIPF |
| 241 | GKHQQLFRNA AVVYDFLSRL IEKASINRKP QSPQHFVDAY LNEMDQGKND PSCTFSKENL |
| 301 | IFSVGELIIA GTETTTNVLR WAILFMALYP NIQGQVQKEI DLIMGPTGKP SWDDKCKMPY |
| 361 | TEAVLHEVLR FCNIVPLGIF HATSEDAVVR GYSIPKGTTV ITNLYSVHFD EKYWRNPEIF |
| 421 | YPERFLDSSG YFAKKEALVP FSLGKRHCLG EQLARMEMFL FFTALLQRFH LHFPHGLVPD |
| 481 | LKPRLGMTLQ PQPYLICAER R |

SEQ ID NO: 18
CYP2R1
Mus musculus
GenBank Accession No.: AAI08963
>gi|80477959|gb|AAI08963.1|Cyp2r1 protein [Mus musculus]

| | |
|---|---|
| 1 | MGDEMDQGQN DPLSTFSKEN LIFSVGELII AGTETTTNVL RWAILFMALY PNIQGQVHKE |
| 61 | IDLIVGHNRR PSWEYKCKMP YTEAVLHEVL RFCNIVPLGI FHATSEDAVV RGYSIPKGTT |
| 121 | VITNLYSVHF DEKYWKDPDM FYPERFLDSN GYFTKKEALI PFSLGRRHCL GEQLARMEMF |
| 181 | LFFTSLLQQF HLHFPHELVP NLKPRLGMTL QPQPYLICAE RR |

SEQ ID NO: 19
CYP152A6
Bacillus halodurans C-125
GenBank Accession No.: NP_242623
>gi|15614320|ref|NP_242623.1|fatty acid alpha hydroxylase [Bacillus
halodurans C-125]

| | |
|---|---|
| 1 | MKSNDPIPKD SPLDHTMNLM REGYEFLSHR MERFQTDLFE TRVMGQKVLC IRGAEAVKLF |
| 61 | YDPERFKRHR ATPKRIQKSL FGENAIQTMD DKAHLHRKQL FLSMMKPEDE QELARLTHET |
| 121 | WRRVAEGWKK SRPIVLFDEA KRVLCQVACE WAEVPLKSTE IDRRAEDFHA MVDAFGAVGP |
| 181 | RHWRGRKGRR RTERWIQSII HQVRTGSLQA REGSPLYKVS YHRELNGKLL DERMAAIELI |
| 241 | NVLRPIVAIA TFISFAAIAL QEHPEWQERL KNGSNEEFHM FVQEVRRYYP FAPLIGAKVR |
| 301 | KSFTWKGVRF KKGRLVFLDM YGTNHDPKLW DEPDAFRPER FQERKDSLYD FIPQGGGDPT |
| 361 | KGHRCPGEGI TVEVMKTTMD FLVNDIDYDV PDQDISYSLS RMPTRPESGY IMANIERKYE |
| 421 | HA |

SEQ ID NO: 20
aryC
Streptomyces parvus
GenBank Accession No.: AFM80022
>gi|392601346|gb|AFM80022.1|cytochrome P450 [Streptomyces parvus]

| | |
|---|---|
| 1 | MYLGGRRGTE AVGESREPGV WEVFRYDEAV QVLGDHRTFS SDMNHFIPEE QRQLARAARG |
| 61 | NFVGIDPPDH TQLRGLVSQA FSPRVTAALE PRIGRLAEQL LDDIVAERGD KASCDLVGEF |
| 121 | AGPLSAIVIA ELFGIPESDH TMIAEWAKAL LGSRPAGELS IADEAAMQNT ADLVRRAGEY |
| 181 | LVHHITERRA RPQDDLTSRL ATTEVDGKRL DDEEIVGVIG MFLIAGYLPA SVLTANTVMA |
| 241 | LDEHPAALAE VRSDPALLPG AIEEVLRWRP PLVRDQRLTT RDADLGGRTV PAGSMVCVWL |
| 301 | ASAHRDPFRF ENPDLFDIHR NAGRHLAFGK GIHYCLGAPL ARLEARIAVE TLLRRFERIE |
| 361 | IPRDESVEFH ESIGVLGPVR LPTTLFARR |

SEQ ID NO: 21
CYP101A1
Pseudomonas putida
Uniprot Accession No.: P00183
>sp|P00183|CPXA_PSEPU Camphor 5-monooxygenase OS = Pseudomonas putida GN = camC
PE = 1 SV = 2
TTETIQSNANLAPLPPHVPEHLVFDFDMYNPSNLSAGVQEAWAVLQESNVPDLVWTRCNGGHWIATRGQLIREAY
EDYRHFSSECPFIPREAGEAYDFIPTSMDPPEQRQFRALANQVVGMPVVDKLENRIQELACSLIESLRPQGQCNF
TEDYAEPFPIRIFMLLAGLPEEDIPHLKYLTDQMTRPDGSMTFAEAKEALYDYLIPIIEQRRQKPGTDAISIVAN
GQVNGRPITSDEAKRMCGLLLVGGLDTVVNFLSFSMEFLAKSPEHRQELIERPERIPAACEELLRRFSLVADGRI
LTSDYEFHGVQLKKGDQILLPQMLSGLDERENACPMHVDFSRQKVSHTTFGHGSHLCLGQHLARREIIVTLKEWL
TRIPDFSIAPGAQIQHKSGIVSGVQALPLVWDPATTKAV
SEQ ID NO: 22
Homo sapiens
CYP2D7
GenBank Accession No.: AAO49806
>gi|37901459|gb|AAO49806.1|cytochrome P450 [Homo sapiens]
GLEALVPLA MIVAIFLLLV DLMHRHQRWA ARYPPGPLPL PGLGNLLHVD FQNTPYCFDQ
LRRRFGDVFN LQLAWTPVVV LNGLAAVREA MVTRGEDTAD RPPAPIYQVL GFGPRSQGVI
LSRYGPAWRE QRRFSVSTLR NLGLGKKSLE QWVTEEAACL CAAFADQAGR PFRPNGLLDK
AVSNVIASLT CGRRFEYDDP RFLRLLDLAQ EGLKEESGFL REVLNAVPVL PHIPALAGKV
LRFQKAFLTQ LDELLTEHRM TWDPAQPPRD LTEAFLAKKE KAKGSPESSF NDENLRIVVG
NLFLAGMVTT LTTLAWGLLL MILHLDVQRG RRVSPGCSPI VGTHVCPVRV QQEIDDVIGQ
VRRPEMGDQV HMPYTTAVIH EVQRFGDIVP LGVTHMTSRD IEVQGFRIPK GTTLITNLSS
VLKDEAVWEK PFRFHPEHFL DAQGHFVKPE AFLPFSAGRR ACLGEPLARM ELFLFFTSLL
QHFSFSVAAG QPRPSHSRVV SFLVTPSPYE LCAVPR
SEQ ID NO: 23
Rattus norvegicus
CYPC27
GenBank Accession No.: AAB02287
>gi|1374714|gb|AAB02287.1|cytochrome P450 [Rattus norvegicus]
AVLSRMRLRWALLDTRVMGHGLCPQGARAKAAIPAALRDHESTEGPGTGQDRPRLRSLAELPGPGTLRF
LFQLFLRGYVLHLHELQALNKAKYGPMWTTTFGTRTNVNLASAPLLEQVMRQEGKYPIRDSMEQWKEHRD
HKGLSYGIFITQGQQWYHLRHSLNQRMLKPAEAALYTDALNEVISDFIARLDQVRTESASGDQVPDVAHL
LYHLALEAICYILFEKRVGCLEPSIPEDTATFIRSVGLMFKNSVYVTFLPKWSRPLLPFWKRYMNNWDNI
FSFGEKMIHQKVQEIEAQLQAAGPDGVQVSGYLHFLLTKELLSPQETVGTFPELILAGVDTTSNTLTWAL
YHLSKNPEIQEALHKEVTGVVPFGKVPQNKDFAHMPLLKAVIKETLRLYPVVPTNSRIITEKETEINGFL
FPKNTQFVLCTYVVSRDPSVFPEPESFQPHRWLRKREDDNSGIQHPFGSVPFGYGVRSCLGRRIAELEMQ
LLLSRLIQKYEVVLSPGMGEVKSVSRIVLVPSKKVSLRFLQRQ
SEQ ID NO: 24
CYP2B4
Oryctolagus cuniculus
GenBank Accession No. AAA65840
>gi|164959|gb|AAA65840.1|cytochrome P-450 [Oryctolagus cuniculus]
MEFSLLLLLAFLAGLLLLLFRGHPKAHGRLPPGPSPLPVLGNLLQMDRKGLLRSFLRLRE
KYGDVFTVYLGSRPVVVLCGTDAIREALVDQAEAFSGRGKIAVVDPIFQGYGVIFANGER
WRALRRFSLATMRDFGMGKRSVEERIQEEARCLVEELRKSKGALLDNTLLFHSITSNIIC
SIVFGKRFDYKDPVFLRLLDLFFQSFSLISSFSSQVFELFPGFLKHFPGTHRQIYRNLQE
INTFIGQSVEKHRATLDPSNPRDFIDVYLLRMEKDKSDPSSEFHHQNLILTVLSLFFAGT
ETTSTTLRYGFLLMLKYPHVTERVQKEIEQVIGSHRPPALDDRAKMPYTDAVIHEIQRLG
DLIPFGVPHTVTKDTQFRGYVIPKNTEVFPVLSSALHDPRYFETPNTFNPGHFLDANGAL
KRNEGFMPFSLGKRICLGEGIARTELFLFFTTILQNFSIASPVPPEDIDLTPRESGVGNV
PPSYQIRFLAR
SEQ ID NO: 25
CYP102A2
Bacillus subtilis
Uniprot Accession No. O08394
>sp|O08394|CYPD_BACSU Probable bifunctional P-450/NADPH-P450 reductase 1
OS = Bacillus subtilis (strain 168) GN = cypD PE = 3 SV = 1
MKETSPIPQPKTFGPLGNLPLIDKDKPTLSLIKLAEEQGPIFQIHTPAGTTIVVSGHELV
KEVCDEERFDKSIEGALEKVRAFSGDGLFTSWTHEPNWRKAHNILMPTFSQRAMKDYHEK
MVDIAVQLIQKWARLNPNEAVDVPGDMTRLTLDTIGLCGFNYRFNSYYRETPHPFINSMV
RALDEAMHQMQRLDVQDKLMVRTKRQFRHDIQTMFSLVDSIIAERRANGDQDEKDLLARM
LNVEDPETGEKLDDENIRFQIITFLIAGHETTSGLLSFATYFLLKHPDKLKKAYEEVDRV
LTDAAPTYKQVLELTYIRMILNESLRLWPTAPAFSLYPKEDTVIGGKFPITTNDRISVLI
PQLHRDRDAWGKDAEEFRPERFEHQDQVPHHAYKPFGNGQRACIGMQFALHEATLVLGMI
LKYFTLIDHENYELDIKQTLTLKPGDFHIRVQSRNQDAIHADVQAVEKAASDEQKEKTEA
KGTSVIGLNNRPLLVLYGSDTGTAEGVARELADTASLHGVRTETAPLNDRIGKLPKEGAV
VIVTSSYNGKPPSNAGQFVQWLQEIKPGELEGVHYAVFGCGDHNWASTYQYVPRFIDEQL
AEKGATRFSARGEGDVSGDFEGQLDEWKKSMWADAIKAFGLELNENADKERSTLSLQFVR
GLGESPLARSYEASHASIAENRELQSADSDRSTRHIEIALPPDVEYQEGDHLGVLPKNSQ
TNVSRILHRFGLKGTDQVTLSASGRSAGHLPLGRPVSLHDLLSYSVEVQEAATRAQIREL
AAFTVCPPHRRELEELSAEGVYQEQILKKRISMLDLLEKYEACDMPFERFLELLRPLKPR
YYSISSSPRVNPRQASITVGVVRGPAWSGRGEYRGVASNDLAERQAGDDVVMFIRTPESR
FQLPKDPETPIIMVGPGTGVAPFRGFLQARDVLKREGKTLGEAHLYFGCRNDRDFIYRDE
LERFEKDGIVTVHTAFSRKEGMPKTYVQHLMADQADTLISILDRGGRLYVCGDGSKMAPD
VEAALQKAYQAVHGTGEQEAQNWLRHLQDTGMYAKDVWAGI
SEQ ID NO: 26
CYP102A3
Bacillus subtilis
Uniprot Accession No. O08336
>sp|O08336|CYPE_BACSU Probable bifunctional P-450/NADPH-P450 reductase 2
OS = Bacillus subtilis (strain 168) GN = cypE PE = 2 SV = 1
MKQASAIPQPKTYGPLKNLPHLEKEQLSQSLWRIADELGPIFRFDFPGVSSVFVSGHNLV
AEVCDESRFDKNLGKGLQKVREFGGDGLFTSWTHEPNWQKAHRILLPSFSQKAMKGYHSM
MLDIATQLIQKWSRLNPNEEIDVADDMTRLTLDTIGLCGFNYRFNSFYRDSQHPFITSML
RALKEAMNQSKRLGLQDKMMVKTKLQFQKDIEVMNSLVDRMIAERKANPDDNIKDLLSLM
LYAKDPVTGETLDDENIRYQIITFLIAGHETTSGLLSFAIYCLLTHPEKLKKAQEEADRV
LTDDTPEYKQIQQLKYTRMVLNETLRLYPTAPAFSLYAKEDTVLGGEYPISKGQPVTVLI
PKLHRDQNAWGPDAEDFRPERFEDPSSIPHHAYKPFGNGQRACIGMQFALQEATMVLGLV
LKHFELINHTGYELKIKEALTIKPDDFKITVKPRKTAAINVQRKEQADIKAETKPKETKP
KHGTPLLVLYGSNLGTAEGIAGELAAQGRQMGFTAETAPLDDYIGKLPEEGAVVIVTASY
NGSPPDNAAGFVEWLKELEEGQLKGVSYAVFGCGNRSWASTYQRIPRLIDDMMKAKGASR
LTEIGEGDAADDFESHRESWENRFWKETMDAFDINEIAQKEDRPSLSIAFLSEATETPVA
KAYGAFEGVVLENRELQTADSTRSTRHIELEIPAGKTYKEGDHIGIMPKNSRELVQRVLS
RFGLQSNHVIKVSGSAHMSHLPMDRPIKVADLLSSYVELQEPASRLQLRELASYTVCPPH
QKELEQLVLDDGIYKEQVLAKRLTMLDFLEDYPACEMPFERFLALLPSLKPRYYSISSSP
KVHANIVSMTVGVVKASAWSGRGEYRGVASNYLAELNTGDAAACFIRTPQSGFQMPDEPE
TPMIMVGPGTGIAPFRGFIQARSVLKKEGSTLGEALLYFGCRRPDHDDLYREELDQAEQE
GLVTIRRCYSRVENESKGYVQHLLKQDSQKLMTLIEKGAHIYVCGDGSQMAPDVEKTLRW
AYETEKGASQEESADWLQKLQDQKRYIKDVWTGN
SEQ ID NO: 27
CYP102A1
B. megaterium DSM 32
Uniprot Accession No. P14779
>sp|P14779|CPXB_BACME Bifunctional P-450/NADPH-P450 reductase OS = Bacillus
megaterium GN = cyp102A1 PE = 1 SV = 2

| | |
|---|---|
| 1 | MTIKEMPQPK TFGELKNLPL LNTDKPVQAL MKIADELGEI FKFEAPGRVT RYLSSQRLIK |
| 61 | EACDESRFDK NLSQALKFVR DFAGDGLFTS WTHEKNWKKA HNILLPSFSQ QAMKGYHAMM |
| 121 | VDIAVQLVQK WERLNADEHI EVPEDMTRLT LDTIGLCGFN YRFNSFYRDQ PHPFITSMVR |
| 181 | ALDEAMNKLQ RANPDDPAYD ENKRQFQEDI KVMNDLVDKI IADRKASGEQ SDDLLTHMLN |
| 241 | GKDPETGEPL DDENIRYQII TFLIAGHETT SGLLSFALYF LVKNPHVLQK AAEEAARVLV |
| 301 | DPVPSYKQVK QLKYVGMVLN EALRLWPTAP AFSLYAKEDT VLGGEYPLEK GDELMVLIPQ |
| 361 | LHRDKTIWGD DVEEFRPERF ENPSAIPQHA FKPFGNGQRA CIGQQFALHE ATLVLGMMLK |
| 421 | HFDFEDHTNY ELDIKETLTL KPEGFVVKAK SKKIPLGGIP SPSTEQSAKK VRKKAENAHN |
| 481 | TPLLVLYGSN MGTAEGTARD LADIAMSKGF APQVATLDSH AGNLPREGAV LIVTASYNGH |
| 541 | PPDNAKQFVD WLDQASADEV KGVRYSVFGC GDKNWATTYQ KVPAFIDETL AAKGAENIAD |
| 601 | RGEADASDDF EGTYEEWREH MWSDVAAYFN LDIENSEDNK STLSLQFVDS AADMPLAKMH |
| 661 | GAFSTNVVAS KELQQPGSAR STRHLEIELP KEASYQEGDH LGVIPRNYEG IVNRVTARFG |
| 721 | LDASQQIRLE AEEEKLAHLP LAKTVSVEEL LQYVELQDPV TRTQLRAMAA KTVCPPHKVE |
| 781 | LEALLEKQAY KEQVLAKRLT MLELLEKYPA CEMKFSEFIA LLPSIRPRYY SISSSPRVDE |
| 841 | KQASITVSVV SGEAWSGYGE YKGIASNYLA ELQEGDTITC FISTPQSEFT LPKDPETPLI |
| 901 | MVGPGTGVAP FRGFVQARKQ LKEQGQSLGE AHLYFGCRSP HEDYLYQEEL ENAQSEGIIT |
| 961 | LHTAFSRMPN QPKTYVQHVM EQDGKKLIEL LDQGAHFYIC GDGSQMAPAV EATLMKSYAD |
| 1021 | VHQVSEADAR LWLQQLEEKG RYAKDVWAG |

SEQ ID NO: 28
CYP102A5
B. cereus ATCC14579
GenBank Accession No. AAP10153
>gi|29896875|gb|AAP10153.1|NADPH-cytochrome P450 reductase [Bacillus
cereus ATCC 14579]

| | |
|---|---|
| 1 | MEKKVSAIPQ PKTYGPLGNL PLIDKDKPTL SFIKIAEEYG PIFQIQTLSD TIIVVSGHEL |
| 61 | VAEVCDETRF DKSIEGALAK VRAFAGDGLF TSETHEPNWK KAHNILMPTF SQRAMKDYHA |
| 121 | MMVDIAVQLV QKWARLNPNE NVDVPEDMTR LTLDTIGLCG FNYRFNSFYR ETPHPFITSM |
| 181 | TRALDEAMHQ LQRLDIEDKL MWRTKRQFQH DIQSMFSLVD NIIAERKSSG DQEENDLLSR |
| 241 | MLNVPDPETG EKLDDENIRF QIITFLIAGH ETTSGLLSFA IYFLLKNPDK LKKAYEEVDR |
| 301 | VLTDPTPTYQ QVMKLKYMRM ILNESLRLWP TAPAFSLYAK EDTVIGGKYP IKKGEDRISV |
| 361 | LIPQLHRDKD AWGDNVEEFQ PERFEELDKV PHHAYKPFGN GQRACIGMQF ALHEATLVMG |
| 421 | MLLQHFELID YQNYQLDVKQ TLTLKPGDFK IRILPRKQTI SHPTVLAPTE DKLKNDEIKQ |
| 481 | HVQKTPSIIG ADNLSLLVLY GSDTGVAEGI ARELADTASL EGVQTEVVAL NDRIGSLPKE |
| 541 | GAVLIVTSSY NGKPPSNAGQ FVQWLEELKP DELKGVQYAV FGCGDHNWAS TYQRIPRYID |
| 601 | EQMAQKGATR FSKRGEADAS GDFEEQLEQW KQNMWSDAMK AFGLELNKNM EKERSTLSLQ |
| 661 | FVSRLGGSPL ARTYEAVYAS ILENRELQSS SSDRSTRHIE VSLPEGATYK EGDHLGVLPV |
| 721 | NSEKNINRIL KRFGLNGKDQ VILSASGRSI NHIPLDSPVS LLALLSYSVE VQEAATRAQI |
| 781 | REMVTFTACP PHKKELEALL EEGVYHEQIL KKRISMLDLL EKYEACEIRF ERFLELLPAL |
| 841 | KPRYYSISSS PLVAHNRLSI TVGVVNAPAW SGEGTYEGVA SNYLAQRHNK DEIICFIRTP |
| 901 | QSNFELPKDP ETPIIMVGPG TGIAPFRGFL QARRVQKQKG MNLGQAHLYF GCRHPEKDYL |
| 961 | YRTELENDER DGLISLHTAF SRLEGHPKTY VQHLIKQDRI NLISLLDNGA HLYICGDGSK |
| 1021 | MAPDVEDTLC QAYQEIHEVS EQEARNWLDR VQDEGRYGKD VWAGI |

SEQ ID NO: 29
CYP102A7
B. licheniformis ATTC1458
GenBank Accession No. YP 079990
>gi|52081199|ref|YP_079990.1|cytochrome P450/NADPH-ferrihemoprotein
reductase [Bacillus licheniformis DSM 13 = ATCC 14580]

| | |
|---|---|
| 1 | MNKLDGIPIP KTYGPLGNLP LLDKNRVSQS LWKIADEMGP IFQFKFADAI GVFVSSHELV |
| 61 | KEVSEESRFD KNMGKGLLKV REFSGDGLFT SWTEEPNWRK AHNILLPSFS QKAMKGYHPM |
| 121 | MQDIAVQLIQ KWSRLNQDES IDVPDDMTRL TLDTIGLCGF NYRFNSFYRE GQHPFIESMV |
| 181 | RGLSEAMRQT KRFPLQDKLM IQTKRRFNSD VESMFSLVDR IIADRKQAES ESGNDLLSLM |
| 241 | LHAKDPETGE KLDDENIRYQ IITFLIAGHE TTSGLLSFAI YLLLKHPDKL KKAYEEADRV |
| 301 | LTDPVPSYKQ VQQLKYIRMI LNESIRLWPT APAFSLYAKE ETVIGGKYLI PKGQSVTVLI |
| 361 | PKLHRDQSVW GEDAEAFRPE RFEQMDSIPA HAYKPFGNGQ RACIGMQFAL HEATLVLGMI |
| 421 | LQYFDLEDHA NYQLKIKESL TLKPDGFTIR VRPRKKEAMT AMPGAQPEEN GRQEERPSAP |
| 481 | AAENTHGTPL LVLYGSNLGT AEEIAKELAE EAREQGFHSR TAELDQYAGA IPAEGAVIIV |
| 541 | TASYNGNPPD CAKEFVNWLE HDQTDDLRGV KYAVFGCGNR SWASTYQRIP RLIDSVLEKK |
| 601 | GAQRLHKLGE GDAGDDFEGQ FESWKYDLWP LLRTEFSLAE PEPNQTETDR QALSVEFVNA |
| 661 | PAASPLAKAY QVFTAKISAN RELQCEKSGR STRHIEISLP EGAAYQEGDH LGVLPQNSEV |
| 721 | LIGRVFQRFG LNGNEQILIS GRNQASHLPL ERPVHVKDLF QHCVELQEPA TRAQIRELAA |
| 781 | HTVCPPHQRE LEDLLKDDVY KDQVLNKRLT MLDLLEQYPA CELPFARFLA LLPPLKPRYY |
| 841 | SISSSPQLNP RQTSITVSVV SGPALSGRGH YKGVASNYLA GLEPGDAISC FIREPQSGFR |
| 901 | LPEDPETPVI MVGPGTGIAP YRGFLQARRI QRDAGVKLGE AHLYFGCRRP NEDFLYRDEL |
| 961 | EQAEKDGIVH LHTAFSRLEG RPKTYVQDLL REDAALLIHL LNEGGRLYVC GDGSRMAPAV |
| 1021 | EQALCEAYRI VQGASREESQ SWLSALLEEG RYAKDVWDGG VSQHNVKADC IART |

SEQ ID NO: 30
CYPX
B. thuringiensis serovar konkukian
str.97-27
GenBank Accession No. YP 037304
>gi|49480099|ref|YP_037304.1|NADPH-cytochrome P450 reductase [Bacillus
thuringiensis serovar konkukian str. 97-27]

| | |
|---|---|
| 1 | MDKKVSAIPQ PKTYGPLGNL PLIDKDKPTL SFIKLAEEYG PIFQIQTLSD TIIVVSGHEL |
| 61 | VAEVCDETRF DKSIEGALAK VRAFAGDGLF TSETDEPNWK KAHNILMPTF SQRAMKDYHA |
| 121 | MMVDIAVQLV QKWARLNPNE NVDVPEDMTR LTLDTIGLCG FNYRFNSFYR ETPHPFITSM |
| 181 | TRALDEAMHQ LQRLDIEDKL MWRTKRQFQH DIQSMFSLVD NIIAERKSSE NQEENDLLSR |
| 241 | MLNVQDPETG EKLDDENIRF QIITFLIAGH ETTSGLLSFA IYFLLKNPDK LKKAYEEVDR |
| 301 | VLTDSTPTYQ QVMKLKYIRM ILNESLRLWP TAPAFSLYAK EDTVIGGKYP IKKGEDRISV |
| 361 | LIPQLHRDKD AWGDDVEEFQ PERFEELDKV PHHAYKPFGN GQRACIGMQF ALHEATLVMG |
| 421 | MLLQHFEFID YEDYQLDVKQ TLTLKPGDFK IRIVPRNQTI SHTTVLAPTE EKLKKHEIKK |
| 481 | QVQKTPSIIG ADNLSLLVLY GSDTGVAEGI ARELADTASL EGVQTEVVAL NDRIGSLPKE |
| 541 | GAVLIVTSSY NGKPPSNAGQ FVQWLEELKP DELKGVQYAV FGCGDHNWAS TYQRIPRYID |
| 601 | EQMAQKGATR FSTRGEADAS GDFEEQLEQW KQSMWSDAMK AFGLELNKNM EKERSTLSLQ |
| 661 | FVSRLGGSPL ARTYEAVYAS ILENRELQSS SSERSTRHIE ISLPEGATYK EGDHLGVLPI |
| 721 | NNEKNVNRIL KRFGLNGKDQ VILSASGRSV NHIPLDSPVR LYDLLSYSVE VQEAATRAQI |
| 781 | REMVTFTACP PHKKELESLL EDGVYQEQIL KKRISMLDLL EKYEACEIRF ERFLELLPAL |
| 841 | KPRYYSISSS PLVAQDRLSI TVGVVNAPAW SGEGTYEGVA SNYLAQRHNK DEIICFIRTP |
| 901 | QSNFQLPENP ETPIIMVGPG TGIAPFRGFL QARRVQKQKG MKVGEAHLYF GCRHPEKDYL |
| 961 | YRTELENDER DGLISLHTAF SRLEGHPKTY VQHVIKEDRI HLISLLDNGA HLYICGDGSK |
| 1021 | MAPDVEDTLC QAYQEIHEVS EQEARNWLDR LQEEGRYGKD VWAGI |

SEQ ID NO: 31
CYP102E1
R. metallidurans CH34
GenBank Accession No. YP 585608
>gi|94312398|ref|YP_585608.1|putative bifunctional P-450: NADPH-P450
reductase 2 [Cupriavidus metallidurans CH34]

| | |
|---|---|
| 1 | MSTATPAAAL EPIPRDPGWP IFGNLFQITP GEVGQHLLAR SRHHDGIFEL DFAGKRVPFV |
| 61 | SSVALASELC DATRFRKIIG PPLSYLRDMA GDGLFTAHSD EPNWGCAHRI LMPAFSQRAM |
| 121 | KAYFDVMLRV ANRLVDKWDR QGPDADIAVA DDMTRLTLDT IALAGFGYDF ASFASDELDP |
| 181 | FVMAMVGALG EAMQKLTRLP IQDRFMGRAH RQAAEDIAYM RNLVDDVIRQ RRVSPTSGMD |
| 241 | LLNLMLEARD PETDRRLDDA NIRNQVITFL IAGHETTSGL LTFALYELLR NPGVLAQAYA |
| 301 | EVDTVLPGDA LPVYADLARM PVLDRVLKET LRLWPTAPAF AVAPFDDVVL GGRYRLRKDR |
| 361 | RISVVLTALH RDPKVWANPE RFDIDRFLPE NEAKLPAHAY MPFGQGERAC IGRQFALTEA |
| 421 | KLALALMLRN FAFQDPHDYQ FRLKETLTIK PDQFVLRVRR RRPHERFVTR QASQAVADAA |
| 481 | QTDVRGHGQA MTVLCASSLG TARELAEQIH AGAIAAGFDA KLADLDDAVG VLPTSGLVVV |
| 541 | VAATYNGRAP DSARKFEAML DADDASGYRA NGMRLALLGC GNSQWATYQA FPRRVFDFFI |
| 601 | TAGAVPLLPR GEADGNGDFD QAAERWLAQL WQALQADGAG TGGLGVDVQV RSMAAIRAET |
| 661 | LPAGTQAFTV LSNDELVGDP SGLWDFSIEA PRTSTRDIRL QLPPGITYRT GDHIAVWPQN |
| 721 | DAQLVSELCE RLDLDPDAQA TISAPHGMGR GLPIDQALPV RQLLTHFIEL QDVVSRQTLR |
| 781 | ALAQATRCPF TKQSIEQLAS DDAEHGYATK VVARRLGILD VLVEHPAIAL TLQELLACTV |
| 841 | PMRPRLYSIA SSPLVSPDVA TLLVGTVCAP ALSGRGQFRG VASTWLQHLP PGARVSASIR |
| 901 | TPNPPFAPDP DPAAPMLLIG PGTGIAPFRG FLEERALRKM AGNAVTPAQL YFGCRHPQHD |
| 961 | WLYREDIERW AGQGVVEVHP AYSVVPDAPR YVQDLLWQRR EQVWAQVRDG ATIYVCGDGR |
| 1021 | RMAPAVRQTL IEIGMAQGGM TDKAASDWFG GLVAQGRYRQ DVFN |

SEQ ID NO: 32
CYP505X
A. fumigatus Af293
GenBank Accession No. EAL92660
>gi|66852335|gb|EAL92660.1|P450 family fatty acid hydroxylase, putative
[Aspergillus fumigatus Af293]

| | |
|---|---|
| 1 | MSESKTVPIP GPRGVPLLGN IYDIEQEVPL RSINLMADQY GPIYRLTTFG WSRVFVSTHE |
| 61 | LVDEVCDEER FTKVVTAGLN QIRNGVHDGL FTANFPGEEN WAIAHRVLVP AFGPLSIRGM |
| 121 | FDEMYDIATQ LVMKWARHGP TVPIMVTDDF TRLTLDTIAL CAMGTRFNSF YHEEMHPFVE |
| 181 | AMVGLLQGSG DRARRPALLN NLPTSENSKY WDDIAFLRNL AQELVEARRK NPEDKKDLLN |
| 241 | ALILGRDPKT GKGLTDESII DNMITFLIAG HETTSGLLSF LFYYLLKTPN AYKKAQEEVD |
| 301 | SVVGRRKITV EDMSRLPYLN AVMRETLRLR STAPLIAVHA HPEKNKEDPV TLGGGKYVLN |
| 361 | KDEPIVIILD KLHRDPQVYG PDAEEFKPER MLDENFEKLP KNAWKPFGNG MRACIGRPFA |
| 421 | WQEALLVVAI LLQNFNFQMD DPSYNLHIKQ TLTIKPKDFH MRATLRHGLD ATKLGIALSG |
| 481 | SADRAPPESS GAASRVRKQA TPPAGQLKPM HIFFGSNTGT CETFARRLAD DAVGYGFAAD |
| 541 | VQSLDSAMQN VPKDEPVVFI TASYEGQPPD NAAHFFEWLS ALKENELEGV NYAVFGCGHH |
| 601 | DWQATFHRIP KAVNQLVAEH GGNRLCDLGL ADAANSDMFT DFDSWGESTF WPAITSKFGG |
| 661 | GKSDEPKPSS SLQVEVSTGM RASTLGLQLQ EGLVIDNQLL SAPDVPAKRM IRFKLPSDMS |
| 721 | YRCGDYLAVL PVNPTSVVRR AIRRFDLPWD AMLTIRKPSQ APKGSTSIPL DTPISAFELL |
| 781 | STYVELSQPA SKRDLTALAD AAITDADAQA ELRYLASSPT RFTEEIVKKR MSPLDLLIRY |
| 841 | PSIKLPVGDF LAMLPPMRVR QYSISSSPLA DPSECSITFS VLNAPALAAA SLPPAERAEA |
| 901 | EQYMGVASTY LSELKPGERA HIAVRPSHSG FKPPMDLKAP MIMACAGSGL APFRGFIMDR |
| 961 | AEKIRGRRSS VGADGQLPEV EQPAKAILYV GCRTKGKDDI HATELAEWAQ LGAVDVRWAY |
| 1021 | SRPEDGSKGR HVQDLMLEDR EELVSLFDQG ARIYVCGSTG VGNGVRQACK DIYLERRRQL |
| 1081 | RQAARERGEE VPAEEDEDAA AEQFLDNLRT KERYATDVFT |

SEQ ID NO: 33
CYP505A8
A. nidulans FGSC A4
GenBank Accession No. EAA58234
>gi|40739044|gb|EAA58234.1|hypothetical protein AN6835.2 [Aspergillus
nidulans FGSC A4]

| | |
|---|---|
| 1 | MAEIPEPKGL PLIGNIGTID QEFPLGSMVA LAEEHGEIYR LRFPGRTVVV VSTHALVNET |
| 61 | CDEKRFRKSV NSALAHVREG VHDGLFTAKM GEVNWEIAHR VLMPAFGPLS IRGMFDEMHD |
| 121 | IASQLALKWA RYGPDCPIMV TDDFTRLTLD TLALCSMGYR FNSYYSPVLH PFIEAMGDFL |
| 181 | TEAGEKPRRP PLPAVFFRNR DQKFQDDIAV LRDTAQGVLQ ARKEGKSDRN DLLSAMLRGV |
| 241 | DSQTGQKMTD ESIMDNLITF LIAGHETTSG LLSFVFYQLL KHPETYRTAQ QEVDNVVGQG |
| 301 | VIEVSHLSKL PYINSVLRET LRLNATIPLF TVEAFEDTLL AGKYPVKAGE TIVNLLAKSH |
| 361 | LDPEVYGEDA LEFKPERMSD ELFNARLKQF PSAWKPFGNG MRACIGRPFA WQEALLVMAM |
| 421 | LLQNFDFSLA DPNYDLKFKQ TLTIKPKDMF MKARLRHGLT PTTLERRLAG LAVESATQDK |
| 481 | IVTNPADNSV TGTRLTILYG SNSGTCETLA RRIAADAPSK GFHVMRFDGL DSGRSALPTD |
| 541 | HPVVIVTSSY EGQPPENAKQ FVSWLEELEQ QNESLQLKGV DFAVFGCFKE WAQTFHRIPK |
| 601 | LVDSLLEKLG GSRLTDLGLA DVSTDELFST FETWADDVLW PRLVAQYGAD GKTQAHGSSA |
| 661 | GHEAASNAAV EVTVSNSRTQ ALRQDVGQAM VVETRLLTAE SEKERRKKHL EIRLPDGVSY |
| 721 | TAGDYLAVLP INPPETVRRA MRQFKLSWDA QITIAPSGPT TALPTDGPIA ANDIFSTYVE |
| 781 | LSQPATRKDL RIMADATTDP DVQKILRTYA NETYTAEILT KSISVLDILE QHPAIDLPLG |
| 841 | TFLLMLPSMR MRQYSISSSP LLTPTTATIT ISVLDAPSRS RSNGSRHLGV ATSYLDSLSV |
| 901 | GDHLQVTVRK NPSSGFRLPS EPETTPMICI AAGSGIAPFR AFLQERAVMM EQDKDRKLAP |
| 961 | ALLFFGCRAP GIDDLYREQL EEWQARGVVD ARWAFSRQSD DTKGCRHVDD RILADREDVV |
| 1021 | KLWRDGARVY VCGSGALAQS VRSAMVTVLR DEMETTGDGS DNGKAEKWFD EQRNVRYVMD |
| 1081 | VFD |

SEQ ID NO: 34
CYP505A3
A. oryzae ATCC42149
Uniprot Accession No. Q2U4F1
>gi|121928062|sp|Q2U4F1|Q2U4F1_ASPOR Cytochrome P450

| | |
|---|---|
| 1 | MRQNDNEKQI CPIPGPQGLP FLGNILDIDL DNGTMSTLKI AKTYYPIFKF TFAGETSIVI |
| 61 | NSVALLSELC DETRFHKHVS FGLELLRSGT HDGLFTAYDH EKNWELAHRL LVPAFGPLRI |
| 121 | REMFPQMHDI AQQLCLKWQR YGPRRPLNLV DDFTRTTLDT IALCAMGYRF NSFYSEGDFH |
| 181 | PFIKSMVRFL KEAETQATLP SFISNLRVRA KRRTQLDIDL MRTVCREIVT ERRQTNLDHK |
| 241 | NDLLDTMLTS RDSLSGDALS DESIIDNILT FLVAGHETTS GLLSFAVYYL LTTPDAMAKA |
| 301 | AHEVDDVVGD QELTIEHLSM LKYLNAILRE TLRLMPTAPG FSVTPYKPEI IGGKYEVKPG |
| 361 | DSLDVFLAAV HRDPAVYGSD ADEFRPERMS DEHFQKLPAN SWKPFGNGKR SCIGRAFAWQ |
| 421 | EALMILALIL QSFSLNLVDR GYTLKLKESL TIKPDNLWAY ATPRPGRNVL HTRLALQTNS |
| 481 | THPEGLMSLK HETVESQPAT ILYGSNSGTC EALAHRLAIE MSSKGRFVCK VQPMDAIEHR |
| 541 | RLPRGQPVII ITGSYDGRPP ENARHFVKWL QSLKGNDLEG IQYAVFGCGL PGHHDWSTTF |
| 601 | YKIPTLIDTI MAEHGGARLA PRGSADTAED DPFAELESWS ERSVWPGLEA AFDLVRHNSS |
| 661 | DGTGKSTRIT IRSPYTLRAA HETAVVHQVR VLTSAETTKK VHVELALPDT INYRPGDHLA |
| 721 | ILPLNSRQSV QRVLSLFQIG SDTILYMTSS SATSLPTDTP ISAHDLLSGY VELNQVATPT |
| 781 | SLRSLAAKAT DEKTAEYLEA LATDRYTTEV RGNHLSLLDI LESYSVPSIE IQHYIQMLPL |
| 841 | LRPRQYTISS SPRLNRGQAS LTVSVMERAD VGGPRNCAGV ASNYLASCTP GSILRVSLRQ |
| 901 | ANPDFRLPDE SCSHPIIMVA AGSGIAPFRA FVQERSVRQK EGIILPPAFL FFGCRRADLD |
| 961 | DLYREELDAF EEQGVVTLFR AFSRAQSESH GCKYVQDLLW MERVRVKTLW GQDAKVFVCG |
| 1021 | SVRMNEGVKA IISKIVSPTP TEELARRYIA ETFI |

SEQ ID NO: 35
CYPX
A. oryzae ATCC42149
Uniprot Accession No. Q2UNA2
>gi|121938553|sp|Q2UNA2|Q2UNA2_ASPOR Cytochrome P450

| | |
|---|---|
| 1 | MSTPKAEPVP IPGPRGVPLM GNILDIESEI PLRSLEMMAD TYGPIYRLTT FGFSRCMISS |
| 61 | HELAAEVFDE ERFTKKIMAG LSELRHGIHD GLFTAHMGEE NWEIAHRVLM PAFGPLNIQN |
| 121 | MFDEMHDIAT QLVMKWARQG PKQKIMVTDD FTRLTLDTIA LCAMGTRFNS FYSEEMHPFV |
| 181 | DAMVGMLKTA GDRSRRPGLV NNLPTTENNK YWEDIDYLRN LCKELVDTRK KNPTDKKDLL |
| 241 | NALINGRDPK TGKGMSYDSI IDNMITFLIA GHETTSGSLS FAFYNMLKNP QAYQKAQEEV |
| 301 | DRVIGRRRIT VEDLQKLPYI TAVMRETLRL TPTAPAIAVG PHPTKNHEDP VTLGNGKYVL |
| 361 | GKDEPCALLL GKIQRDPKVY GPDAEEFKPE RMLDEHFNKL PKHAWKPFGN GMRACIGRPF |
| 421 | AWQEALLVIA MLLQNFNFQM DDPSYNIQLK QTLTIKPNHF YMRAALREGL DAVHLGSALS |
| 481 | ASSSEHADHA AGHGKAGAAK KGADLKPMHV YYGSNTGTCE AFARRLADDA TSYGYSAEVE |
| 541 | SLDSAKDSIP KNGPVVFITA SYEGQPPDNA AHFFEWLSAL KGDKPLDGVN YAVFGCGHHD |
| 601 | WQTTFYRIPK EVNRLVGENG ANRLCEIGLA DTANADIVTD FDTWGETSFW PAVAAKFGSN |
| 661 | TQGSQKSSTF RVEVSSGHRA TTLGLQLQEG LVVENTLLTQ AGVPAKRTIR FKLPTDTQYK |
| 721 | CGDYLAILPV NPSTVVRKVM SRFDLPWDAV LRIEKASPSS SKHISIPMDT QVSAYDLFAT |
| 781 | YVELSQPASK RDLAVLADAA AVDPETQAEL QAIASDPARF AEISQKRISV LDLLLQYPSI |
| 841 | NLAIGDFVAM LPPMRVRQYS ISSSPLVDPT ECSITFSVLK APSLAALTKE DEYLGVASTY |
| 901 | LSELRSGERV QLSVRPSHTG FKPPTELSTP MIMACAGSGL APFRGFVMDR AEKIRGRRSS |
| 961 | GSMPEQPAKA ILYAGCRTQG KDDIHADELA EWEKIGAVEV RRAYSRPSDG SKGTHVQDLM |
| 1021 | MEDKKELIDL FESGARIYVC GTPGVGNAVR DSIKSMFLER REEIRRIAKE KGEPVSDDDE |
| 1081 | ETAFEKFLDD MKTKERYTTD IFA |

SEQ ID NO: 36
CYP505A1
F. oxysporum
Uniprot Accession No. Q9Y8G7
>gi|22653677|sp|Q9Y8G7.1|C505_FUSOX RecName: Full = Bifunctional P-450: NADPH-
P450 reductase; AltName: Full = Cytochrome P450foxy; AltName: Full = Fatty acid
omega-hydroxylase; Includes: RecName: Full = Cytochrome P450 505; Includes:
RecName: Full = NADPH--cytochrome P450 reductase

| | |
|---|---|
| 1 | maesvpipep pgyplignlg eftsnplsdl nrladtygpi frlrlgakap ifvssnslin |
| 61 | EVCDEKRFKK TLKSVLSQVR EGVHDGLFTA FEDEPNWGKA HRILVPAFGP LSIRGMFPEM |
| 121 | HDIATQLCMK FARHGPRTPI DTSDNFTRLA LDTLALCAMD FRFYSYYKEE LHPFIEAMGD |
| 181 | FLTESGNRNR RPPFAPNFLY RAANEKFYGD IALMKSVADE VVAARKASPS DRKDLLAAML |
| 241 | NGVDPQTGEK LSDENITNQL ITFLIAGHET TSGTLSFAMY QLLKNPEAYS KVQKEVDEVV |
| 301 | GRGPVLVEHL TKLPYISAVL RETLRLNSPI TAFGLEAIDD TFLGGKYLVK KGEIVTALLS |
| 361 | RGHVDPVVYG NDADKFIPER MLDDEFARLN KEYPNCWKPF GNGKRACIGR PFAWQESLLA |
| 421 | MVVLFQNFNF TMTDPNYALE IKQTLTIKPD HFYINATLRH GMTPTELEHV LAGNGATSSS |
| 481 | THNIKAAANL DAKAGSGKPM AIFYGSNSGT CEALANRLAS DAPSHGFSAT TVGPLDQAKQ |
| 541 | NLPEDRPVVI VTASYEGQPP SNAAHFIKWM EDLDGNDMEK VSYAVFACGH HDWVETFHRI |
| 601 | PKLVDSTLEK RGGTRLVPMG SADAATSDMF SDFEAWEDIV LWPGLKEKYK ISDEESGGQK |
| 661 | GLLVEVSTPR KTSLRQDVEE ALVVAEKTLT KSGPAKKHIE IQLPSAMTYK AGDYLAILPL |
| 721 | NPKSTVARVF RRFSLAWDSF LKIQSEGPTT LPTNVAISAF DVFSAYVELS QPATKRNILA |
| 781 | LAEATEDKDT IQELERLAGD AYQAEISPKR VSVLDLLEKF PAVALPISSY LAMLPPMRVR |
| 841 | QYSISSSPFA DPSKLTLTYS LLDAPSLSGQ GRHVGVATNF LSHLTAGDKL HVSVRASSEA |
| 901 | FHLPSDAEKT PIICVAAGTG LAPLRGFIQE RAAMLAAGRT LAPALLFFGC RNPEIDDLYA |
| 961 | EEFERWEKMG AVDVRRAYSR ATDKSEGCKY VQDRVYHDRA DVFKVWDQGA KVFICGSREI |
| 1021 | GKAVEDVCVR LAIEKAQQNG RDVTEEMARA WFERSRNERF ATDVFD |

SEQ ID NO: 37
CYPX
G. moniliformis
GenBank Accession No. AAG27132
>gi|11035011|gb|AAG27132.1|Fum6p [Fusarium verticillioides]

| | |
|---|---|
| 1 | MSATALFTRR SVSTSNPELR PIPGPKPLPL LGNLFDFDFD NLTKSLGELG KIHGPIYSIT |
| 61 | FGASTEIMVT SREIAQELCD ETRFCKLPGG ALDVMKAVVG DGLFTAETSN PKWAIAHRII |
| 121 | TPLFGAMRIR GMFDDMKDIC EQMCLRWARF GPDEPLNVCD NMTKLTLDTI ALCTIDYRFN |
| 181 | SFYRENGAAH PFAEAVVDVM TESFDQSNLP DFVNNYVRFR AMAKFKRQAA ELRRQTEELI |
| 241 | AARRQNPVDR DDLLNAMLSA KDPKTGEGLS PESIVDNLLT FLIAGHETTS SLLSFCFYYL |
| 301 | LENPHVLRRV QQEVDTVVGS DTITVDHLSS MPYLEAVLRE TLRLRDPGPG FYVKPLKDEV |
| 361 | VAGKYAVNKD QPLFIVFDSV HRDQSTYGAD ADEFRPERML KDGFDKLPPC AWKPFGNGVR |
| 421 | ACVGRPFAMQ QAILAVAMVL HKFDLVKDES YTLKYHVTMT VRPVGFTMKV RLRQGQRATD |
| 481 | LAMGLHRGHS QEASAAASPS RASLKRLSSD VNGDDTDHKS QIAVLYASNS GSCEALAYRL |
| 541 | AAEATERGFG IRAVDVVNNA IDRIPVGSPV ILITASYNGE PADDAQEFVP WLKSLESGRL |
| 601 | NGVKFAVFGN GHRDWANTLF AVPRLIDSEL ARCGAERVSL MGVSDTCDSS DPFSDFERWI |
| 661 | DEKLFPELET PHGPGGVKNG DRAVPRQELQ VSLGQPPRIT MRKGYVRAIV TEARSLSSPG |
| 721 | VPEKRHLELL LPKDFNYKAG DHVYILPRNS PRDVVRALSY FGLGEDTLIT IRNTARKLSL |
| 781 | GLPLDTPITA TDLLGAYVEL GRTASLKNLW TLVDAAGHGS RAALLSLTEP ERFRAEVQDR |
| 841 | HVSILDLLER FPDIDLSLSC FLPMLAQIRP RAYSFSSAPD WKPGHATLTY TVVDFATPAT |
| 901 | QGINGSSKSK AVGDGTAVVQ RQGLASSYLS SLGPGTSLYV SLHRASPYFC LQKSTSLPVI |
| 961 | MVGAGTGLAP FRAFLQERRM AAEGAKQRFG PALLFFGCRG PRLDSLYSVE LEAYETIGLV |
| 1021 | QVRRAYSRDP SAQDAQGCKY VTDRLGKCRD EVARLWMDGA QVLVCGGKKM ANDVLEVLGP |
| 1081 | MLLEIDQKRG ETTAKTVVEW RARLDKSRYV EEVYV |

SEQ ID NO: 38
CYP505A7
G. zeae PH1
GenBank Accession No. EAA67736
>gi|42544893|gb|EAA67736.1|C505_FUSOX Bifunctional P-450: NADPH-P450
reductase (Fatty acid omega-hydroxylase) (P450foxy) [Gibberella zeae PH-1]

| | |
|---|---|
| 1 | MAESVPIPEP PGYPLIGNLG EFKTNPLNDL NRLADTYGPI FRLHLGSKTP TFVSSNAFIN |
| 61 | EVCDEKRFKK TLKSVLSVVR EGVHDGLFTA FEDEPNWGKA HRILIPAFGP LSIRNMFPEM |
| 121 | HEIANQLCMK LARHGPHTPV DASDNFTRLA LDTLALCAMD FRFNSYYKEE LHPFIEAMGD |
| 181 | FLLESGNRNR RPAFAPNFLY RAANDKFYAD IALMKSVADE VVATRKQNPT DRKDLLAAML |
| 241 | EGVDPQTGEK LSDDNITNQL ITFLIAGHET TSGTLSFAMY HLLKNPEAYN KLQKEIDEVI |
| 301 | GRDPVTVEHL TKLPYLSAVL RETLRISSPI TGFGVEAIED TFLGGKYLIK KGETVLSVLS |
| 361 | RGHVDPVVYG PDAEKFVPER MLDDEFARLN KEFPNCWKPF GNGKRACIGR PFAWQESLLA |
| 421 | MALLFQNFNF TQTDPNYELQ IKQNLTIKPD NFFFNCTLRH GMTPTDLEGQ LAGKGATTSI |
| 481 | ASHIKAPAAS KGAKASNGKP MAIYYGSNSG TCEALANRLA SDAAGHGFSA SVIGTLDQAK |
| 541 | QNLPEDRPVV IVTASYEGQP PSNAAHFIKW MEDLAGNEME KVSYAVFGCG HHDWVDTFLR |
| 601 | IPKLVDTTLE QRGGTRLVPM GSADAATSDM FSDFEAWEDT VLWPSLKEKY NVTDDEASGQ |
| 661 | RGLLVEVTTP RKTTLRQDVE EALVVSEKTL TKTGPAKKHI EIQLPSGMTY KAGDYLAILP |
| 721 | LNPRKTVSRV FRRFSLAWDS FLKIQSDGPT TLPINIAISA FDVFSAYVEL SQPATKRNIL |
| 781 | ALSEATEDKA TIQELEKLAG DAYQEDVSAK KVSVLDLLEK YPAVALPISS YLAMLPPMRV |
| 841 | RQYSISSSPF ADPSKLTLTY SLLDAPSLSG QGRHVGVATN FLSQLIAGDK LHISVRASSA |
| 901 | AFHLPSDPET TPIICVAAGT GLAPFRGFIQ ERAAMLAAGR KLAPALLFFG CRDPENDDLY |
| 961 | AEELARWEQM GAVDVRRAYS RATDKSEGCK YVQDRIYHDR ADVFKVWDQG AKVFICGSRE |
| 1021 | IGKAVEDICV RLAMERSEAT QEGKGATEEK AREWFERSRN ERFATDVFD |

SEQ ID NO: 39
CYP505C2
G. zeae PH1a
GenBank Accession No. EAA77183
>gi|42554340|gb|EAA77183.1|hypothetical protein FG07596.1 [Gibberella zeae
PH-1]

| | |
|---|---|
| 1 | MAIKDGGKKS GQIPGPKGLP VLGNLFDLDL SDSLTSLINI GQKYAPIFSL ELGGHREVMI |
| 61 | CSRDLLDELC DETRFHKIVT GGVDKLRPLA GDGLFTAQHG NHDWGIAHRI LMPLFGPLKI |
| 121 | REMFDDMQDV SEQLCLKWAR LGPSATIDVA NDFTRLTLDT IALCTMGYRF NSFYSNDKMH |
| 181 | PFVDSMVAAL IDADKQSMFP DFIGACRVKA LSAFRKHAAI MKGTCNELIQ ERRKNPIEGT |
| 241 | DLLTAMMEGK DPKTGEGMSD DLIVQNLITF LIAGHETTSG LLSFAFYYLL ENPHTLEKAR |
| 301 | AEVDEVVGDQ ALNVDHLTKM PYVNMILRET LRLMPTAPGF FVTPHKDEII GGKYAVPANE |
| 361 | SLFCFLHLIH RDPKVWGADA EEFRPERMAD EFFEALPKNA WKPFGNGMRG CIGREFAWQE |
| 421 | AKLITVMILQ NFELSKADPS YKLKIKQSLT IKPDGFNMHA KLRNDRKVSG LFKAPSLSSQ |
| 481 | QPSLSSRQSI NAINAKDLKP ISIFYGSNTG TCEALAQKLS ADCVASGFMP SKPLPLDMAT |
| 541 | KNLSKDGPNI LLAASYDGRP SDNAEEFTKW AESLKPGELE GVQFAVFGCG HKDWVSTYFK |
| 601 | IPKILDKCLA DAGAERLVEI GLTDASTGRL YSDFDDWENQ KLFTELSKRQ GVTPTDDSHL |
| 661 | ELNVTVIQPQ NNDMGGNFKR AEVVENTLLT YPGVSRKHSL LLKLPKDMEY TPGDHVLVLP |
| 721 | KNPPQLVEQA MSCFGVDSDT ALTISSKRPT FLPTDTPILI SSLLSSLVEL SQTVSRTSLK |
| 781 | RLADFADDDD TKACVERIAG DDYTVEVEEQ RMSLLDILRK YPGINMPLST FLSMLPQMRP |
| 841 | RTYSFASAPE WKQGHGMLLF SVVEAEEGTV SRPGGLATNY MAQLRQGDSI LVEPRPCRPE |
| 901 | LRTTMMLPEP KVPIIMIAVG AGLAPFLGYL QKRFLQAQSQ RTALPPCTLL FGCRGAKMDD |
| 961 | ICRAQLDEYS RAGVVSVHRA YSRDPDSQCK YVQGLVTKHS ETLAKQWAQG AIVMVCSGKK |
| 1021 | VSDGVMNVLS PILFAEEKRS GMTGADSVDV WRQNVPKERM ILEVFG |

SEQ ID NO: 40
CYP505A5
M. grisea 70-15 syn
GenBank Accession No. XP 365223
>gi|145601517|ref|XP_365223.2|hypothetical protein MGG_01925
[Magnaporthe oryzae 70-15]

| | |
|---|---|
| 1 | MFFLSSSLAY MAATQSRDWA SFGVSLPSTA LGRHLQAAMP FLSEENHKSQ GTVLIPDAQG |
| 61 | PIPFLGSVPL VDPELPSQSL QRLARQYGEI YRFVIPGRQS PILVSTHALV NELCDEKRFK |
| 121 | KKVAAALLGL REAIHDGLFT AHNDEPNWGI AHRILMPAFG PMAIKGMFDE MHDVASQMIL |
| 181 | KWARHGSTTP IMVSDDFTRL TLDTIALCSM GYRFNSFYHD SMHEFIEAMT CWMKESGNKT |
| 241 | RRLLPDVFYR TTDKKWHDDA EILRRTADEV LKARKENPSG RKDLLTAMIE GVDPKTGGKL |
| 301 | SDSSIIDNLI TFLIAGHETT SGMLSFAFYL LLKNPTAYRK AQQEIDDLCG REPITVEHLS |
| 361 | KMPYITAVLR ETLRLYSTIP AFVVEAIEDT VVGGKYAIPK NHPIFLMIAE SHRDPKVYGD |
| 421 | DAQEFEPERM LDGQFERRNR EFPNSWKPFG NGMRGCIGRA FAWQEALLIT AMLLQNFNFV |
| 481 | MHDPAYQLSI KENLTLKPDN FYMRAILRHG MSPTELERSI SGVAPTGNKT PPRNATRTSS |
| 541 | PDPEDGGIPM SIYYGSNSGT CESLAHKLAV DASAQGFKAE TVDVLDAANQ KLPAGNRGPV |
| 601 | VLITASYEGL PPDNAKHFVE WLENLKGGDE LVDTSYAVFG CGHQDWTKTF HRIPKLVDEK |
| 661 | LAEHGAVRLA PLGLSNAAHG DMFVDFETWE FETLWPALAD RYKTGAGRQD AAATDLTAAL |
| 721 | SQLSVEVSHP RAADLRQDVG EAVVVAARDL TAPGAPPKRH MEIRLPKTGG RVHYSAGDYL |
| 781 | AVLPVNPKST VERAMRRFGL AWDAHVTIRS GGRTTLPTGA PVSAREVLSS YVELTQPATK |
| 841 | RGIAVLAGAV TGGPAAEQEQ AKAALLDLAG DSYALEVSAK RVGVLDLLER FPACAVPFGT |
| 901 | FLALLPPMRV RQYSISSSPL WNDEHATLTY SVLSAPSLAD PARTHVGVAS SYLAGLGEGD |
| 961 | HLHVALRPSH VAFRLPSPET PVVCVCAGSG MAPFRAFAQE RAALVGAGRK VAPLLLFFGC |
| 1021 | REPGVDDLYR EELEGWEAKG VLSVRRAYSR RTEQSEGCRY VQDRLLKNRA EVKSLWSQDA |
| 1081 | KVFVCGSREV AEGVKEAMFK VVAGKEGSSE EVQAWYEEVR NVRYASDIFD |

SEQ ID NO: 41
CYP505A2
N. crassa OR74 A
GenBank Accession No. XP 961848
>gi|85104987|ref|XP_961848.1|bifunctional P-450: NADPH-P450 reductase
[Neurospora crassa OR74A]

| | |
|---|---|
| 1 | MSSDETPQTI PIPGPPGLPL VGNSFDIDTE FPLGSMLNFA DQYGEIFRLN FPGRNTVFVT |
| 61 | SQALVHELCD EKRFQKTVNS ALHEIRHGIH DGLFTARNDE PNWGIAHRIL MPAFGPMAIQ |
| 121 | NMFPEMHEIA SQLALKWARH GPNQSIKVTD DFTRLTLDTI ALCSMDYRFN SYYHDDMHPF |
| 181 | IDAMASFLVE SGNRSRRPAL PAFMYSKVDR KFYDDIRVLR ETAEGVLKSR KEHPSERKDL |
| 241 | LTAMLDGVDP KTGGKLSDDS IIDNLITFLI AGHETTSGLL SFAFVQLLKN PETYRKAQKE |
| 301 | VDDVCGKGPI KLEHMNKLHY IAAVLRETLR LCPTIPVIGV ESKEDTVIGG KYEVSKGQPF |
| 361 | ALLFAKSHVD PAVYGDTAND FDPERMLDEN FERLNKEFPD CWKPFGNGMR ACIGRPFAWQ |
| 421 | EALLVMAVCL QNFNFMPEDP NYTLQYKQTL TTKPKGFYMR AMLRDGMSAL DLERRLKGEL |
| 481 | VAPKPTAQGP VSGQPKKSGE GKPISIYYGS NTGTCETFAQ RLASDAEAHG FTATIIDSLD |
| 541 | AANQNLPKDR PVVFITASYE GQPPDNAALF VGWLESLTGN ELEGVQYAVF GCGHHDWAQT |
| 601 | FHRIPKLVDN TVSERGGDRI CSLGLADAGK GEMFTEFEQW EDEVFWPAME EKYEVSRKED |
| 661 | DNEALLQSGL TVNFSKPRSS TLRQDVQEAV VVDAKTITAP GAPPKRHIEV QLSSDSGAYR |
| 721 | SGDYLAVLPI NPKETVNRVM RRFQLAWDTN ITIEASRQTT ILPTGVPMPV HDVLGAYVEL |
| 781 | SQPATKKNIL ALAEAADNAE TKATLRQLAG PEYTEKITSR RVSILDLLEQ FPSIPLPFSS |
| 841 | FLSLLPPMRV RQYSISSSPL WNPSHVTLTY SLLESPSLSN PDKKHVGVAT SYLASLEAGD |
| 901 | KLNVSIRPSH KAFHLPVDAD KTPLIMIAAG SGLAPFRGFV QERAAQIAAG RSLAPAMLFY |
| 961 | GCRHPEQDDL YRDEFDKWES IGAVSVRRAF SRCPESQETK GCKYVGDRLW EDREEVTGLW |
| 1021 | DRGAKVYVCG SREVGESVKK VVVRIALERQ KMIVEAREKG ELDSLPEGIV EGLKLKGLTV |
| 1081 | EDVEVSEERA LKWFEGIRNE RYATDVFD |

SEQ ID NO: 42
CYP97C
Oryza sativa
GenBank Accession No. ABB47954
>gi|78708979|gb|ABB47954.1|Cytochrome P450 family protein, expressed
[Oryza sativa Japonica Group]

| | |
|---|---|
| 1 | MAAAAAAAVP CVPFLCPPPP PLVSPRLRRG HVRLRLRPPR SSGGGGGGGA GGDEPPITTS |
| 61 | WVSPDWLTAL SRSVATRLGG GDDSGIPVAS AKLDDVRDLL GGALFLPLFK WFREEGPVYR |
| 121 | LAAGPRDLVV VSDPAVARHV LRGYGSRYEK GLVAEVSEFL FGSGFAIAEG ALWTVRRRSV |
| 181 | VPSLHKRFLS VMVDRVFCKC AERLVEKLET SALSGKPVNM EARFSQMTLD VIGLSLFNYN |
| 241 | FDSLTSDSPV IDAVYTALKE AELRSTDLLP YWKIDLLCKI VPRQIKAEKA VNIIRNTVED |
| 301 | LITKCKKIVD AENEQIEGEE YVNEADPSIL RFLLASREEV TSVQLRDDLL SMLVAGHETT |
| 361 | GSVLTWTIYL LSKDPAALRR AQAEVDRVLQ GRLPRYEDLK ELKYLMRCIN ESMRLYPHPP |
| 421 | VLIRRAIVDD VLPGNYKIKA GQDIMISVYN IHRSPEVWDR ADDFIPERFD LEGPVPNETN |
| 481 | TEYRFIPFSG GPRKCVGDQF ALLEAIVALA VVLQKMDIEL VPDQKINMTT GATIHTTNGL |
| 541 | YMNVSLRKVD REPDFALSGS R |

SEQ ID NO: 43
Chimeric heme enzyme C2G9
MKETSPIPQPKTFGPLGNLPLIDKDKPTLSLIKLAEEQGPIFQIHTPAGTTIVVSGHELVKEVCDEERFDKSIEG
ALEKVRAFSGDGLATSWTHEPNWRKAHNILMPTFSQRAMKDYHEKMVDIAVQLIQKWARLNPNEAVDVPGDMTRL
TLDTIGLCGFNYRFNSYYRETPHPFINSMVRALDEAMHQMQRLDVQDKLMVRTKRQFRYDIQTMFSLVDRMIAER
KANPDENIKDLLSLMLYAKDPVTGETLDDENIRYQIITFLIAGHETTSGLLSFALYFLVKNPHVLQKAAEEAARV
LVDPVPSYKQVKQLKYVGMVLNEALRLWPTAPAFSLYAKEDTVLGGEYPISKGQPVTVLIPKLHRDQNAWGPDAE
DFRPERFEDPSSIPHHAYKPFGNGQRACIGMQFALHEATLVLGMILKYFTLIDHENYELDIKQTLTLKPGDFHIS
VQSRHQEAIHADVQAAE
SEQ ID NO: 44
Chimeric heme enzyme X7
MKETSPIPQPKTFGPLGNLPLIDKDKPTLSLIKLAEEQGPIFQIHTPAGTTIVVSGHELVKEVCDEERFDKSIEG
ALEKVRAFSGDGLATSWTHEPNWRKAHNILMPTFSQRAMKDYHEKMVDIATQLIQKWSRLNPNEEIDVADDMTRL
TLDTIGLCGFNYRFNSFYRDQPHPFITSMVRALDEAMNKLQRANPDDPAYDENKRQFQEDIKVMNDLVDSIIAER
RANGDQDEKDLLARMLNVEDPETGEKLDDENIRFQIITFLIAGHETTSGLLSFAIYCLLTHPEKLKKAQEEADRV
LTDDTPEYKQIQQLKYIRMVLNETLRLYPTAPAFSLYAKEDTVLGGEYPISKGQPVTVLIPKLHRDQNAWGPDAE
DFRPERFEDPSSIPHHAYKPFGNGQRACIGMQFALQEATMVLGLVLKHFELINHTGYELKIKEALTIKPDDFKIT
VKPRKTAAINVQRKEQA
SEQ ID NO: 45
Chimeric heme enzyme X7-12
MTIKEMPQPKTFGELKNLPLLNTDKPVQALMKIADELGEIFKFEAPGRVTRYLSSQRLIKEACDEERFDKSIEGA
LEKVRAFSGDGLATSWTHEPNWRKAHNILMPTFSQRAMKDYHEKMVDIAVQLVQKWERLNADEHIEVPEDMTRLT
LDTIGLCGFNYRFNSFYRDQPHPFITSMVRALDEAMNKLQRANPDDPAYDENKRQFQEDIKVMNDLVDSIIAERR
ANGDQDEKDLLARMLNVEDPETGEKLDDENIRFQIITFLIAGHETTSGLLSFAIYCLLTHPEKLKKAQEEADRVL
TDDTPEYKQIQQLKYIRMVLNETLRLYPTAPAFSLYAKEDTVLGGEYPISKGQPVTVLIPKLHRDQNAWGPDAED
FRPERFEDPSSIPHHAYKPFGNGQRACIGMQFALQEATMVLGLVLKHFELINHTGYELKIKEALTIKPDDFKITV
KPRKTAAINVQRKEQA
SEQ ID NO: 46
Chimeric heme enzyme C2E6
MTIKEMPQPKTFGELKNLPLLNTDKPVQALMKIADELGEIFKFEAPGRVTRYLSSQRLIKEACDESRFDKNLSQA
LKFVRDFAGDGLFTSWTHEKNWKKAHNILLPSFSQQAMKGYHAMMVDIAVQLVQKWERLNADEHIEVPEDMTRLT
LDTIGLCGFNYRFNSFYRDQPHPFITSMVRALDEAMNKLQRANPDDPAYDENKRQFQEDIKVMNDLVDRMIAERK
ANPDENIKDLLSLMLYAKDPVTGETLDDENIRYQIITFLIAGHETTSGLLSFAIYCLLTHPEKLKKAQEEADRVL
TDDTPEYKQIQQLKYIRMVLNETLRLYPTAPAFSLYAKEDTVLGGEYPLEKGDELMVLIPQLHRDKTIWGDDVEE
FRPERFENPSAIPQHAFKPFGNGQRACIGQQFALHEATLVLGMMLKHFDFEDHTNYELDIKETLTLKPEGFVVKA
KSKKIPLGGIPSPST
SEQ ID NO: 47
Chimeric heme enzyme X7-9
MKQASAIPQPKTYGPLKNLPHLEKEQLSQSLWRIADELGPIFRFDFPGVSSVFVSGHNLVAEVCDEERFDKSIEG
ALEKVRAFSGDGLATSWTHEPNWRKAHNILMPTFSQRAMKDYHEKMVDIATQLIQKWSRLNPNEEIDVADDMTRL
TLDTIGLCGFNYRFNSFYRDQPHPFITSMVRALDEAMNKLQRANPDDPAYDENKRQFQEDIKVMNDLVDSIIAER
RANGDQDEKDLLARMLNVEDPETGEKLDDENIRFQIITFLIAGHETTSGLLSFAIYCLLTHPEKLKKAQEEADRV
LTDDTPEYKQIQQLKYIRMVLNETLRLYPTAPAFSLYAKEDTVLGGEYPISKGQPVTVLIPKLHRDQNAWGPDAE
DFRPERFEDPSSIPHHAYKPFGNGQRACIGMQFALQEATMVLGLVLKHFELINHTGYELKIKEALTIKPDDFKIT
VKPRKTAAINVQRKEQA
SEQ ID NO: 48
Chimeric heme enzyme C2B12
MKQASAIPQPKTYGPLKNLPHLEKEQLSQSLWRIADELGPIFRFDFPGVSSVFVSGHNLVAEVCDEERFDKSIEG
ALEKVRAFSGDGLATSWTHEPNWRKAHNILMPTFSQRAMKDYHEKMVDIATQLIQKWSRLNPNEEIDVADDMTRL
TLDTIGLCGFNYRFNSFYRDQPHPFITSMVRALDEAMNKLQRANPDDPAYDENKRQFQEDIKVMNDLVDRMIAER
KANPDENIKDLLSLMLYAKDPVTGETLDDENIRYQIITFLIAGHETTSGLLSFATYFLLKHPDKLKKAYEEVDRV
LTDAAPTYKQVLELTYIRMILNESLRLWPTAPAFSLYAKEDTVLGGEYPISKGQPVTVLIPKLHRDQNAWGPDAE
DFRPERFEDPSSIPHHAYKPFGNGQRACIGMQFALQEATMVLGLVLKHFELINHTGYELKIKEALTIKPDDFKIT
VKPRKTAAINVQRKEQA
SEQ ID NO: 49
Chimeric heme enzyme TSP234
MKETSPIPQPKTFGPLGNLPLIDKDKPTLSLIKLAEEQGPIFQIHTPAGTTIVVSGHELVKEVCDEERFDKSIEG
ALEKVRAFSGDGLATSWTHEPNWRKAHNILMPTFSQRAMKDYHEKMVDIATQLIQKWSRLNPNEEIDVADDMTRL
TLDTIGLCGFNYRFNSFYRDQPHPFITSMVRALDEAMNKLQRANPDDPAYDENKRQFQEDIKVMNDLVDRMIAER
KANPDENIKDLLSLMLYAKDPVTGETLDDENIRYQIITFLIAGHETTSGLLSFAIYCLLTHPEKLKKAQEEADRV
LTDDTPEYKQIQQLKYIRMVLNETLRLYPTAPAFSLYAKEDTVLGGEYPISKGQPVTVLIPKLHRDQNAWGPDAE
DFRPERFEDPSSIPHHAYKPFGNGQRACIGMQFALQEATMVLGLVLKHFELINHTGYELKIKEALTIKPDDFKIT
VKPRKTAAINVQRKEQA
SEQ ID NO: 50
CYP101A1 mutant (C357S)
Cytochrome P450cam mutant
TTETIQSNA NLAPLPPHVP EHLVFDFDMY NPSNLSAGVQ EAWAVLQESN VPDLVWTRCN
GGHWIATRGQ LIREAYEDYR HFSSECPFIP REAGEAYDFI PTSMDPPEQR QFRALANQVV
GMPVVDKLEN RIQELACSLI ESLRPQGQCN FTEDYAEPFP IRIFMLLAGL PEEDIPHLKY
LTDQMTRPDG SMTFAEAKEA LYDYLIPIIE QRRQKPGTDA ISIVANGQVN GRPITSDEAK
RMCGLLLVGG LDTVVNFLSF SMEFLAKSPE HRQELIERPE RIPAACEELL RRFSLVADGR
ILTSDYEFHG VQLKKGDQIL LPQMLSGLDE RENACPMHVD FSRQKVSHTT FGHGSHLSLG
QHLARREIIV TLKEWLTRIP DFSIAPGAQI QHKSGIVSGV QALPLVWDPA TTKAV
SEQ ID NO: 51
CYP101A1 mutant (T252A)
Cytochrome P450cam mutant
TTETIQSNA NLAPLPPHVP EHLVFDFDMY NPSNLSAGVQ EAWAVLQESN VPDLVWTRCN
GGHWIATRGQ LIREAYEDYR HFSSECPFIP REAGEAYDFI PTSMDPPEQR QFRALANQVV
GMPVVDKLEN RIQELACSLI ESLRPQGQCN FTEDYAEPFP IRIFMLLAGL PEEDIPHLKY
LTDQMTRPDG SMTFAEAKEA LYDYLIPIIE QRRQKPGTDA ISIVANGQVN GRPITSDEAK
RMCGLLLVGG LDAVVNFLSF SMEFLAKSPE HRQELIERPE RIPAACEELL RRFSLVADGR
ILTSDYEFHG VQLKKGDQIL LPQMLSGLDE RENACPMHVD FSRQKVSHTT FGHGSHLCLG
QHLARREIIV TLKEWLTRIP DFSIAPGAQI QHKSGIVSGV QALPLVWDPA TTKAV
SEQ ID NO: 52
CYP101A1 mutant (C357S and T252A)
Cytochrome P450cam mutant
TTETIQSNA NLAPLPPHVP EHLVFDFDMY NPSNLSAGVQ EAWAVLQESN VPDLVWTRCN
GGHWIATRGQ LIREAYEDYR HFSSECPFIP REAGEAYDFI PTSMDPPEQR QFRALANQVV
GMPVVDKLEN RIQELACSLI ESLRPQGQCN FTEDYAEPFP IRIFMLLAGL PEEDIPHLKY
LTDQMTRPDG SMTFAEAKEA LYDYLIPIIE QRRQKPGTDA ISIVANGQVN GRPITSDEAK
RMCGLLLVGG LDAVVNFLSF SMEFLAKSPE HRQELIERPE RIPAACEELL RRFSLVADGR
ILTSDYEFHG VQLKKGDQIL LPQMLSGLDE RENACPMHVD FSRQKVSHTT FGHGSHLSLG
QHLARREIIV TLKEWLTRIP DFSIAPGAQI QHKSGIVSGV QALPLVWDPA TTKAV
SEQ ID NO: 53
CYP2B4 (C436S)
Cytochrome P450 2B4 mutant

| | |
|---|---|
| 1 | MEFSLLLLLA FLAGLLLLLF RGHPKAHGRL PPGPSPLPVL GNLLQMDRKG LLRSFLRLRE |
| 61 | KYGDVFTVYL GSRPVVVLCG TDAIREALVD QAEAFSGRGK IAVVDPIFQG YGVIFANGER |
| 121 | WRALRRFSLA TMRDFGMGKR SVEERIQEEA RCLVEELRKS KGALLDNTLL FHSITSNIIC |
| 181 | SIVFGKRFDY KDPVFLRLLD LFFQSFSLIS SFSSQVFELF PGFLKHFPGT HRQIYRNLQE |
| 241 | INTFIGQSVE KHRATLDPSN PRDFIDVYLL RMEKDKSDPS SEFHHQNLIL TVLSLFFAGT |
| 301 | ETTSTTLRYG FLLMLKYPHV TERVQKEIEQ VIGSHRPPAL DDRAKMPYTD AVIHEIQRLG |
| 361 | DLIPFGVPHT VTKDTQFRGY VIPKNTEVFP VLSSALHDPR YFETPNTFNP GHFLDANGAL |
| 421 | KRNEGFMPFS LGKRISLGEG IARTELFLFF TTILQNFSIA SPVPPEDIDL TPRESGVGNV |
| 481 | PPSYQIRFLA R |

SEQ ID NO: 54
CYP2B4 (T302A)
Cytochrome P450 2B4 mutant

| | |
|---|---|
| 1 | MEFSLLLLLA FLAGLLLLLF RGHPKAHGRL PPGPSPLPVL GNLLQMDRKG LLRSFLRLRE |
| 61 | KYGDVFTVYL GSRPVVVLCG TDAIREALVD QAEAFSGRGK IAVVDPIFQG YGVIFANGER |
| 121 | WRALRRFSLA TMRDFGMGKR SVEERIQEEA RCLVEELRKS KGALLDNTLL FHSITSNIIC |
| 181 | SIVFGKRFDY KDPVFLRLLD LFFQSFSLIS SFSSQVFELF PGFLKHFPGT HRQIYRNLQE |
| 241 | INTFIGQSVE KHRATLDPSN PRDFIDVYLL RMEKDKSDPS SEFHHQNLIL TVLSLFFAGT |
| 301 | EATSTTLRYG FLLMLKYPHV TERVQKEIEQ VIGSHRPPAL DDRAKMPYTD AVIHEIQRLG |
| 361 | DLIPFGVPHT VTKDTQFRGY VIPKNTEVFP VLSSALHDPR YFETPNTFNP GHFLDANGAL |
| 421 | KRNEGFMPFS LGKRICLGEG IARTELFLFF TTILQNFSIA SPVPPEDIDL TPRESGVGNV |
| 481 | PPSYQIRFLA R |

SEQ ID NO: 55
CYP2B4 (C436S and T302A)
Cytochrome P450 2B4 mutant

| | |
|---|---|
| 1 | MEFSLLLLLA FLAGLLLLLF RGHPKAHGRL PPGPSPLPVL GNLLQMDRKG LLRSFLRLRE |
| 61 | KYGDVFTVYL GSRPVVVLCG TDAIREALVD QAEAFSGRGK IAVVDPIFQG YGVIFANGER |
| 121 | WRALRRFSLA TMRDFGMGKR SVEERIQEEA RCLVEELRKS KGALLDNTLL FHSITSNIIC |
| 181 | SIVFGKRFDY KDPVFLRLLD LFFQSFSLIS SFSSQVFELF PGFLKHFPGT HRQIYRNLQE |
| 241 | INTFIGQSVE KHRATLDPSN PRDFIDVYLL RMEKDKSDPS SEFHHQNLIL TVLSLFFAGT |
| 301 | EATSTTLRYG FLLMLKYPHV TERVQKEIEQ VIGSHRPPAL DDRAKMPYTD AVIHEIQRLG |
| 361 | DLIPFGVPHT VTKDTQFRGY VIPKNTEVFP VLSSALHDPR YFETPNTFNP GHFLDANGAL |
| 421 | KRNEGFMPFS LGKRISLGEG IARTELFLFF TTILQNFSIA SPVPPEDIDL TPRESGVGNV |
| 481 | PPSYQIRFLA R |

SEQ ID NO: 56
WT-AxA (heme)
TIKEMPQPKTFGELKNLPLLNTDKPVQALMKIADELGEIFKFEAPGRVTRYLSSQRLIKEACDESRFDKNLSQAL
KFVRDFAGDGLFTSWTHEKNWKKAHNILLPSFSQQAMKGYHAMMVDIAVQLVQKWERLNADEHIEVPEDMTRLTL
DTIGLCGFNYRFNSFYRDQPHPFITSMVRALDEAMNKLQRANPDDPAYDENKRQFQEDIKVMNDLVDKIIADRKA
SGEQSDDLLTHMLNGKDPETGEPLDDENIRYQIITFLIAGHETTSGLLSFALYFLVKNPHVLQKAAEEAARVLVD
PVPSYKQVKQLKYVGMVLNEALRLWPTAPAFSLYAKEDTVLGGEYPLEKGDELMVLIPQLHRDKTIWGDDVEEFR
PERFENPSAIPQHAFKPFGNGQRAAIGQQFALHEATLVLGMMLKHFDFEDHTNYELDIKETLSLKPKGFVVKAKS
KKIPLGGIPSPST
SEQ ID NO: 57
WT-AxD (heme)
TIKEMPQPKTFGELKNLPLLNTDKPVQALMKIADELGEIFKFEAPGRVTRYLSSQRLIKEACDESRFDKNLSQAL
KFVRDFAGDGLFTSWTHEKNWKKAHNILLPSFSQQAMKGYHAMMVDIAVQLVQKWERLNADEHIEVPEDMTRLTL
DTIGLCGFNYRFNSFYRDQPHPFITSMVRALDEAMNKLQRANPDDPAYDENKRQFQEDIKVMNDLVDKIIADRKA
SGEQSDDLLTHMLNGKDPETGEPLDDENIRYQIITFLIAGHETTSGLLSFALYFLVKNPHVLQKAAEEAARVLVD
PVPSYKQVKQLKYVGMVLNEALRLWPTAPAFSLYAKEDTVLGGEYPLEKGDELMVLIPQLHRDKTIWGDDVEEFR
PERFENPSAIPQHAFKPFGNGQRADIGQQFALHEATLVLGMMLKHFDFEDHTNYELDIKETLSLKPKGFVVKAKS
KKIPLGGIPSPST
SEQ ID NO: 58
WT-AxH (heme)
TIKEMPQPKTFGELKNLPLLNTDKPVQALMKIADELGEIFKFEAPGRVTRYLSSQRLIKEACDESRFDKNLSQAL
KFVRDFAGDGLFTSWTHEKNWKKAHNILLPSFSQQAMKGYHAMMVDIAVQLVQKWERLNADEHIEVPEDMTRLTL
DTIGLCGFNYRFNSFYRDQPHPFITSMVRALDEAMNKLQRANPDDPAYDENKRQFQEDIKVMNDLVDKIIADRKA
SGEQSDDLLTHMLNGKDPETGEPLDDENIRYQIITFLIAGHETTSGLLSFALYFLVKNPHVLQKAAEEAARVLVD
PVPSYKQVKQLKYVGMVLNEALRLWPTAPAFSLYAKEDTVLGGEYPLEKGDELMVLIPQLHRDKTIWGDDVEEFR
PERFENPSAIPQHAFKPFGNGQRAHIGQQFALHEATLVLGMMLKHFDFEDHTNYELDIKETLSLKPKGFVVKAKS
KKIPLGGIPSPST
SEQ ID NO: 59
WT-AxK (heme)
TIKEMPQPKTFGELKNLPLLNTDKPVQALMKIADELGEIFKFEAPGRVTRYLSSQRLIKEACDESRFDKNLSQAL
KFVRDFAGDGLFTSWTHEKNWKKAHNILLPSFSQQAMKGYHAMMVDIAVQLVQKWERLNADEHIEVPEDMTRLTL
DTIGLCGFNYRFNSFYRDQPHPFITSMVRALDEAMNKLQRANPDDPAYDENKRQFQEDIKVMNDLVDKIIADRKA
SGEQSDDLLTHMLNGKDPETGEPLDDENIRYQIITFLIAGHETTSGLLSFALYFLVKNPHVLQKAAEEAARVLVD
PVPSYKQVKQLKYVGMVLNEALRLWPTAPAFSLYAKEDTVLGGEYPLEKGDELMVLIPQLHRDKTIWGDDVEEFR
PERFENPSAIPQHAFKPFGNGQRAKIGQQFALHEATLVLGMMLKHFDFEDHTNYELDIKETLSLKPKGFVVKAKS
KKIPLGGIPSPST
SEQ ID NO: 60
WT-AxM (heme)
TIKEMPQPKTFGELKNLPLLNTDKPVQALMKIADELGEIFKFEAPGRVTRYLSSQRLIKEACDESRFDKNLSQAL
KFVRDFAGDGLFTSWTHEKNWKKAHNILLPSFSQQAMKGYHAMMVDIAVQLVQKWERLNADEHIEVPEDMTRLTL
DTIGLCGFNYRFNSFYRDQPHPFITSMVRALDEAMNKLQRANPDDPAYDENKRQFQEDIKVMNDLVDKIIADRKA
SGEQSDDLLTHMLNGKDPETGEPLDDENIRYQIITFLIAGHETTSGLLSFALYFLVKNPHVLQKAAEEAARVLVD
PVPSYKQVKQLKYVGMVLNEALRLWPTAPAFSLYAKEDTVLGGEYPLEKGDELMVLIPQLHRDKTIWGDDVEEFR
PERFENPSAIPQHAFKPFGNGQRAMIGQQFALHEATLVLGMMLKHFDFEDHTNYELDIKETLSLKPKGFVVKAKS
KKIPLGGIPSPST
SEQ ID NO: 61
WT-AxN (heme)
TIKEMPQPKTFGELKNLPLLNTDKPVQALMKIADELGEIFKFEAPGRVTRYLSSQRLIKEACDESRFDKNLSQAL
KFVRDFAGDGLFTSWTHEKNWKKAHNILLPSFSQQAMKGYHAMMVDIAVQLVQKWERLNADEHIEVPEDMTRLTL
DTIGLCGFNYRFNSFYRDQPHPFITSMVRALDEAMNKLQRANPDDPAYDENKRQFQEDIKVMNDLVDKIIADRKA
SGEQSDDLLTHMLNGKDPETGEPLDDENIRYQIITFLIAGHETTSGLLSFALYFLVKNPHVLQKAAEEAARVLVD
PVPSYKQVKQLKYVGMVLNEALRLWPTAPAFSLYAKEDTVLGGEYPLEKGDELMVLIPQLHRDKTIWGDDVEEFR
PERFENPSAIPQHAFKPFGNGQRANIGQQFALHEATLVLGMMLKHFDFEDHTNYELDIKETLSLKPKGFVVKAKS
KKIPLGGIPSPST
SEQ ID NO: 62
BM3-CIS-T438S-AxA
TIKEMPQPKTFGELKNLPLLNTDKPVQALMKIADELGEIFKFEAPGRVTRYLSSQRLIKEACDESRFDKNLSQAL
KFARDFAGDGLVTSWTHEKNWKKAHNILLPSFSQQAMKGYHAMMVDIAVQLVQKWERLNADEHIEVSEDMTRLTL
DTIGLCGFNYRFNSFYRDQPHPFIISMVRALDEVMNKLQRANPDDPAYDENKRQFQEDIKVMNDLVDIIADRKAR
GEQSDDLLTQMLNGKDPETGEPLDDGNIRYQIITFLIAGHEATSGLLSFALYFLVKNPHVLQKVAEEAARVLVDP
VPSYKQVKQLKYVGMVLNEALRLWPTAPAFSLYAKEDTVLGGEYPLEKGDEVMVLIPQLHRDKTVWGDDVEEFRP
ERFENPSAIPQHAFKPFGNGQRAAIGQQFALHEATLVLGMMLKHFDFEDHTNYELDIKETLSLKPKGFVVKAKSK
KIPLGGIPSPSTEQSAKKVRKKAENAHNTPLLVLYGSNMGTAEGTARDLADIAMSKGFAPQVATLDSHAGNLPRE
GAVLIVTASYNGHPPDNAKQFVDWLDQASADEVKGVRYSVFGCGDKNWATTYQKVPAFIDETLAAKGAENIADRG
EADASDDFEGTYEEWREHMWSDVAAYFNLDIENSEDNKSTLSLQFVDSAADMPLAKMHGAFSTNVVASKELQQPG
SARSTRHLEIELPKEASYQEGDHLGVIPRNYEGIVNRVTARFGLDASQQIRLEAEEEKLAHLPLAKTVSVEELLQ
YVELQDPVTRTQLRAMAAKTVCPPHKVELEALLEKQAYKEQVLAKRLTMLELLEKYPACEMKFSEFIALLPSIRP
RYYSISSSPRVDEKQASITVSVVSGEAWSGYGEYKGIASNYLAELQEGDTITCFISTPQSEFTLPKDPETPLIMV
GPGTGVAPFRGFVQARKQLKEQGQSLGEAHLYFGCRSPHEDYLYQEELENAQSEGIITLHTAFSRMPNQPKTYVQ
HVMEQDGKKLIELLDQGAHFYICGDGSQMAPAVEATLMKSYADVHQVSEADARLWLQQLEEKGRYAKDVWAG
SEQ ID NO: 63
BM3-CIS-T438S-AxD
TIKEMPQPKTFGELKNLPLLNTDKPVQALMKIADELGEIFKFEAPGRVTRYLSSQRLIKEACDESRFDKNLSQAL
KFARDFAGDGLVTSWTHEKNWKKAHNILLPSFSQQAMKGYHAMMVDIAVQLVQKWERLNADEHIEVSEDMTRLTL
DTIGLCGFNYRFNSFYRDQPHPFIISMVRALDEVMNKLQRANPDDPAYDENKRQFQEDIKVMNDLVDITADRKAR
GEQSDDLLTQMLNGKDPETGEPLDDGNIRYQIITFLIAGHEATSGLLSFALYFLVKNPHVLQKVAEEAARVLVDP
VPSYKQVKQLKYVGMVLNEALRLWPTAPAFSLYAKEDTVLGGEYPLEKGDEVMVLIPQLHRDKTVWGDDVEEFRP
ERFENPSAIPQHAFKPFGNGQRADIGQQFALHEATLVLGMMLKHFDFEDHTNYELDIKETLSLKPKGFVVKAKSK
KIPLGGIPSPSTEQSAKKVRKKAENAHNTPLLVLYGSNMGTAEGTARDLADIAMSKGFAPQVATLDSHAGNLPRE
GAVLIVTASYNGHPPDNAKQFVDWLDQASADEVKGVRYSVFGCGDKNWATTYQKVPAFIDETLAAKGAENIADRG
EADASDDFEGTYEEWREHMWSDVAAYFNLDIENSEDNKSTLSLQFVDSAADMPLAKMHGAFSTNVVASKELQQPG
SARSTRHLEIELPKEASYQEGDHLGVIPRNYEGIVNRVTARFGLDASQQIRLEAEEEKLAHLPLAKTVSVEELLQ
YVELQDPVTRTQLRAMAAKTVCPPHKVELEALLEKQAYKEQVLAKRLTMLELLEKYPACEMKFSEFIALLPSIRP
RYYSISSSPRVDEKQASITVSVVSGEAWSGYGEYKGIASNYLAELQEGDTITCFISTPQSEFTLPKDPETPLIMV
GPGTGVAPFRGFVQARKQLKEQGQSLGEAHLYFGCRSPHEDYLYQEELENAQSEGIITLHTAFSRMPNQPKTYVQ
HVMEQDGKKLIELLDQGAHFYICGDGSQMAPAVEATLMKSYADVHQVSEADARLWLQQLEEKGRYAKDVWAG
SEQ ID NO: 64
BM3-CIS-T438S-AxM
TIKEMPQPKTFGELKNLPLLNTDKPVQALMKIADELGEIFKFEAPGRVTRYLSSQRLIKEACDESRFDKNLSQAL
KFARDFAGDGLVTSWTHEKNWKKAHNILLPSFSQQAMKGYHAMMVDIAVQLVQKWERLNADEHIEVSEDMTRLTL
DTIGLCGFNYRFNSFYRDQPHPFIISMVRALDEVMNKLQRANPDDPAYDENKRQFQEDIKVMNDLVDITADRKAR
GEQSDDLLTQMLNGKDPETGEPLDDGNIRYQIITFLIAGHEATSGLLSFALYFLVKNPHVLQKVAEEAARVLVDP
VPSYKQVKQLKYVGMVLNEALRLWPTAPAFSLYAKEDTVLGGEYPLEKGDEVMVLIPQLHRDKTVWGDDVEEFRP
ERFENPSAIPQHAFKPFGNGQRAMIGQQFALHEATLVLGMMLKHFDFEDHTNYELDIKETLSLKPKGFVVKAKSK
KIPLGGIPSPSTEQSAKKVRKKAENAHNTPLLVLYGSNMGTAEGTARDLADIAMSKGFAPQVATLDSHAGNLPRE
GAVLIVTASYNGHPPDNAKQFVDWLDQASADEVKGVRYSVFGCGDKNWATTYQKVPAFIDETLAAKGAENIADRG
EADASDDFEGTYEEWREHMWSDVAAYFNLDIENSEDNKSTLSLQFVDSAADMPLAKMHGAFSTNVVASKELQQPG
SARSTRHLEIELPKEASYQEGDHLGVIPRNYEGIVNRVTARFGLDASQQIRLEAEEEKLAHLPLAKTVSVEELLQ
YVELQDPVTRTQLRAMAAKTVCPPHKVELEALLEKQAYKEQVLAKRLTMLELLEKYPACEMKFSEFIALLPSIRP
RYYSISSSPRVDEKQASITVSVVSGEAWSGYGEYKGIASNYLAELQEGDTITCFISTPQSEFTLPKDPETPLIMV
GPGTGVAPFRGFVQARKQLKEQGQSLGEAHLYFGCRSPHEDYLYQEELENAQSEGIITLHTAFSRMPNQPKTYVQ
HVMEQDGKKLIELLDQGAHFYICGDGSQMAPAVEATLMKSYADVHQVSEADARLWLQQLEEKGRYAKDVWAG
SEQ ID NO: 65
BM3-CIS-T438S-AxY
TIKEMPQPKTFGELKNLPLLNTDKPVQALMKIADELGEIFKFEAPGRVTRYLSSQRLIKEACDESRFDKNLSQAL
KFARDFAGDGLVTSWTHEKNWKKAHNILLPSFSQQAMKGYHAMMVDIAVQLVQKWERLNADEHIEVSEDMTRLTL
DTIGLCGFNYRFNSFYRDQPHPFIISMVRALDEVMNKLQRANPDDPAYDENKRQFQEDIKVMNDLVDITADRKAR
GEQSDDLLTQMLNGKDPETGEPLDDGNIRYQIITFLIAGHEATSGLLSFALYFLVKNPHVLQKVAEEAARVLVDP
VPSYKQVKQLKYVGMVLNEALRLWPTAPAFSLYAKEDTVLGGEYPLEKGDEVMVLIPQLHRDKTVWGDDVEEFRP
ERFENPSAIPQHAFKPFGNGQRAYIGQQFALHEATLVLGMMLKHFDFEDHTNYELDIKETLSLKPKGFVVKAKSK
KIPLGGIPSPSTEQSAKKVRKKAENAHNTPLLVLYGSNMGTAEGTARDLADIAMSKGFAPQVATLDSHAGNLPRE
GAVLIVTASYNGHPPDNAKQFVDWLDQASADEVKGVRYSVFGCGDKNWATTYQKVPAFIDETLAAKGAENIADRG
EADASDDFEGTYEEWREHMWSDVAAYFNLDIENSEDNKSTLSLQFVDSAADMPLAKMHGAFSTNVVASKELQQPG
SARSTRHLEIELPKEASYQEGDHLGVIPRNYEGIVNRVTARFGLDASQQIRLEAEEEKLAHLPLAKTVSVEELLQ
YVELQDPVTRTQLRAMAAKTVCPPHKVELEALLEKQAYKEQVLAKRLTMLELLEKYPACEMKFSEFIALLPSIRP
RYYSISSSPRVDEKQASITVSVVSGEAWSGYGEYKGIASNYLAELQEGDTITCFISTPQSEFTLPKDPETPLIMV
GPGTGVAPFRGFVQARKQLKEQGQSLGEAHLYFGCRSPHEDYLYQEELENAQSEGIITLHTAFSRMPNQPKTYVQ
HVMEQDGKKLIELLDQGAHFYICGDGSQMAPAVEATLMKSYADVHQVSEADARLWLQQLEEKGRYAKDVWAG
SEQ ID NO: 66
BM3-CIS-T438S-AxT
TIKEMPQPKTFGELKNLPLLNTDKPVQALMKIADELGEIFKFEAPGRVTRYLSSQRLIKEACDESRFDKNLSQAL
KFARDFAGDGLVTSWTHEKNWKKAHNILLPSFSQQAMKGYHAMMVDIAVQLVQKWERLNADEHIEVSEDMTRLTL
DTIGLCGFNYRFNSFYRDQPHPFIISMVRALDEVMNKLQRANPDDPAYDENKRQFQEDIKVMNDLVDITADRKAR
GEQSDDLLTQMLNGKDPETGEPLDDGNIRYQIITFLIAGHEATSGLLSFALYFLVKNPHVLQKVAEEAARVLVDP
VPSYKQVKQLKYVGMVLNEALRLWPTAPAFSLYAKEDTVLGGEYPLEKGDEVMVLIPQLHRDKTVWGDDVEEFRP
ERFENPSAIPQHAFKPFGNGQRATIGQQFALHEATLVLGMMLKHFDFEDHTNYELDIKETLSLKPKGFVVKAKSK
KIPLGGIPSPSTEQSAKKVRKKAENAHNTPLLVLYGSNMGTAEGTARDLADIAMSKGFAPQVATLDSHAGNLPRE
GAVLIVTASYNGHPPDNAKQFVDWLDQASADEVKGVRYSVFGCGDKNWATTYQKVPAFIDETLAAKGAENIADRG
EADASDDFEGTYEEWREHMWSDVAAYFNLDIENSEDNKSTLSLQFVDSAADMPLAKMHGAFSTNVVASKELQQPG
SARSTRHLEIELPKEASYQEGDHLGVIPRNYEGIVNRVTARFGLDASQQIRLEAEEEKLAHLPLAKTVSVEELLQ
YVELQDPVTRTQLRAMAAKTVCPPHKVELEALLEKQAYKEQVLAKRLTMLELLEKYPACEMKFSEFIALLPSIRP
RYYSISSSPRVDEKQASITVSVVSGEAWSGYGEYKGIASNYLAELQEGDTITCFISTPQSEFTLPKDPETPLIMV
GPGTGVAPFRGFVQARKQLKEQGQSLGEAHLYFGCRSPHEDYLYQEELENAQSEGIITLHTAFSRMPNQPKTYVQ
HVMEQDGKKLIELLDQGAHFYICGDGSQMAPAVEATLMKSYADVHQVSEADARLWLQQLEEKGRYAKDVWAG

1. A method for catalyzing a carbene insertion into a N—H bond to produce a product having a new C—N bond, the method comprising:
providing a N—H containing substrate, a diazo carbene precursor and an engineered heme enzyme; and
allowing the reaction to proceed for a time sufficient to form a product having a new C—N bond.

2. The method of claim 1, wherein the N—H containing substrate is an aryl amine.

3. The method of claim 1, wherein the N—H containing substrate is an aliphatic amine.

4. The method of claim 1, wherein the diazo carbene precursor is an aryl diazo carbene precursor.

5. The method of claim 1, wherein the diazo carbene precursor is an aliphatic diazo carbene precursor.

6. The method of claim 1, wherein the engineered heme enzyme is a cytochrome P450 enzyme or a variant thereof.

7. The method of claim 6, wherein the cytochrome P450 enzyme is expressed in a bacterial, archaeal or fungal host organism.

8. The method of claim 6, wherein the cytochrome P450 enzyme is a P450 BM3 enzyme or a variant thereof.

9. The method of claim 8, wherein the cytochrome P450 BM3 enzyme comprises the amino acid sequence set forth in SEQ ID NO:1 or a variant thereof.

10. The method of claim 6, wherein the cytochrome P450 enzyme variant comprises a mutation at the axial position of the heme coordination site.

11. The method of claim 10, wherein the mutation is an amino acid substitution of Cys with a member selected from the group consisting of Ala, Asp, Arg, Asn, Glu, Gln, Gly, His, Ile, Lys, Leu, Met, Phe, Pro, Ser, Thr, Trp, Tyr and Val at the axial position.

12. The method of claim 10, wherein the mutation is an amino acid substitution of Cys with Asp or Ser at the axial position.

13. The method of claim 8, wherein the P450 BM3 enzyme variant comprises at least one, two, three, four, five, six, seven, eight, nine, ten, eleven, twelve, or all thirteen of the following amino acid substitutions in SEQ ID NO: 1: V78A, F87V, P142S, T175I, A184V, S226R, H236Q, E252G, T268A, A290V, L353V, 1366V, and E442K.

14. The method of claim 6, wherein the cytochrome P450 enzyme variant comprises a T268A mutation and/or a C400X mutation in SEQ ID NO:1, wherein X is any amino acid other than Cys.

15. The method of claim 6, wherein the cytochrome P450 enzyme variant comprises a T438S mutation and/or a C400X mutation in SEQ ID NO:1, wherein X is any amino acid other than Cys.

16. The method of claim 6, wherein the engineered heme enzyme comprises a fragment of the cytochrome P450 enzyme or variant thereof.

17. The method of claim 6, wherein the engineered heme enzyme is a cytochrome P450 BM3 enzyme variant selected from Table 4, Table 5, Table 6 and Table 9.

18. The method of claim 1, wherein the product is a compound of Formula Ia: 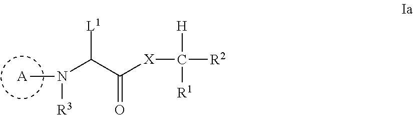
wherein: the dotted circle A is an optionally substituted aryl group, wherein the nitrogen represents an endocyclic nitrogen atom which is part of ring A or an exocyclic nitrogen atom bonded to a ring atom of A;
R¹ is a member selected from the group consisting of hydrogen, an optionally substituted alkyl, and cyano;
R² is a member selected from the group consisting of hydrogen, an optionally substituted alkyl, an optionally substituted aryl, an optionally substituted heteroaryl, and an optionally substituted heterocyclyl;
R³ is a member selected from the group consisting of hydrogen and an optionally substituted alkyl,
X is a heteroatom selected form the group consisting of S, O and NR, wherein R is hydrogen or optionally substituted alkyl; and
L¹ is an optionally substituted alkyl or hydrogen.

19. The method of claim 18, wherein R² is an optionally substituted aryl group.

20. The method of claim 19, wherein R² is an optionally substituted phenoxybenzyl.

21. The method of claim 18, wherein A is an optionally substituted aryl group and the nitrogen is exocyclic.

22. The method of claim 18, wherein L¹ is an isopropyl group.

23. The method of claim 21, wherein A is an analinyl group optionally substituted with 1 to 5 substituents, which may be the same or different, selected from the group consisting of a halogen atom, an alkyl, haloalkyl, phenyl, alkoxy, haloalkoxy, cycloalkoxy, phenoxy, alkenyl, haloalkenyl, alkynyl, haloalkynyl, alkoxyalkyl, alkenyloxy, haloalkenyloxy, alkynyloxy, haloalkynyloxy, alkylthio, haloalkylthio, alkylsulfoxyl, acyl, alkoxyalkoxy, alkenylthio, alkoxycarbonyl, haloalkoxycarbonyl, alkynyloxycarbonyl, alkenyloxycarbonyl, nitro, and haloalkenylthio.

24. The method of claim 23, wherein the compound is a member selected from the group consisting of cyano(3-phenoxyphenyl)methyl 2-((2-fluoro-4-(trifluoromethyl)phenyl)amino)-3-methylbutanoate; cyano(3-fluoro-5-phenoxyphenyl)methyl 2-((2-chloro-4-(trifluoromethyl)phenyl)amino)-3-methylbutanoate; cyano(4-fluoro-3-phenoxyphenyl)methyl 2-((2-chloro-4-(trifluoromethyl)phenyl)amino)-3-methylbutanoate; cyano(2-fluoro-5-phenoxyphenyl)methyl 2-((2-chloro-4-(trifluoromethyl)phenyl)amino)-3-methylbutanoate; cyano(3-phenoxyphenyl)methyl 24(2-fluoro-4-((trifluoromethyl)thio)phenyl)amino)-3-methylbutanoate; and (2,5-dioxo-3-(prop-2-yn-1-yl)imidazolidin-1-yl)methyl 3-methyl-2-((4-(trifluoromethyl)phenyl)amino)butanoate.

25. The method of claim 18, wherein A is an optionally substituted aryl group and the nitrogen is endocylic.

26. The method of claim 25, wherein A is an optionally substituted pyrroyl group optionally substituted with 1 to 4 substituents, which may be the same or different, selected from the group consisting of a halogen atom, an alkyl, haloalkyl, phenyl, alkoxy, haloalkoxy, cycloalkoxy, phenoxy, alkenyl, haloalkenyl, alkynyl, haloalkynyl, alkoxyalkyl, alkenyloxy, haloalkenyloxy, alkynyloxy, haloalkynyloxy, alkylthio, haloalkylthio, alkylsulfoxyl, acyl, alkoxyalkoxy, alkenylthio, alkoxycarbonyl, haloalkoxycarbonyl, alkynyloxycarbonyl, alkenyloxycarbonyl, nitro, and haloalkenylthio.

27. The method of claim 26, wherein R² has the formula: 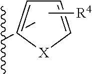
wherein X is a member selected from the group consisting of O, S and NR, wherein R is hydrogen or optionally substituted alkyl; and
R⁴ is a member selected from the group consisting an alkyl, haloalkyl, alkoxy, haloalkoxy, cycloalkoxy, phenoxy, alkenyl, haloalkenyl, alkynyl, haloalkynyl, alkoxyalkyl, alkenyloxy, haloalkenyloxy, alkynyloxy, haloalkynyloxy, phenyl, phenyoxy, thiophenyl, benzyl and furyl.

28. The method of claim 26, wherein the compound is a member selected from the group consisting 3-phenoxybenzyl 3-methyl-2-(1H-pyrrol-1-yl)butanoate, cyano(3-phenoxyphenyl)methyl 3-methyl-2-(1H-pyrrol-1-yl)butanoate.

29. The method of claim 18, wherein R² is an optionally substituted benzylpyrrolyl.

30. The method of claim 29, wherein the compound is (3-benzyl-1H-pyrrol-1-yl)methyl 2-((2-chloro-4-(trifluoromethyl)phenyl)amino)-3-methylbutanoate.

31. The product made according to claim 1.

32. A method for catalyzing a carbene insertion into a C—H bond to produce a product with a new C—C bond, the method comprising:
providing a C—H containing substrate, a diazo carbene precursor and an engineered heme enzyme; and
allowing the reaction to proceed for a time sufficient to form a product having a new C—C bond.

33-74. (canceled)

75. A method for catalyzing a nitrene insertion into a C—H bond to produce a product having a new C—N bond, the method comprising:
providing a C—H containing substrate, a nitrene precursor and an engineered heme enzyme; and
allowing the reaction to proceed for a time sufficient to form a product having a new C—N bond.

76-97. (canceled)

98. A method for catalyzing a carbene insertion into a O—H bond to produce a product having a new C—O bond, the method comprising:
providing a O—H containing substrate, a diazo carbene precursor and an engineered heme enzyme; and
allowing the reaction to proceed for a time sufficient to form a product having a new C—O bond.

99-112. (canceled)

113. A method for catalyzing a carbene insertion into a Si—H bond to produce a product having a new C—Si bond, the method comprising:
providing a Si—H containing substrate, a diazo carbene precursor and an engineered heme enzyme; and
allowing the reaction to proceed for a time sufficient to form a product having a new C—Si bond.

114-147. (canceled)
